SEQUENCE LISTING (2 sequences)

SEQ ID NO: 1 - DNA, 20 bp, Artificial Sequence
  36B4 forward primer
gcaatgttgccagtgtctgt

SEQ ID NO: 2 - DNA, 20 bp, Artificial Sequence
  36B4 reverese primer
gccttgaccttttcagcaag

(19) United States
(12) Patent Application Publication
(10) Pub. No.: US 2014/0094462 A1
(43) Pub. Date: Apr. 3, 2014

(54) SONIC HEDGEHOG MODULATORS

(71) Applicants: Sara Buhrlage, Cambridge, MA (US); Chris Dockendorff, Arlington, MA (US); Mike Foley, Cambridge, MA (US); Han-Je Kim, Gongju City (KP); Andrew Germain, Somerville, MA (US); Lawrence MacPherson, Marlborough, MA (US); Partha Nag, Somerville, MA (US); Stuart Schreiber, Boston, MA (US); Amal Ting, Newton, MA (US); Michel Weiwer, Cambridge, MA (US); Willmen Youngsaye, Cumberland, RI (US)

(72) Inventors: Sara Buhrlage, Cambridge, MA (US); Chris Dockendorff, Arlington, MA (US); Mike Foley, Cambridge, MA (US); Han-Je Kim, Gongju City (KP); Andrew Germain, Somerville, MA (US); Lawrence MacPherson, Marlborough, MA (US); Partha Nag, Somerville, MA (US); Stuart Schreiber, Boston, MA (US); Amal Ting, Newton, MA (US); Michel Weiwer, Cambridge, MA (US); Willmen Youngsaye, Cumberland, RI (US)

(21) Appl. No.: 13/781,000

(22) Filed: Feb. 28, 2013

Related U.S. Application Data
(63) Continuation of application No. 13/553,612, filed on Jul. 19, 2012, now abandoned.
Application No. 13/553,612 is a continuation of application No. 13/309,690, filed on Dec. 2, 2011, now abandoned.
Application No. 13/309,690 is a continuation of application No. 13/008,214, filed on Jan. 18, 2011, now abandoned.
Application No. 13/008,214 is a continuation of application No. 12/764,855, filed on Apr. 21, 2010, now abandoned.
(60) Provisional application No. 61/171,245, filed on Apr. 21, 2009.

(51) Int. Cl. C07D 498/04; C07D 413/04; C07D 273/01

(52) U.S. Cl. CPC C07D 498/04; C07D 273/01; C07D 413/04
USPC 514/236.8; 540/454; 514/450; 540/455; 514/413; 514/359; 514/444; 514/340; 514/406; 514/252.2; 514/326; 435/375

(57) ABSTRACT

Sonic Hedgehog modulators and methods of use thereof are provided for.

### RELATED APPLICATIONS

This application is a continuation application of U.S. patent application Ser. No. 13/553,612, filed Jul. 19, 2012, which is a continuation application of U.S. patent application Ser. No. 13/309,690, filed Dec. 2, 2011, which is a continuation of U.S. patent application Ser. No. 13/008,214, filed Jan. 18, 2011, which is a continuation of U.S. patent application Ser. No. 12/764,855, filed Apr. 21, 2010; which claims priority to U.S. Provisional Patent Application No. 61/171,245, filed Apr. 21, 2009. The contents the foregoing applications are hereby incorporated by reference in their entirety.

### FIELD OF THE APPLICATION

The present invention relates to macrocyclic small molecule modulators of the Sonic Hedgehog signaling pathway, syntheses thereof, and intermediates thereto. The invention also provides pharmaceutical compositions comprising compounds of the present invention and methods of using said compounds in the treatment of proliferative diseases (e.g., benign neoplasm, cancers, inflammatory diseases, autoimmune diseases, diabetic retinopathy) and developmental disorders.

### BACKGROUND OF THE INVENTION

The Hedgehog (Hh) pathway has been implicated in many developmental processes, including organogenesis in most animals (Ingham, P. W., McMahon, A. P. Genes and Dev. 15, 3059-3087, 2001; Frank-Kamenetsky, M., et al. J. Biol. 10, 1-19, 2002). First identified in Drosophila in 1980, three mammalian homologues of Hh proteins, Sonic Hedgehog (Shh), Desert Hedgehog, (Dhh) and Indian Hedgehog (Ihh), are all key regulators of anterior/posterior patterning in limb development, the induction of polarity in the central nervous system, and the differentiation of numerous cell types (Nusslein-Volhard, C., Wieschaus, E. Nature 287, 795-801, 1980); Weitzman, J. B. J. Biol. 7, 1-5, 2002; Pepinsky, R. B. et al. J. Biol. Chem. 275, 10995-11001, 2000; Stecca, B., Altaba, A. R. J. Biol. 9, 1-4, 2002). Shh is the most widely characterized of the Hh homologues and is essential for proper embryonic development. The Shh pathway involves the auto-cleavage of full length Shh into an active 20 kD N-terminal fragment (ShhN), which binds to its 7-pass transmembrane receptor, Patched (Ptc 1), reversing its inhibitory effect on Smoothened (Smo) (Goetz, J. A., Singh, S., Suber, L. M., Robbins, D. J. J. Biol. Chem. 281, 4087-4093, 2006). One effect of this de-repression is the activation of Gli transcription factors, which regulate the transcription of target genes that include GliI and Ptc1.

There have been several reports of both synthetic and natural small-molecule modulators of the Shh signaling pathway, discovered through cell-based phenotypic screens (Rubin, L., de Sauvage, F. J. Nature 5, 1026-1033, 2006; Chen, J. K., Tapaile, J., Young, K. E., Maiti, T., Beachy, P. A. PNAS 99, 14071-14076, 2002; Mahindroo, N., Punchihewa, C., Fujii, A. J. Med. Chem., 2009, 52, 3829-3845). Reported Shh signaling antagonists include teratogenic natural products such as cyclopamine, jervine, AY9944 and tripanol, as well as synthetic molecules such as SANTI and Cur-6141 (Cooper, M. K., Porter, J. A., Young, K. E., Beachy, P. A. Science 280, 1603-1607, 1998; Tapaile, J., et al. Nature 406, 1005-1009, 2000). There have also been reports of synthetic small-molecule agonists of the Shh pathway, including purmorphamine, Hh-Ag1.2 (Ding, S., Schultz, P. G. Nat. Biotechnol. 7, 833-840, 2004; Wu, X. et al. Chem. Biol. 11, 1229-1238, 2004), cyclopamine (Beachy et al., Nature 2004, 432, 324-331), IPI-926 (Kinzler et al., Science, 1987, 236, 70-73), CUR-61414 (Xie et al., Nature, 1998, 391, 90-92), HhAntag (International Application Publication No. WO 01/027135), GDC-0449 (Tremblay et al., J. Med. Chem., 2008, 51, 6646-6649), Gant61 (von Hoff et al., AACR Meet. Abstr., 2008, LB-138) and Physalin F (International Application Publication No. WO 06/050351). The discovery of chemical modulators of Shh signaling provides a potential means to regulate the activity of a pathway that can result in medulloblastoma, basal cell carcinomas, pancreatic cancer, and developmental disorders (Wang, B., Fallon, J. F., Beachy, P. A. Cell 100, 423-434, 2000; Borycki, A. G., Mendham, L., Emerson, C. P., Jr. Development 125, 777-790, 1998; Chiang, C. et al. Nature 383, 407-413, 1996; Kayed, H. et al. Pancreas 32, 119-129, 2006; Furukawa, T., Sunumura, M., Hori, A. Cancer Sci., 97, 17, 2006; Thayer, S. P. et al. Nature 425, 851-856, 2003). However, the aforementioned chemical modulators of the Shh signaling pathway have targeted those receptors, such as Smo and Ptc1, that are down stream of Shh in this signaling pathway. Accordingly, chemical modulators that directly target Shh may be useful in the treatment of diseases and disorders such as proliferative diseases and developmental disorders.

### SUMMARY OF THE INVENTION

The present invention encompasses the recognition that small molecule modulators of the Shh signaling pathway are useful in the treatment of proliferative diseases and developmental disorders. Non-natural macrocycles are provided that inhibit Sonic Hedgehog induced-protein transcription.

Accordingly, the present invention provides compounds of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2), and pharmaceutically acceptable salts thereof.

In another aspect, the invention provides pharmaceutical compositions comprising a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, Hit*, HIM, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2 and a pharmaceutically acceptable carrier. In some embodiments, the pharmaceutical composition comprises an effective amount of the compound to treat a proliferative disease or a developmental disorder.

In other aspects, the invention provides a method for inhibiting Sonic Hedgehog protein-induced transcription in cells by contacting the cells with an effective amount of a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2. In some embodiments, a compound of the invention inhibits the Shh pathway upstream of Ptc1. In some embodiments, a compound of the invention destabilizes a dimeric Shh complex.

In another embodiment, the present invention provides a method of treating a Sonic Hedgehog associated disorder in a subject by administering to the subject an effective amount of a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, HIM, IV, IVa, IVa-1, IVb, IVb-1, V, Va, VI, VIa, VII, X, Xa, Xb, Xc, Xd, XIa, XIb, XII, XIIa, XIII), XIII or XIV or of Table 1 or Table 2.

In yet another aspect, the present invention provides a method of treating a proliferative disease in a subject by administering to the subject an effective amount of a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2. In some embodiments, the proliferative disease is cancer. In some embodiments, the cancer is a neoplasm. In some embodiments, the cancer is basal cell carcinoma, Gorlin syndrome, medulloblastoma or pancreatic cancer.

In one aspect, the present invention provides a method of treating a developmental disorder in a subject by administering to the subject an effective amount of a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2. In some embodiments, the developmental disorder is phocomelia or cyclopia.

In yet another aspect, the present invention provides a method of controlling stem cell differentiation by contacting one or more stem cells with an effective amount of a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2.

### BRIEF DESCRIPTION OF THE DRAWINGS

FIG. 1 is a ¹H NMR spectrum of compound AK in CDCl₃.

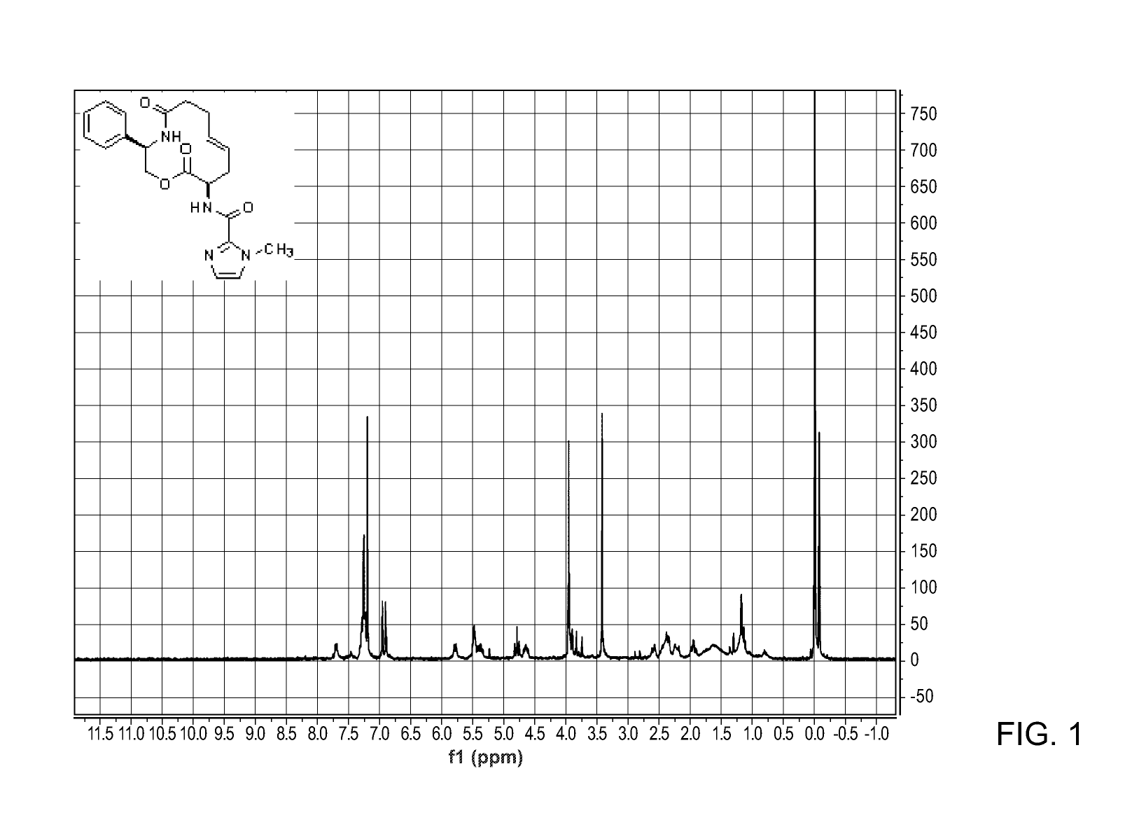

FIG. 2 is a ¹H NMR spectrum of compound AN in d₆-DMSO.

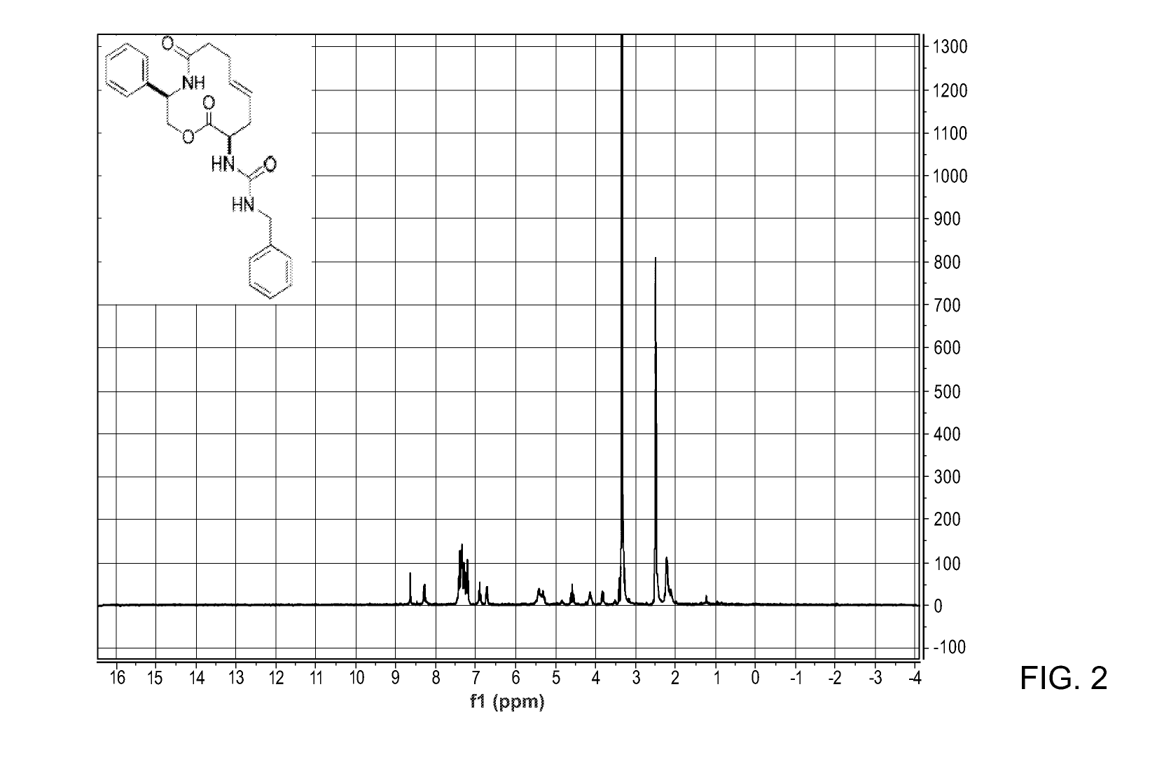

FIG. 3 is a ¹H NMR spectrum of compound B is d₆-DMSO.

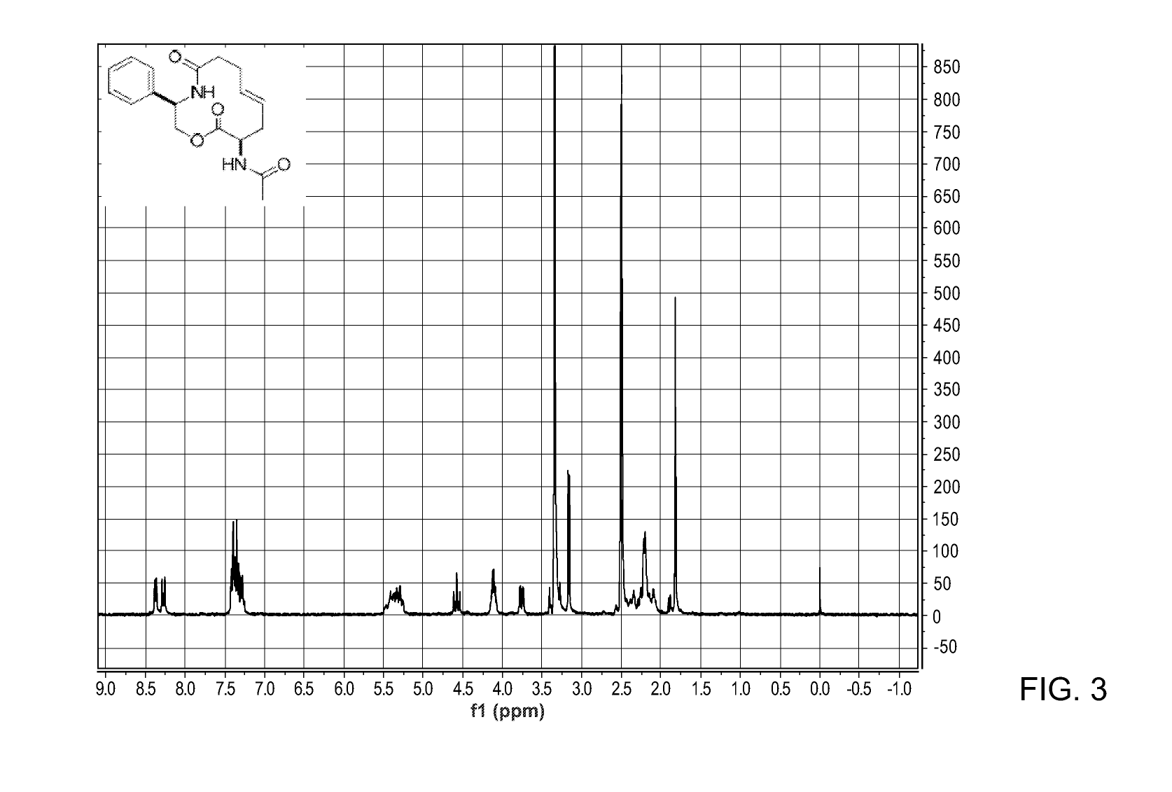

FIG. 4 is a ¹H NMR spectrum of compound AV in CD₃OD.

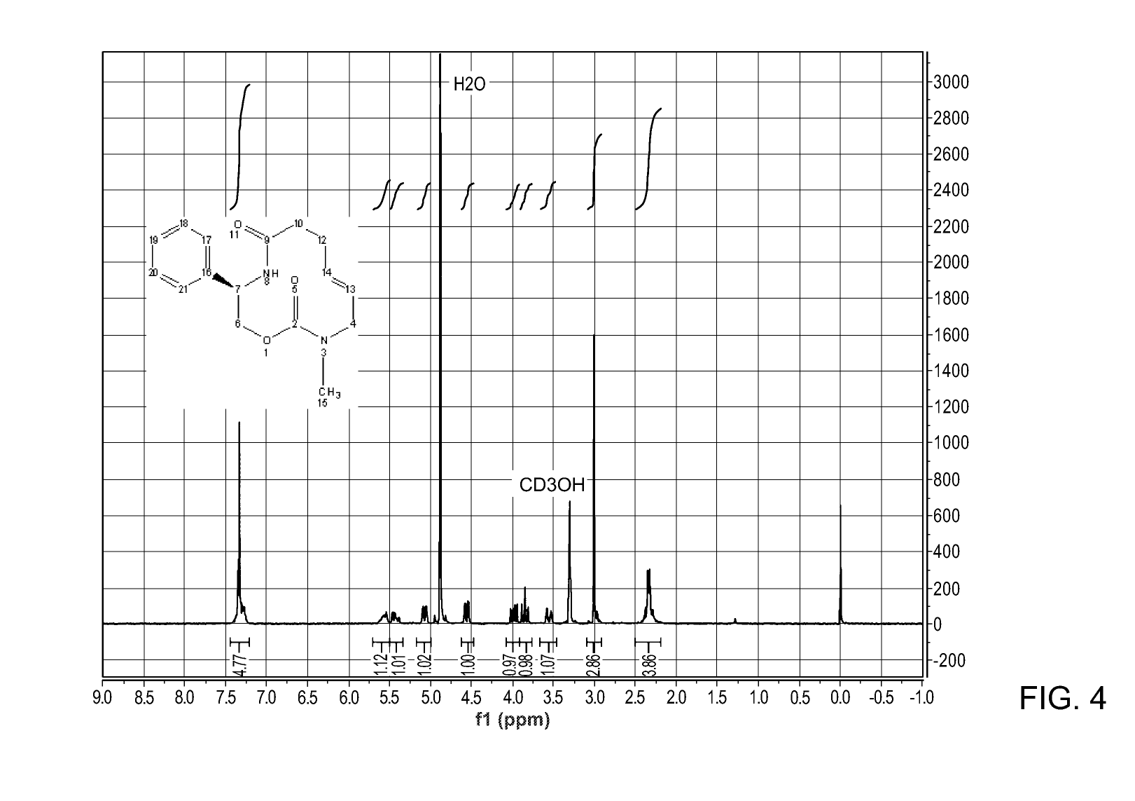

FIG. 5 is a ¹H NMR spectrum of compound BZ.

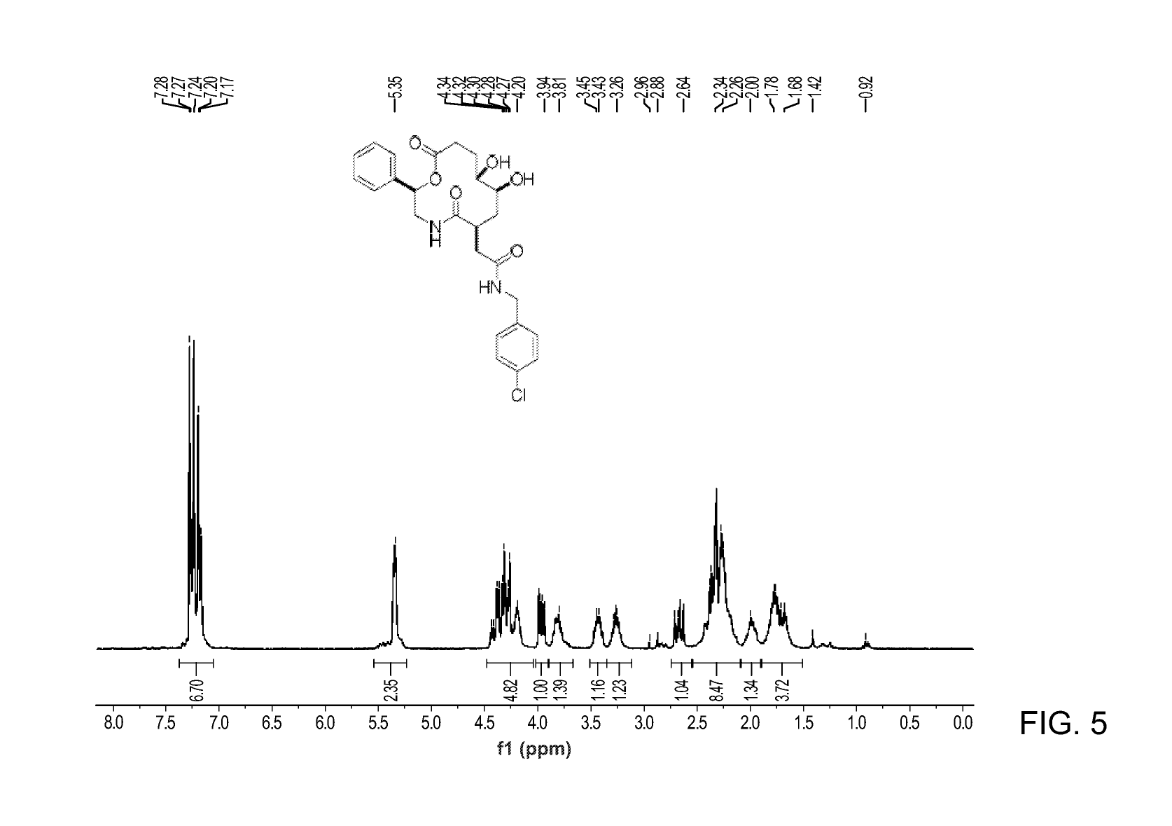

FIG. 6 is a ¹H NMR spectrum of compound FO.

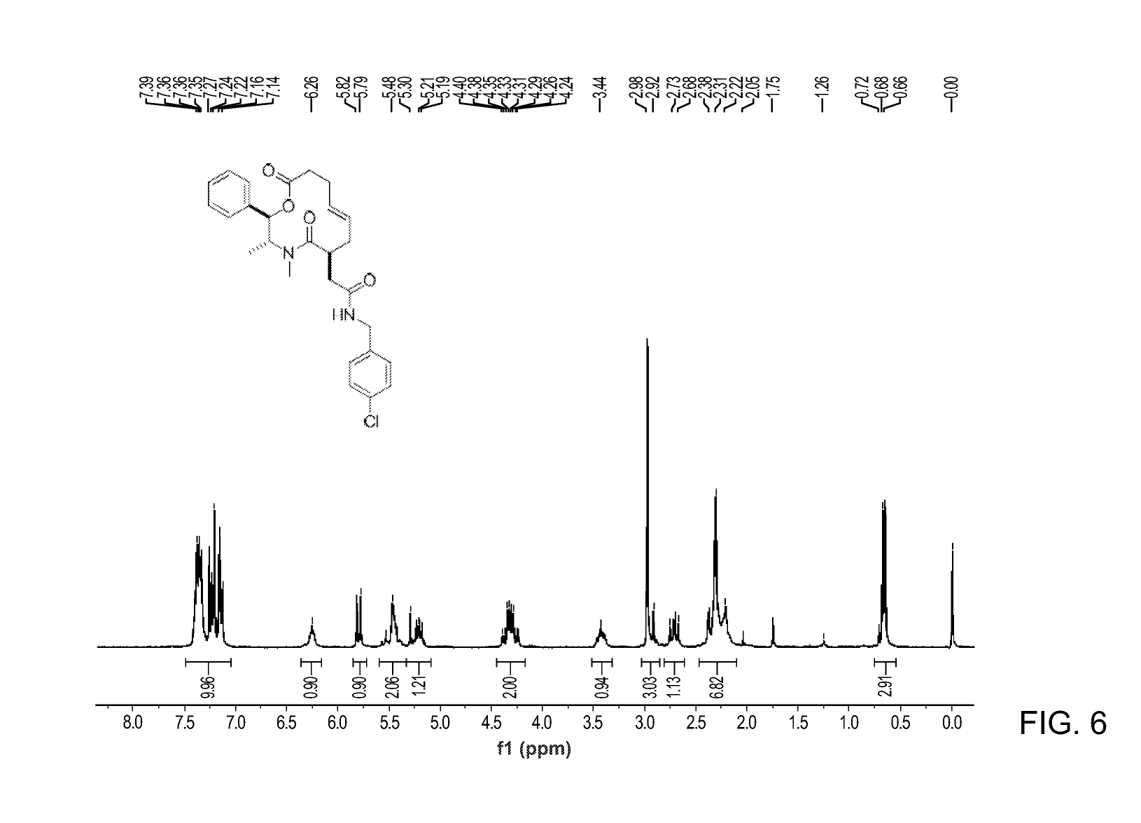

FIG. 7 is a ¹H NMR spectrum of compound C.

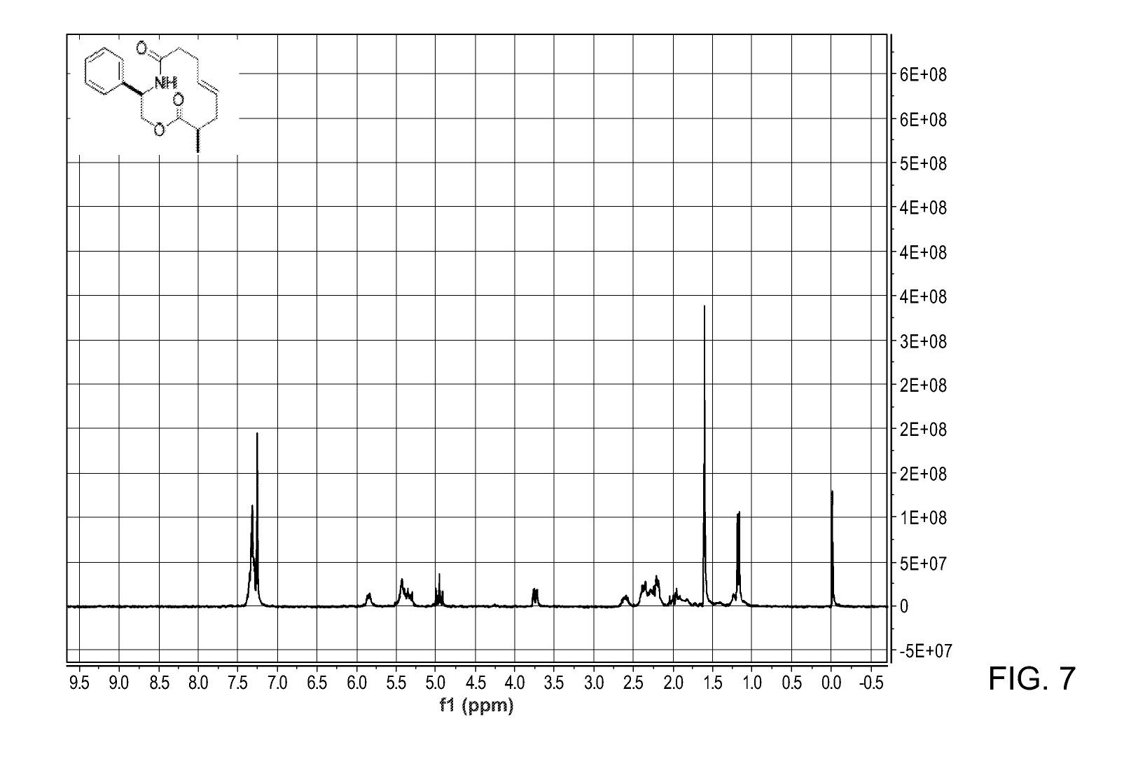

FIG. 8 is a ¹H NMR spectrum of compound BU.

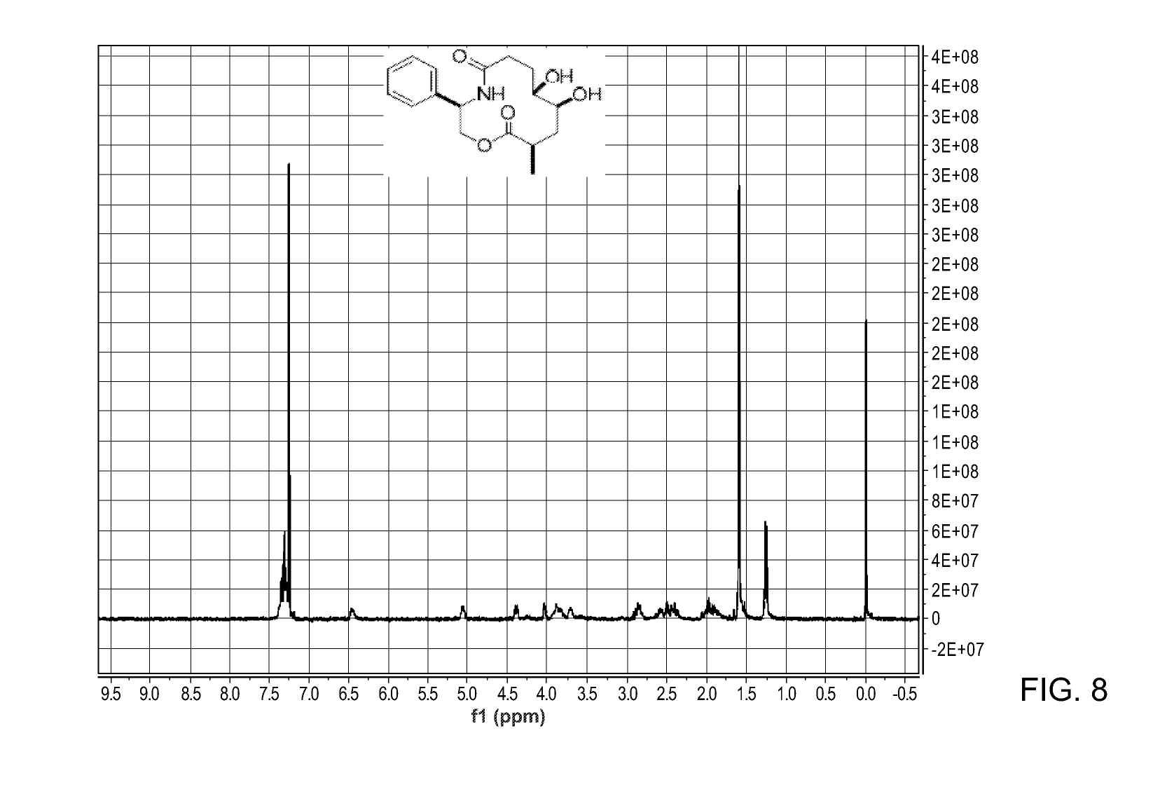

FIG. 9 is a ¹H NMR spectrum of compound BX.

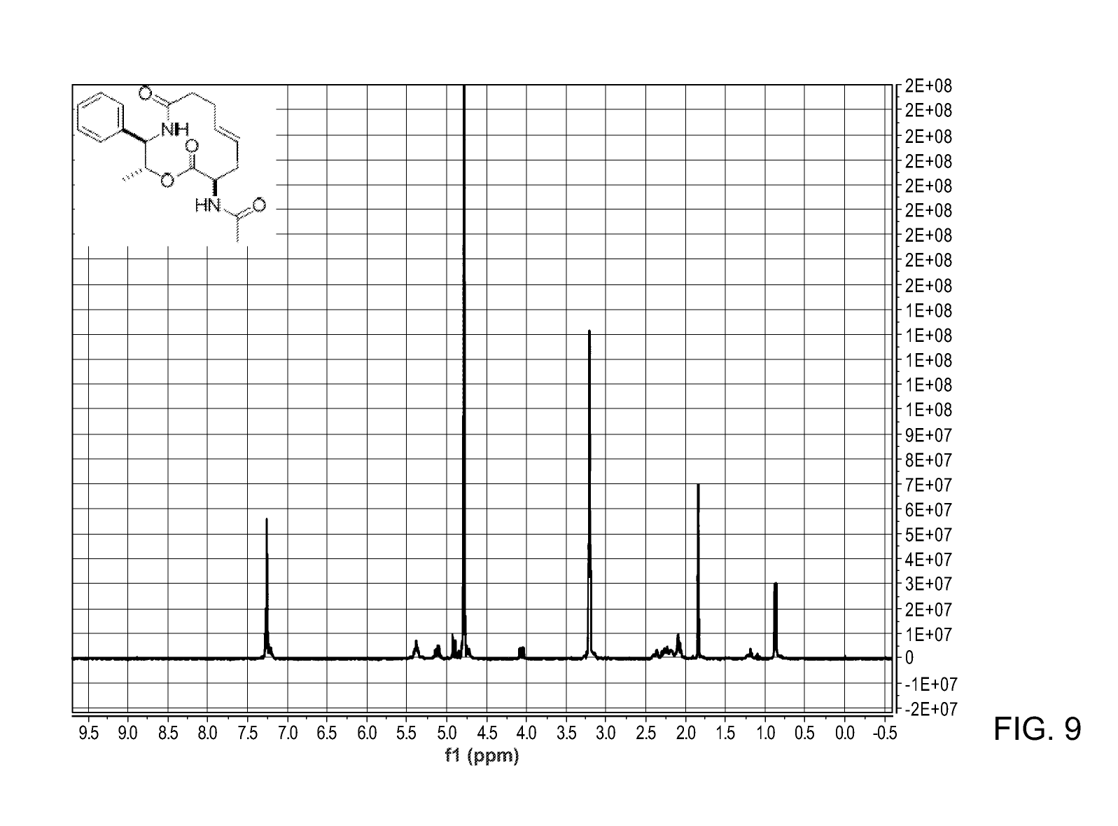

FIG. 10 is a ¹H NMR spectrum of compound CI in CD₃OD.

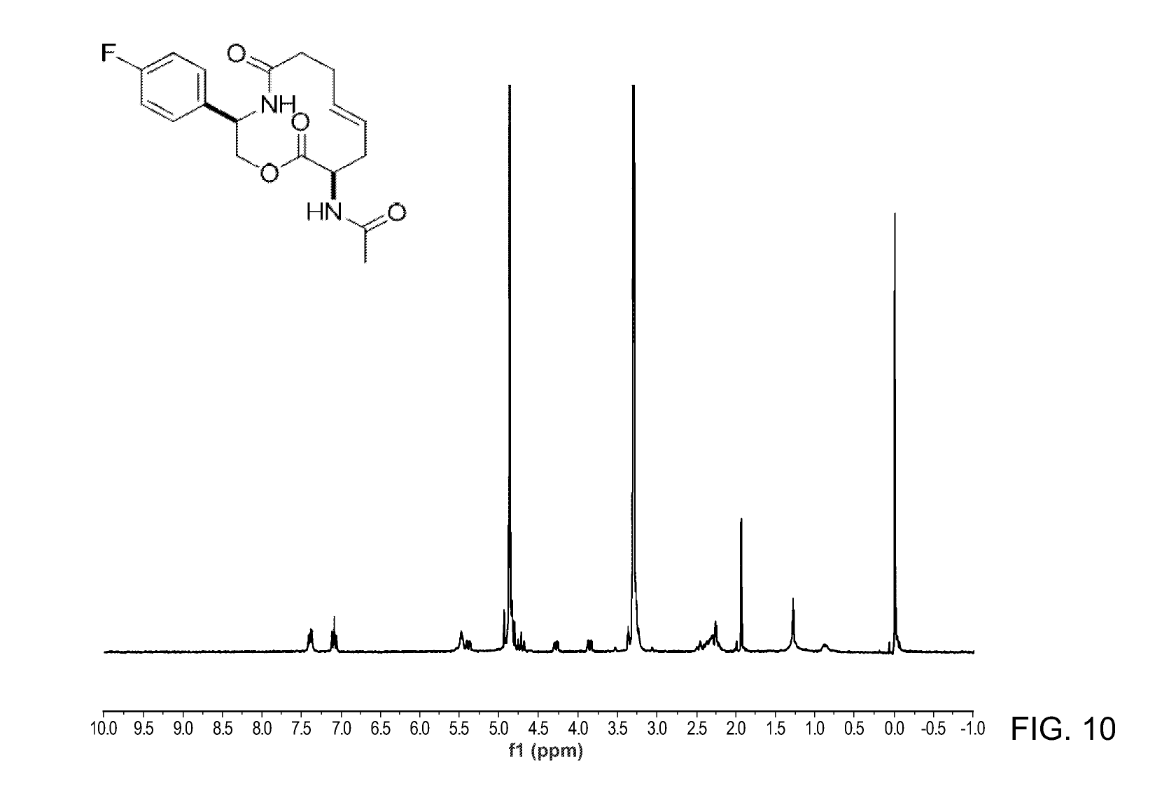

### DETAILED DESCRIPTION OF THE INVENTION

Nature has evolved many macrolactone natural products with unique activities and specificities, many of which are derived from the polyketide synthase family of enzymes (Kittendorf, J. D.; Beck, B. J.; Buchholz, T. J.; Seufert, W.; Sherman, D. H. Cell 2007, 14, 944954; Gokhale, R. S.; Hunziker, D.; Cane, D. E.; Khosla, C. Chem. And Biol. 1999, 6, 117-125). Pertinent examples include erythromycin (antibiotic), amphotericin B (anti-fungal), (Matsumori, N.; Sawada, Y.; Murata, M. J. Am. Chem. Soc. 2005, 127, 10667-10675), bryostatin-1 (anti-cancer) (Wender, P. A.; Horan, J. C.; Verma, V. A. Org. Lett. 2006, 8, 5299-5302) and FK506 (immunosuppressant) (Harding, M. W.; Galat, A.; Uehling, D. E.; Schreiber, S. L. Nature, 1989, 341, 758-760; Nakatsuka, M.; Ragan, J. A.; Sammakia, T.; Smith, D. B.; Uehling, D. E.; Schreiber, S. L. J. Am. Chem. Soc., 1990, 112, 5583-5601; Rosen, M. K.; Standaert, R. F.; Galat, A.; Nakatsuka, M.; Schreiber, S. L. Science, 1990, 248, 863-866). The present disclosure describes a class of non-naturally occurring macrocycles capable of binding to Sonic Hedgehog (Shh) protein and repressing target gene expression.

To our knowledge, the previously reported synthetic Shh pathway inhibitors are not known to target the Shh protein itself, and were intended to target any particular constituent of the pathway. The reported examples of discoveries of small-molecule Shh signaling modulators resulted from the use of cell-based phenotypic assays. Target-based discovery of modulators of Shh signaling was expected to provide a complementary approach. Small-molecule microarray (SMM) technology has enabled the discovery of small molecules that bind target proteins of interest (Macbeath, G., Koehler, A. N., Schreiber, S. L. J. Am. Chem. Soc. 121, 7967-7968, 1999; Barnes-Seeman, D., Park, S. B., Koehler, A. N., Schreiber, S. L. Angew. Chem. Int. Ed. 42, 2376-2379, 2003; Koehler, A. N., Shamji, A. F., Schreiber, S. L. J. Am. Chem. Soc. 125, 8420-8421, 2003). It has been reported that molecules from multiple diversity-oriented synthesis (DOS) pathways, which had been covalently linked to a glass surface, could be screened for binding to a given protein in a tandem high-throughput fashion using SMMs (Burke, M. D., Berger, E. M., Schreiber, S. L. Science 302, 613-618, 2003; Burke, M. D., Berger, E. M., Schreiber, S. L. J. Am. Chem. Soc. 126, 14095-14104, 2004; Chen, C. et al. Angew. Chem. Int. Ed. 44, 2249-2252, 2005; Kumar, N., Kiuchi, M., Tallarico, J. A., Schreiber, S. L. Org. Lett. 7, 2535-2538, 2005; Lo, M. et al. J. Am. Chem. Soc. 126, 16077-16086, 2004; Stavenger, R. A., Schreiber, S. L. Angew. Chem. Int. Ed. 40, 3417-3421, 2001; Wong, J. C. et al. Chem. Biol. 11, 1279-1291, 2004).

Accordingly, in one embodiment, the invention provides for methods of treating a Sonic Hedgehog associated disorder in subject by administering to the subject an effective amount of a compound of the invention (e.g., a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2).

The language “Sonic Hedgehog associated disorder” includes those disorders that arise upon the upregulation or downregulation of the Sonic Hedgehog signaling pathway. In one embodiment, the Sonic Hedgehog associated disorder is a proliferative disease or a developmental disorder.

The language “proliferative disease” includes disorders in which cells increase in number rapidly. Examples of proliferative disorders include a benign neoplasm and cancer.

The language “benign neoplasm” includes tumors that lack the malignant properties of cancer. Examples of benign neoplasms include uterine fibroids and moles.

In some embodiments, the proliferative disorder is cancer. In one embodiment, the cancer is a hematological malignancy. In another embodiment, the cancer is a solid tumor. Exemplary cancers that may be treated using compounds of the invention (e.g., a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2) include colon cancer, lung cancer, bone cancer, pancreatic cancer, stomach cancer, esophageal cancer, skin cancer, brain cancer, liver cancer, ovarian cancer, cervical cancer, uterine cancer, testicular cancer, prostate cancer, bladder cancer, kidney cancer, neuroendocrine cancer, breast cancer, gastric cancer, eye cancer, gallbladder cancer, laryngeal cancer, oral cancer, penile cancer, glandular tumors, rectal cancer, small intestine cancer, sarcoma, carcinoma, melanoma, urethral cancer, vaginal cancer, to name but a few. In one embodiment, the cancer is pancreatic cancer. In another embodiment, the cancer is medulloblastoma. In other embodiments, the cancer is Gorlin syndrome. In yet another embodiment, the cancer is basal cell carcinoma.

The language “developmental disorder” includes disorders that occur during a child's development. Examples of development disorders include phocomelia or cyclopia.

The term “subject” includes animals (e.g., vertebrates, amphibians, fish, mammals, e.g., cats, dogs, horses, pigs, cows, sheep, rodents, rabbits, squirrels, bears, primates (e.g., chimpanzees, gorillas, and humans) which are capable of suffering from a Sonic Hedgehog associated disorder. In some embodiments, the subject is suffering from or at risk of suffering from a Sonic Hedgehog associated disorder (e.g., a proliferative disease or developmental disorder). In one embodiment, the subject is a mammal. In another embodiment, the subject is a human.

As used herein, “treating” or “treatment” includes any effect e.g., lessening, reducing, modulating, or eliminating, that results in the improvement of the condition, disease, disorder, etc. “Treating” or “treatment” includes: (a) inhibiting a Sonic Hedgehog associated disorder, for example, arresting its development or its clinical symptoms; and/or (b) relieving the Sonic Hedgehog associated disorder, for example, causing regression or elimination of the Sonic Hedgehog associated disorder. The language “treat” or “treating” includes the administration to a subject a compound of the invention (e.g., a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2) in an amount effective to treat the subject for a Sonic Hedgehog associated disorder.

In one embodiment, the invention provides methods of treating cancer (e.g., pancreatic cancer) in a subject comprising administering to the subject an effective amount of a compound of the invention (e.g., a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2).

In one embodiment, the invention provides methods of treating a developmental disorder (e.g., phocomelia or cyclopia) in a subject comprising administering to the subject an effective amount of a compound of the invention (e.g., a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2). In some embodiments, the developmental disorder is treated at a pre-natal stage. In some embodiments, the developmental disorder is treated in an intrauterine fashion. In some embodiments, the developmental disorder is treated at a post-natal stage. In some embodiments, the subject is human. In some embodiments, the subject is a human embryo.

In other aspects, the invention provides a method for inhibiting Sonic Hedgehog protein-induced transcription in cells by contacting the cells with an effective amount of a compound of the invention (e.g., a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2).

As used herein, the language “inhibiting transcription” includes suppressing or completely eliminating the process of creating an equivalent RNA copy of a DNA sequence. In some embodiments, a compound of the invention inhibits transcription in the Shh pathway upstream of Ptc1. In some embodiments, a compound of the invention inhibits transcription by destabilizing a dimeric Shh complex. In one embodiment, the inhibition includes repressed Gli1 activity.

In yet another aspect, the present invention provides a method of controlling stem cell differentiation by contacting one or more stem cells with an effective amount of a compound of the invention (e.g., a compound of formulae I, II, IIa, IIb, IIb-1, IIc, IIc-1, IId-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2). In one embodiment, the differentiation is terminal. In one embodiment, the differentiation is from a stem cell to an endoderm. In one embodiment, the invention relates to a method, wherein the differentiation is from an endoderm to a pancreatic precursor. In some embodiments, the resulting differentiation is terminal. In some embodiments, the resulting differentiation is non-terminal. In some embodiments, differentiation is from a stem cell to an intermediate cell stage (such as a progenitor cell). In some embodiments, differentiation is from a progenitor cell to a more specialized cell. In some embodiments, differentiation is from a definitive endoderm cell to a pancreatic precursor cell. In some embodiments, the present invention provides a method of controlling cell dedifferentiation by contacting one or more cells with an effective amount of a compound of the invention (e.g., a compound of formulae I, II, IIa, IIb, IIb-1, IIc, IIc-1, lid-1, IId-2, IId-3, IId-4, III, IIIb, IIIb-1, IIIc, IIIc-1, IIId, IIId-1, IIIe, IIIe-1, IIIe-2, IIIe-3, IIIf, IIIf-1, IV, IVa, IVa-1, IVb, IVb-1, V, Va, VI, VIa, VII, X, Xa, Xb, Xc, Xd, XIa, XIb, XII, XIIa, XIIb, XIII or XIV or of Table 1 or Table 2), thereby resulting in a stem cell.

In some embodiments, compounds of the invention are used to control cell differentiation. In some embodiments, the differentiation is related to cellular development. In some embodiments, the differentiation is related to organ development. In some embodiments, the differentiation is related to organ system development. In some embodiments, the differentiation is related to organism development.

In some embodiments, the compounds of the invention include compounds formulae I, II, IIa, IIb, IIb-1, IIc, IIc-1, IId-1, IId-2, IId-3, IId-4, III, IIIb, IIIb-1, IIIc, Inc-1, IIId, IIId-1, IIIe, IIIe-1, IIIe-2, IIIe-3, IIIf, IIIf-1, IV, IVa, IVa-1, IVb, IVb-1, V, Va, VI, VIa or VII:

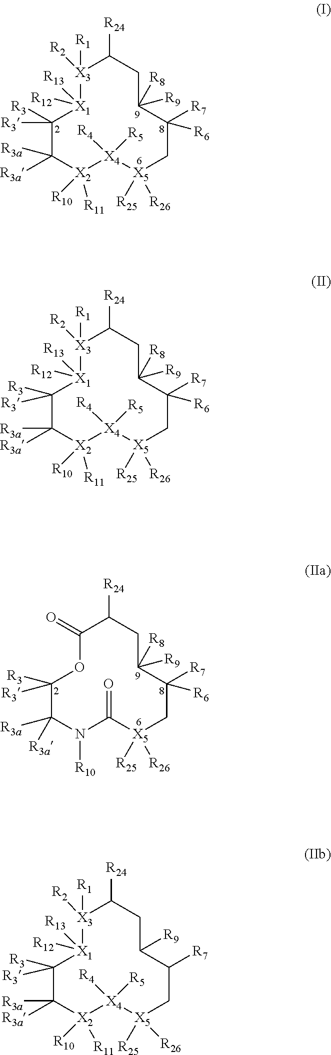

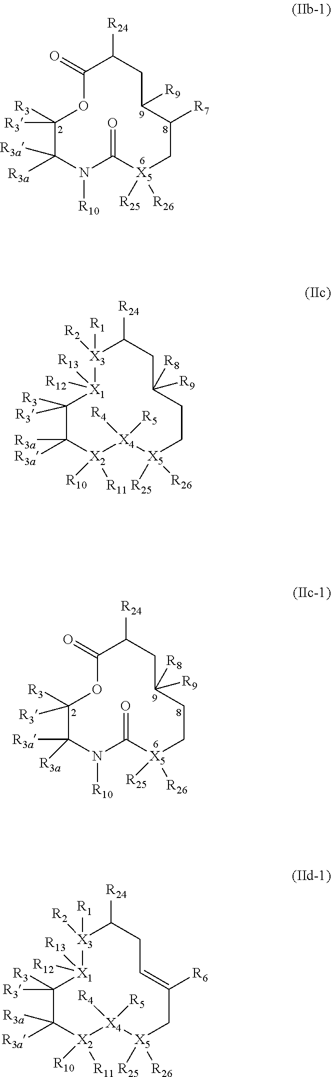

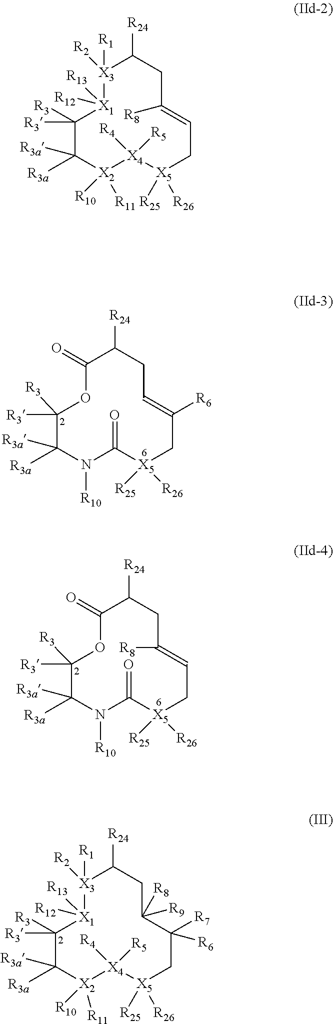

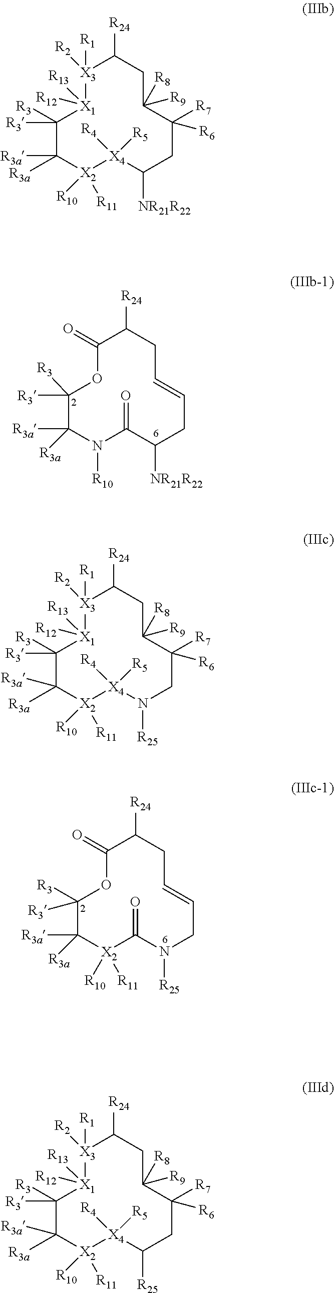

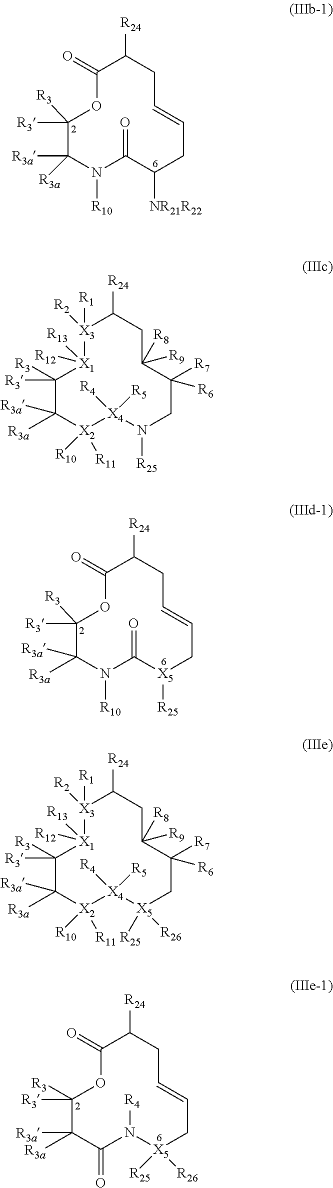

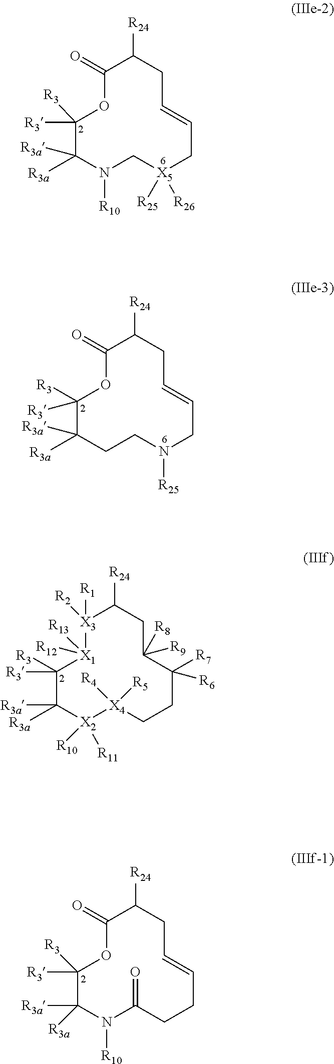

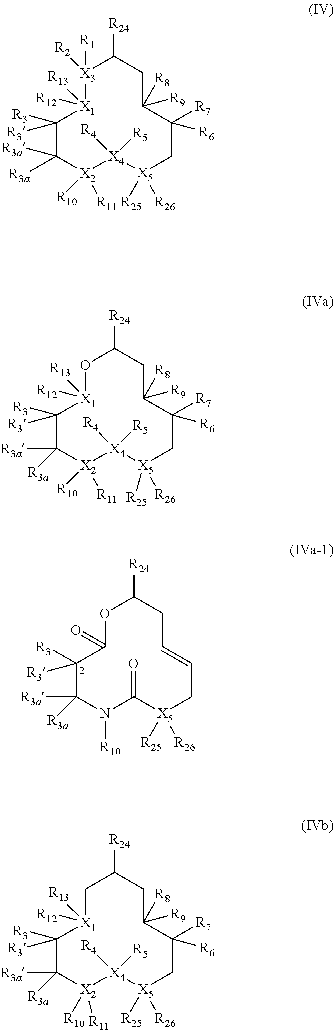

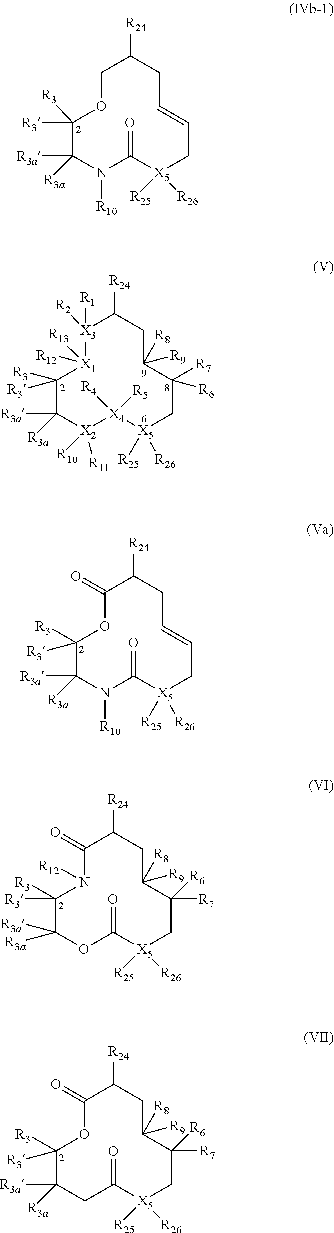

or a pharmaceutically acceptable salt thereof,

Formula I:

In one embodiment, the invention provides for compounds of formula I, wherein R₁ and R₂ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₃ is C, together R₁ and R₂ form ═O;

R₃ and R₃′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ and R₃′ are taken together with their intervening atoms to form a 3-8-membered ring;

when X₁ is N, one of R₃ or R₃′ and one of R₁₂ or R₁₃ taken together with their intervening atoms to form a 3-8 membered ring; or

R₃ₐ and R₃ₐ′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ₐ and R₃ₐ′ taken together with their intervening atoms form a 3-8-membered ring; or

when X₂ is N, one of R₃ₐ or R₃ₐ′ and one of R₁₀ or R₁₁ taken together with their intervening atoms to form a 3-8 membered ring; or

one of R₃ or R₃′ and one of R₃ₐ or R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₄ and R₅ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₄ is C, together R₄ and R₅ form ═O;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₀ and R₁₁ are each independently hydrogen, halogen, —CN, —SCN, —NO₂, —COR₁₄, —CO₂R₁₄, —SOR₂₃, —SO₂R₂₃, —NR₂₁R₂₂, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₂ is C, together R₁₀ and R₁₁ form ═O;

R₁₂ and R₁₃ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₁ is C, together R₁₂ and R₁₃ form ═O;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₁ is O, N, or C;

X₂ is O, N, or C;

X₃ is O or C;

X₄ is O, N, or C;

X₅ is N or C;

P is —CH₂(C═O)ᵤNR₂₁R₂₂;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —NR₁₅COR₁₆, —CH₂(C═O)—NR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

In one embodiment, the invention relates to a compound, or a salt thereof having the formula I provided that the compound is not

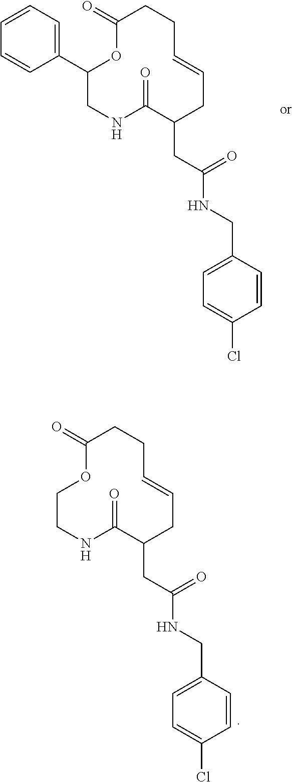 [SMILES: O=C(CC1C/C=C/CCC(=O)OC(c2ccccc2)CNC1=O)NCc1ccc(Cl)cc1 ; O=C(CC1C/C=C/CCC(=O)OCCNC1=O)NCc1ccc(Cl)cc1]

In one embodiment, the invention relates to a compound or a salt thereof having the formula I, provided that when one of R₃ or R₃′ and one of R₃ₐ and R₃ₐ′ taken together with their intervening atoms form a 6-membered heterocyclic ring containing one oxygen atom adjacent to the carbon atom at the 2-position of the 12-membered ring, then at least one of R₂₅, R₂₆, R₁₀ or R₁₁ is not hydrogen.

In one embodiment, the invention relates to a compound or a salt thereof having the formula I, provided that when R₃ₐ or R₃ₐ′ is —C(O)OCH₃ and R₃ or R_{3′} is H, then at least one of R₂₅, R₂₆, R₁₀, or R₁₁ is not hydrogen.

In one embodiment, the invention relates to a compound or a salt thereof having the formula I, provided that when R₃ or R₃′ is unsubstituted phenyl and the stereochemistry of the carbon atom adjacent to said phenyl is in the R-configuration, X₂ is N and one of R₁₀ or R₁₁ is CH₃, then R₂₄, one of R₃ₐ or R₃ₐ′, and one of R₂₅ or R₂₆ are not each methyl.

In one embodiment, the invention relates to a compound or a salt thereof having the formula I, provided that when R₃ₐ or R₃ₐ′ is —C(O)OCH₃, R₃ and R₃′ are H and X₂ is N, then one of R₂₅ and R₂₆ is not NHC(O)OC(CH₃)₃ and the other is H.

In one embodiment, the invention relates to a compound or a salt thereof having the formula I, provided that when one of R₃ or R₃′ and one of R₃ₐ or R₃ₐ′ taken together with their intervening atoms form a 6-membered cyclohexyl ring and X₂ is N, then one of R₁₀ or R₁₁ is not —CH₂(C₆H₄)(4-hydroxymethyl) when X₅ and R₂₅ and R₂₆ taken together are —CH₂—.

In one embodiment, the invention relates to a compound or a salt thereof having the formula I, provided that when X₂ is N, one of R₁₀ or R₁₁ is hydrogen and the other is absent, then R and R₃′ are not both methyl.

Formula II:

In one embodiment, the invention provides for compounds of formula II, wherein R₁ and R₂ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₃ is C, together R₁ and R₂ form ═O;

R₃ and R₃′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ and R₃′ are taken together with their intervening atoms to form a 3-8-membered ring; or

when X₁ is N, one of R₃ or R₃′ and one of R₁₂ or R₁₃ taken together with their intervening atoms to form a 3-8 membered ring;

R₃ₐ and R₃ₐ′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ₐ and R₃ₐ′ taken together with their intervening atoms form a 3-8-membered ring; or

when X₂ is N, one of R₃ₐ or R₃ₐ′ and one of R_{E}) or R₁₁ taken together with their intervening atoms to form a 3-8 membered ring; or

one of R₃ or R₃′ and one of R₃ₐ and R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₄ and R₅ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₄ is C, together R₄ and R₅ form ═O;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₀ and R₁₁ are each independently hydrogen, halogen, —CN, —SCN, —NO₂, —COR₁₄, —CO₂R₁₄, —SOR₂₃, —SO₂R₂₃, —NR₂₁R₂₂, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, P, or an optionally substituted group selected from C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₂ is C, together R₁₀ and R₁₁ form ═O;

R₁₂ and R₁₃ are each independently hydrogen, halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₁ is C, together R₁₂ and R₁₃ form ═O;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₁ is O, N, or C;

X₂ is O, N, or C;

X₃ is O or C;

X₄ is O, N, or C;

X₅ is N or C;

P is —CH₂(C═O)ᵤNR₂₁R₂₂;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —NR₁₅COR₁₆, —CH₂(C═O)—NR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5. In one embodiment, the invention relates to a compound or a salt thereof having the formula II and provided that (1) at least two of R₆, R₇, R₈, and R₉ form a ring, or (2) R₈ and R₉ form ═O or ═N—OR, or (3) at least one of R₆, R₇, R₈, and R₉ is not hydrogen.

Formula IIa:

In one embodiment, the invention provides compounds of formula IIa, wherein R₃, R₃′, R₃ₐ, R₃ₐ′, R₆, R₇, R₈, R₉, X₅, R₂₄, R₂₅, and R₂₆ are as described for formula II, further wherein

R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or R₁₅ and R₁₆ taken together with their intervening atoms form a 3-8-membered ring;

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5. In one embodiment, the invention relates to a compound or salt thereof having the formula IIa, provided that

(1) at least two of R₆, R₇, R₈, and R₉ form a ring, or
(2) R₈ and R₉ form ═O or ═N—OR, or
(3) at least one of R₆, R₇, R₈, and R₉ is not hydrogen.

Formula IIb:

In one embodiment, the invention provides for compounds of formula IIb, wherein R₁, R₂, R₃, R₃′, R₃ₐ, R₃ₐ′, R₄, R₅, R₇, R₉, R₁₀, R₁₁, R₁₂, R₁₃, R₂₄, R₂₅, R₂₆, X₁, X₂, X₃, and X₄ as described for formula II. In one embodiment, the invention relates to a compound or salt thereof having the formula IIb, provided that at least one of R₇ or R₉ is not hydrogen.

Formula IIb-1:

In one embodiment, the invention provides for compounds of formula IIb-1, wherein R₃, R₃′, R₃ₐ, R₃ₐ′, R₇, R₉, X₅, R₂₄, R₂₅, and R₂₆ are as described for formula II,

R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or R₁₅ and R₁₆ taken together with their intervening atoms form a 3-8-membered ring;

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

In one embodiment, the invention relates to a compound or salt thereof having the formula IIb-1, provided that at least one of R₇ or R₉ is not hydrogen.

Formula IIc:

In one embodiment, the invention provides for compounds of formula IIc, wherein R₁, R₂, R₃, R₃′, R₃ₐ, R₃ₐ′, R₄, R₅, R₈, R₉, R₁₀, R₁₁, R₁₂, R₁₃, R₂₄, R₂₅, R₂₆, X₁, X₂, X₃, X₄, and X₅ as described for formula II. In one embodiment, the invention relates to a compound or salt thereof having the formula IIc, provided that at least one of R₈ and R₉ is not hydrogen.

Formula IIc-1:

In one embodiment, the invention provides compounds of formula IIc-1, wherein R₃, R₃′, R₃ₐ, R^{3a′}, R₈, R₉, X₅, R₂₄, R₂₅, and R₂₆ are as described for formula II, further wherein

R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or R₁₅ and R₁₆ taken together with their intervening atoms form a 3-8-membered ring;

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5. In one embodiment, the invention relates to a compound or salt thereof having the formula IIc-1, provided that at least one of R₈ and R₉ is not hydrogen.

Formula IId-1:

In one embodiment, the invention provides compounds of formula IId-1, wherein R₁, R₂, R₃, R₃′, R₃ₐ, R₃ₐ′, R₄, R₅, R₁₀, R₁₁, R₁₂, R₁₃, R₂₄, R₂₅, R₂₆, X₁, X₂, X₃, X₄, and X₅ as described in for formula II and further wherein,

R₆ is halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring.

Formula IId-2:

In one embodiment, the invention provides compounds of formula IId-2, wherein R₁, R₂, R₃, R₃′, R₃ₐ, R₃ₐ′, R₄, R₅, R₁₀, R₁₁, R₁₂, R₁₃, R₂₄, R₂₅, R₂₆, X₁, X₂, X₃, X₄, and X₅ as described for formula II and further wherein,

R₈ is halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring.

Formula IId-3:

In one embodiment, the invention compounds of formula IId-3, wherein R₃, R₃′, R₃ₐ, R₃ₐ′, R₁₀, R₂₄, R₂₅, R₂₆, and X₅ are as described for formula II and further wherein, R₆ is halogen, —CN, —SCN, —NO₂, —COR₁₄, —CO₂R₁₄, —SOR₂₃, —SO₂R₂₃, —NR₁₅R₁₆, —NR₁₅COR₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₀ is hydrogen, —COR₁₄, —CO₂R₁₄, —SOR₂₃, —SO₂R₂₃, —C(O)NR₁₅R₁₆, P, or an optionally substituted group selected from the group consisting of acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula IId-4:

In one embodiment, the invention provides compounds of formula IId-4, wherein R₃, R₃′, R^{3a}, R₃ₐ′, R₂₄, R₂₅, R₂₆, and X₅ are as described for formula II and further wherein, R₈ is halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula III:

In one embodiment, the invention provides compounds of formula III, wherein R₁ and R₂ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₃ is C, together R₁ and R₂ form ═O;

R₃ and R₃′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ and R₃′ are taken together with their intervening atoms to form a 3-8-membered ring;

when X₁ is N, one of R₃ or R₃′ and one of R₁₂ or R₁₃ taken together with their intervening atoms to form a 3-8 membered ring; or

R₃ₐ and R₃ₐ′ are each independently hydrogen, halogen, —CN, —SCN, —NO₂, —-COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ₐ and R₃ₐ′ taken together with their intervening atoms form a 3-8-membered ring; or

when X₂ is N, one of R₃ₐ or R₃ₐ′ and one of R₁₀ or R₁₁ taken together with their intervening atoms to form a 3-8 membered ring; or

one of R₃ or R₃′ and one of R₃ₐ or R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₄ and R₅ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₄ is C, together R₄ and R₅ form ═O;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₀ and R₁₁ are each independently hydrogen, halogen, —CN, —SCN, —NO₂, —COR₁₄, —CO₂R₁₄, —SOR₂₃, —SO₂R₂₃, —NR₂₁R₂₂, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₂ is C, together R₁₀ and R₁₁ form ═O;

R₁₂ and R₁₃ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₁ is C, together R₁₂ and R₁₃ form ═O;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₁ is O, N, or C;

X₂ is O, N, or C;

X₃ is O or C;

X₄ is O, N, or C;

X₅ is N or C;

P is —CH₂(C═O)ᵤNR₂₁R₂₂;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —CH₂(C═O)ᵤNR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

In one embodiment, the invention relates to a compound or salt thereof having the formula III, provided that when X₅ is C, one of R₂₅ or R₂₆ is —N₂₁R₂₂. In one embodiment, the invention relates to a compound or salt thereof having the formula III, provided that when X₄ is N, one of R₄ or R₅ is H and the other is absent. In one embodiment, the invention relates to a compound or salt thereof having the formula III, provided that when X₅ is N. In one embodiment, the invention relates to a compound or salt thereof having the formula III, provided that when X₅ is C and R₂₅ and R₂₆ are both H. In one embodiment, the invention relates to a compound or salt thereof having the formula III, provided that when X₅ is C, then at least one of R₂₅ or R₂₆ is an optionally substituted group selected from C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S. In one embodiment, the invention relates to a compound or salt thereof having the formula III, provided that when X₄ is C, R₄ and R₅ are not taken together to form ═O.

In one embodiment, the invention relates to a compound or salt thereof having the formula III, provided that the compound is not

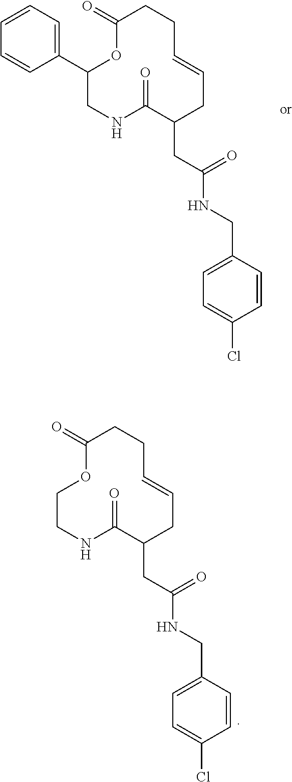 [SMILES: O=C(CC1C/C=C/CCC(=O)OC(c2ccccc2)CNC1=O)NCc1ccc(Cl)cc1 ; O=C(CC1C/C=C/CCC(=O)OCCNC1=O)NCc1ccc(Cl)cc1]

In one embodiment, the invention relates to a compound or salt thereof having the formula III, provided that when one of R₃ or R₃′ and one of R₃ₐ or R₃ₐ′ taken together with their intervening atoms form a 6-membered heterocyclic ring containing one oxygen atom adjacent to the carbon atom at the 2-position of the 12-membered ring, then at least one of R₂₅, R₂₆, R₁₀ or R₁₁ is not hydrogen. In one embodiment, the invention relates to a compound or salt thereof having the formula III, provided that when one of R₃ₐ or R₃ₐ′ is —C(O)OCH₃ and one of R₃ and R₃′ are H, then at least one of R₂₅, R₂₆, R₁₀, or R₁₁ is not hydrogen. In one embodiment, the invention relates to a compound or salt thereof having the formula III, provided that when R₃ or R₃′ is unsubstituted phenyl and the stereochemistry of the carbon atom adjacent to said phenyl is in the R-configuration, X₂ is N and one of R₁₀ or R₁₁ is CH₃, then R₂₄, R₃ₐ or R₃ₐ′, R₂₅, and R₂₆ are not each methyl. In one embodiment, the invention relates to a compound or salt thereof having the formula III, provided that when one of R₃ₐ or R₃ₐ′ is —C(O)OCH₃, R₃ and R₃′ are H and X₂ is N, then one of R₂₅ and R₂₆ is not NHC(O)OC(CH₃)₃ and the is other is H. In one embodiment, the invention relates to a compound or salt thereof having the formula III, provided that when one of R₃ or R₃′ and one of R₃ₐ or R₃ₐ′ taken together with their intervening atoms form a 6-membered cyclohexyl ring and X₂ is N, then one of R₁₀ or R₁₁ is not —CH₂(C₆H₄)(4-hydroxymethyl) when X₅ and R₂₅ and R₂₆ taken together are —CH₂—.

Formula IIIb:

In one embodiment, the invention provides compounds of formula IIIb, wherein R₁, R₂, R₃, R₃′, R₃ₐ, R₃ₐ′, R₄, R₅, R₆, R₇, R₈, R₉, R₁₀, R₁₁, R₁₂, R₁₃, R₂₄, X₁, X₂, X₃, and X₄ are as described for formula III. In one embodiment, the invention relates to a compound or salt thereof having a formula IIIb, provided that when R₃ₐ or R₃ₐ′ is —C(O)OCH₃, R₃ and R₃′ are H, and X₂ is N, then one of R₂₁ or R₂₂ is not —C(O)OC(CH₃)₃ and the other H.

Formula IIIb-1:

In one embodiment, the invention provides compounds of formula IIIb-1, wherein R₃, R₃′, R₃ₐ, R₃ₐ′, R₁₀, R₂₁, R₂₂, and R₂₄ are as described for formula III. In one embodiment, the invention relates to a compound or a salt thereof having the formula IIIb-1, provided that when R₃ₐ or R₃ₐ′ is —C(O)OCH₃, R₃ and R₃′ are H, and R₁₀ is H, then one of R₂₁ or R₂₂ is not —C(O)OC(CH₃)₃ and the other is H.

Formula IIIc:

In one embodiment, the invention provides compounds of formula IIIc, wherein R₁, R₂, R₃, R₃′, R₃ₐ, R₃ₐ′, R₄, R₅, R₆, R₇, R₈, R₉, R₁₀, R₁₁, R₁₂, R₁₃, R₂₄, X₁, X₂, X₃, and X₄ as described for formula III and further wherein,

R₂₅ is hydrogen, —COR₁₄, —CO₂R₁₄, —SOR₂₃, —SO₂R₂₃, —C(O)NR₁₅R₁₆, —CH₂(C═O)—NR₂₁R₂₂, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula IIIc-1:

In one embodiment, the invention provides compounds of formula IIIc-1, wherein R₃, R₃′, R₃ₐ, R₃ₐ′, R₁₀, R₁₁, R₂₄, and X₂ are as described for formula III and further wherein

R₂₅ is hydrogen, —COR₁₄, —CO₂R₁₄, —SOR₂₃, —SO₂R₂₃, —C(O)NR₁₅R₁₆, —CH₂(C═O)—NR₂₁R₂₂, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring; and

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula IIId:

In one embodiment, the invention provides compounds of formula IIId, wherein R₁, R₂, R₃′, R₃, R₃ₐ, R₃ₐ′, R₄, R₅, R₆, R₇, R₈, R₉, R₁₀, R₁₁, R₁₂, R₁₃, R₂₄, X₁, X₂, X₃, and X₄ are as described for formula III and further wherein

R₂₅ is an optionally substituted group selected from C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S, and provided that when one of R₃ or R₃′ is unsubstituted phenyl and the stereochemistry of the carbon atom adjacent to said phenyl is in the R-configuration, X₂ is N and one of R₁₀ or R₁₁ is CH₃, then R₂₄, R₃ₐ or R₃ₐ′ and one of R₂₅ or R₂₆ are not each methyl.

Formula IIId-1:

In one embodiment, the invention provides compounds of formula IIId-1, wherein R₃, R₃′, R₃ₐ, R₃ₐ′, R₂₄, and X₅ are as described for formula III and further wherein

R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1;

k is 0, 1, 2, 3, 4, or 5; and

R₂₅ is an optionally substituted group selected from C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S. In one embodiment, the invention relates to a compound or salt thereof having the formula IIId-1, provided that when one of R₃ or R₃′ is unsubstituted phenyl and the stereochemistry of the carbon atom adjacent to said phenyl is in the R-configuration and R₁₀ is CH₃, then R₂₄, R₃ₐ or R₃ₐ′ and R₂₅ are not each methyl.

Formula IIIe:

In one embodiment, the invention provides compounds of formula IIIe, wherein R₁, R₂, R₃, R_{3′}, R₃ₐ, R₃ₐ′, R₆, R₇, R₈, R₉, R₁₀, R₁₁, R₁₂, R₁₃, R₂₄, R₂₅, R₂₆, X₁, X₂, X₃, X₄, and X₅ are as described for formula III and further wherein

R₄ is hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₅ is hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring.

Formula IIIe-1:

In one embodiment, the invention provides compounds of formula IIIe-1, wherein R₃, R₃′, R₃ₐ, R₃ₐ′, R₂₄, R₂₅, R₂₆, and X₅ are as described for formula III and further wherein

R₄ is hydrogen, —COR₁₄, —CO₂R₁₄, —SOR₂₃, —SO₂R₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring.

Formula IIIe-2:

In one embodiment, the invention provides compounds of formula IIIe-2, wherein R₃, R₃′, R₃ₐ, R₃ₐ′, R₂₄, R₂₅, R₂₆, and X₅ are as described for formula III and further wherein R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring,

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula IIIe-3:

In one embodiment, the invention provides compounds of formula IIIe-3, wherein R₃, R₃′, R₃ₐ, R₃ₐ′ and R₂₄ are as described for formula III further wherein R₂₅ is hydrogen, —COR₁₄, —CO₂R₁₄, —SOR₂₃, —SO₂R₂₃, —C(O)NR₁₅R₁₆, —CH₂(C═O)—NR₂₁R₂₂, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula IIIf:

In one embodiment, the invention provides compounds of formula IIIf, wherein R₁, R₂, R₃, R₃′, R₃ₐ, R₃ₐ′, R₄, R₅, R₆, R₇, R₈, R₉, R₁₀, R₁₁, R₁₂, R₁₃, R₂₄, X₁, X₂, X₃, and X₄ are as described for formula III. In one embodiment, the invention relates to a compound or salt thereof having the formula R_{1f}, provided that when one of R₃ or R₃′ and one of R₃ₐ or R₃ₐ′ taken together with their intervening atoms form a 6-membered heterocyclic ring containing one oxygen atom adjacent to the carbon atom at the 2-position of the 12-membered ring, then at least one of R₁₀ or R₁₁ is not hydrogen when X₂ is N.

Formula IIIf-1:

In one embodiment, the invention provides compounds of formula IIIf-1, wherein R₃, R₃′, R₃ₐ, R₃ₐ′ and R₂₄ are as described for formula III and further wherein

R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring,

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5. In one embodiment, the invention relates to a compound or salt thereof having the formula IIIf-1, provided that when one of R₃ or R₃′ and one of R₃ₐ or R₃ₐ′ taken together with their intervening atoms form a 6-membered heterocyclic ring containing one oxygen atom adjacent to the carbon atom at the 2-position of the 12-membered ring, then R₁₀ is not hydrogen.

Formula IV:

In one embodiment, the invention provides compounds of formula IV, wherein R₁ and R₂ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₃ and R₃′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ and R₃′ are taken together with their intervening atoms to form a 3-8-membered ring;

when X₁ is N, one of R₃ or R₃′ and one of R₁₂ or R₁₃ taken together with their intervening atoms to form a 3-8 membered ring; or

R₃ₐ and R₃ₐ′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ₐ or R₃ₐ′ taken together with their intervening atoms form a 3-8-membered ring; or

when X₂ is N, one of R₃ₐ or R₃ₐ′ and one of R₁₀ or R₁₁ taken together with their intervening atoms to form a 3-8 membered ring; or

one of R₃ or R₃′ and one of R₃ₐ or R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₄ and R₅ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₄ is C, together R₄ and R₅ form ═O;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₀ and R₁₁ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₂ is C, together R₁₀ and R₁₁ form ═O;

R₁₂ and R₁₃ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₁ is C, together R₁₂ and R₁₃ form ═O;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₁ is O, N, or C;

X₂ is O, N, or C;

X₃ is O or C;

X₄ is O, N, or C;

X₅ is N or C;

P is —CH₂(C═O)ᵤNR₂₁R₂₂;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —NR₁₅C(O)R₁₆, —CH₂(C═O)ᵤNR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula IVa:

In one embodiment, the invention provides compounds of formula IVa, wherein R₃, R₃′, R₃ₐ, R₃ₐ′, R₄, R₅, R₆, R₇, R₈, R₉, R₁₀, R₁₁, R₁₂, R₁₃, R₂₄, R₂₅, R₂₆, X₁, X₂, X₄, and X₅ are as described for formula IV.

Formula IVa-1:

In one embodiment, the invention provides compounds of formula IVa-1, wherein R₃, R₃′, R₃ₐ, R₃ₐ′, X₅, R₂₄, R₂₅, and R₂₆, are as described for formula IV and further wherein R₁₀ is hydrogen, halogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1;

k is 0, 1, 2, 3, 4, or 5; and

P is —CH₂(C═O)ᵤNR₂₁R₂₂.

Formula IVb:

In one embodiment, the invention provides compounds of formula IVb, wherein R₃, R₃′, R₃ₐ, R₃ₐ′, R₄, R₅, R₆, R₇, R₈, R₉, R₁₀, R₁₁, R₁₂, R₁₃, R₂₄, R₂₅, R₂₆, X₁, X₂, X₄, and X₅ are as described for formula IV.

Formula IVb-1:

In one embodiment, the invention provides compounds of formula IVb-1, wherein R₃, R₃′, R₃ₐ, R₃ₐ′, X₅, R₂₄, R₂₅, and R₂₆ are as described formula IVb-1 and further wherein R₁₀ is hydrogen, halogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1;

k is 0, 1, 2, 3, 4, or 5; and

P is —CH₂(C═O)ᵤNR₂₁R₂₂.

Formula V:

In one embodiment, the invention provides compounds of formula V, wherein R₁ and R₂ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₃ is C, together R₁ and R₂ form ═O;

one of R₃ or R₃′ and one of R₃ₐ or R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₄ and R₅ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₄ is C, together R₄ and R₅ form ═O;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₀ and R₁₁ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₂ is C, together R₁₀ and R₁₁ form ═O;

R₁₂ and R₁₃ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S, or when X₁ is C, together R₁₂ and R₁₃ form ═O;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₁ is O, N, or C;

X₂ is O, N, or C;

X₃ is O or C;

X₄ is O, N, or C;

X₅ is N or C;

P is —CH₂(C═O)ᵤNR₂₁R₂₂;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —NR₁₅C(O)R₁₆, —CH₂(C═O)ᵤNR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5. In one embodiment, the invention relates to a compound or salt thereof having the formula V, provided that when one of R₃ or R₃′ and one of R₃ₐ or R₃ₐ′ taken together with their intervening atoms form a 6-membered heterocyclic ring containing one oxygen atom adjacent to the carbon atom at the 2-position of the 12-membered ring, then at least one of R₁₀ or R₁₁ is not hydrogen when X₂ is N.

Formula Va:

In one embodiment, the invention provides compounds of formula Va, wherein R₃, R₃′, R₃ₐ, R₃ₐ′, X₅, R₂₄, R₂₅, and R₂₆ are as described for formula V, further wherein R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

P is —CH₂(C═O)ᵤNR₂₁R₂₂;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5. In one embodiment, the invention relates to a compound or salt thereof having the formula Va, provided that when one of R₃ or R₃′ and one of R₃ₐ or R₃ₐ′ form a 6-membered ring containing one oxygen heteroatom adjacent to the carbon atom at the 2-position of the 12-membered ring, then at least one of R₁₀, R₂₅, or R₂₆ is not hydrogen.

Formula VI:

In one embodiment, the invention provides compounds of formula VI, wherein R₃ and R₃′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ and R₃′ are taken together with their intervening atoms to form a 3-8-membered ring; or

one of R₃ or R₃′ and R₁₂ taken together with their intervening atoms to form a 3-8 membered ring; or

R₃ₐ and R₃ₐ′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ₐ or R₃ₐ′ taken together with their intervening atoms form a 3-8-membered ring; or

one of R₃ or R₃′ and one of R₃ₐ or R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₂ is hydrogen, —COR₁₄, —CO₂R₁₄, —SOR₂₃, —SO₂R₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₅ is N or C;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —NR₁₅C(O)R₁₆, —CH₂(C═O)—NR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula VIa:

In one embodiment, the invention provides compounds of formula VIa, wherein R₃ and R₃′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ and R₃′ are taken together with their intervening atoms to form a 3-8-membered ring;

R₃ₐ and R₃ₐ′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ₐ or R₃ₐ′ taken together with their intervening atoms form a 3-8-membered ring; or

R₃ₐ or R₃ₐ′ and R₁₀ taken together with their intervening atoms to form a 3-8 membered ring; or

one of R₃ or R₃′ and one of R₃ₐ or R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₀ is hydrogen, —COR₁₄, —CO₂R₁₄, —SOR₂₃, —SO₂R₂₃, —C(O)NR₁₅R₁₆, P, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S or when X₂ is C, together R₁₀ and R₁₁ form ═O;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₅ is N or C;

P is —CH₂(C═O)ᵤNR₂₁R₂₂;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —NR₁₅C(O)R₁₆, —CH₂(C═O)ᵤNR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

Formula VII:

In one embodiment, the invention provides compounds of formula VII, wherein R₃ and R₃′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ and R₃′ are taken together with their intervening atoms to form a 3-8-membered ring;

R₃ₐ and R₃ₐ′ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O) OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₃ₐ and R₃ₐ′ taken together with their intervening atoms form a 3-8-membered ring; or

one of R₃ or R₃′ and one of R₃ₐ or R₃ₐ′ taken together with their intervening atoms form a 3-8-membered ring, wherein said ring optionally contains one or more heteroatoms selected from O, S, and N and said ring is optionally substituted with one or more B;

B is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₆, R₇, R₈, and R₉ are each independently hydrogen, halogen, —CN, —SCN, —NO₂, —COR₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

or together one of R₆ or R₇ and one of R₈ or R₉ form a bond;

or one of R₆ or R₇ and one of R₈ or R₉ taken together with their intervening atoms form a 3-6-membered ring, wherein said ring optionally contains one or more heteroatoms selected from O, N, or S; or

together R₈ and R₉ form ═O or ═N—OR;

R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or

R₁₅ and R₁₆ taken together with their intervening atoms form a 3-8-membered ring;

R₂₁ and R₂₂ are each independently hydrogen, —C(O)ᵥ(CH₂)ₖ-J or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R₂₄ is hydrogen, halogen, —CN, —SCN, —NO₂, —C(O)R₁₄, —CO₂R₁₄, —SOR₂₃, —SO₂R₂₃, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

R is hydrogen, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S;

X₅ is N or C;

R₂₅ and R₂₆ are each independently hydrogen, halogen, —CN, —SCN, —NO₂, —COR₁₄, —CO₂R₁₄, —SOR₂₃, —SO₂R₂₃, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —C(O)NR₁₅R₁₆, —NR₂₁R₂₂, —NR₁₅C(O)R₁₆, —CH₂(C═O)—NR₂₁R₂₂, absent, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

J is 6-10-membered aryl or 5-10-membered heteroaryl having 1-4 heteroatoms selected from N, O, and S, or 4-7-membered heterocyclyl having 1-2 heteroatoms independently selected from N, O, and S; further wherein J is optionally substituted with one or more T;

T is halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —C(O)NR₁₅R₁₆, —COR₁₄, —CO₂R₁₄, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S;

u is 0 or 1;

v is 0 or 1; and

k is 0, 1, 2, 3, 4, or 5.

In one embodiment, the invention relates to a compound of the invention or salt thereof, wherein X₃ is C and together R₁ and R₂ form ═O. In one embodiment, the invention relates to a compound of the invention or salt thereof, wherein X₃ is C and R₁ and R₂ are both hydrogen.

In one embodiment, the invention relates to a compound of the invention or salt thereof, wherein X₁ is O and R₁₂ and R₁₃ are both absent. In one embodiment, the invention relates to a compound of the invention or salt thereof, wherein X₁ is N and one of R₁₂ or R₁₃ are hydrogen and the other is absent. In one embodiment, the invention relates to a compound of the invention or salt thereof, wherein X₁ is C and together R₁₂ and R₁₃ form ═O. In one embodiment, the invention relates to a compound of the invention or salt thereof, wherein X₁ is C and R₁₂ and R₁₃ are both hydrogen.

In one embodiment, the invention relates to a compound of the invention or salt thereof, wherein when X₂ is N, one of R₃ₐ or R₃ₐ′ and one of R₁₀ or R₁₁ taken together with their intervening atoms forms a 3, 4, 5, 6, 7, or 8-membered ring. In one embodiment, the invention relates to a compound of the invention or salt thereof, wherein one of R₃ or R₃′ is unsubstituted phenyl and at least one of R₃ₐ or R₃ₐ′ is hydrogen. In one embodiment, the invention relates to a compound of the invention or salt thereof, wherein one of R₃ or R₃′ is substituted phenyl, wherein said phenyl is substituted with one or more substituents selected from halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or R₁₅ and R₁₆ taken together with their intervening atoms form a 3-8-membered ring.

In one embodiment, the invention relates to a compound of the invention or salt thereof, wherein R₃ or R₃′ is said phenyl is substituted with one substituent. In one embodiment, the invention relates to a compound of the invention or salt thereof, wherein said phenyl is substituted with two substituents. In one embodiment, the invention relates to a compound of the invention or salt thereof, wherein said phenyl is substituted with three substituents. In one embodiment, the invention relates to a compound of the invention or salt thereof, wherein said phenyl is substituted with four substituents. In one embodiment, the invention relates to a compound of the invention or salt thereof, wherein said phenyl is substituted with five substituents.

In one embodiment, the invention relates to a compound of the invention or salt thereof, wherein the stereochemistry of the carbon atom adjacent to said phenyl is in the R-configuration. For example,

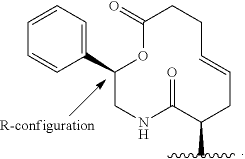 [SMILES: *[C@@H]1C/C=C/CCC(=O)O[C@H](c2ccccc2)CNC1=O]

In one embodiment, the invention relates to a compound of the invention or salt thereof, wherein the stereochemistry of the carbon atom adjacent to said phenyl is in the S-configuration. For example,

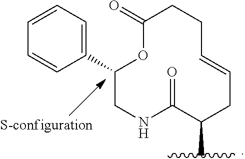 [SMILES: *[C@@H]1C/C=C/CCC(=O)O[C@@H](c2ccccc2)CNC1=O]

In one embodiment, the invention relates to a compound of the invention or salt thereof, wherein R₃ₐ and R₃ₐ′ are both hydrogen.

In one embodiment, the invention relates to a compound of the invention or salt thereof, wherein together one of R₃ or R₃′ and one of R₃ₐ or R₃ₐ′ with their intervening atoms form a 3, 4, 5, 6, 7, or 8-membered ring and said ring optionally contains one of more heteroatoms selected from O, S, and N. In one embodiment, the invention relates to a compound of the invention or salt thereof, wherein said ring formed is a 5-membered ring. In one embodiment, the invention relates to a compound of the invention or salt thereof, wherein said 5-membered ring is a cyclopentyl ring. In one embodiment, the invention relates to a compound of the invention or salt thereof, wherein said ring formed is a 6-membered ring. In one embodiment, the invention relates to a compound of the invention or salt thereof, wherein said 6-membered ring is a cyclohexyl ring. In one embodiment, the invention relates to a compound of the invention or salt thereof, wherein said 6-membered ring is a phenyl ring.

In one embodiment, the invention relates to a compound of the invention or salt thereof, wherein said ring formed by one of R₃ or R₃′ and one of R₃ₐ or R₃ₐ′ is substituted with one or more substituents selected from halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or R₁₅ and R₁₆ taken together with their intervening atoms form a 3, 4, 5, 6, 7, or 8-membered ring.

In one embodiment, the invention relates to a compound of the invention or salt thereof, wherein X₁ is N and one of R₃ or R₃′ and one of R₁₂ or R₁₃ taken together with their intervening atoms form a 3, 4, 5, 6, 7, or 8 membered ring. In one embodiment, the invention relates to a compound of the invention or salt thereof, wherein said ring is a 5-membered ring. In one embodiment, the invention relates to a compound of the invention or salt thereof, wherein said ring is a 6-membered ring.

In one embodiment, the invention relates to a compound of the invention or salt thereof, wherein X₂ is O and R₁₀ and R₁₁ are absent.

In one embodiment, the invention relates to a compound of the invention or salt thereof, wherein X₂ is N. In one embodiment, the invention relates to a compound of the invention or salt thereof, wherein X₂ is N and one of R₁₀ or R₁₁ is hydrogen and the other is absent. In one embodiment, the invention relates to a compound of the invention or salt thereof, wherein X₂ is N and one of R₁₀ or R₁₁ is C₁₋₁₂ aliphatic and the other is absent.

In one embodiment, the invention relates to a compound of the invention or salt thereof, wherein X₂ is N and one of R₁₀ or R₁₁ is P and the other is absent. In one embodiment, the invention relates to a compound of the invention or salt thereof, wherein P is —CH₂C(O)NR₂₁R₂₂. In one embodiment, the invention relates to a compound of the invention or salt thereof, wherein one of R₂₁ or R₂₂ is hydrogen and the other is —C(O)ᵥ(CH₂)ₖ-J. In one embodiment, the invention relates to a compound of the invention or salt thereof, wherein v is 0. In one embodiment, the invention relates to a compound of the invention or salt thereof, wherein v is 1. In one embodiment, the invention relates to a compound of the invention or salt thereof, wherein k is 1. In one embodiment, the invention relates to a compound of the invention or salt thereof, wherein J is phenyl. In one embodiment, the invention relates to a compound of the invention or salt thereof, wherein said J is substituted with at least one T. In one embodiment, the invention relates to a compound of the invention or salt thereof, wherein said T is halogen.

In one embodiment, the invention relates to a compound of the invention or salt thereof, wherein X₂ is C. In one embodiment, the invention relates to a compound of the invention or salt thereof, wherein X₂ is C and together R₁₀ and R₁₁ form ═O. In one embodiment, the invention relates to a compound of the invention or salt thereof, wherein X₂ is C and R₁₀ and R₁₁ are both hydrogen.

In one embodiment, the invention relates to a compound of the invention or salt thereof, wherein X₄ is C and together R₄ and R₅ form ═O. In one embodiment, the invention relates to a compound of the invention or salt thereof, wherein X₄ is C and R₄ and R₅ are both hydrogen.

In one embodiment, the invention relates to a compound of the invention or salt thereof, wherein X₄ is N. In one embodiment, the invention relates to a compound of the invention or salt thereof, wherein X₄ is N and one of R₄ or R₅ is hydrogen and the other is absent.

In one embodiment, the invention relates to a compound of the invention or salt thereof, wherein X₂ and X₄ are C.

In one embodiment, the invention relates to a compound of the invention or salt thereof, wherein X₅ is C.

In one embodiment, the invention relates to a compound of the invention or salt thereof, wherein X₅ is C and one of R₂₅ or R₂₆ is —NR₂₁R₂₂. In one embodiment, the invention relates to a compound of the invention or salt thereof, wherein one of R₂₁ or R₂₂ is —C(O)ᵥ(CH₂)ₖ-J. In one embodiment, the invention relates to a compound of the invention or salt thereof, wherein v is 1. In one embodiment, the invention relates to a compound of the invention or salt thereof, wherein v is 0. In one embodiment, the invention relates to a compound of the invention or salt thereof, wherein k is 1. In one embodiment, the invention relates to a compound of the invention or salt thereof, wherein k is 2. In one embodiment, the invention relates to a compound of the invention or salt thereof, wherein J is phenyl. In one embodiment, the invention relates to a compound of the invention or salt thereof, wherein said J is substituted with at least one T. In one embodiment, the invention relates to a compound of the invention or salt thereof, wherein said T is halogen.

In one embodiment, the invention relates to a compound of the invention or salt thereof, wherein X₅ is C and one of R₂₅ or R₂₆ is —CH₂(C═O)ᵤNR₂₁R₂₂ and the other is hydrogen. In one embodiment, the invention relates to a compound of the invention or salt thereof, wherein u is 1. In one embodiment, the invention relates to a compound of the invention or salt thereof, wherein one of R₂₁ or R₂₂ is —C(O)ᵥ(CH₂)ₖ-J and the other is hydrogen. In one embodiment, the invention relates to a compound of the invention or salt thereof, wherein v is 0. In one embodiment, the invention relates to a compound of the invention or salt thereof, wherein k is 1. In one embodiment, the invention relates to a compound of the invention or salt thereof, wherein J is phenyl. In one embodiment, the invention relates to a compound of the invention or salt thereof, wherein said J is substituted with at least one T. In one embodiment, the invention relates to a compound of the invention or salt thereof, wherein said T is halogen.

In one embodiment, the invention relates to a compound of the invention or salt thereof, wherein X₅ is N and one of R₂₅ or R₂₆ is —CH₂(C═O)ᵤNR₂₁R₂₂. In one embodiment, the invention relates to a compound of the invention or salt thereof, wherein u is 1. In one embodiment, the invention relates to a compound of the invention or salt thereof, wherein one of R₂₁ or R₂₂ is hydrogen and the other is —C(O)ᵥ(CH₂)ₖ-J. In one embodiment, the invention relates to a compound of the invention or salt thereof, wherein v is 0. In one embodiment, the invention relates to a compound of the invention or salt thereof, wherein k is 1. In one embodiment, the invention relates to a compound of the invention or salt thereof, wherein J is phenyl. In one embodiment, the invention relates to a compound of the invention or salt thereof, wherein said J is substituted with at least one T. In one embodiment, the invention relates to a compound of the invention or salt thereof, wherein said T is halogen.

In one embodiment, the invention relates to a compound of the invention or salt thereof, wherein X₅ is C and one of R₂₅ or R₂₆ is C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, or 4-7-membered heterocyclyl having 1-2 heteroatoms independently selected from N, O, and S and the other R₂₅ or R₂₆ is hydrogen.

In one embodiment, the invention relates to a compound of the invention or salt thereof, wherein said R₂₅ or R₂₆ is substituted with one or more substituents selected from halogen, —CN, —SCN, —NO₂, —NR₁₅R₁₆, —NR₁₅C(O)R₁₆, —NR₁₅C(O)NR₁₅R₁₆, —OR₁₇, —OC(O)OR₁₇, —OC(O)NR₁₅R₁₆, —NR₁₅C(O)OR₁₇, —SR₂₃, —SOR₂₃, —SO₂R₂₃, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; and R₁₄, R₁₅, R₁₆, R₁₇, and R₂₃ are each independently hydrogen, or an optionally substituted group selected from acyl, C₁₋₁₂ aliphatic, C₁₋₁₂ heteroaliphatic, C₇₋₁₅ arylalkyl, C₃₋₁₅ heteroarylalkyl, 6-10-membered aryl, 5-10-membered heteroaryl having 1-4 heteroatoms independently selected from N, O, and S, and 4-7-membered heterocyclyl having 1-2 heteroatoms independently selected from N, O, and S; or R₁₅ and R₁₆ taken together with their intervening atoms form a 3, 4, 5, 6, 7, or 8-membered ring.

In one embodiment, the invention relates to a compound of the invention or salt thereof, wherein X₅ is C and the stereochemistry of the carbon atom at X₅ is the S-configuration e.g., example,

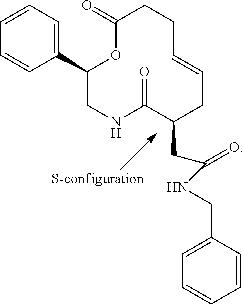 [SMILES: O=C(C[C@@H]1C/C=C/CCC(=O)O[C@H](c2ccccc2)CNC1=O)NCc1ccccc1]

In one embodiment, the invention relates to a compound of the invention or salt thereof, wherein X₅ is C and the stereochemistry of the carbon atom at X₅ is the R-configuration e.g.,

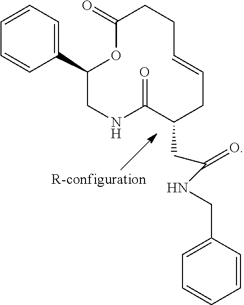 [SMILES: O=C(C[C@H]1C/C=C/CCC(=O)O[C@H](c2ccccc2)CNC1=O)NCc1ccccc1]

In one embodiment, the invention relates to a compound of the invention or salt thereof, wherein together R₈ and R₉ form ═O. In one embodiment, the invention relates to a compound of the invention or salt thereof, wherein one of R₈ or R₉ is OH and the other is C₁₋₁₂ aliphatic.

In one embodiment, the invention relates to a compound of the invention or salt thereof, wherein together one of R₆ or R₇ and one of R₈ or R₉ form a bond. In one embodiment, the invention relates to a compound of the invention or salt thereof, wherein the remaining R₆ or R₇ is selected from halogen, methyl, ethyl, —C(O)R₁₄, —NR₁₅R₁₆, and —OR₁₇. In one embodiment, the invention relates to a compound of the invention or salt thereof, wherein the remaining R₈ or R₉ is selected from halogen, methyl, ethyl, —C(O)R₁₄, —NR₁₅R₁₆, and —OR₁₇. In one embodiment, the invention relates to a compound of the invention or salt thereof, wherein R₁₄ is selected from methyl, ethyl, and benzyl. In one embodiment, the invention relates to a compound of the invention or salt thereof, wherein R₁₅ and R₁₆ are each independently selected from hydrogen, methyl, ethyl, propyl, and benzyl. In one embodiment, the invention relates to a compound of the invention or salt thereof, wherein R₁₇ is selected from hydrogen, methyl, ethyl, and benzyl.

In one embodiment, the invention relates to a compound of the invention or salt thereof, wherein together one of R₆ or R₇ and one of R₈ or R₉ together with their intervening atoms form a 3, 4, 5, or 6-membered ring. In one embodiment, the invention relates to a compound of the invention or salt thereof, wherein together one of R₆ or R₇ and one of R₈ or R₉ together with their intervening atoms form a 3-membered ring. In one embodiment, the invention relates to a compound of the invention or salt thereof, wherein the 3-membered ring is an epoxide ring. In one embodiment, the invention relates to a compound of the invention or salt thereof, wherein the 3-membered ring is a cyclopropyl ring.

In one embodiment, the invention relates to a compound of the invention or salt thereof, wherein at least one of R₆, R₇, R₈, or R₉ is OH. In one embodiment, the invention relates to a compound of the invention or salt thereof, wherein two of R₆, R₇, R₈, or R₉ are OH. In one embodiment, the invention relates to a compound of the invention or salt thereof, wherein one of R₆, R₇, R₈, or R₉ is OH, another is NH₂, and the remaining two are hydrogen. In one embodiment, the invention relates to a compound of the invention or salt thereof, wherein R₆, R₇, R₈, and R₉ are each hydrogen. In one embodiment, the invention relates to a compound of the invention or salt thereof, wherein together one of R₆ or R₇ and one of R₈ or R₉ form a 5-membered ring.

In one embodiment, the invention relates to a compound of the invention or salt thereof, wherein the 5-membered ring is

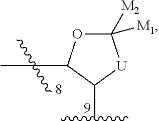

wherein U is O, NH or NR₁₄ and M₁ and M₂ are each independently selected from hydrogen or C₁₋₆ aliphatic or 6-10-membered aryl or together M₁ and M₂ form ═O.

In one embodiment, the invention provides compounds selected from Table 1. In one embodiment, the invention provides a compound selected from Compounds 9, 10, 11, 19, 20, 21, 22, and 23. In one embodiment, the invention provides a compound selected from Compounds 2, 4, 6, 7, 8, 14, and 17.

TABLE 1

| No. | Compound Structure |
|---|---|
| 1 | 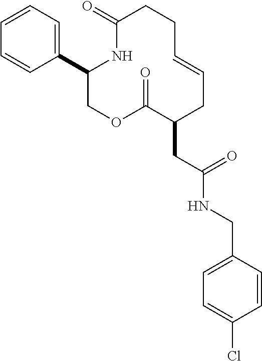 |
| 2 | 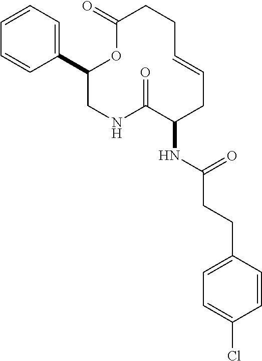 |
| 3 | 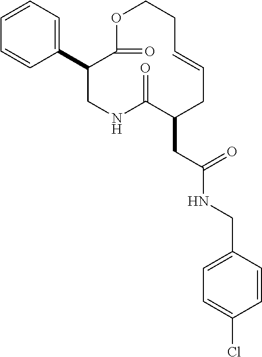 |
| 4 | 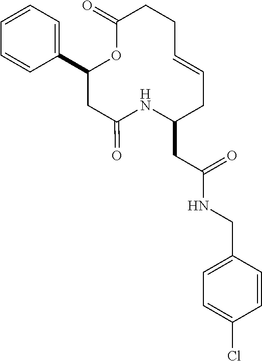 |
| 5 | 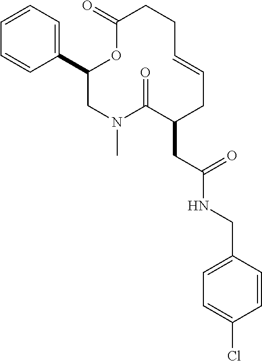 |
| 6 | 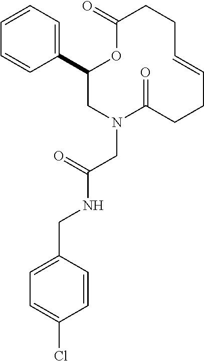 |
| 7 | 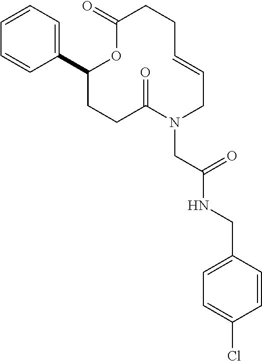 |
| 8 | 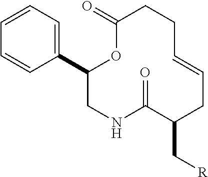 R = alkyl, aryl |
| 9 | 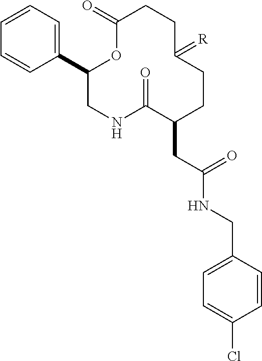 R = O, NOH, NOR′ |
| 10 | 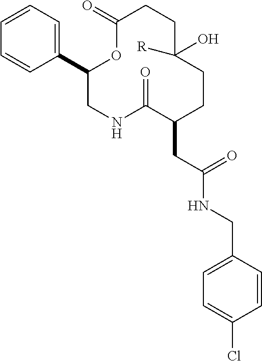 R = alkyl, aryl |
| 11 | 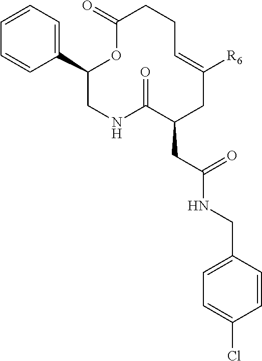 R₆ = Cl, —C(O)R₁₄, —NHR₁₅, —NR₁₅R₁₆, —OR₁₇ |
| 12 | 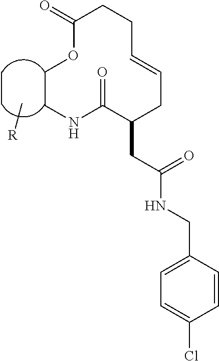 |
| 13 | 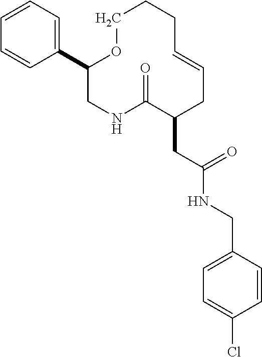 |
| 14 | 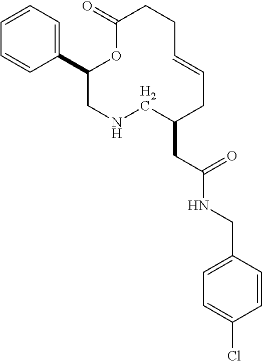 |
| 15 | 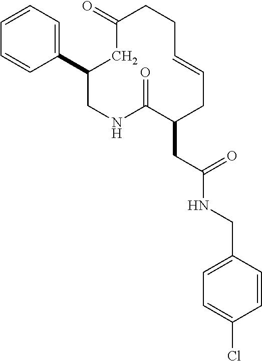 |
| 16 | 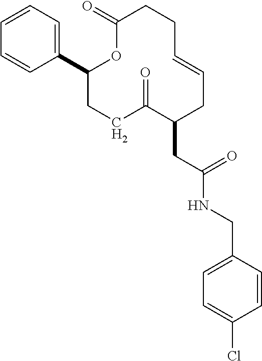 |
| 17 | 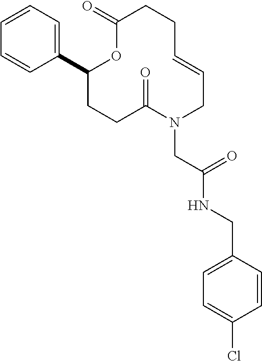 |
| 18 | 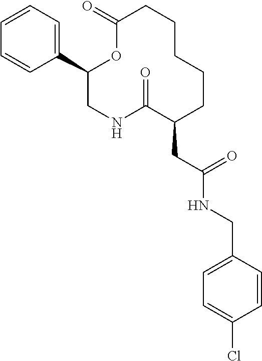 [SMILES: O=C(C[C@@H]1CCCCCC(=O)O[C@H](c2ccccc2)CNC1=O)NCc1ccc(Cl)cc1] |
| 19 | 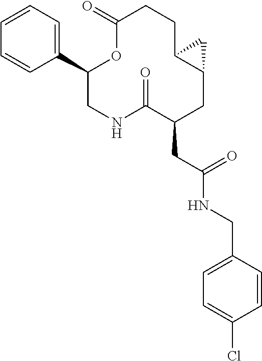 [SMILES: O=C(C[C@@H]1C[C@H]2C[C@@H]2CCC(=O)O[C@H](c2ccccc2)CNC1=O)NCc1ccc(Cl)cc1] |
| 20 | 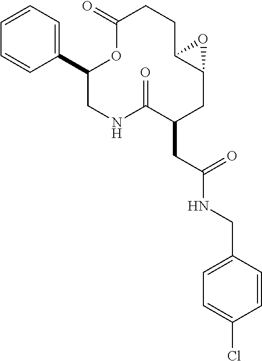 |
| 21 | 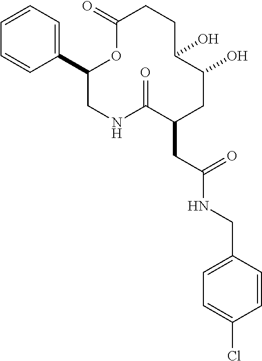 |
| 22 | 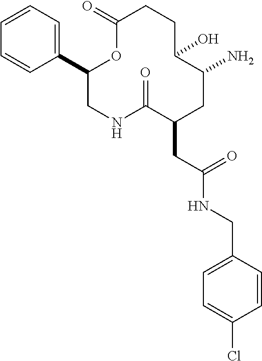 |
| 23 | 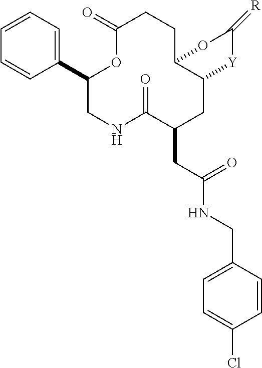 Y = OH, NH R = H, O, alkyl, aryl |

In some embodiments, the compounds of the invention are compounds of formulae X, Xa, Xb, Xd, Xd, XIa, XIb, XII, XIIa, XIIb, XIII and XIV and pharmaceutically acceptable salts thereof. In some embodiments, the compounds of the invention include the compounds of Table 2.

Formula X:

In one embodiment, the invention provides compounds of formula X:

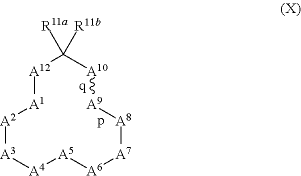

wherein

A¹ is CR^{1a}R^{1b}; O, C═O or NR^{1c};

A² is CR^{2a}R^{2b}, O, NR^{2c} or C═O;

A³ is (CR^{3a}R^{3b})ₐ—(CR^{3c}R^{3d})_{b};

A⁴ is CR^{4a}R^{4b}, O, C═O or NR^{4c};

A⁵ is CR^{5a}R^{5b}, O, C═O or NR^{5c};

A⁶ is CR^{6a}R^{6b}, O, C═O or NR^{6c};

A⁷ is (CR^{7a}R^{7b}), —(CR^{7c}R^{7d})_{f};

A⁸ is CR^{8a}R^{8b}, C═O or C═NOR^{8c};

A⁹ is CR^{9a}R^{9b}, C═O or C═NOR^{9c};

A¹⁰ is (CR^{10a}R^{10b})_{c}—(CR^{10c}R^{10d})_{d} or NR^{10e};

A¹² is CR^{12a}R^{12b}, O, C═O or NR^{12c};

a, b, c, d, e and f are each independently 0 or 1; p is a single bond when R^{8a} and R^{9a} are present or a double bond when R^{8a} and R^{9a} are absent;

q is a single bond when R^{9a} and R^{10a} are present or a double bond when R^{9a} and R^{10a} are absent; provided that both p and q are not both double bonds;

q is cis or trans to bond p when q is a single bond and p is a double bond;

p is cis or trans to bond q when p is a single bond and q is a double bond;

R^{1a}, R^{1b}, R^{2a}, R^{2b}, R^{4a}, R^{4b}, R^{5a}, R^{5b}, R^{6a}, R^{7a}; R^{7b}, R^{7c}, R^{7d}, R^{8b}, R^{9b}, R^{11a}, R^{11b}, R^{12a} and R^{12b} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{6b} is hydrogen, hydroxyl, alkyl alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{3a}, R^{3b}, R^{3c}, R^{3d}, R^{8a}, R^{9a}, R^{10a}, R^{10b}, R^{10c} and R^{10d} are each independently absent or hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{1c}, R^{4c}, R^{5c}, R^{6c}, R^{8c}, R^{9c}, R^{10e} and R^{12c} are each independently hydrogen, hydroxy, alkoxy, alkyl, alkenyl, alkynyl, aryl, carbonyl, carboxy, acyl or amino; or

R^{1c} and R^{2a}, or R^{2a} and R^{3a}, or R^{3a} and R^{4c}, or R^{8b} and R^{9b}, or R^{8b} and R^{10e}, together with the atoms to which they are attached, are linked to form a 3 to 10-membered carbocyclic, heterocyclic or aryl ring and pharmaceutically acceptable salt thereof;

provided that said alkyl is not substituted with C═XR^{6d}, wherein X is O, NR^{6e} or S; and R^{6d} is NR^{6′}R^{6″}, OR^{6′} or SR^{6′}; wherein R^{6e}, R^{6′} and R^{6″} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether.

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1; b is 0; R^{3a} is hydrogen; R^{3b} is hydrogen or alkyl (e.g., methyl); R^{3c}; R^{3d} are each absent; A⁴ is O; A⁵ is C═O; A⁷ is (CR^{7a}R^{7b})ₑ—(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond; R^{8a} and R^{9a} are each absent; R^{8b} and R^{9b} are each hydrogen, q is a single bond; q is trans to p; A¹⁰ is (CR^{10a}R^{10b})_{c}—(CR^{10c}R^{10d})_{d}, c is 1; d is 0; R^{10a} and R^{10b} are each hydrogen; R^{10c} and R^{10d} are absent; A⁶ is CR^{6a}R^{6b}; R^{6a} is hydrogen or alkyl (e.g., methyl); R^{11a} and R^{11b} are each hydrogen; A¹² is C═O; R^{2b} is aryl, for example, heteroaryl, such as pyridine or thiophene; or phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is amino, for example, NR^{6d}R^{6e}; R^{6d} is hydrogen or alkyl (e.g., methyl); and R^{6e} is hydrogen, alkylsulfonyl (e.g., methysulfonyl, isopropylsulfonyl or trifluoromethyethylsulfonyl); (C═O)R^{13a} or (C═S)R^{13a}, wherein R^{13a} is alkyl (methyl, isopropyl, aryl substituted alkyl, such as 4-chloroethylbenzyl), aryl (e.g., heteroaryl, such as imidazole, N-methylimidazole or pyrazole), or NHR^{14a}, wherein R^{14a} is alkyl (e.g., ethyl or benzyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is; b is 0; R^{3a} and R^{3b} are hydrogen; R^{3c} and R^{3d} are each absent; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} is hydrogen; R^{9b} is hydrogen or alkyl; q is a single bond; q is trans to p; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{10a} is hydrogen; R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen, alkoxy, aryloxy amino, nitro, cyano, alkyl, carboxy or carbonyl; and R^{6b} is hydrogen, hydroxyl, alkyl (e.g., methyl, isopropyl or hydroxyl-substituted alkyl such as C(CH₃)₂OH), alkoxy (e.g., methoxy), azido, heteroaryl (e.g., triazole, pyrazole, 4-chlorophenyltriazole, indazole), or a heterocyclic moiety (e.g., morpholine).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} and R^{3b} are hydrogen and R^{3c} and R^{3d} are each absent; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b} are each hydrogen, q is a single bond; q is trans to p; A¹⁰ is (CR^{10a}R^{10b})_{c}—(CR^{10a}R^{10b})_{d}, c is 1 and d is 0; R^{10a} and R^{10b} are each hydrogen; R^{10c} and R^{10d} are absent; A⁶ is CR^{6a}R^{6b}; R^{11a} is hydrogen; R^{11a} and R^{11b} are each hydrogen; A¹² is C═O; R^{2b} is hydrogen and R^{6b} is hydrogen.

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1; b is 0; R^{3a} and R^{3b} are hydrogen; R^{3c} and R^{3d} are each absent; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond; R^{8a} and R^{9a} are each absent; R^{8b} is hydrogen; R^{9b} is hydrogen or alkyl (e.g., methyl); q is a single bond; q is trans to p; A¹⁰ is (CR^{10a}R^{10b})_{c}—(CR^{10c}R^{10d})_{d}, c is 1; d is 0; R^{10a} and R^{10b} are each hydrogen; R^{10c} and R^{10d} are absent; A⁶ is CR^{6a}R^{6b}; is hydrogen or alkyl (e.g., methyl); R^{11a} and R^{11b} are each hydrogen; A¹² is C═O; R^{2b} is alkyl (e.g., methyl) and R^{6b} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} and R^{3b} are hydrogen; R^{3c} and R^{3d} are each absent; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b} are each hydrogen, q is a single bond; q is trans to p; A¹⁰ is (CR^{10a}R^{10b})_{c}—(CR^{10c}R^{10d})_{d}, c is 1; d is 0; R^{10a} and R^{10b} are each hydrogen; R^{10c} and R^{10d} are absent; A⁶ is CR^{6a}R^{6b}; R^{10a} is hydrogen or alkyl (e.g., methyl); R^{11a} is hydrogen, halogen (e.g., fluorine) or alkyl (e.g., methyl) and R^{11b} is alkyl (e.g., methyl), halogen (e.g., fluorine) hydroxyl, alkoxy (e.g., methoxy) or amino, for example, unsubstituted or acyl substituted amino; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}, a is 1; b is 0; R^{3a} is hydrogen; R^{3c} and R^{3d} are each absent; R^{2b} and R^{3b} are linked to form a ring (e.g., a 5-membered heterocyclic ring or a bicyclic ring, for example, dihydroindene); A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b} are each hydrogen, q is a single bond; q is trans to p; A¹⁰ is (CR^{10a}R^{10b})_{c}—(CR^{10c}R^{10d})_{d}, c is 1; d is 0; R^{10a} and R^{10b} are each hydrogen; R^{10c} and R^{10d} are absent; A⁶ is CR^{6a}R^{6b}; R^{6a} is hydrogen; R^{11a} is hydrogen; R^{11b} is alkyl (e.g., methyl); A¹² is C═O; and R^{6b} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1; b is 1; R^{3a} is hydrogen; R^{3b} is hydrogen; R^{3c} and R^{3d} are each hydrogen; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b} are each hydrogen, q is a single bond; q is trans to p; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen or alkyl (e.g., methyl); R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is amino, for example, NR^{6d}R^{6e}; R^{6d} is hydrogen or alkyl (e.g., methyl); and R^{6e} is (C═O)R^{13a}; R^{13a} is alkyl. (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1; b is 0; R^{3a} is hydrogen; R^{3b} is hydrogen; R^{3c} and R^{3d} are each absent; A⁴ is O; A⁵ is C═O; A⁷ is (CR^{7a}R^{7b})ₑ—(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b} are each hydrogen, q is a single bond; q is trans to p; A¹⁰ is (CR^{10a}R^{10b})_{c}—(CR^{10a}R^{10b})_{d}, c is 1; d is 1; R^{10a} and R^{10b} are each hydrogen; R^{10c} and R^{10d} are each hydrogen; A⁶ is CR^{6a}R^{6b}; R^{6a} is hydrogen; R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen, alkoxy, aryloxy amino, nitro, cyano, alkyl, carboxy or carbonyl, and R^{6b} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen; R^{3c} and R^{3d} are each absent; A⁴ is O; A⁵ is C═O; A⁷ is (CR^{7a}R^{7b})ₑ—(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b} are each hydrogen, q is a single bond; q is trans to p; A¹⁰ is (CR^{10a}R^{10b})_{c}—(CR^{10c}R^{10d})_{d}, c is 1; d is 0; R^{10a} and R^{10b} are each hydrogen; R^{10c} and R^{10d} are absent; A⁶ is NR^{6c}; R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen alkoxy, aryloxy, amino, nitro, cyano, alkyl, carboxy or carbonyl; and R^{6c} is alkyl, for example, unsubstituted alkyl (e.g., methyl) or substituted alkyl (e.g., 4-chlorobenzyl or aminocarbonyl substituted alkyl, such as methyl substituted with piperidinyl substituted carbonyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen; R^{3c} and R^{3d} are each absent; A⁴ is O; A⁵ is C═O; A⁷ is (CR^{7a}R^{7b})ₑ—(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{9b} is hydrogen; R^{8b} and R^{10e} are linked to form a 5-membered heterocyclic ring (e.g., a triazole ring), q is a single bond; q is trans to p; A¹⁰ is R^{10e}; A⁶ is NR^{6c}; R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6c} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1; b is 0; R^{3a} is hydrogen; R^{3b} is hydrogen; R^{3c} and R^{3d} are each absent; A⁴ is NR^{4c}; R^{4c} is alkyl (e.g., methyl); A⁵ is C═O; A⁷ is (CR^{7a}R^{7b})ₑ—(CR^{7c}R^{7d})_{f}; e is 1 and f is 0; 1 is 1 and m is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b}, are each hydrogen, q is a single bond; q is trans to p; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen or alkyl (e.g., methyl); R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen; R^{3c} and R^{3d} are each absent; A⁴ is O; A⁵ is CR^{5a}R^{5b}; R^{5a} and R^{5b} are each hydrogen; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond and R^{8a} and R^{9a} are each absent; R^{8b} and R^{9b} are each hydrogen, q is a single bond; q is trans to p; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen; R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen; R^{3c} and R^{3d} are each absent; A⁴ is C═O; A⁵ is O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{11a}R^{11b}; p is a double bond and R^{8a} and R^{11a} are each absent; R^{8b} and R^{9b} are each hydrogen, q is a single bond; q is trans to p; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen; R^{11a} and R^{11b} are each hydrogen; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is alkyl (e.g., methyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen; R^{3c} and R^{3d} are each absent; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a single bond and R^{8a} and R^{9a} are each hydrogen; R^{8b} and R^{9b} are each hydrogen, q is a single bond; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{11a} is hydrogen; R^{11a} is hydrogen or halogen (e.g., fluorine); R^{11b} is hydrogen or halogen (e.g., fluorine); A¹² is C═O; R^{2b} is alkyl (e.g., methyl) or aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is alkyl (e.g., methyl) or amino (e.g., amino substituted with acyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen; R^{3c} and R^{3d} are each absent; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a single bond and R^{8a} and R^{9a} are each hydrogen; R^{8b} is hydrogen or hydroxyl; and R^{9b} is alkyl (e.g., methyl), amino or hydroxyl, or R^{8b} and R^{9b} are linked to form a 5-membered heterocyclic ring (e.g. an oxazolidinone ring), q is a single bond; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen; R^{11a} is hydrogen or halogen (e.g., fluorine); R^{11b} is hydrogen or halogen (e.g., fluorine); A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is alkyl (e.g., methyl) or amino (e.g., amino substituted with acyl).

In one embodiment, A¹ is NR^{1c}; R^{1c} is hydrogen; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen; R^{3c} and R^{3d} are each absent; A⁴ is O; A⁵ is C═O; A⁷ is (CR^{7a}R^{7b}), —(CR^{7c}R^{7d})_{f}; e is 1 and f is 0; R^{7a} and R^{7b} are each hydrogen; A⁸ is CR^{8a}R^{8b}; A⁹ is C═O or N═OR^{9c}; p is a single bond and R^{8a} is hydrogen; R^{8b} is hydrogen; R^{9c} is hydroxyl; q is a single bond; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is alkyl (e.g., methyl); R^{11a} is hydrogen; R^{11b} is hydrogen; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is alkyl (e.g., methyl).

In one embodiment, A¹ is O; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3c} and R^{3d} are each absent; A⁴ is NR^{4c}; R^{3b} and R^{4c} are linked to form a 5-membered heterocyclic ring; A⁵ is C═O; A⁷ is (CR^{7a}R^{7b})ₑ—(CR⁷CR^{7d})_{f}; e is 1 and f is 0; R^{7a} and R^{7b} are each hydrogen; A⁸ is CR^{8a}R^{8b}; A⁹ is CR^{9a}R^{9b}; p is a double bond; R^{8a} is absent; R^{8b} is hydrogen; R^{9a} is absent; R^{9b} is hydrogen; q is a single bond; q is cis or trans to p; A¹⁰ is (CR^{10a}R^{10b})_{c}—(CR^{10c}R^{10d})_{d}, c is 1; d is 0; R^{10a} and R^{10b} are each hydrogen; R^{10c} and R^{10d} are absent; A⁶ is CR^{6a}R^{6b}; R^{6a} is hydrogen; R^{11a} is hydrogen; R^{11b} is hydrogen; A¹² is C═O; R^{2b} is hydrogen; and R^{6b} is alkyl (e.g., methyl) or amino (e.g., amino substituted with acyl).

In one embodiment, A¹ is O; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3b} is hydrogen or alkyl (e.g., methyl); R^{3c} and R^{3d} are each absent; A⁴ is NR^{4c}; R^{4c} is hydrogen or alkyl (e.g., methyl); A⁵ is C═O; A⁷ is (CR^{7a}R^{7b})ₑ—(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond; R^{8a} is absent; R^{8b} is hydrogen; R^{9a} is absent; R^{9b} is hydrogen; q is a single bond; q is trans to p; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen; R^{11a} is hydrogen; R^{11b} is hydrogen; A¹² is C═O; R^{2b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; and R^{6b} is hydrogen, alkyl (e.g., unsubstituted alkyl, for example, methyl or substituted alkyl, for example, 4-chlorobenzyl or 4-chloroethylbenzyl) or amino (e.g., amino substituted with acyl).

In one embodiment, A¹ is O; A² is CR^{2a}R^{2b}; R^{2a} is hydrogen; A³ is (CR^{3a}R^{3b})ₐ—(CR^{3c}R^{3d})_{b}; a is 1 and b is 0; R^{3a} is hydrogen; R^{3c} and R^{3d} are each absent; A⁴ is NR^{4c} R^{3b} and R^{4c} are linked to form a 5-membered heterocyclic ring; A⁵ is C═O; A⁷ is (CR^{7a}R^{7b})ₑ—(CR⁷CR^{7d})_{f}; e is 1 and f is 0; R^{7a} and R^{7b} are each hydrogen; A⁸ is CR^{8a}R^{8b}; A⁹ is CR^{9a}R^{9b}; p is a single bond; R^{8a} is hydrogen; R^{8b} is hydrogen; R^{9a} is hydrogen; R^{9b} is hydrogen; q is a single bond; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen; R^{11a} is hydrogen; R^{11b} is hydrogen; A¹² is C═O; R^{2b} is hydrogen; and R^{6b} is alkyl (e.g., methyl).

In one embodiment, A¹ is NR^{1c}; R^{1c} is hydrogen′ A² is C═O; A³ is (CR^{3a}R^{3b})ₐ—(CR^{3c}R^{3d})_{b}; a is 1 and b is 0; R^{3a} is hydrogen; R^{3b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{3c} and R^{3d} are each absent; A⁴ is CR^{4a}R^{4b}; R^{4a} is hydrogen; R^{4b} is hydrogen; A⁵ is O; A⁶ is C═O; A⁷ is (CR^{7a}R^{7b})ₑ—(CR^{7c}R^{7d})_{f}; e is 1 and f is 0; R^{7a} is hydrogen; R^{7b} is alkyl (e.g., methyl); A⁸ is CR^{8a}R^{8b}; A⁹ is CR^{9a}R^{9b}; p is a single bond; R^{8a} is hydrogen; R^{8b} is hydrogen; R^{9a} is absent; R^{9b} is hydrogen; q is a double bond; A¹⁰ is (CR^{10a}R^{10b})_{c}—(CR^{10c}R^{10d})_{d}, c is 1 and d is 0; R^{10a} is absent; R^{10b} is hydrogen; R^{10c} and R^{10d} are absent; R^{11a} is hydrogen; R^{11b} is hydrogen; A¹² is CR^{12a}R^{12b}; and R^{12a} and R^{12b} are each hydrogen.

In one embodiment, A¹ is O; A² is C═O; A³ is (CR^{3a}R^{3b})ₐ—(CR^{3c}R^{3d})_{b}; a is 1 and b is 0; R^{3a} is hydrogen; R^{3c} and R^{3d} are each absent; A⁴ is NR^{4c}; R^{3b} and R^{4c} are linked to form a 5-membered heterocyclic ring; A⁵ is C═O; A⁷ is (CR^{7a}R^{7b})ₑ—(CR^{7c}R^{7d})_{f}; e is 1 and f is 0; R^{7a} and R^{7b} are each hydrogen; A⁸ is CR^{8a}R^{8b}; A⁹ is CR^{9a}R^{9b}; p is a double bond; R^{8a} is absent; R^{8b} is hydrogen; R^{9a} is absent; R^{9b} is hydrogen; q is a single bond; q is trans to p; A¹⁰ is (CR^{10a}R^{10b})_{c}—(CR^{10c}R^{10d})_{d}, c is 1 and d is 0; R^{10a} and R^{10b} are each hydrogen; R^{10c} and R^{10d} are absent; A⁶ is CR^{6a}R^{6b}; R^{6a} is hydrogen; R^{11a} is hydrogen; R^{11b} is hydrogen; A¹² is CR^{12a}R^{12b}; R^{12a} and R^{12b} are each hydrogen; and R^{6b} is amino (e.g., amino substituted with acyl).

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

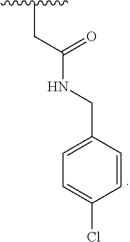 [SMILES: *CC(=O)NCc1ccc(Cl)cc1]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(CR^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

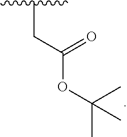 [SMILES: *CC(=O)OC(C)(C)C]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

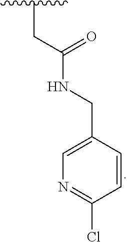 [SMILES: *CC(=O)NCc1ccc(Cl)nc1]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

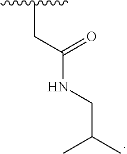 [SMILES: *CC(=O)NCC(C)C]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

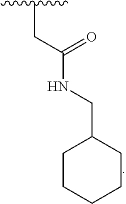 [SMILES: *CC(=O)NCC1CCCCC1]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

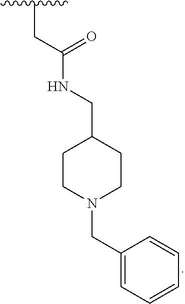 [SMILES: *CC(=O)NCC1CCN(Cc2ccccc2)CC1]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

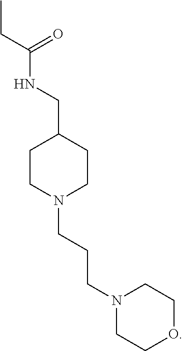 [SMILES: CCC(=O)NCC1CCN(CCCN2CCOCC2)CC1]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

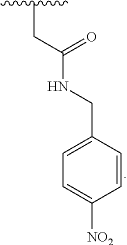 [SMILES: *CC(=O)NCc1ccc([N+](=O)[O-])cc1]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is hydrogen; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{1a} is hydrogen; R^{11b} is hydrogen; A¹² is C═O; then R^{6b} is not

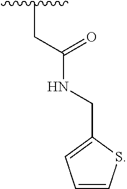 [SMILES: *CC(=O)NCc1cccs1]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is methyl; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

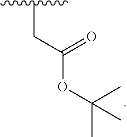 [SMILES: *CC(=O)OC(C)(C)C]

In one embodiment, when A¹ is O; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is NR^{4c}; R^{4c} is methyl; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

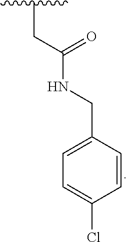 [SMILES: *CC(=O)NCc1ccc(Cl)cc1]

In one embodiment, when A¹ is NR^{1c}; R^{1c} is hydrogen; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is O; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

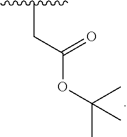 [SMILES: *CC(=O)OC(C)(C)C]

In one embodiment, when A¹ is NR^{1c}; R^{1c} is hydrogen; A² is CR^{2a}R^{2b}; R^{2a} is hydrogen; R^{2b} is unsubstituted phenyl; a is 1; b is 0; R^{3a} is hydrogen; R^{3b} is hydrogen; A⁴ is O; A⁵ is C═O; A⁶ is CR^{6a}R^{6b}; R^{6a} is hydrogen; e is 1; f is 0; R^{7a} is hydrogen; R^{7b} is hydrogen; p is a double bond; q is trans to p; A⁸ is CR^{8a}R^{8b}; R^{8a} is absent; R^{8b} is hydrogen; A⁹ is CR^{9a}R^{9b}; R^{9a} is absent; R^{9b} is hydrogen; A¹⁰ is (CR^{10a}R^{10b})_{c}—(C^{10c}R^{10d})_{d}; c is 1; d is 0; R^{10a} is hydrogen; R^{10b} is hydrogen; R^{11a} is hydrogen; R^{11b} is hydrogen; A¹² is C═O; then R^{6b} is not

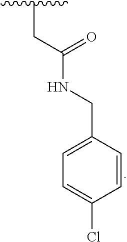 [SMILES: *CC(=O)NCc1ccc(Cl)cc1]

Formula Xa:

In one embodiment, the compounds of formula X are a compound of formula Xa:

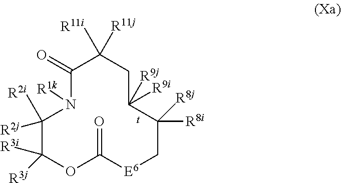

wherein

E⁶ is CR⁶ⁱR^{6j};

R^{1k} is hydrogen or alkyl;

R²ⁱ, R^{2j}, R³ⁱ, R^{3j}, R⁶ⁱ, R¹¹ⁱ and R^{11k} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;

R^{6j} is hydrogen, hydroxyl, C₁-C₁₂ unsubstituted alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;

R^{2j} and R^{3j}, together with the atoms to which they are attached, are linked to form a 3 to 10-membered carbocyclic, heterocyclic or aryl ring;

t is a single or double bond;

R⁸ⁱ and R⁹ⁱ are absent when t is a double bond or hydrogen when t is a single bond;

R^{8j} is hydrogen, hydroxyl, alkyl or amino;

R^{9j} is hydrogen, hydroxyl, or alkyl; or

R^{8j} and R^{9j}, together with the atoms to which they are attached, are linked to form a 3 to 10-membered carbocyclic, heterocyclic or aryl ring; or a pharmaceutically acceptable salt thereof.

In one embodiment, R^{1k} is hydrogen; R²ⁱ is hydrogen; R^{2j} is aryl (e.g., phenyl) or alkyl (e.g., methyl); t is a single bond; R³ⁱ and R^{3j} are each hydrogen; R⁶ⁱ is hydrogen; R^{6k} is C₁-C₁₂ alkyl (e.g., methyl) or amino (e.g., acyl substituted amino); R⁸ⁱ is hydrogen; R^{8j} is hydrogen or hydroxyl; R⁹ⁱ is hydrogen; R^{9j} is hydrogen, hydroxyl, or alkyl (e.g., methyl) or R^{8j} and R^{9j} are linked to form a 5-membered heterocyclic ring (e.g., oxazolidinone); and R¹¹ⁱ and R^{11j} are each independently hydrogen or halogen (e.g., fluorine).

In one embodiment, R^{1k} is hydrogen; R²ⁱ is hydrogen; R² is hydrogen, alkyl (e.g., methyl) or aryl (e.g., phenyl, thiophenyl or pyridine); R³ⁱ is hydrogen; R^{3j} is hydrogen or alkyl; or R² and R³ are linked to form a carbocyclic ring (e.g., pentyl or dihydroindene); t is a double bond; R⁶ⁱ is hydrogen or alkyl (e.g., methyl); R⁶ is hydrogen, hydroxyl, alkoxy (e.g., methoxy), azido, C₁-C₁₂ alkyl (e.g., methyl, cyclopropyl) or amino, for example, acyl amino; R⁸ⁱ and R⁹ⁱ are absent; R^{8j} is hydrogen; R^{9j} is hydrogen or alkyl (e.g., methyl); R¹¹ⁱ is hydrogen, halogen (e.g., fluorine) or alkyl (e.g., methyl) and R^{11j} is hydrogen, hydroxyl, alkoxy (e.g., methoxy), alkyl (e.g., methyl), halogen (fluorine) or amino (e.g., unsubstituted amino or amino substituted with acyl).

Formula Xb:

In one embodiment, the compound of formula X is a compound of formula Xb:

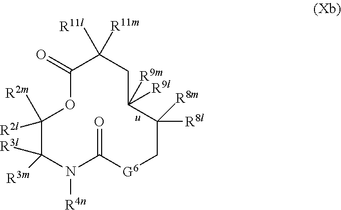

wherein

G⁶ is CR^{6l}R^{6m}.

u is a single or double bond;

R^{2l}, R^{2m}, R^{3l}, R^{3m} R^{6l}, R^{8m}, R^{9m}, R^{11l} and R^{11m} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;

R^{6m} is hydrogen, hydroxyl, C₁-C₁₂ unsubstituted alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;

R^{8l} and R^{9l} are absent when u is a double bond or hydrogen when u is a single bond;

R⁴ⁿ is hydrogen or alkyl; or R^{3m} and R⁴ⁿ, together with the atoms to which they are attached are linked to form a 3 to 10-membered heterocyclic or aryl ring, or a pharmaceutically acceptable salt thereof.

In one embodiment, R^{2l} is hydrogen; R^{2m} is hydrogen or alkyl (e.g., methyl) or aryl (e.g., phenyl such as unsubstituted phenyl or halogen substituted phenyl, for example, 4-fluorophenyl); R^{3l} is hydrogen; R^{3m} is hydrogen or alkyl (e.g., methyl); R⁴ⁿ is hydrogen or alkyl (e.g., methyl) or R^{3m} and R⁴ⁿ are linked to form a 5-membered heterocyclic ring (e.g., pyrrolidine); R^{6l} is hydrogen; R^{6m} is hydrogen, C₁-C₁₂ alkyl (e.g., methyl or isopropyl) or amino (e.g., acyl substituted amino); u is a double bond; R^{8l} and R^{9l} are absent and R^{8m} and R^{9m} are hydrogen; and R^{11l} and R^{1m} are each hydrogen.

Formula Xc:

In one embodiment, the compound of X is a compound of Xd:

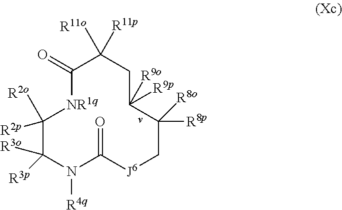

wherein

J⁶ is CR^{6o}R^{6p};

v is a single or double bond;

R^{2o}, R^{2p}, R^{3o}, R^{3p}, R^{6o}, R^{8p}, R^{9p}, R^{11o} and R^{11p} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;

R^{6p} is hydrogen, hydroxyl, C₁-C₁₂ unsubstituted alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;

R^{8o} and R^{9o} are absent when u is a double bond or hydrogen when u is a single bond;

R^{1q} and R^{4q} are each independently hydrogen or alkyl; or R^{3p} and R^{4q}, together with the atoms to which they are attached are linked to form a 3 to 10-membered heterocyclic or aryl ring, or a pharmaceutically acceptable salt thereof.

In one embodiment, R^{1q} is hydrogen; R^{2o} is hydrogen; R^{2p} is aryl (e.g., phenyl); R^{3o} and R^{3p} are each hydrogen; R^{4q} is alkyl (e.g., methyl); R^{6o} is hydrogen; R^{6p} is alkyl (e.g., methyl), v is a double bond; R^{8o} and R^{9o} are absent; R^{8p} and R^{9p} are hydrogen and R^{11o} and R^{11p} are each hydrogen.

Formula Xd:

In one embodiment, the compound of formula X is a compound of formula Xd:

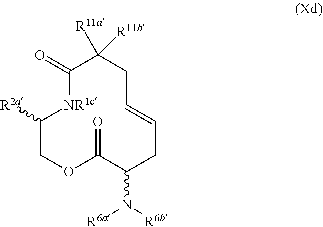

wherein

R^{1c′} is hydrogen or alkyl;

R^{2a′}, R^{11a′} and R^{11b′} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether; and

R^{6a′} and R^{6b′} are each independently hydrogen, hydroxy, alkoxy, alkyl, alkenyl, alkynyl, aryl, carbonyl, carboxy, acyl or amino, or R^{6a′} and R^{6b′} together with the nitrogen to which they are attached are linked to form a 3 to 10-membered heterocyclic or aryl ring; and pharmaceutically acceptable salts thereof.

In one embodiment, R^{1c′} is hydrogen; R^{2a′} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{11a′} and R^{11b′} are each hydrogen; R^{6a′} is hydrogen and R^{6b′} is acyl. In another embodiment, the R^{2a′} moiety is in the (R) or (S) position and NR^{6a′}R^{6b′} is in the (R) or (S) position.

In one embodiment, R^{1c′} is hydrogen; R^{2a′} is aryl (e.g., phenyl, for example, unsubstituted or phenyl substituted with halogen, such as fluorine); R^{11a′} and R^{11b′} are each hydrogen; R^{6a′} is hydrogen or alkyl (e.g., methyl) and R^{6b′} is hydrogen; alkylsulfonyl (e.g., methylsulfonyl, isopropylsulfonyl or trifluoroethylsulfonyl); acyl; carbonyl, for example, alkylcarbonyl (e.g., isopropylcarbonyl, 4-chloroethylbenzylcarbonyl), arylcarbonyl (e.g., heteroarylcarbonyl such as imidizole or N-methylimidizole substituted carbonyl or pyrazole substituted carbonyl), aminocarbonyl (e.g., CONHCH₂CH₃ or CONHCH₂C₆H₅).

In one embodiment, R^{1c′} is hydrogen, R^{2a′} is aryl (e.g., phenyl); R^{11a′} and R^{11b′} are each hydrogen and R^{6a′} and R^{6b′} are linked to form a heterocyclic ring (e.g., morpholine) or an aryl ring (e.g., a heteroaryl ring, such as triazole, 4-chlorophenyltriazole, pyrazole or indazole).

Formula XI:

In one embodiment, the invention provides compounds of formula XI:

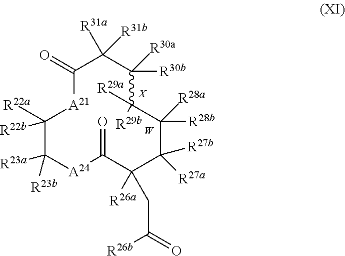

wherein

A²¹ is O or NR^{21c};

A²⁴ is O or NR^{24c};

w is a single bond when R^{28a} and R^{29a} are present or a double bond when R^{28a} and R^{29a} are absent;

x is cis or trans to w when w is a double bond;

R^{22a}, R^{22b}, R^{23a}, R^{23b}, R^{26a}, R^{27a}, R^{27b}, R^{28b}, R^{29b}, R^{30a}; R^{30b}, R^{31b} and R^{31b} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{26b} is NR^{26c}R^{26d} or OR^{26e};

R^{28a} and R^{29a} are each independently absent when w is a double bond or hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether when w is a single bond;

R^{21c}, R^{24c}, R^{26c} and R^{26d} are each independently hydrogen, hydroxy, alkoxy, alkyl, alkenyl, alkynyl, aryl, carbonyl, carboxy, acyl, amino or a carbocyclic or heterocyclic moiety, or R^{26c} and R^{26d} are linked to form a 3 to 10-membered heterocyclic or aryl ring;

R^{26e} is hydrogen, alkyl, alkenyl, alkynyl, aryl, amino, carbonyl or a heterocyclic moiety; or

R^{21c} and R^{22a}, or R^{22a} and R^{23a}, or R^{23a} and R^{24c}, or R^{28b} and R^{29b}, together with the atoms to which they are attached, are linked to form a 3 to 10-membered carbocyclic, heterocyclic or aryl ring; and pharmaceutically acceptable salts thereof;

provided that when w is a double bond, then R^{21c} and R^{22a} or R^{22a} and R^{23a} together with the atoms to which they are attached, are linked to form a 3 to 10-membered carbocyclic, heterocyclic or aryl ring; or

provided that when w is a double bond, then x is cis to w; or

provided that when w is a single bond, one of R^{28a} or R^{29a} is not hydrogen.

In one embodiment, A²¹ is NR^{21c}, R^{21c} is hydrogen; R^{22a} is hydrogen; R^{22b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{23a} and R^{23b} are each hydrogen; A²⁴ is O; R^{26a} is hydrogen; R^{26b} is NR^{26c}R^{26d}; R^{26c} is hydrogen; R^{26d} is alkyl (e.g., 4-chlorobenzyl); R^{27a} and R^{27b} are each hydrogen; w is a single bond; x is a single bond; R^{28a} is hydrogen; R^{28b} is hydroxy; R^{29a} is hydrogen; R^{29b} is hydroxyl; or R^{28b} and R^{29b} are linked to form a 3-membered heterocyclic ring (e.g., an epoxide ring); R^{30a} and R^{30b} are each hydrogen; and R^{31a} and R^{31b} are each hydrogen.

In one embodiment, A²¹ is NR^{21c}, R^{21c} is hydrogen; R^{22a} is hydrogen; R^{22b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{23a} and R^{23b} are each hydrogen; A²⁴ is O; R^{26a} is hydrogen; R^{26b} is NR^{26c}R^{26d}; R^{26c} is hydrogen; R^{26d} is alkyl (e.g., 4-chlorobenzyl); R^{27a} and R^{27b} are each hydrogen; w is a double bond; x is a single bond; x is cis to w; R^{28a} is absent; R^{28b} is hydrogen; R^{29a} is absent; R^{29b} is hydrogen; R^{30a} and R^{30b} are each hydrogen; R^{31a} is hydrogen and R^{31b} is alkyl (e.g., methyl).

In one embodiment, A²¹ is NR^{21c}, R^{21c} is hydrogen; R^{22a} is hydrogen; R^{23a} is hydrogen; R^{22b} and R^{23b} are linked to form a bicyclic ring (e.g., dihydroindene); A²⁴ is O; R^{26a} is hydrogen; R^{26b} is NR^{26c}R^{26d}; R^{26c} is hydrogen; R^{26d} is alkyl (e.g., 4-chlorobenzyl); R^{27a} and R^{27b} are each hydrogen; w is a double bond; x is a single bond; x is trans to w; R^{28a} is absent; R^{28b} is hydrogen; R^{29a} is absent; R^{29b} is hydrogen; R^{30a} and R^{30b} are each hydrogen; R^{31a} is hydrogen and R^{31b} is hydrogen.

In one embodiment, A²¹ is NR^{21c}, R^{21c} is hydrogen; R^{22a} is hydrogen; R^{23a} is hydrogen; R^{22b} and R^{23b} are linked to form a bicyclic ring (e.g., dihydroindene); A²⁴ is O; R^{26a} is hydrogen; R^{26b} is OR^{26e}; R^{26e} is alkyl (e.g., t-butyl); R^{27a} and R^{27b} are each hydrogen; w is a double bond; x is a single bond; x is trans to w; R^{28a} is absent; R^{28b} is hydrogen; R^{29a} is absent; R^{29b} is hydrogen; R^{30a} and R^{30b} are each hydrogen; R^{31a} is hydrogen and R^{31b} is hydrogen.

In one embodiment, A²¹ is NR^{21c}, R^{22a} is hydrogen; R^{21c} and R^{22b} are linked to form a 5-membered heterocyclic ring; R^{23a} and R^{23b} are each hydrogen; A²⁴ is O; R^{26a} is hydrogen; R^{26b} is NR^{26c}R^{26d}; R^{26c} is hydrogen; R^{26d} is alkyl (e.g., 4-chlorobenzyl); R^{27a} and R^{27b} are each hydrogen; w is a double bond; x is a single bond; x is trans to w; R^{28a} is absent; R^{28b} is hydrogen; R^{29a} is absent; R^{29b} is hydrogen; R^{30a} and R^{30b} are each hydrogen; R^{31a} is hydrogen and R^{31b} is hydrogen.

In one embodiment, A²¹ is O, R^{22a} is hydrogen; R^{22b} is hydrogen; R₂₃ₐ is hydrogen; A²⁴ is NR^{24c}; R^{23b} and R^{24c} are linked to form a 5-membered heterocyclic ring; R^{26a} is hydrogen; R^{26b} is NR^{26c}R^{26d}; R^{26c} is hydrogen; R^{26d} is alkyl (e.g., 4-chlorobenzyl); R^{27a} and R^{27b} are each hydrogen; w is a single bond; x is a single bond; R^{28a} is hydrogen; R^{28b} is hydroxy; R^{29a} is hydrogen; R^{29b} is hydroxyl; R^{30a} and R^{30b} are each hydrogen; and R^{31a} and R^{31b} are each hydrogen.

In one embodiment, A²¹ is O, R^{22a} is hydrogen; R^{22b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{23a} is hydrogen; R^{23b} is hydrogen or alkyl (e.g., methyl) A²⁴ is NR^{24c}; R^{24c} is hydrogen or alkyl (e.g., methyl); R^{26a} is hydrogen; R^{26b} is NR^{26c}R^{26d}; R^{26c} is hydrogen; R^{26d} is alkyl (e.g., 4-chlorobenzyl or thiophenyl substituted methyl) or a bicyclic ring (e.g., tetrahydronaphthalene); R^{27a} and R^{27b} are each hydrogen; w is a single bond; x is a single bond; R^{28a} is hydrogen; R^{28b} is hydroxy; R^{29a} is hydrogen; R^{29b} is hydroxyl; or R^{28b} and R^{29b} are linked to form a 3- or 5-membered heterocyclic ring (e.g., epoxide or a dioxolane ring); R^{30a} and R^{30b} are each hydrogen; and R^{31a} and R^{31b} are each hydrogen.

In one embodiment, A²¹ is O, R^{22a} is hydrogen; R^{22b} is hydrogen or alkyl (e.g., methyl); R^{23a} is hydrogen; A²⁴ is NR^{24c}; R^{23b} and R^{24c} are linked to form a 5-membered heterocyclic ring; R^{26a} is hydrogen; R^{26b} is NR^{26c}R^{26d}; R^{26c} is hydrogen; R^{26d} is alkyl (e.g., 4-chlorobenzyl); R^{27a} and R^{27b} are each hydrogen; w is a double bond; x is a single bond; x is cis to w; R^{28a} is absent; R^{28b} is hydrogen; R^{29a} is absent; R^{29b} is hydrogen; R^{30a} and R^{30b} are each hydrogen; and R^{31a} is hydrogen and R^{31b} is hydrogen or alkyl (e.g., methyl).

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

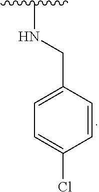 [SMILES: *NCc1ccc(Cl)cc1]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

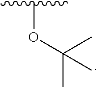 [SMILES: *OC(C)(C)C]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

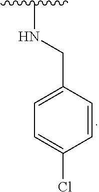 [SMILES: *NCc1ccc(Cl)cc1]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

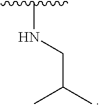 [SMILES: *NCC(C)C]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

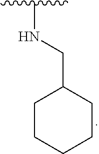 [SMILES: *NCC1CCCCC1]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

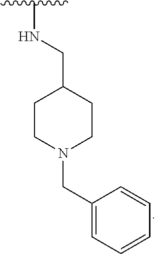 [SMILES: *NCC1CCN(Cc2ccccc2)CC1]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

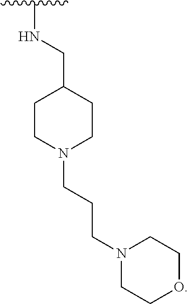 [SMILES: *NCC1CCN(CCCN2CCOCC2)CC1]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

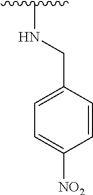 [SMILES: *NCc1ccc([N+](=O)[O-])cc1]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is hydrogen; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

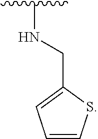 [SMILES: *NCc1cccs1]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is methyl; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

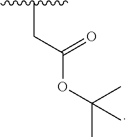 [SMILES: *CC(=O)OC(C)(C)C]

In one embodiment, when A²¹ is O; R^{22a} is hydrogen; R^{22b} is unsubstituted phenyl; R^{23a} is hydrogen; R^{23b} is hydrogen; A²⁴ is NR^{24c}; R^{24c} is methyl; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

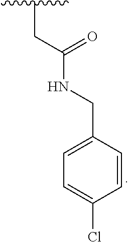 [SMILES: *CC(=O)NCc1ccc(Cl)cc1]

In one embodiment, when A²¹ is NR^{21c}; R^{21c} is hydrogen; R^{22a} is hydrogen; R^{22b} is unsubstituted phenyl; R^{23a} is hydrogen; R^{23b} is hydrogen; A²⁴ is O; R^{24c} is methyl; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

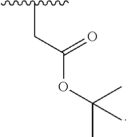 [SMILES: *CC(=O)OC(C)(C)C]

In one embodiment, when A²¹ is NR^{21c}; R^{21c} is hydrogen; R^{22a} is hydrogen; R^{22b} is unsubstituted phenyl; R^{23a} is hydrogen; R^{23b} is hydrogen; A²⁴ is O; R^{24c} is methyl; R^{26a} is hydrogen; R^{27a} is hydrogen; R^{27b} is hydrogen; p is a double bond; q is trans to p; R^{28a} is absent; R^{28b} is hydrogen; R^{29a} is absent; R^{29b} is hydrogen; R^{30a} is hydrogen; R^{30b} is hydrogen; R^{31a} is hydrogen; R^{31b} is hydrogen; then R^{26b} is not

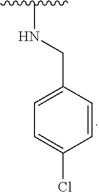 [SMILES: *NCc1ccc(Cl)cc1]

Formula XIa:

In one embodiment, the compound of formula XI is a compound of formula XIa:

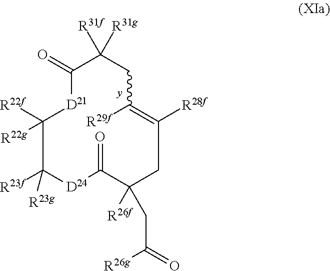

wherein

D²¹ is O or NR^{21h};

D²⁴ is O or NR^{24h};

R^{22f}, R^{23f}, R^{26f}, R^{28f}, R^{29f}, R^{31f} and R^{31g} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

y is cis or trans;

R^{22g} and R^{23g} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether when y is cis; or

R^{22g} and R^{23g} together with the atoms to which they are attached are linked to form a 3 to 10-membered carbocyclic, heterocyclic or aryl ring when y is cis or trans;

R^{21h} and R^{24h} are each independently hydrogen or alkyl;

R^{26g} is NR²⁶ⁱR^{26j} or OR^{26k};

R²⁶ⁱ and R^{26j} are each independently hydrogen, hydroxy, alkoxy, alkyl, alkenyl, alkynyl, aryl, carbonyl, carboxy, acyl, amino or a carbocyclic or heterocyclic moiety, or R²⁶ⁱ and R^{26j} are linked to form a 3-10 membered heterocyclic ring; and

R^{26k} is hydrogen, alkyl, alkenyl, alkynyl, aryl, amino, carbonyl or a heterocyclic moiety; or a pharmaceutically acceptable salt thereof.

In one embodiment, D²¹ is NR^{21h}; R^{21h} is hydrogen; R^{22f} and R^{23f} are each hydrogen; R^{22g} and R^{23g} are linked to join a carbocyclic ring (e.g., dihydroindene); D²⁴ is O; R^{26g} is NR²⁶ⁱR^{26j}; R²⁶ⁱ is hydrogen; R^{26j} is alkyl (e.g., 4-chlorobenzyl); y is trans; and R^{28f}, R^{29f}, R^{31f} and R^{31g} are each hydrogen.

In one embodiment, D²¹ is O; R^{22f} is hydrogen; R^{22g} is aryl (e.g., phenyl); R^{23f} is hydrogen; R^{23g} is alkyl (e.g., methyl); R^{26g} is NR²⁶ⁱR^{26j}; R²⁶ⁱ is hydrogen; R^{26j} is alkyl (e.g., 4-chlorobenzyl); y is cis and R^{28f}, R^{29f}, R^{31f} and R^{31g} are each hydrogen.

Formula XIb:

In one embodiment, the compound of formula XI is a compound of formula XIb:

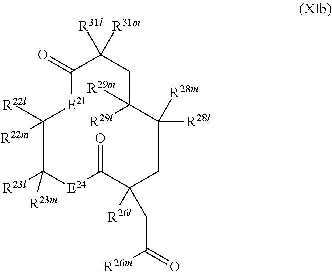

wherein

E²¹ is O or NR²¹ⁿ;

E²⁴ is O or NR²⁴ⁿ;

R^{22l}, R^{22m}, R^{23l}, R^{23m}, R^{26l}, R^{28l}, R^{28m}, R^{29l}, R^{29m}, R^{31l} and R^{31m} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether, provided that one of R^{28l} or R^{29l} are not hydrogen; or R^{28l} and R^{29l}, together with the atoms to which they are attached, are linked to form a 3 to 10-membered carbocyclic, heterocyclic or aromatic ring;

R²¹ⁿ and R²⁴ⁿ are each independently hydrogen or alkyl;

R^{26m} is NR^{26o}R^{26p} or OR^{26q};

R^{26o} and R^{26p} are each independently hydrogen, hydroxy, alkoxy, alkyl, alkenyl, alkynyl, aryl, carbonyl, carboxy, acyl, amino or a carbocyclic or heterocyclic moiety, or R^{26o} and R^{26p} together with the atoms to which they are attached are linked to form a 3 to 10 membered heterocyclic or aryl ring; and

R^{26q} is hydrogen, alkyl, alkenyl, alkynyl, aryl, amino, carbonyl or a heterocyclic moiety; or a pharmaceutically acceptable salt thereof.

In one embodiment, E²¹ is O; R^{22l} is hydrogen; R^{22m} is hydrogen or aryl (e.g., phenyl); R^{23l} is hydrogen or alkyl (e.g., methyl); E²⁴ is NR²⁴ⁿ; R²⁴ⁿ is hydrogen or alkyl (e.g., methyl); or R^{23m} and R²⁴ⁿ are linked to form a 5-membered heterocyclic ring (e.g., pyrrolidine); R^{26m} is NR^{26o}R^{26p}; R^{26o} is hydrogen; R^{26p} is alkyl (e.g., 4-chlorobenzyl or thiophenyl substituted methyl); R^{28l} is hydrogen; R^{28m} is hydroxyl; R^{29l} is hydrogen; R^{29m} is hydroxyl; or R^{28l} and R^{29l} are linked to form a 3- or 5-membered heterocyclic ring (e.g., epoxide or dioxalane) and R^{31l} and R^{31m} are each hydrogen.

In one embodiment, E²¹ is NR²¹ⁿ; R^{22l} is hydrogen; R^{22m} is aryl (e.g., phenyl); R^{23l} and R^{23m} are each hydrogen; E²⁴ is O; R^{26m} is NR^{26o}R^{26p}; R^{26o} is hydrogen; R^{26p} is alkyl (e.g., 4-chlorobenzyl); R^{28l} is hydrogen; R^{28m} is hydroxyl; R^{29l} is hydrogen; R^{29m} is hydroxyl; or R^{28l} and R^{29l} are linked to form a 3-membered heterocyclic ring (e.g., epoxide) and R^{31l} and R^{31m} are each hydrogen.

Formula XII:

In one embodiment, the invention provides compound for formula XII:

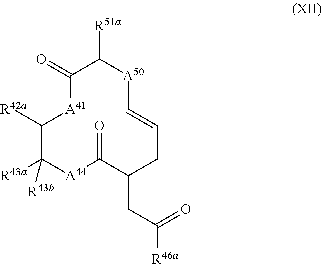

wherein

A⁴¹ is O or NR^{41c};

A⁴⁴ is O or NR^{44c};

R^{42a}, R^{43a}, R^{43b}, R^{46e} and R^{51a} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether; or

R^{43b} and R^{44c}, together with the atoms to which they are attached, are linked to form a 3-10-membered heterocyclic or aryl ring;

R^{46a} is NR^{46b}R^{46c} or OR^{46d};

R^{41c}, R^{44c}, R^{46b}, R^{46c} are each independently hydrogen, hydroxy, alkoxy, alkyl, alkenyl, alkynyl, aryl, carbonyl, carboxy, acyl, amino or CH₂R^{46e}; or R^{46b} and R^{46c} are linked to form a 3 to 10 membered heterocyclic or aryl ring;

R^{46d} is hydrogen, alkyl, alkenyl, alkynyl, aryl, amino, carbonyl or a heterocyclic moiety;

A⁵⁰ is (CH₂)ₑ—(CH₂)_{f}; and

e and f are each independently 0 or 1; and pharmaceutically acceptable salts thereof,

provided that when A⁴¹ is O; R^{42a} is unsubstituted phenyl; A⁴⁴ is NR^{44c}; e is 1; f is 0; R^{46a} is NR^{46b}R^{46c}; R^{43a}, R^{43b}, R^{44c}, R^{46b} and R^{51a} are each hydrogen and R^{46c} is CH₂R^{46e}, then R^{46e} is not

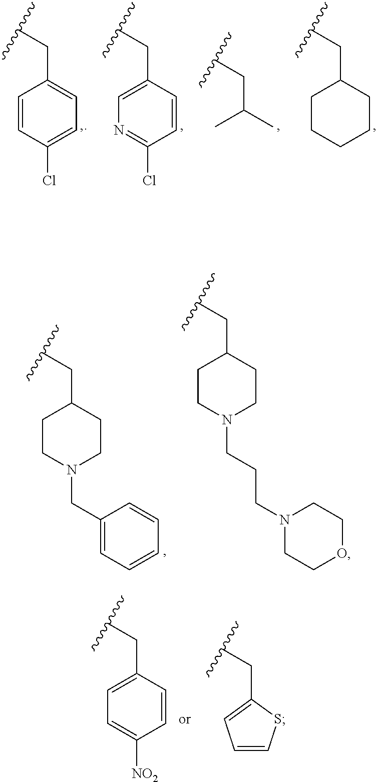

provided that when A⁴¹ is O; R^{42a} is unsubstituted phenyl; A⁴⁴ is NR^{44c}; R^{44c} is hydrogen or methyl; e is 1; f is 0; R^{46a} is OR^{46d}; R^{43a}, R^{43b} and R^{51a} are each hydrogen, then R^{46d} is not

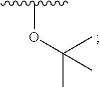 [SMILES: *OC(C)(C)C]

provided that when A⁴¹ is O; R^{42a} is unsubstituted phenyl; A⁴⁴ is NR^{44c}; R^{44c} is methyl; e is 1; f is 0; R^{46a} is NR^{46b}R^{46c}; R^{43a}, R^{43b}, R^{46b} and R^{51a} are each hydrogen and R^{46c} is CH₂R^{46e}, then R^{46e} is not

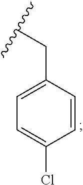 [SMILES: *Cc1ccc(Cl)cc1]

provided that when A⁴¹ is NR^{41c}; R^{41c} is hydrogen; R^{42a} is unsubstituted phenyl; A⁴⁴ is O; e is 1; f is 0; R^{46a} is OR^{46d}; R^{43a}, R^{43b} and R^{51a} are each hydrogen, then R^{46d} is not

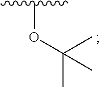 [SMILES: *OC(C)(C)C]

and

provided that when A⁴¹ is NR^{41c}; R^{41c} is hydrogen; R^{42a} is unsubstituted phenyl; A⁴⁴ is O; e is 1; f is 0; R^{46a} is NR^{46b}R^{46c}; R^{43a}, R^{43b}, R^{46b} and R^{51a} are each hydrogen and R^{46c} is CH₂R^{46e}, then R^{46e} is not

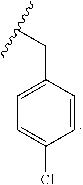 [SMILES: *Cc1ccc(Cl)cc1]

In one embodiment, A⁴¹ is NR^{41c}; R^{41c} is hydrogen or alkyl (e.g., methyl); R^{42a} is alkyl (e.g., isopropyl, n-butyl, benzyl or cyclohexyl) or aryl, for example, phenyl such as unsubstituted phenyl or phenyl substituted at the 2-, 3- or 4-position with one or more of halogen (e.g., fluorine or chlorine), alkoxy (e.g., methoxy), aryloxy, amino, nitro, cyano, alkyl, carboxy or carbonyl; R^{43a} and R^{43b} are each hydrogen; A⁴⁴ is O; R^{46a} is NR^{46b}R^{46c}; R^{46b} is hydrogen; R^{46c} is alkyl (e.g., 4-chlorobenzyl, 2-chloromethylpyridine, aminoalkyl, such as aminobutyl, or heteroaryl substituted alkyl, such as tetrahydropyran substituted methyl) or aryl (e.g., heteroaryl, such as pyrazole or imidizole); e is 1, f is 0; and R^{51a} is hydrogen or alkyl (e.g., methyl or benzyl).

In one embodiment, A⁴¹ is NR^{41c}; R^{41c} is hydrogen; R^{42a} is aryl, for example, phenyl such as unsubstituted phenyl or phenyl substituted at the 2-, 3- or 4-position with one or more of halogen (e.g., fluorine or chlorine), alkoxy (e.g., methoxy), aryloxy, amino, nitro, cyano, alkyl, carboxy or carbonyl; R^{43a} and R^{43b} are each hydrogen; A⁴⁴ is O; R^{46a} is OR^{46d}; R^{46d} is alkyl (e.g., t-butyl); e is 1, f is 0; and R^{51a} is hydrogen.

In one embodiment, A⁴¹ is NR^{41c}; R^{41c} is hydrogen; R^{42a} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{43a} and R^{43b} are each hydrogen; A⁴⁴ is NR^{44c}; R^{44c} is alkyl (e.g., methyl); R^{46a} is NR^{46b}R^{46c}; R^{46b} is hydrogen; R^{46c} is alkyl (e.g., 4-chlorobenzyl) e is 1, f is 0; and R^{51a} is hydrogen.

In one embodiment, A⁴¹ is O; R^{42a} is hydrogen, alkyl (e.g., methyl) or aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{43a} is hydrogen; R^{43b} is hydrogen or alkyl (e.g., methyl); or R^{43b} and R^{44c} are linked to form a 5-membered heterocyclic ring (e.g., pyrrolidine or hydroxyl-substituted pyrrolidine); A⁴⁴ is NR^{44c}; R^{44c} is hydrogen or alkyl (e.g., methyl or benzyl); R^{46a} is NR^{46b}R^{46c}; R^{46b} is hydrogen or alkyl (e.g., methyl or ethyl); R^{46c} is alkyl (e.g., diphenylmethyl, ethyl, 4-chlorobenzyl, 4-methoxybenzyl, 4-isopropylbenzyl, benzyl, 4-phenylbenzyl, 3-chlorobenzyl, 4-chloroethylbenzyl, 4-chloro-3-trifluoromethylbenzyl, trifluorobutane, heteroaryl substituted alkyl, such as tetrahydropyran substituted methyl, alkoxy substituted alkyl, such as ethoxyethyl, aminoalkyl, such as diethylaminopropyl, heterocyclic substituted alkyl, such as morpholine, n-methylpiperidine and n-butylpiperidine substituted methyl) or aryl (e.g., heteroaryl, such as pyrazole or imidizole) or aryl (e.g., 4-chlorophenyl) or R^{46b} and R^{46c} are linked to form a heterocyclic ring (e.g., piperidine, morpholine, N-phenylpiperazine, N-methylpiperazine, N-pyrimidinylpiperazine); e is 1, f is 0 or 1; and R^{51a} is hydrogen, amino (unsubstituted amino or amino substituted with alkyl, for example, isopropyl), hydroxyl or alkyl (e.g., methyl).

In one embodiment, A⁴¹ is O; R^{42a} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; R^{43a} and R^{43b} are each hydrogen; A⁴⁴ is NR^{44c}; R^{44c} is hydrogen; R^{46a} is OR^{46d}; R^{46d} is hydrogen; e is 1, f is 0; and R^{51a} is hydrogen.

Formula XIIa:

In one embodiment, the compound is formula XII is a compound of formula XIIa:

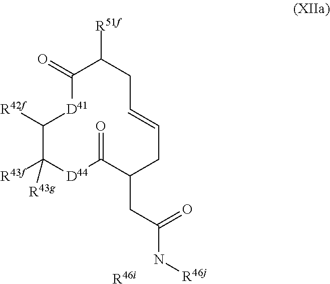

wherein

D⁴¹ is O or NR^{41h};

D⁴⁴ is O or NR^{44h};

R^{42f}, R^{43f}, R^{43g} and R^{51f} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether; or

R^{43h} and R^{44h} are linked to form a 3 to 10-membered heterocyclic or aryl ring;

R^{41h} and R^{44h} are each independently hydrogen or alkyl; and

R⁴⁶ⁱ and R^{46j} are each independently hydrogen, hydroxy, alkoxy, alkyl, alkenyl, alkynyl, aryl, carbonyl, carboxy, acyl, amino or a carbocyclic or heterocyclic moiety, or R⁴⁶ⁱ and R^{46j} are linked to form a 3 to 10-membered heterocyclic or aryl ring; and pharmaceutically acceptable salts thereof,

provided that when D⁴¹ is O; R^{42f} is unsubstituted phenyl; D⁴⁴ is NR^{44h}; R^{43f}, R^{43g}, R^{44h}, R⁴⁶ⁱ and R^{51f} are each hydrogen, then R^{46j} is not

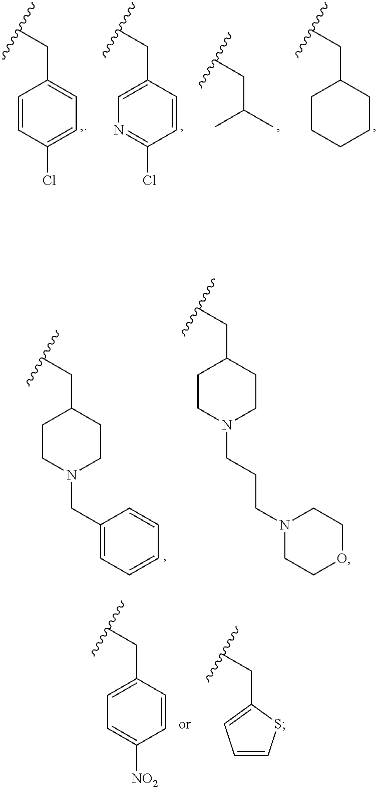

provided that when D⁴¹ is O; R^{42f} is unsubstituted phenyl; D⁴⁴ is NR^{44h}; R^{44h} is methyl; R^{43f}, R^{43g}, R⁴⁶ⁱ and R^{51f} are each hydrogen, then R^{46j} is not

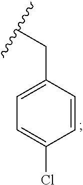 [SMILES: *Cc1ccc(Cl)cc1]

and

provided that when D⁴¹ is NR^{41h}; R^{41h} is hydrogen; R^{42f} is unsubstituted phenyl; D⁴⁴ is O; R^{43f}, R^{43g}, R⁴⁶ⁱ and R^{51f} are each hydrogen, then R^{46j} is not

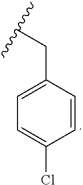 [SMILES: *Cc1ccc(Cl)cc1]

In one embodiment, D⁴¹ is NR^{41h}; R^{41h} is hydrogen; R^{42f} is aryl (e.g., phenyl); R^{43f} and R^{43g} are each hydrogen; D⁴⁴ is R^{44h}; R^{44h} is alkyl (e.g., methyl); or R^{43g} and R^{44h} are linked to form a 5-membered heterocyclic ring (e.g., pyrrolidine); R⁴⁶ⁱ is hydrogen; R^{46j} is alkyl (e.g., 4-chlorobenzyl); and R^{51f} is hydrogen or alkyl (e.g., methyl).

In one embodiment, D⁴¹ is NR^{41h}; R^{41h} is hydrogen or alkyl (e.g., methyl); R^{42f} is aryl, for example, phenyl (e.g., unsubstituted phenyl or phenyl substituted with halogen, such as fluorine or chlorine, or alkoxy, such as methoxy) or alkyl (e.g., cycloalkyl, isopropyl, N-butyl or benzyl); D⁴⁴ is O; R⁴⁶ⁱ is hydrogen; R^{46j} is alkyl (e.g., 4-chlorobenzyl, -2-chloropyridine substituted methyl; aminobutyl) or aryl (e.g., heteroaryl such as methylimidizole or pyrazole) and R^{51f} is hydrogen or alkyl (e.g., methyl or benzyl).

In one embodiment, D⁴¹ is O; R^{42f} is hydrogen, alkyl (e.g., methyl) or aryl, (e.g., phenyl); R^{43f} is hydrogen, R^{43g} is hydrogen or alkyl (e.g., methyl); D⁴⁴ is NR^{44h}, R^{44h} is hydrogen or alkyl (e.g., methyl or benzyl); or R^{43g} and R^{44h} are linked to form a 5-membered heterocyclic ring (e.g., pyrrolidine or hydroxyl substituted pyrrolidine); R⁴⁶ is hydrogen or alkyl (e.g., methyl or ethyl); R^{46j} is a carbocyclic ring (e.g., tetrahydronaphthlene), aryl (e.g., 4-chlorophenyl), alkyl (e.g., benzyl, trifluoropropyl, 4-chlorobenzyl, N-methylpiperidine substituted methyl, ethyl, 4-nitrobenzyl, ethoxyethyl, tetrahydropyran substituted methyl, diethylaminopropyl, N-butylpiperidine substituted methyl, 4-isopropylbenzyl, 3-chlorobenzyl, 4-chloro-3-trifluoromethyl substituted benzyl, 4-chloroethylbenzyl, 4-methoxybenzyl, diphenylmethyl, 4-phenylbenzyl, 2-chloropyridine substituted methyl) or R⁴⁶ⁱ and R^{46j} are linked to form a heterocyclic ring (e.g., piperidene, morpholine, N-phenylpiperazine, N-methylpiperazine, N-pyrimidinylpiperazine) or an aryl ring; and R^{51f} is hydrogen, hydroxyl, alkyl (e.g., methyl) or amino.

Formula XIIb:

In one embodiment, the compound of formula XII is a compound of formula XIIb:

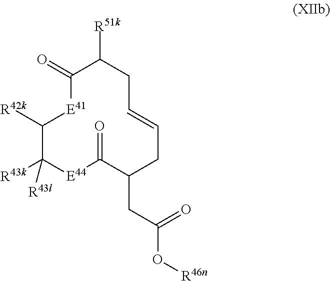

wherein

E⁴¹ is O or NR^{41m};

E⁴⁴ is O or NR^{44m};

R^{42k}, R^{43k}, R^{43l} and R^{51k} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether; or

R^{42k} and R^{43k} are linked to form a 3- to 10-membered carbocyclic, heterocyclic or aryl ring;

R⁴⁶ⁿ is hydrogen, alkyl, alkenyl, alkynyl, aryl, amino, carbonyl or a heterocyclic moiety; or a pharmaceutically acceptable salt thereof;

provided that when E⁴¹ is O; R^{42k} is unsubstituted phenyl; E⁴⁴ is NR^{44m}; R^{44m} is hydrogen or methyl and R^{51k} is hydrogen, then R⁴⁶ⁿ is not

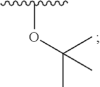 [SMILES: *OC(C)(C)C]

and

provided that when E⁴¹ is NR⁴¹ⁿ; R^{41m} is hydrogen; R^{42k} is unsubstituted phenyl; E⁴⁴ is O and R^{51k} is hydrogen, then R⁴⁶ⁿ is not

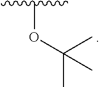 [SMILES: *OC(C)(C)C]

In one embodiment E⁴¹ is O, R^{42k} is aryl (e.g., phenyl), R^{43k} and R^{43l} are each hydrogen; E⁴⁴ is NR⁴⁴, 44 is hydrogen; R⁴⁶ⁿ is hydrogen and R^{51k} is hydrogen.

In one embodiment, E⁴¹ is NR^{41m}; R^{41m} is hydrogen; R^{42k} is aryl (e.g., phenyl, such as unsubstituted phenyl, halogen substituted phenyl (e.g., 4-chlorophenyl or 4-fluorophenyl) or alkoxy substituted phenyl (e.g., 2-methoxyphenyl); R^{43k} is hydrogen;

R^{44l} is hydrogen or R^{42k} and R^{43k} are linked to form carbocyclic ring (e.g., dihydroindene); R⁴⁶ⁿ is alkyl (e.g., t-butyl) and R^{51k} is hydrogen.

Formula XIII:

In one embodiment, the invention provides compounds of formula XIII:

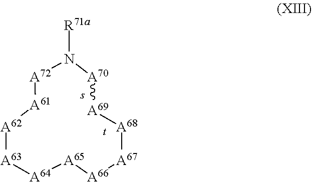

wherein

A⁶¹ is CR^{61a}R^{61b}; O, C═O or NR^{61c};

A⁶² is CR^{62a}R^{62b}, O, NR^{62c} or C═O;

A⁶³ is (CR^{63a}R^{63b} k-(CR^{63c}R^{63d});

A⁶⁴ is CR^{64a}R^{64b}, O, C═O or NR⁶⁴;

A⁶⁵ is CR^{65a}R^{65b}, O, C═O, SO₂ or NR^{65c};

A⁶⁶ is CR^{66a}R^{66b}, O, C═O or NR^{66c};

A⁶³ is (CR^{63a}R^{63b})ₖ—(CR^{63c}R^{63d})ₗ;

A⁶⁸ is CR^{68a}R^{68b}, C═O or C═NOR^{68c};

A⁶⁷ is (CR^{67a}R^{67b})ᵤ—(CR^{67c}R^{67d})ᵥ;

A⁶⁹ is CR^{69a}R^{69b}, C═O or C═NOR^{69c};

A⁷⁰ is (CR^{70a}R^{70b})ₘ—(CR^{70c}R^{70d})ₙ or NR^{70e};

A⁷² is CR^{72a}R^{72b}, O, C═O or NR^{72c};

k, l, m, n u and v are each independently 0 or 1;

t is a single bond when R^{68a} and R^{69a} are present or a double bond when R^{68a} and R^{69a} are absent;

s is a single bond when R^{69a} and R^{70a} are present or a double bond when R^{69a} and R^{70a} are absent; provided that both t and s are not both double bonds;

t is cis or trans to bond s when t is a single bond and s is a double bond;

s is cis or trans to bond t when s is a single bond and t is a double bond;

R^{61a}, R^{61b}, R^{62a}, R^{62b}, R^{64a}, R^{64b}, R^{65a}, R^{65b}, R^{66a}, R^{66b}, R^{67a}; R^{67b}, R^{67c}, R^{67d}, R^{68b} R^{69b}, R^{72a} and R^{72b} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{63a}, R^{63b}, R^{63c}, R^{63d}, R^{68a}, R^{69a}, R^{70a}, R^{70b}, R^{70c} and R^{70d} are each independently absent or hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{61c}, R^{62c}, R^{64c}, R^{65c}, R^{66c}, R^{68c}, R^{69c}, R^{70e}, R^{71c} and R^{72c} are each independently hydrogen, hydroxy, alkoxy, alkyl, alkenyl, alkynyl, aryl, carbonyl, carboxy, acyl or amino; or

R^{61c} and R^{62a}, R^{62a} and R^{63a}, R^{63a} and R^{64c}, or R^{68b} and R^{69b}, or R^{68b} and R^{70e}, together with the atoms to which they are attached, are linked to form a 3 to 10-membered carbocyclic, heterocyclic or aryl ring, and pharmaceutically acceptable salts thereof.

Formula XIV:

In one embodiment, the invention provides compounds of formula XIV:

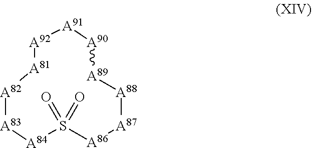

wherein

A⁸¹ is CR^{81a}R^{81b}; O, C═O or NR^{81c};

A⁸² is CR^{82a}R^{82b}, O, NR^{82c} or C═O;

A⁸³ is (CR^{83a}R^{83b})ₒ—(CR^{83c}R^{83d})ₚ;

A⁸⁴ is CR^{84a}R^{84b}, O, C═O or NR^{84c};

A⁸⁶ is CR^{86a}R^{86b}, O, C═O or NR^{86c};

A⁸⁷ is (CR^{87a}R^{87b})_{w}—(CR^{87c}R^{87d})ₓ;

A⁸⁸ is CR^{88a}R^{88b}, C═O or C═NOR^{88c};

A⁸⁹ is CR^{89a}R^{89b}, C═O or C═NOR^{89c};

A⁹⁰ is (CR^{9a}R^{9b})_{q}—(CR^{90c}R^{90d})ᵣ or NR^{90e};

A⁹¹ is CR^{91a}R^{91b}, O, C═O or NR^{91c};

A⁹² is CR^{92a}R^{92b}, O, C═O or NR^{92c};

o, p, q, r, w and x are each independently 0 or 1;

m is a single bond when R^{88a} and R^{89a} are present or a double bond when R^{88a} and R^{89a} are absent;

n is a single bond when R^{89a} and R^{80s} are present or a double bond when R^{89a} and R^{80a} are absent; provided that both t and s are not both double bonds;

m is cis or trans to bond n when m is a single bond and n is a double bond;

n is cis or trans to bond m when n is a single bond and m is a double bond;

R^{81a}, R^{81b}, R^{82a}, R^{82b}, R^{84a}, R^{84b}, R^{86a}, R^{86b}, R^{87a}; R^{87b}, R^{87c}, R^{87d}, R^{88b}, R^{89b}, R^{91a}, R^{91b}, R^{92a} and R^{92b} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{83a}, R^{83b}, R^{83c}, R^{83d}, R^{88a}, R^{89a}, R^{90a}, R^{90b}, R^{90c} and R^{90d} are each independently absent or hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;

R^{81c}, R^{82c}, R^{84c}, R^{86c}, R^{88c}, R^{89c}, R^{90e}, R^{91c} and R^{92c} are each independently hydrogen, hydroxy, alkoxy, alkyl, alkenyl, alkynyl, aryl, carbonyl, carboxy, acyl or amino; or

R^{91c} and R^{92a}, or R^{92a} and R^{93a}, or R^{93a} and R^{94c}, or R^{98b} and R^{99b}, or R^{98b} and R^{90e}, together with the atoms to which they are attached, are linked to form a 3 to 10-membered carbocyclic, heterocyclic or aryl ring, and pharmaceutically acceptable salts thereof.

In one embodiment A⁸¹ is O; A⁸² is CR^{82a}R^{82b}, R^{82a} is hydrogen; R^{82b} is aryl, for example, phenyl such as unsubstituted phenyl or phenyl substituted at the 2-, 3- or 4-position with one or more of halogen, alkoxy, aryloxy, amino, nitro, cyano, alkyl, carboxy or carbonyl; A^{83a} is A⁸³ is (CR^{83a}R^{83b})ₒ—(CR^{83c}R^{83d})ₚ; o is 1 and p is 0; R^{83a} and R^{83b} are each hydrogen; R^{83c} and R^{83d} are each absent; A⁸⁴ is NR^{84c}; R^{84c} is hydrogen; A⁸⁶ is CR^{86a}R^{86b}; R^{86a} is hydrogen; R^{86b} is alkyl (e.g., methyl); A⁸⁷ is (CR^{87a}R^{87b})_{w}—(CR^{87c}R^{87d})ₓ; w is 1 and x is 0; R^{87a} and R^{87b} are each hydrogen; A⁸⁸ is CR^{88a}R^{88b}, m is a double bond, n is a single bond, n is trans to m; R^{88a} is absent; R^{88b} is hydrogen; A⁸⁹ is CR^{89a}R^{89b}; R^{89a} is absent; R^{89b} is hydrogen; A⁹⁰ is (CR^{90a}R^{90b})_{q}—(CR^{90c}R^{90d})ᵣ; q is 1 and r is 0; R^{90a} and R^{90b} are each hydrogen; R^{90c} and R^{90d} are each absent; A⁹¹ is CR^{91a}R^{91b}; R^{91a} and R^{91b} are each hydrogen and A⁹² is C═O.

In at least one embodiment, the compounds of the invention are not a compound disclosed in International Application Publication No. WO 2009/132032. In another embodiment, the compounds of the invention are not:

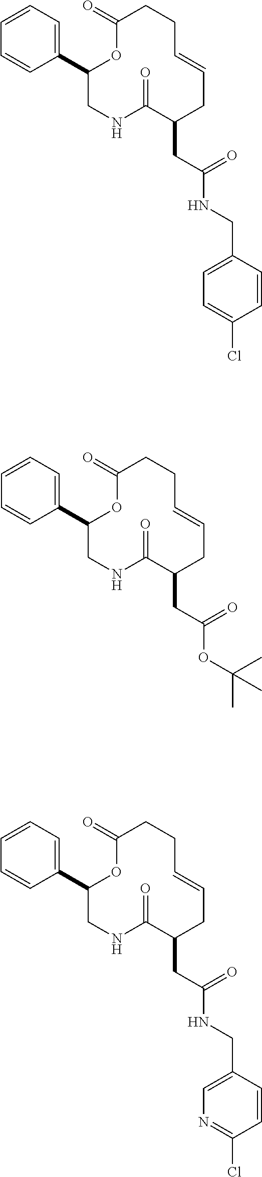

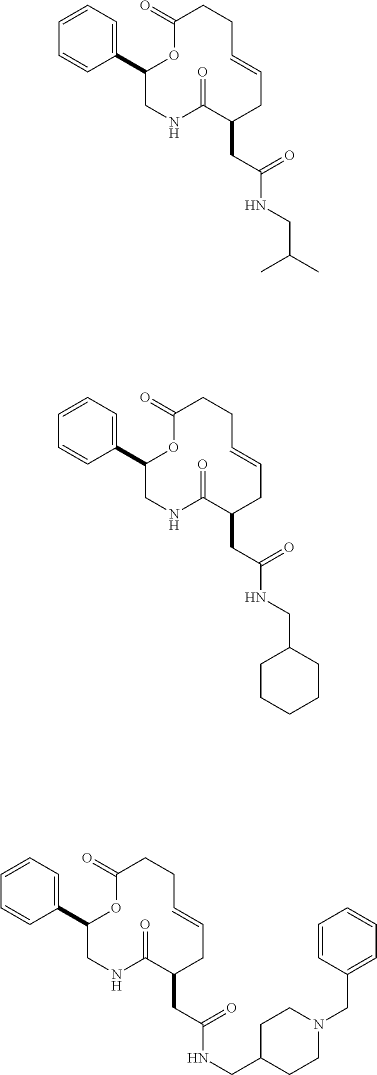

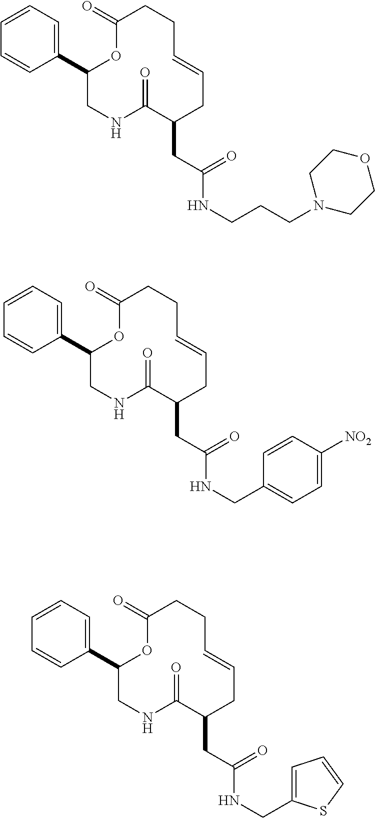

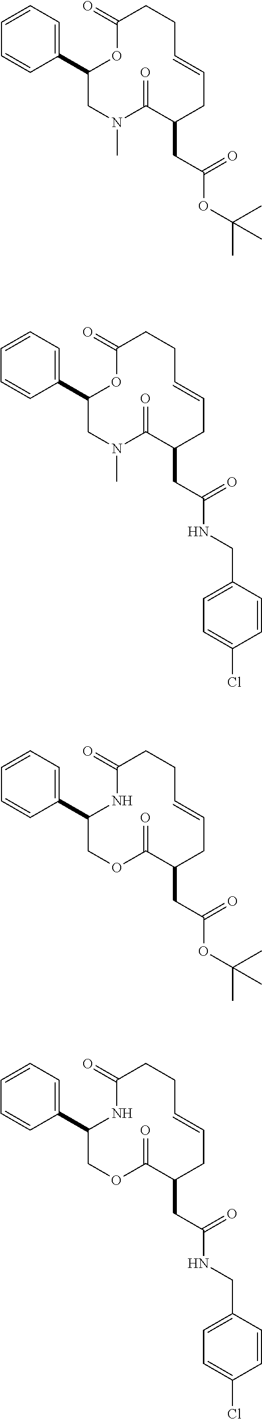

In another embodiment, the compounds of the invention are not:
N-(4-chlorobenzyl)-2-((2R,6S,E)-5,12-dioxo-2-phenyl-1-oxa-4-azacyclododec-8-en-6-yl)acetamide (robotnikinin);
tert-butyl 2-((2R,6S,E)-5,12-dioxo-2-phenyl-1-oxa-4-azacyclododec-8-en-6-yl)acetate;
N-((6-chloropyridin-3-yl)methyl)-2-((2R,6S,E)-5,12-dioxo-2-phenyl-1-oxa-4-azacyclododec-8-en-6-yl)acetamide;
2-((2R,6S,E)-5,12-dioxo-2-phenyl-10oxa-4-azacyclododec-8-en-6-yl)-N-isobutylacetamide;
N-(cyclohexylmethyl)-2-((2R,6S,E)-5,12-dioxo-2-phenyl-1-oxa-4-azacyclododec-8-en-6-yl)acetamide;
N-((1-benzylpiperidin-4-yl)methyl)-2-((2R,6S,E)-5,12-dioxo-2-phenyl-1-oxa-4-azacyclododec-8-en-6-yl)acetamide;
2-((2R,6S,E)-5,12-dioxo-2-phenyl-1-oxa-4-azacyclododec-8-en-6-yl)-N-(3-morpholinopropyl)acetamide;
2-((2R,6S,E)-5,12-dioxo-2-phenyl-1-oxa-4-azacyclododec-8-en-6-yl)-N-(4-nitrobenzyl)acetamide;
2-((2R,6S,E)-5,12-dioxo-2-phenyl-1-oxa-4-azacyclododec-8-en-6-yl)-N-(thiophen-2-ylmethyl)acetamide;
tert-butyl 2-(2R,6S,E)-4-methyl-5,12-dioxo-2-phenyl-1-oxa-4-azacyclododec-8-en-6-yl)acetate;
N-(4-chlorobenzyl)-2-((2R,6S,E)-4-methyl-5,12-dioxo-2-phenyl-1-oxa-4-azacyclodode-8en-6-yl)acetamide;
tert-butyl 2((3R,11S,E)-5,12-dioxo-3-phenyl-1-oxa-4-azacyclododec-8-en-11-yl)acetate; and
N-(4-chlorobenzyl)-2-((3R,11S,E)-5,12-dioxo-3-phenyl-1-oxa-4-azacyclododec-8-en-11-yl)acetamide.

Definitions of specific functional groups and chemical terms are described in more detail below. For purposes of this invention, the chemical elements are identified in accordance with the Periodic Table of the Elements, CAS version, Handbook of Chemistry and Physics, 75″ Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference.

The term aliphatic, as used herein, includes both saturated and unsaturated, straight chain (i.e., unbranched), branched, acyclic, cyclic, or polycyclic aliphatic hydrocarbons, which are optionally substituted with one or more functional groups. As will be appreciated by one of ordinary skill in the art, “aliphatic” is intended herein to include, but is not limited to, alkyl, alkenyl, alkynyl, cycloalkyl, cycloalkenyl, and cycloalkynyl moieties.

The term “alkyl” includes saturated aliphatic groups, including straight-chain alkyl groups (e.g., methyl, ethyl, propyl, butyl, pentyl, hexyl, heptyl, octyl, nonyl, decyl, etc.), branched-chain alkyl groups (isopropyl, tert-butyl, isobutyl, etc.), cycloalkyl or carbocyclic (alicyclic) groups (cyclopropyl, cyclobutyl, cyclopentyl, cyclohexyl, cycloheptyl, cyclooctyl), alkyl substituted cycloalkyl groups, and cycloalkyl substituted alkyl groups. The term alkyl further includes alkyl groups that may include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain). Likewise, cycloalkyls may have from 3-8 carbon atoms in their ring structure. The term “C₁-C₆” includes alkyl groups containing 1 to 6 carbon atoms.

Moreover, the term alkyl includes both “unsubstituted alkyls” and “substituted alkyls,” the latter of which refers to alkyl moieties having substituents replacing a hydrogen on one or more carbons of the hydrocarbon backbone. Such substituents can include, for example, alkyl, alkenyl, alkynyl, halogen, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonate, sulfamoyl, sulfonamido, nitro, trifluoromethyl, cyano, azido, heterocyclyl, alkylaryl, or an aromatic or heteroaromatic moiety. Cycloalkyls can be further substituted, e.g., with the substituents described above. An “alkylaryl” or an “arylalkyl” moiety is an alkyl substituted with an aryl (e.g., phenylmethyl (benzyl)). The term “alkyl” also includes the side chains of natural and unnatural amino acids.

The term “aryl” includes groups, e.g., 5- and 6-membered single-ring aromatic groups, that may include from zero to four heteroatoms, for example, benzene, phenyl, pyrrole, furan, thiophene, thiazole, isothiaozole, imidazole, triazole, tetrazole, pyrazole, oxazole, isooxazole, pyridine, pyrazine, pyridazine, and pyrimidine, and the like. Furthermore, the term “aryl” includes multicyclic aryl groups, e.g., tricyclic, bicyclic, e.g., naphthalene, benzoxazole, benzodioxazole, benzothiazole, benzoimidazole, benzothiophene, methylenedioxyphenyl, quinoline, isoquinoline, napthridine, indole, benzofuran, purine, benzofuran, deazapurine, or indolizine. Those aryl groups having heteroatoms in the ring structure may also be referred to as “aryl heterocycles,” “heteroaryls” or “heteroaromatics.” The aromatic ring can be substituted at one or more ring positions with such substituents as described above, as for example, alkyl, alkenyl, alkynyl, halogen, hydroxyl, alkoxy, aryl, alkylcarbonyloxy, arylcarbonyloxy, alkoxycarbonyloxy, aryloxycarbonyloxy, —COOH, alkylcarbonyl, alkylaminoacarbonyl, arylalkyl aminocarbonyl, alkenylaminocarbonyl, alkylcarbonyl, arylcarbonyl, arylalkylcarbonyl, alkenylcarbonyl, alkoxycarbonyl, aminocarbonyl, alkylthiocarbonyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety. Aryl groups can also be fused or bridged with alicyclic or heterocyclic rings which are not aromatic so as to form a polycycle (e.g., tetralin). The term heteroaryl includes unsaturated cyclic compounds such as azirine, oxirene, dithiete, pyrroline, pyrrole, furan, dihydrofuran, dihydrothiophene, thiophene, pyrazole, imidazole, oxazole, thiazole, isothiazole, 12,2,3-triazole, 1,2,4, triazole, dithiazole, tetrazole, pyridine, pyran, pyrimidine, pyran, thiapyrane, diazine, thiazine, dioxine, triazine and tetrazene.

The term heteroaliphatic, as used herein, refers to aliphatic moieties that contain one or more oxygen, sulfur, nitrogen, phosphorus, or silicon atoms, e.g., in place of carbon atoms. Heteroaliphatic moieties may be branched, unbranched, cyclic or acyclic and include saturated and unsaturated heterocycles such as morpholino, pyrrolidinyl, etc. In certain embodiments, heteroaliphatic moieties are substituted by independent replacement of one or more of the hydrogen atoms thereon with one or more moieties including, but not limited to aliphatic; heteroaliphatic; aryl; heteroaryl; arylalkyl; heteroarylalkyl; alkoxy; aryloxy; heteroalkoxy; heteroaryloxy; alkylthio; arylthio; heteroalkylthio; —F; —Cl; —Br; —I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; N(Rₓ)₂; —S(O)₂Rₓ; —NRₓ(CO)Rₓ, wherein each occurrence of Rₓ, independently includes, but is not limited to, aliphatic, heteroaliphatic, aryl, heteroaryl, arylalkyl, or heteroarylalkyl, wherein any of the aliphatic, heteroaliphatic, arylalkyl, or heteroarylalkyl substituents described above and herein may be substituted or unsubstituted, branched or unbranched, cyclic or acyclic, and wherein any of the aryl or heteroaryl substituents described above and herein may be substituted or unsubstituted.

The term “heterocyclic moiety” includes saturated cyclic (e.g., monocyclic, bicyclic and tricyclic) moieties having a closed ring of atoms in which at least one atom is not a carbon. As used herein, heterocyclic moieties do not include heteroaryl moieties, in which the closed ring of atoms is both heterocyclic and aromatic and/or unsaturated. Examples of heterocyclic moieties include aziridine, ethylene oxide, thiirane, dioxirane, azetidine, oxetane, thietane, dioxetane, dithietane, pyrrolidine, tetrahydrofuran, tetrahydrothiophene, imidazolidine, oxazolidine, thiazolidine, dioxolane, dithiolane, piperidine, tetrahydropyran, thiane, piperzine, pyrazine, dithiane, dioxane and trioxane.

The term “heterocyclic moiety” includes both “unsubstituted heterocyclic moieties” and “substituted heterocyclic moieties,” the latter of which includes moieties having substituents replacing a hydrogen on one or more of the atoms on the closed ring.

Such substituents can include, for example, alkyl, alkenyl, alkynyl, halogens, hydroxyl, aryl alkylcarbonyloxy, arylcarbonyloxy, alkoxycarbonyl oxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

Specific heterocyclic and heteroaryl groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(biS-(2-propenyl)amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2 furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term “alkenyl” includes unsaturated aliphatic groups analogous in length and possible substitution to the alkyls described above, but that contain at least one double bond. For example, the term “alkenyl” includes straight-chain alkenyl groups (e.g., ethylenyl, propenyl, butenyl, pentenyl, hexenyl, heptenyl, octenyl, nonenyl, decenyl, etc.), branched-chain alkenyl groups, cycloalkenyl (alicyclic) groups (cyclopropenyl, cyclobutenyl, cyclopentenyl, cyclohexenyl, cycloheptenyl, cyclooctenyl), alkyl or alkenyl substituted cycloalkenyl groups, and cycloalkyl or cycloalkenyl substituted alkenyl groups. The term “alkenyl” further includes alkenyl groups which include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ or straight chain, C₃-C₆ for branched chain). Likewise, cycloalkenyl groups may have from 3-8 carbon atoms in their ring structure. The term C₂-C₆ includes alkenyl groups containing 2 to 6 carbon atoms.

Moreover, the term “alkenyl” includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl, alkenyl, alkynyl, halogens, hydroxyl, aryl alkylcarbonyloxy, arylcarbonyloxy, alkoxycarbonyl oxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “alkynyl” includes unsaturated aliphatic groups analogous in length and possible substitution to the alkyls described above, but which contain at least one triple bond. For example, the term “alkynyl” includes straight-chain alkynyl groups (e.g., ethynyl, propynyl, butynyl, pentynyl, hexynyl, heptynyl, octynyl, nonynyl, decynyl, etc.), branched-chain alkynyl groups, and cycloalkyl or cycloalkenyl substituted alkynyl groups. The term “alkynyl” further includes alkynyl groups which include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

Moreover, the term “alkynyl” includes both “unsubstituted alkynyls” and “substituted alkynyls,” the latter of which refers to alkynyl moieties having substituents replacing a hydrogen on one or more carbons of the hydrocarbon backbone. Such substituents can include, for example, alkyl, alkenyl, alkynyl, halogens, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term unsaturated, as used herein, means that a moiety has one or more units of unsaturation.

As used herein, the term partially unsaturated refers to a ring moiety that includes at least one double or triple bond. The term “partially unsaturated” is intended to encompass rings having multiple sites of unsaturation, but is not intended to include aryl or heteroaryl moieties, as herein defined.

The term “acyl” includes compounds and moieties which contain the acyl radical (CH₃CO—). It also includes substituted acyl moieties. The term “substituted acyl” includes acyl groups where one or more of the hydrogen atoms are replaced by for example, alkyl, alkenyl, alkynyl, halogens, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moiety.

The term “acylamino” includes moieties wherein: an acyl moiety is bonded to an amino group. For example, the term includes alkylcarbonylamino, arylcarbonylamino, carbamoyl and ureido groups.

The terms “alkoxyalkyl,” “alkylaminoalkyl” and “thioalkoxyalkyl” include alkyl groups, as described above, which further include oxygen, nitrogen or sulfur atoms replacing one or more carbons of the hydrocarbon backbone, e.g., oxygen, nitrogen or sulfur atoms.

The term “alkoxy” includes substituted and unsubstituted alkyl, alkenyl, and alkynyl groups covalently linked to an oxygen atom. Examples of alkoxy groups include methoxy, ethoxy, isopropyloxy, propoxy, butoxy, and pentoxy groups.

Examples of substituted alkoxy groups include halogenated alkoxy groups. he alkoxy groups can be substituted with groups such as alkyl, alkenyl, alkynyl, halogen, hydroxyl, aryl, alkylcarbonyloxy, arylcarbonyloxy, alkoxycarbonyloxy, aryloxycarbonyloxy, —COOH, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or an aromatic or heteroaromatic moieties. Examples of halogen substituted alkoxy groups include, but are not limited to, fluoromethoxy, difluoromethoxy, trifluoromethoxy, chloromethoxy, dichloromethoxy, trichloromethoxy, etc.

The term “amine” or “amino” includes compounds where a nitrogen atom is covalently bonded to at least one carbon or heteroatom. The term includes “alkyl amino” which comprises groups and compounds wherein: the nitrogen is bound to at least one additional alkyl group. The term “dialkyl amino” includes groups wherein: the nitrogen atom is bound to at least two additional alkyl groups. The term “arylamino” and “diarylamino” include groups in which the nitrogen is bound to at least one or two aryl groups, respectively. The term “alkylarylamino,” “alkylaminoaryl” or “arylaminoalkyl” refers to an amino group which is bound to at least one alkyl group and at least one aryl group. The term “alkaminoalkyl” refers to an alkyl, alkenyl, or alkynyl group bound to a nitrogen atom which is also bound to an alkyl group.

In one embodiment, the term amino includes compounds of the formula —NR^{6d}R^{6e}, in which R^{6d} and R^{6e} are each independently hydrogen, hydroxy, alkoxy, alkyl, alkenyl, alkynyl, aryl, carbonyl, carboxy, acyl or amino.

The term “amide,” “amido” or “aminocarbonyl” includes compounds or moieties which contain a nitrogen atom which is bound to the carbon of a carbonyl or a thiocarbonyl group. The term includes “alkaminocarbonyl” or “alkylaminocarbonyl” groups which include alkyl, alkenyl, aryl or alkynyl groups bound to an amino group bound to a carbonyl group. It includes arylaminocarbonyl and arylcarbonylamino groups, which include aryl or heteroaryl moieties bound to an amino group that is bound to the carbon of a carbonyl or thiocarbonyl group. The terms “alkylaminocarbonyl,” “alkenylaminocarbonyl,” “alkynylaminocarbonyl,” “arylaminocarbonyl,” “alkylcarbonylamino,” “alkenyl carbonylamino,” “alkynylcarbonylamino,” and “arylcarbonylamino” are included in term “amide.” Amides also include urea groups (aminocarbonylamino) and carbamates (oxycarbonylamino).

The term “carbonyl” or “carboxy” includes compounds and moieties which contain a carbon connected with a double bond to an oxygen atom. The carbonyl can be further substituted with any moiety which allows the compounds of the invention to perform its intended function. For example, carbonyl moieties may be substituted with alkyls, alkenyls, alkynyls, aryls, alkoxy, aminos, etc. Examples of moieties which contain a carbonyl include aldehydes, ketones, carboxylic acids, amides, esters, anhydrides, etc. The term “carboxy” further includes the structure of —COOH and —COO—.

The term “oximyl” includes compounds and moieties that contain a carbon connected with a double bond to a nitrogen atom, which is, in turn connected to a hydroxyl or an alkoxyl group. The term “hydrazinyl” includes compounds and moieties that contain a carbon connected with a double bond to a nitrogen atom, which is, in turn, connected to an amino group.

The term “thiocarbonyl” or “thiocarboxy” includes compounds and moieties which contain a carbon connected with a double bond to a sulfur atom.

The term “ether” includes compounds or moieties which contain an oxygen bonded to two different carbon atoms or heteroatoms. For example, the term includes “alkoxyalkyl” which refers to an alkyl, alkenyl, or alkynyl group covalently bonded to an oxygen atom which is covalently bonded to another alkyl group.

The term “thioether” includes compounds and moieties which contain a sulfur atom bonded to two different carbon or hetero atoms. Examples of thioethers include, but are not limited to, alkthioalkyls, alkthioalkenyls, and alkthioalkynyls. The term “alkthioalkyls” include compounds with an alkyl, alkenyl, or alkynyl group bonded to a sulfur atom which is bonded to an alkyl group. Similarly, the term “alkthioalkenyls” and “alkthioalkynyl” refer to compounds or moieties in which an alkyl, alkenyl or alkynyl group is bonded to a sulfur atom that is covalently bonded to an alkenyl or alkynyl group, respectively.

The term “sulfonyl” includes moieties containing a sulfonyl functional group (e.g., SO₂) attached to two carbons via a covalent bond to the sulfur atom of the sulfonyl functional group.

The term “hydroxyl” or “hydroxyl” includes groups with an —OH or —O—.

The term “halogen” includes fluorine, bromine, chlorine, iodine, etc.

The term “heteroatom” includes atoms of any element other than carbon or hydrogen. Preferred heteroatoms are nitrogen, oxygen, sulfur and phosphorus.

In some embodiments, any hydrogen in the compounds of the invention may be replaced with deuterium.

Certain compounds of the present invention may exist in particular geometric or stereoisomeric forms. The present invention contemplates all such compounds, including cis- and trans-isomers, E- and Z-isomers, R- and S-enantiomers, diastereomers, (D)-isomers, (L)-isomers, (−)- and (+)-isomers, racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, chiral chromatography, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are all contemplated by the present invention. Those of ordinary skill in the art will readily appreciate that analogous ratios are contemplated for more complex isomer mixtures.

As referred to herein, the positions of the atoms in the macrocyclic ring structure of the compounds of the present invention are as follows:

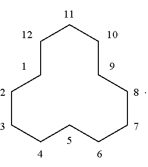 [SMILES: C1CCCCCCCCCCC1]

In some embodiments, various positions of the macrocyclic ring can have specific stereochemical configurations. For example, when the atoms at the 2-position, the 3-position, the 6-position, the 10-position and the 11-position are each carbon, they may each independently be configured in the (R)- or (S)-position. Moreover, when the atoms at the 8- and 9-position are carbon and the bond between the 8 and 9 position is a double bond, then the bonds between the 7 and 8 position and the 9 and 10 position may independently be cis or trans to the double bond. In yet another embodiment, when the bond between the 8 and 9 position is a single bond, the carbon atoms at the 8 and 9 position may independently be configured in the (R)- or (S)-position.

Without being bound by theory, the compounds of the invention are thought to bind directly to the active, N-terminal fragment of the Sonic Hedgehog protein (ShhN). This activity has been observed in the Shh-LIGHT2 reporter cell line when stimulated with ShhN-conditioned medium (HCM), but it is not observed under the same conditions when a constitutively active Cell line is used that lacks both alleles of the ShhN receptor Patchedl (Ptcl). These findings are consistent with compounds of the invention inhibiting Shh signaling in Shh-LIGHT2 cells by preventing ShhN from forming a functional complex with Ptc1.

While not wishing to be bound by any particular theory, it is proposed that compounds of the invention inhibit Shh signaling in a concentration-dependant manner without exhibiting inhibitory activity in a cell line lacking the Ptcl receptor, and without exhibiting an inhibitory effect in the presence of the well-characterized Smo agonist, purmorphamine. In light of the ShhN-binding properties of such macrocycles and the results of our epistasis analyses (its lack of significant Shh pathway inhibition, using Gli activity as a surrogate for pathway activity, in the Ptc1 cell line and the ability of an agonist of the downstream Smoothened to override its effects), it is a possibility that the mechanism of action involves direct targeting of the ShhN protein complex. Recent evidence has indicated that Hh signaling is facilitated by HhN binding partners Ihog, Boi, and heparin in Drosophila, and Shh binding partners (Ihog orthologs) Cdo and Boc in vertebrates (Zhang, F. et al. J. Biochem. 46, 3933-3941, 2007; Yao, S., Lum., L., Beachy, P. A. Cell 125, 343-357, 2006; Wilson, C. W., Chuang, P.-T. Cell 125, 435-438, 2006; Tenzen, T. et al. Dev. Cell 10, 647-656, 2006; Maity, T., Fuse, N., Beachy, P. A. PNAS 102, 17026-17031, 2005). The compounds of the invention may interfere with the ability of the ShhN protein complex to relay its signal efficiently to Ptcl in the Shh-LIGHT2 cell line. Pathway inhibition at the Shh level illustrates an opportunity for the development of probes and therapeutics through which one may gain a better understanding of diseases associated with aberrant Shh-pathway activity.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

In other cases, the compounds of the present invention may contain one or more acidic functional groups and, thus, are capable of forming pharmaceutically-acceptable salts with pharmaceutically-acceptable bases. The term “pharmaceutically-acceptable salts” in these instances refers to the relatively non-toxic, inorganic and organic base addition salts of compounds of the present invention. These salts can likewise be prepared in situ in the administration vehicle or the dosage form manufacturing process, or by separately reacting the purified compound in its free acid form with a suitable base, such as the hydroxide, carbonate or bicarbonate of a pharmaceutically-acceptable metal cation, with ammonia, or with a pharmaceutically-acceptable organic primary, secondary, tertiary, or quaternary amine. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Representative organic amines useful for the formation of base addition salts include ethylamine, diethylamine, ethylenediamine, ethanolamine, diethanolamine, piperazine and the like. (See, for example, Berge et al., supra).

Compounds of the present invention may be combined with a pharmaceutically acceptable carrier to form a pharmaceutical composition. In certain embodiments, the pharmaceutical composition includes a pharmaceutically acceptable amount of a compound of the invention. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, this amount will range from about 1% to about 99% of active ingredient, from about 5% to about 70%, or from about 10% to about 30%.

In some embodiments, the pharmaceutical compositions comprises an amount of a compound of the invention effective to treat a Sonic Hedgehog associated disorder.

Wetting agents, emulsifiers, and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, alpha-tocopherol, and the like; and metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. In certain embodiments, a formulation of the present invention comprises an excipient selected from the group consisting of cyclodextrins, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically-acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, potato or tapioca starch, alginic acid, certain silicates, and sodium carbonate; solution retarding agents, such as paraffin; absorption accelerators, such as quaternary ammonium compounds; wetting agents, such as, for example, acetyl alcohol, glycerol monostearate, and non-ionic surfactants; absorbents, such as kaolin and bentonite clay; lubricants, such as talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made in a suitable machine in which a mixture of the powdered compound is moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Dissolving or dispersing the compound in the proper medium can make such dosage forms. Absorption enhancers can also be used to increase the flux of the compound across the skin. Either providing a rate controlling membrane or dispersing the compound in a polymer matrix or gel can control the rate of such flux.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and non-aqueous carriers, which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions, which are compatible with body tissue.

In certain embodiments, a compound or pharmaceutical preparation is administered orally. In other embodiments, the compound or pharmaceutical preparation is administered intravenously. Alternative routs of administration include sublingual, intramuscular, and transdermal administrations.

When the compounds of the present invention are administered as pharmaceuticals, to humans and animals, they can be given per se or as a pharmaceutical composition containing, for example, 0.1% to 99.5%, or 0.5% to 90%, of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

The language “effective amount” includes that amount of a compound of the invention that is effective to treat a subject suffering from or at risk of suffering from a Sonic Hedgehog associated disorder. Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required to achieve the desired therapeutic effect and then gradually increasing the dosage until the desired effect is achieved.

In some embodiments, a compound or pharmaceutical composition of the invention is provided to a subject chronically. Chronic treatments include any form of repeated administration for an extended period of time, such as repeated administrations for one or more months, between a month and a year, one or more years, or longer. In many embodiments, a chronic treatment involves administering a compound or pharmaceutical composition of the invention repeatedly over the life of the subject. Preferred chronic treatments involve regular administrations, for example one or more times a day, one or more times a week, or one or more times a month. In general, a suitable dose such as a daily dose of a compound of the invention will be that amount of the compound that is the lowest dose effective to produce a therapeutic effect. Such an effective dose will generally depend upon the factors described above. Generally doses of the compounds of this invention for a patient, when used for the indicated effects, will range from about 0.0001 to about 100 mg per kg of body weight per day. Preferably the daily dosage will range from 0.001 to 50 mg of compound per kg of body weight, and even more preferably from 0.01 to 10 mg of compound per kg of body weight. However, lower or higher doses can be used. In some embodiments, the dose administered to a subject may be

modified as the physiology of the subject changes due to age, disease progression, weight, or other factors.

If desired, the effective daily dose of the active compound may be administered as two, three, four, five, six, or more sub-doses administered separately at appropriate intervals throughout the day, optionally, in unit dosage forms.

While it is possible for a compound of the present invention to be administered alone, it is preferable to administer the compound as a pharmaceutical formulation (composition) as described above.

The compounds according to the invention may be formulated for administration in any convenient way for use in human or veterinary medicine, by analogy with other pharmaceuticals.

In certain embodiments, compounds and pharmaceutical compositions of the present invention can be employed in combination therapies, that is, the compounds and pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another anticancer agent), or they may achieve different effects (e.g., control of any adverse effects).

For example, other therapies or anticancer agents that may be used in combination with the inventive anticancer agents of the present invention include surgery, radiotherapy (y-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. Additionally, the present invention also encompasses the use of certain cytotoxic or anticancer agents currently in clinical trials and which may ultimately be approved by the FDA (including, but not limited to, epothilones and analogues thereof and geldanamycins and analogues thereof). For a more comprehensive discussion of updated cancer therapies see, www.nci.nih.gov, a list of the FDA approved oncology drugs at www.fda.gov/cder/cancer/druglistframe.htm, and The Merck Manual, Seventeenth Ed. 1999, the entire contents of which are hereby incorporated by reference.

In certain embodiments, inventive compounds are useful in treating a subject in clinical remission. In some embodiments, the subject has been treated by surgery and may have limited unresected disease.

The invention provides kits comprising pharmaceutical compositions of an inventive compound. In certain embodiments, such kits including the combination of a compound of the present invention and another chemotherapeutic agent. The agents may be packaged separately or together. The kit optionally includes instructions for prescribing the medication. In certain embodiments, the kit includes multiple doses of each agent. The kit may include sufficient quantities of each component to treat a subject for a week, two weeks, three weeks, four weeks, or multiple months. The kit may include a full cycle of chemotherapy. In certain embodiments, the kit includes multiple cycles of chemotherapy.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

### EXAMPLES

### Example 1

### Synthesis of Select Compounds of the Invention

### Synthesis of Compounds 20 and 21

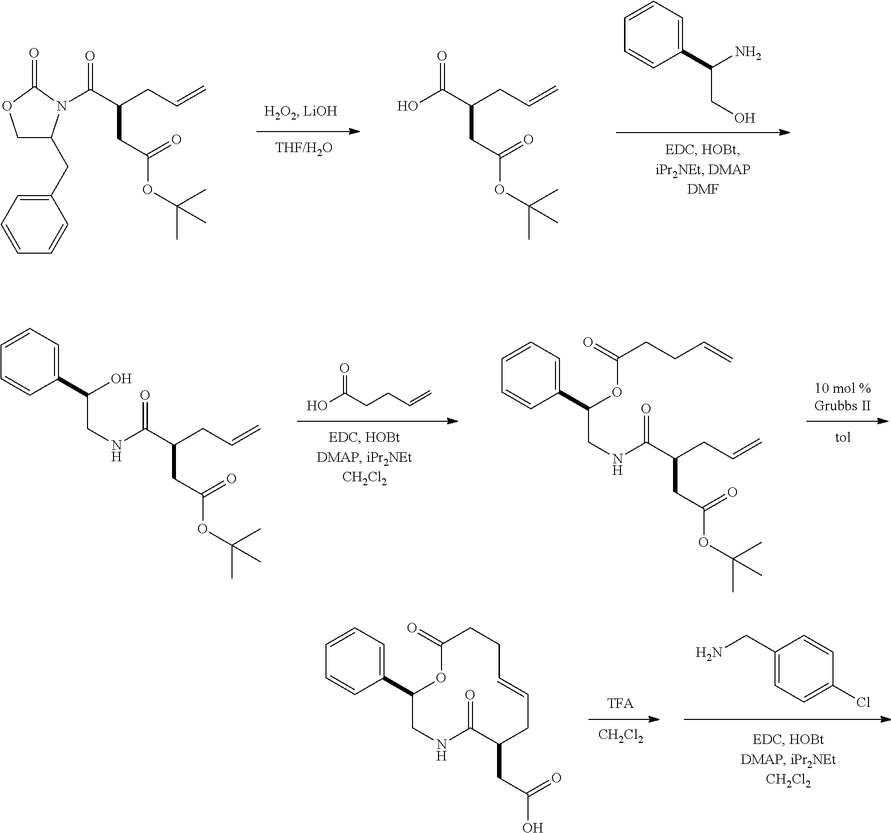

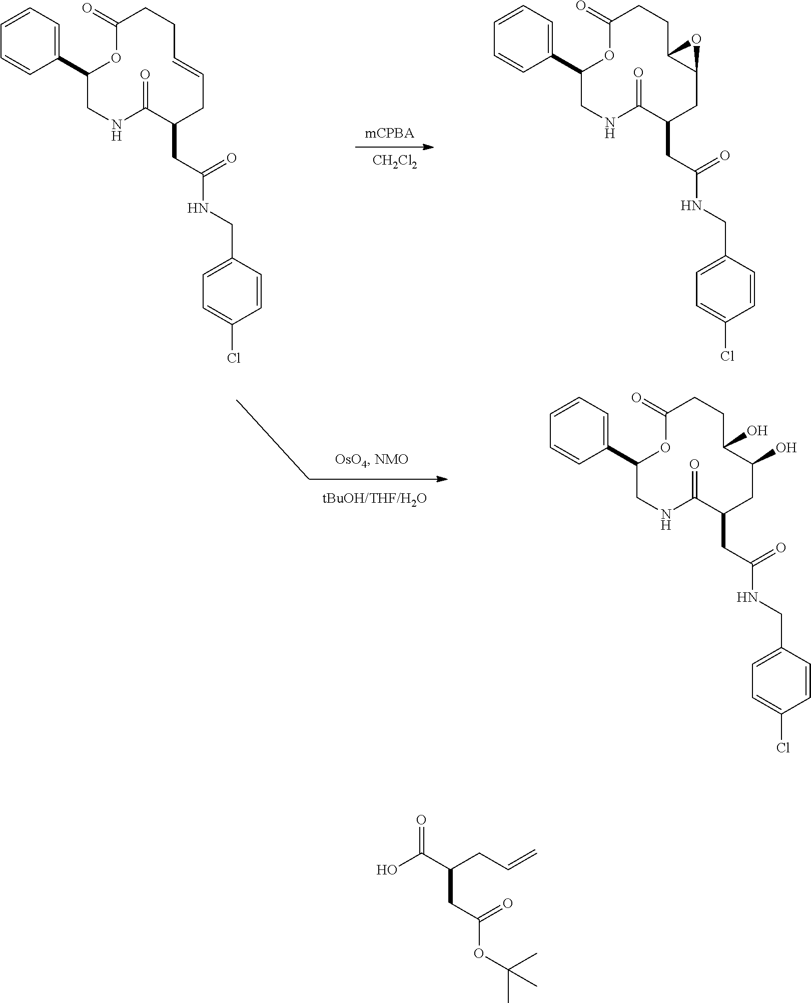

### (S)-2-(2-tert-butoxy-2-oxoethyl)pent-4-enoic acid

To a solution of (S)-tert-butyl 3-((R)-4-benzyl-2-oxooxazolidine-3-carbonyl)hex-5-enoate (22.0 g, 58.9 mmol) in THF (491 ml) and water (123 ml) cooled in an ice/water bath was added H₂O₂ (24.3 mL, 236 mmol) followed by dropwise addition of a solution of LiOH (2.82 g, 118 mmol) in water (123 ml). The reaction mixture was stirred 1 h at which point TLC analysis indicated complete consumption of starting material. The reaction mixture was diluted with 250 mL sat. Na₂SO₃ and stirred 1 h. The THF was subsequently removed by rotary evaporation and the remaining aqueous fraction was extracted 3× with CH₂Cl₂. The aqueous layer was acidified with 2N HCl and extracted 3× with CH₂Cl₂. The combined organic extracts were washed with brine, dried over Na₂SO₄, filtered, and concentrated to yield 7.7 g of product as a light yellow oil in 61% yield. LRMS (M+HCO₂H)—: 259.5.

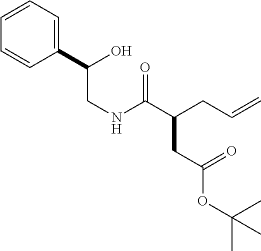

### (S)-tert-butyl 3-((R)-2-hydroxy-2-phenylethylcarbamoyl)hex-5-enoate

To a solution of (S)-2-(2-tert-butoxy-2-oxoethyl)pent-4-enoic acid (0.947 g, 4.42 mmol) in DMF (31.6 mL) cooled in an ice/water bath was added EDC (1.27 g, 6.63 mmol) and HOBt (0.896 g, 6.63 mmol) and the resulting solution was stirred 30 min. (R)-2-amino-phenylethanol (0.667 g, 4.86 mmol), iPr₂NEt (2.31 mL, 13.3 mmol) and DMAP (0.108 g, 0.884 mmol) were subsequently added and the reaction mixture was slowly warmed to rt and stirred for 16 h at which point LC-MS analysis indicated complete consumption of starting material. The reaction mixture was diluted with saturated NH₄Cl and EtOAc and the layers separated. The aqueous was extracted 2× with EtOAc and the combined organic extracts were washed with 1N HCl and brine, dried over Na₂SO₄, filtered, and concentrated. The crude product was purified using silica gel chromatography (MeOH/CH₂Cl₂ gradient) to yield 1.25 g of desired product as a light yellow oil in 85% yield. LRMS (M+Na)⁺: 356.2.

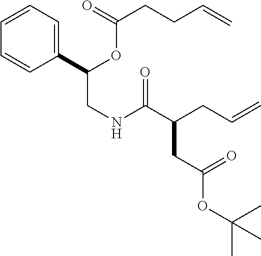

### (S)-tert-butyl 3-((R)-2-(pent-4-enoyloxy)-2-phenylethylcarbamoyl)hex-5-enoate

To a solution of 4-pentenoic acid (0.335 mL, 3.28 mmol) in DMF (31.6 mL) cooled in an ice/water bath was added EDC (630 mg, 3.28 mmol) and the resulting solution was stirred 30 min. (S)-tert-butyl 3-((R)-2-hydroxy-2-phenylethylcarbamoyl)hex-5-enoate (730 mg, 2.19 mmol), iPr₂NEt (1.14 mL, 6.57 mmol) and DMAP (26.7 mg, 0.219 mmol) were subsequently added and the reaction mixture was slowly warmed to rt and stirred for 16 h at which point LC-MS analysis indicated complete consumption of starting material. The reaction mixture was diluted with saturated NH₄Cl and EtOAc and the layers separated. The aqueous was extracted 2× with EtOAc and the combined organic extracts were washed with 1N HCl and brine, dried over Na₂SO₄, filtered, and concentrated. The crude product was purified using silica gel chromatography (MeOH/CH₂Cl₂ gradient) to yield 670 mg of desired product as a colorless oil in 74% yield. LRMS (M+Na)⁺: 430.0.

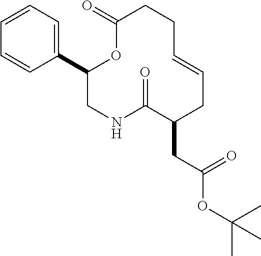

### tert-butyl 2-((2R,6S,E)-5,12-dioxo-2-phenyl-1-oxa-4-azacyclododec-8-en-6-yl)acetate

To a solution of (S)-tert-butyl 3-((R)-2-(pent-4-enoyloxy)-2-phenylethylcarbamoyl)hex-5-enoate (470 mg, 1.131 mmol) in toluene (113 mL) was added Grubbs 2nd Generation catalyst (96 mg, 0.113 mmol) and the reaction mixture was heated to 65° C. Upon stirring 7 h with continued heating, TLC analysis indicated complete consumption of starting material. The reaction mixture was cooled to rt, diluted with degassed CH₂Cl₂ (11 ml) and Pb(OAc)₄ (251 mg, 0.566 mmol) was added. The resulting mixture was stirred for 12 h at which point the mixture was passed through a short plug of silica then concentrated using rotary evaporation. The resulting purple solid was triturated with methanol to further remove ruthenium complexes. The off white solid that remained was purified using silica gel chromatography (MeOH/CH₂Cl₂ gradient) to yield 320 mg of desired product as a white solid in 73% yield. LRMS (M+Na)⁺: 410.4.

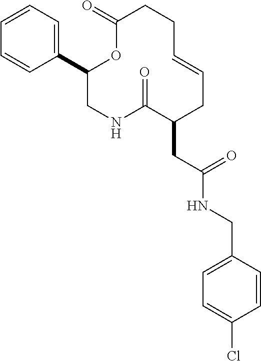

### N-(4-chlorobenzyl)-2-((2R,6S,E)-5,12-dioxo-2-phenyl-1-oxa-4-azacyclododec-8-en-6-yl)acetamide

To a solution of tert-butyl 2-((2R,6S,E)-5,12-dioxo-2-phenyl-1-oxa-4-azacyclododec-8-en-6-yl)acetate (0.140 g, 0.361 mmol) in CH₂Cl₂ (125 μL) cooled in an ice/water bath was added trifluoroacetic acid (125 μL, 1.63 mmol) and the reaction mixture was stirred 1 h at which point LC-MS analysis indicated complete consumption of starting material. The reaction mixture was concentrated and the crude carboxylic acid was utilized immediately. To a solution of crude 2-((2R,6S,E)-5,12-dioxo-2-phenyl-1-oxa-4-azacyclododec-8-en-6-yl)acetic acid (0.120 g, 0.362 mmol) in CH₂Cl₂ (4.5 mL) cooled in an ice/water bath was added EDC (0.104 g, 0.543 mmol) and HOBt (0.073 g, 0.543 mmol) and the resulting solution was stirred 30 min. 4-chlorobenzylamine (0.049 mL, 0.398 mmol), iPr₂NEt (0.194 mL, 1.086 mmol) and DMAP (4 mg, 0.44 mmol) were subsequently added and the reaction mixture was slowly warmed to rt and stirred for 16 h at which point LC-MS analysis indicated complete consumption of starting material. The reaction mixture was diluted with saturated NH₄Cl and EtOAc and the layers separated. The aqueous was extracted 2× with EtOAc and the combined organic extracts were washed with 1N HCl and brine, dried over Na₂SO₄, filtered, and concentrated. The crude product was purified using silica gel chromatography (MeOH/CH₂Cl₂ gradient) to yield the desired product as a white solid. LRMS (M+H)⁺: 455.2.

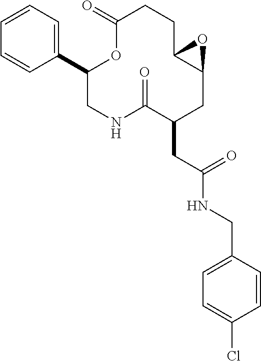

### N-(4-chlorobenzyl)-2-((1R,6R,10S,12R)-4,9-dioxo-6-phenyl-5,13-dioxa-8-azabicyclo[10.1.0]tridecan-10-yl)acetamide

To a solution of N-(4-chlorobenzyl)-2-((2R,6S,E)-5,12-dioxo-2-phenyl-1-oxa-4-azacyclododec-8-en-6-yl)acetamide (150 mg, 0.330 mmol) in CH₂Cl₂ (2061 μL) cooled in an ice-water bath was added mCPBA (155 mg, 0.692 mmol). The reaction mixture was slowly warmed to rt and stirred 6 h at which point TLC analysis indicated complete consumption of starting material. The reaction mixture was diluted with sat. Na₂SO₃ and CH₂Cl₂ and the layers separated. The aqueous was extracted 2× with CH₂Cl₂ and the combined organic extracts were washed with brine, dried over Na₂SO₄, filtered, and concentrated. The crude mixture was purified using silica gel chromatography (MeOH/CH₂Cl₂ gradient) to yield the desired product as a white solid. LRMS (M+H)⁺: 471.1.

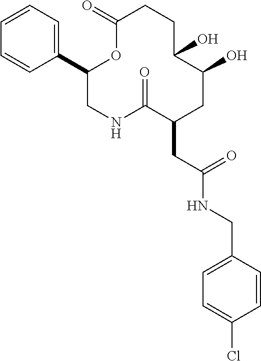

### N-(4-chlorobenzyl)-2-((2R,6S,8R,9R)-8,9-dihydroxy-5,12-dioxo-2-phenyl-1-oxa-4-azacyclododecan-6-yl)acetamide

To a solution of N-(4-chlorobenzyl)-2-((2R,6S,E)-5,12-dioxo-2-phenyl-1-oxa-4-azacyclododec-8-en-6-yl)acetamide (150 mg, 0.330 mmol) in tBuOH (2473 μL), THF (659 μL), and water (72 μL) was added NMO (93 μL of a 50 wt % solution in water, 0.396 mmol) followed by OsO₄ (433 μL of a 2.5 wt % solution in tBuOH). The reaction mixture was stirred at rt for 6 h at which point LC-MS analysis indicated complete consumption of starting material. The reaction mixture was diluted with sat. Na₂SO₃ and stirred 1 h. The mixture was then further diluted with EtOAc and the layers separated. The aqueous was extracted 2× with EtOAc and the combined organic extracts were washed with brine, dried over Na₂SO₄, filtered, and concentrated. The crude product was purified using silica gel chromatography (MeOH/CH₂Cl₂ gradient) to yield the desired product as a white solid. LRMS (M+H)⁺: 489.1.

### General Procedures for Amide or Ester Bond Formation

### A) EDCI, HOBt, DIPEA in an Appropriate Solvent

To a flask containing the carboxylic acid (˜1 eq.) sealed under nitrogen was added an appropriate dry solvent (DMF or DCM) to give a concentration of 0.1 molar (or greater).

To the resulting mixture was added diisopropylethylamine (˜2 eq.), the amine (˜1.3 eq.), HOBt (˜1.3 eq.) and EDCI (˜1.3 eq.). The reaction was stirred at room temperature until complete (typically 4-6 h). If the product had precipitated, it was filtered and dried, otherwise the mixture was diluted with an appropriate organic solvent (e.g. ethyl acetate) and washed twice with aqueous HCl, then aqueous sodium bicarbonate, then brine. The resulting solution was dried over magnesium sulfate, filtered, and concentrated.

### B) EDCI, HOBt, DIPEA, DMAP in an Appropriate Solvent

As in Method A, but with DMAP (0.1 eq.) additionally added to the reaction.

### C) EDCI, DMAP

The alcohol (1 eq.) and carboxylic acid (1.1 eq.) were added to a flask, sealed under nitrogen, and dissolved with dry solvent (typically DCM), to which was added EDCI (1.2 eq.) and DMAP (0.1 eq.). The reaction was stirred at room temperature until complete (typically 4-6 h), then it was diluted with an appropriate organic solvent (e.g. ethyl acetate) and washed twice with aqueous HCl, then twice with aqueous sodium bicarbonate, then brine. The resulting solution was dried over magnesium sulfate, filtered, and concentrated.

D) EDCI only in THF

To a flask containing the carboxylic acid (1 eq.) sealed under nitrogen was added THF, to give a concentration of 0.2 molar. The solution was cooled on ice and then the amino alcohol (1.1 eq.) and EDCI (1.2 eq.) were quickly added as solids. The mixture was stirred for 20 min., then the ice bath was removed and the reaction was stirred at room temperature until complete (typically 4-6 h). It was then poured slowly onto water (five times the volume of THF used) in an Erlenmeyer flask, and stirred well for 2 h. If the product had precipitated, it was filtered and dried, otherwise the product was diluted with an appropriate organic solvent (e.g. ethyl acetate) and washed twice with aqueous HCl, then aqueous sodium bicarbonate (if there was carboxylic acid remaining), then brine. The resulting solution was dried over magnesium sulfate, filtered, and concentrated.

### General Procedure for Ring Closing Metathesis

To a flask containing the diene (1.0 eq.) sealed under nitrogen was added an appropriate dry solvent (toluene or DCM) to give a concentration of 0.02 molar. Grubbs II catalyst (5 mol %) was added and the reaction was stirred at room temperature until complete (typically 8-12 h). 2-mercaptonicotinic acid (50 mol %) was then added to the reaction mixture and stirred vigorously for 2 hours at room temperature then washed with aqueous sodium bicarbonate, then brine. The resulting pale yellow organic layer was dried with sodium sulfate and decolorized with activated carbon for 10 minutes. The mixture was then filtered through celite and concentrated under vacuum.

### Example A

### N-((3R,11R,E)-5,12-dioxo-3-phenyl-1-oxa-4-azacyclododec-8-en-11yl)-methane sulfonamide

The TFA salt form of (3R,11R,E)-11-amino-3-phenyl-1-oxa-4-azacyclododec-8 ene-5,12-dione (30.0 mg, 0.075 mmol) was dissolved in DMF (4.0 ml), cooled to 0° C. and triethylamine (31.0 μl, 0.224 mmol) was added under nitrogen. The reaction mixture was treated with a solution of methanesulfonyl chloride (7.53 μl, 0.097 mmol) in 1 ml DMF. The mixture was allowed to warm to room temperature and the crude reaction was stopped at 2 hours. The crude reaction was diluted with EtOAc, washed with water, then brine, and dried over Na₂SO₄. The crude product was purified using silica gel chromatography using an ISCO automated system and a MeOH/DCM gradient. The product (15.2 mg) was isolated as a white solid in 56% yield. LRMS (ESI+) (M+H): 367.09

### Example B

### N-((3R,11R,E)-5,12-dioxo-3-phenyl-1-oxa-4-azacyclododec-8-en-11-yl)acetamide

tert-butyl ((3R,11R,E)-5,12-dioxo-3-phenyl-1-oxa-4-azacyclododec-8-en-11-yl)carbamate: Using General Procedure for Ring-Closing Metathesis using DCM as solvent, tert-butyl ((4R,8S)-5-oxo-8-(pent-4-enamido)-8-phenyloct-1-en-4-yl)carbamate (2.50 g. 6.0 mmol) and 5 mol % Grubs II (255.0 mg, 0.3 mmol) was added to the reaction and stirred for 3 hours at room temperature. After work-up steps from the general procedure for ring-closing metathesis, the crude product was obtained as an off-white solid (with trace amounts of ruthenium still present) which was triturated in EtOAc (×2), filtered, and washed with EtOAc to afford a white solid after filtration. Isolated yield is 64%. LRMS (ESI+) (M+H): 289.19.

(3R,11R,E)-11-amino-3-phenyl-1-oxa-4-azacyclododec-8-ene-5,12-dione: 2,2,2-Trifluoroacetic acid (11.51 ml, 154.0 mmol) and triethylsilane (12.34 ml, 77.0 mmol) were added to tert-butyl ((3R,11R,E)-5,12-dioxo-3-phenyl-1-oxa-4-azacyclododec-8-en-11-yl)carbamate (3.00 g, 7.72 mmol) in DCM (5 ml) and stirred at room temp for 4 hours. The reaction was concentrated under high vacuum. The crude mixture was triturated in EtOAc (×2) and the product was filtered-off. The isolated product was a white solid in 95-99% yield. LRMS (ESI+) (M+H): 289.21

TFA salt form of (3R,11S,E)-11-amino-3-phenyl-1-oxa-4-azacyclododec-8 ene-5,12-dione: This analog was prepared analogously to (3R,11R,E)-11-amino-3-phenyl-1-oxa-4-azacyclododec-8-ene-5,12-dione except using dicyclohexylammonium (S)-2-((tert-butoxycarbonyl)amino)pent-4-enoate instead of dicyclohexylammonium (R)-2-((tert-butoxycarbonyl)amino)pent-4-enoate.

N-((3R,11R,E)-5,12-dioxo-3-phenyl-1-oxa-4-azacyclododec-8-en-11-yl)acetamide: Under nitrogen, triethylamine (6.582 ml, 47.2 mmol) was added to the TFA salt form of (3R,11R,E)-11-amino-3-phenyl-1-oxa-4-azacyclododec-8 ene-5,12-dione (95.0 mg, 0.236 mmol). After stirring for one minute, acetic anhydride (4.46 ml, 47.2 mmol) was added to the mixture, upon which the reaction turned slightly clear and colorless. The reaction was stopped after 8 hours. The crude reaction was dried under high vacuum and product was purified by trituration with EtOAc (×2), followed by filtration and washing with DCM. The isolated product (77.2 mg) is 99% yield in the form of a white solid. LRMS (ESI+) (M+H): 331.14.

### Example C

### (3R,11R,E)-11-methyl-3-phenyl-1-oxa-4-azacyclododec-8-ene-5,12-dione

(R)—N-(2-hydroxy-1-phenylethyl)pent-4-enamide: Using general procedure B for amide bond formation with DMF as solvent (R)—N-(2-hydroxy-1-phenylethyl)pent-4-enamide was prepared from (R)-2-amino-2-phenylethanol (4.5 g, 33 mmol) and pent-4-enoic acid (2.6 mL, 25 mmol). Product (5.3 g) was obtained as a colorless oil in 97% yield after column chromatography (MeOH/DCM gradient). LRMS (ESI+) (M+H): 220.14.

(3R,11R,E)-11-methyl-3-phenyl-1-oxa-4-azacyclododec-8-ene-5,12-dione: Using general procedure C for ester bond formation using DCM as solvent (R)—((R)-2-pent-4-enamido-2-phenylethyl) 2-methylpent-4-enoate was prepared from (R)—N-(2-hydroxy-1-phenylethyl)pent-4-enamide (1.0 g, 4.6 mmol) and (R)-2-methylpent-4-enoic acid (570 mg, 5.0 mmol), prepared according to a literature procedure (Chakraborty, T. K. et al. Synlett 2002, 2039-2040). The product (1.4 g) was obtained as a white solid in 97% yield after column chromatography (MeOH/DCM gradient). Using the general produce for ring closing metathesis with toluene as solvent (3R,11R,E)-11-methyl-3-phenyl-1-oxa-4-azacyclododec-8-ene-5,12-dione was prepared from (R)—((R)-2-pent-4-enamido-2-phenylethyl) 2-methylpent-4-enoate (1.4 g, 4.4 mmol) to yield 470 mg of product as a white solid in 37% yield after column chromatography (MeOH/DCM gradient). LRMS (ESI+) (M+H): 288.19.

### Example D

### (3R,6S,11R,E)-6,11-dimethyl-3-phenyl-1-oxa-4-azacyclododec-8-ene-5,12-dione

(S)—N—((R)-2-hydroxy-1-phenylethyl)-2-methylpent-4-enamide: Using general procedure A for amide bond formation with DCM as solvent, the desired product was prepared from (R)-2-amino-2-phenylethanol (154.5 mg, 1.12 mmol) and (S)-2-methylpent-4-enoic acid (93.0 mg, 0.81 mmol), prepared according to a literature procedure (Chakraborty, T. K. et al. Synlett 2002, 2039-2040). The product was obtained as a white solid (134.0 mg, 72%) after column chromatography (EtOAc/hexane gradient). LRMS (ESI+) (M+H): 234.22.

(R)—(R)-2-((S)-2-methylpent-4-enamido)-2-phenylethyl 2-methylpent-4-enoate: Using general procedure C for ester bond formation with DCM as solvent, the desired product was prepared from (S)—N—((R)-2-hydroxy-1-phenylethyl)-2-methylpent-4-enamide (134.0 mg, 0.57 mmol) and (R)-2-methylpent-4-enoic acid (114.14 mg, 0.632 mmol), according to a literature procedure (Chakraborty, T. K. et al. Synlett 2002, 2039-2040). The product was obtained as a white solid (140.0 mg, 74.0% yield) after column chromatography (EtOAc/hexane gradient). LRMS (ESI+) (M+H): 330.37.

(3R,6S,11R,E)-6,11-dimethyl-3-phenyl-1-oxa-4-azacyclododec-8-ene-5,12-dione: Using the general procedure for ring closing metathesis using toluene as solvent, the desired product was prepared from (R)—(R)-2-((S)-2-methylpent-4-enamido)-2-phenylethyl 2-methylpent-4-enoate (140.0 mg, 0.425 mmol), yielding a white solid (100.0 mg, 78.0% yield) after column chromatography (EtOAc/hexanes gradient). LRMS (ESI+) (M+H): 302.25.

### Example E

### N-(4-chlorobenzyl)-2-((3R,8S,9S,11S)-8,9-dihydroxy-5,12-dioxo-3-phenyl-1-oxa-4-azacyclododecan-11-yl)acetamide

To a solution of N-(4-chlorobenzyl)-2-((3R,11S,E)-5,12-dioxo-3-phenyl-1-oxa-4-azacyclododec-8-en-11-yl)acetamide (62 mg, 0.14 mmol) in tBuOH (1.0 mL), THF (2701), and water (72 μL) was added N-methyl morpholine N-oxide (50 wt % solution in H₂O) (38 μl, 0.16 mmol) followed by OsO4 (2.5 wt % solution in tBuOH) (180 μl, 0.01 mmol). The reaction mixture was stirred at room temperature 6 h at which point TLC analysis indicated complete consumption of starting material. The reaction mixture was diluted with sat. Na₂SO₃ and stirred 1 h. The solution was further diluted with EtOAc and water and the layers separated. The aqueous layer was extracted 2× with EtOAc and the combined organic extracts were washed with brine, dried over Na₂SO₄, filtered, and concentrated. The crude product was purified by silica gel chromatography (MeOH/DCM gradient) to yield 59 mg of product as a white solid in 89% yield. LRMS (ESI+) (M+H): 489.35.

### Example G

### N-(4-chlorobenzyl)-2-((6S,7S,9S,14aS)-6,7-dihydroxy-3,10-dioxododecahydro-1H-pyrrolo[2,1-c][1,4]oxaazacyclododecin-9-yl)acetamide

N-(4-chlorobenzyl)-2-((9S,14aS,E)-3,10-dioxo-3,4,5,8,9,10,12,13,14,14a-decahydro-1H-pyrrolo[2,1-c][1,4]oxaazacyclododecin-9-yl)acetamide (40 mg, 0.095 mmol) was dissolved in a mixture of t-BuOH/THF/H₂O 15/4/1 (955 μL). To this solution were added successively osmium tetroxide (2.5 wt. % solution in t-BuOH, 125 μL, 9.6 mol) and N-methylmorpholine-N-oxide (50 wt. % solution in water, 27 μL, 0.115 mmol). The mixture was stirred for 4 h, after which a solution of saturated sodium sulfite is added and stirred for 1 h. After extraction with ethyl acetate, the organic phase was dried and concentrated. The product (21 mg) was obtained in 49% yield after column chromatography (MeOH/DCM gradient). LRMS (ESI+) (M+H): 453.33.

### Example H

### (3R,11R,E)-11-hydroxy-3-phenyl-1-oxa-4-azacyclododec-8-ene-5,12-dione

(R)-2-((tert-butyldimethylsilyl)oxy)pent-4-enoic acid: D-allylglycine HCl (1.31 g, 8.64 mmol), water (69 mL), and acetic acid (17 mL) were added to a flask, forming a colorless solution. The flask was cooled on ice, then a solution of sodium nitrite (1 M in water) (1.19 g, 17.3 mmol) was added over 3 min. The reaction was removed from the ice bath and stirred for 20 h, during which time the progression of the reaction was gauged by ninhydrin stain of the solution spotted on a TLC plate. The reaction was quenched by adding 2 M MeNH₂ in MeOH (8 mL, 16 mmol) and stirring for 1 h. The reaction was acidified with 2 M aq. HCl, extracted with Et₂O (3×50 mL), washed with brine, dried over MgSO₄, filtered, and concentrated (no lower than 7 torr), to give 663 mg (50%) of the intermediate 2-hydroxyacid, together with 0.6 eq. of acetic acid. 648 mg (4.24 mmol 2-hydroxyacid) of this liquid was dissolved with DMF (15 mL) and cooled on ice, before imidizole (924 mg, 13.6 mmol), and TBSCl (2.05 g, 13.57 mmol) were added quickly as solids. The flask was removed from the ice bath after 10 min., and stirred for an additional 17 h. The reaction was then diluted with 1 M HCl (˜60 mL) and EtOAc (˜60 mL). The layers were separated and the organics were washed three times with pH 2 water, then brine. The combined organics were concentrated, then re-dissolved with MeOH (˜8 mL) and water (˜3 mL). K₂CO₃ (2.93 g, 21.2 mmol) was added and the mixture was stirred vigorously for 3.5 h to hydrolyze the intermediate silyl ester. The mixture was concentrated to remove most of the MeOH, then diluted with water and washed with ether (3×50 mL). The aqueous layer was acidified to pH 2 with HCl (giving a colorless solution), then extracted with Et₂O (3×50 mL). The combined organics were washed with brine, dried over MgSO₄, filtered, and concentrated (10 torr or above) to give the desired product as a yellow liquid (660 mg, 68%). LRMS (ESI−) (M−H): 229.22.

(R)—(R)-2-(pent-4-enamido)-2-phenylethyl 2-((tert-butyldimethylsilyl)oxy)pent-4-enoate: Using general procedure C for ester bond formation with DCM as solvent, the desired product was prepared from (R)—N-(2-hydroxy-1-phenylethyl)pent-4-enamide (250 mg, 1.14 mmol) and (R)-2-((tert-butyldimethylsilyl)oxy)pent-4-enoic acid. The product was obtained as a colorless oil (228 mg, 46%) after work-up with EtOAc as solvent and column chromatography (EtOAc/hexanes gradient). LRMS (ESI+) (M+H): 553.23.

(3R,11R,E)-11-((tert-butyldimethylsilyl)oxy)-3-phenyl-1-oxa-4-azacyclododec-8-ene-5,12-dione: Using the general procedure for ring closing metathesis using toluene as solvent, the desired product was prepared from (R)—(R)-2-(pent-4-enamido)-2-phenylethyl 2-((tert-butyldimethylsilyl)oxy)pent-4-enoate (228 mg, 0.53 mmol), yielding a colorless oil (138 mg, 65%) after column chromatography (EtOAc/hexanes gradient). LRMS (ESI+) (M+H): 404.35.

(3R,11R,E)-11-hydroxy-3-phenyl-1-oxa-4-azacyclododec-8-ene-5,12-dione: (3R,11R,E)-11-((tert-butyldimethylsilyl)oxy)-3-phenyl-1-oxa-4-azacyclododec-8-ene-5,12-dione (86 mg, 0.21 mmol) was dissolved with THF (2.1 mL) and TBAF (1 M in THF, 0.43 mL, 0.43 mmol) was added. The reaction was stirred for 0.5 h, then it was diluted with aq. NH₄Cl and EtOAc, and the layers were separated. The combined organics were washed with water, then brine, then dried over MgSO₄, filtered, and concentrated. The resulting solid was purified by column chromatography (EtOAc/hexanes gradient), yielding a white solid (37 mg, 60%). LRMS (ESI+) (M+H): 290.20.

### Example I

### N-(4-chlorobenzyl)-2-((3aS,8R,12S,13aS)-2,2-dimethyl-6,11-dioxo-8-phenyldecahydro-3aH-[1,3]dioxolo[4,5-h][1,4]oxaazacyclododecin-12-yl)acetamide

To a solution of N-(4-chlorobenzyl)-2-((2R,6S,8S,9S)-8,9-dihydroxy-5,12-dioxo-2-phenyl-1-oxa-4-azacyclododecan-6-yl)acetamide (48 mg, 0.10 mmol) in DMF (980 μL) cooled in an ice/water bath was added 2,2-dimethoxypropane (120 μL, 0.98 mmol) and pyridinium p-toluenesulfonate (25 mg, 0.10 mmol). The reaction mixture was slowly warmed to rt and monitored by LC-MS. After 4 h an additional 10 eq of 2,2-dimethoxypropane was added and the solution was stirred 1 h at which point LC-MS indicated complete consumption of starting material. The reaction mixture was diluted with H₂O, extracted 3× with EtOAc, and the combined organic extracts were washed with brine, dried over Na₂SO₄, filtered, and concentrated. The crude product was purified by silica gel chromatography (MeOH/DCM) gradient to yield 35 mg of product as a white solid in 67% yield. LRMS (ESI+) (M+H): 529.36.

### Example J

### (3R,11R)-11-methyl-3-phenyl-1-oxa-4-azacyclododecane-5,12-dione

To a solution of (3R,11R,E)-11-methyl-3-phenyl-1-oxa-4-azacyclododec-8-ene-5,12-dione (110 mg, 0.38 mmol) in MeOH (1.9 mL) was added activated 10% palladium on carbon (40 mg) and the solution was thoroughly flushed with nitrogen. Hydrogen was then introduced to the solution via a balloon. After stirring under the hydrogen atmosphere for 1 hour, LC-MS indicated complete consumption of starting material. The reaction mixture was flushed with nitrogen then filtered through a pad of celite and the filter cake was washed with methanol. The methanol layer was concentrated and the crude product was purified by column chromatography (MeOH/DCM gradient) to yield 71 mg of product as a white solid in 64% yield. LRMS (ESI+) (M+H): 290.31.

### Example K

### (3R,11R,E)-6,6-difluoro-11-methyl-3-phenyl-1-oxa-4-azacyclododec-8-ene-5,12-dione

(R)-2,2-difluoro-N-(2-hydroxy-1-phenylethyl)pent-4-enamide: Using general procedure A for amide bond formation with DCM as solvent, the desired product was prepared from (R)-2-amino-2-phenylethanol and 2,2-difluoropent-4-enoic acid (68 mg, 0.50 mmol). The product was obtained as a white solid (78 mg, 61%) after work-up using EtOAc as solvent. LRMS (ESI+) (M+H): 256.06.

(R)—(R)-2-(2,2-difluoropent-4-enamido)-2-phenylethyl 2-methylpent-4-enoate: Using general procedure C for ester bond formation with DCM as solvent, the desired product was prepared from (R)-2,2-difluoro-N-(2-hydroxy-1-phenylethyl)pent-4-enamide (77 mg, 0.30 mmol) and (R)-2-methylpent-4-enoic acid, prepared according to a literature procedure (Chakraborty, T. K. et al. Synlett 2002, 2039-2040). The product was obtained as a colorless oil (105 mg, 99%) after work-up using EtOAc as solvent. LRMS (ESI+) (M+Na): 375.27.

(3R,11R,E)-6,6-difluoro-1-methyl-3-phenyl-1-oxa-4-azacyclododec-8-ene-5,12-dione: Using the general procedure for ring closing metathesis with toluene as solvent, the desired product was prepared from (R)—(R)-2-(2,2-difluoropent-4-enamido)-2-phenylethyl 2-methylpent-4-enoate (105 mg, 0.30 mmol) to yield the desired product as a white solid (81 mg, 84%) after column chromatography (EtOAc/hexanes gradient). LRMS (ESI+) (M+H): 324.17.

### Example L

### (3R,11R)-6,6-difluoro-11-methyl-3-phenyl-1-oxa-4-azacyclododecane-5,12-dione

(3R,11R,E)-6,6-difluoro-11-methyl-3-phenyl-1-oxa-4-azacyclododec-8-ene-5,12-dione (10 mg, 0.031 mmol) and 10% palladium on carbon (6.58 mg, 0.006 mmol) were added to a flask with stir bar and sealed under nitrogen. MeOH (1 mL) was added, and the sealed flask was flushed with hydrogen from a balloon, then stirred under the hydrogen balloon for 20 h. The reaction was filtered through a syringe filter and washed with MeOH, then concentrated to provide the desired product as a white solid (9.7 mg, 96%). LRMS (ESI+) (M+H): 326.18.

### Example M

### (3R,6R,11R,E)-6-amino-11-methyl-3-phenyl-1-oxa-4-azacyclododec-8-ene-5,12-dione

(9H-fluoren-9-yl)methyl ((R)-1-(((R)-2-hydroxy-1-phenylethyl)amino)-1-oxopent-4-en-2-yl)carbamate: Using general procedure A for amide bond formation with DMF as solvent, the desired product was prepared from (R)—N-Fmoc-allylglycine (500 mg, 1.48 mmol), synthesized according to a literature procedure (Hruby, V. J. et al. Org. Lett. 6, 3285-3288, 2004), and (R)-2-amino-2-phenylethanol. A fraction of the desired product precipitated from the reaction, and this was isolated by Buchner funnel. The remainder of the product was isolated according to the work-up described in the general procedure, using EtOAc as solvent and column chromatography (EtOAc/hexanes gradient). The combined product was a white solid (279 mg, 41%). LRMS (ESI+) (M+H): 457.38.

(R)—(R)-2-((R)-2-((((9H-fluoren-9-yl)methoxy)carbonyl)amino)pent-4-enamido)-2-phenylethyl 2-methylpent-4-enoate: Using general procedure C for ester bond formation with DMF as solvent, the desired product was prepared from (9H-fluoren-9-yl)methyl ((R)-1-(((R)-2-hydroxy-1-phenylethyl)amino)-1-oxopent-4-en-2-yl)carbamate (279 mg, 0.61 mmol) and (R)-2-methylpent-4-enoic acid, prepared according to a literature procedure (Chakraborty, T. K. et al. Synlett 2002, 2039-2040). The product was obtained as a white solid (190 mg, 56%) after work-up with EtOAc as solvent and column chromatography (EtOAc/hexanes gradient). LRMS (ESI+) (M+H): 553.23.

(9H-fluoren-9-yl)methyl ((3R,6R,11R,E)-11-methyl-5,12-dioxo-3-phenyl-1-oxa-4-azacyclododec-8-en-6-yl)carbamate: Using the general procedure for ring closing metathesis using DCM as solvent, the desired product was prepared from (R)—(R)-2-((R)-2-((((9H-fluoren-9-yl)methoxy)carbonyl)amino)pent-4-enamido)-2-phenylethyl 2-methylpent-4-enoate (190 mg, 0.34 mmol), yielding the product as a white solid (135 mg, 75%) after filtration by Buchner funnel. LRMS (ESI+) (M+H): 525.38.

(3R,6R,11R,E)-6-amino-1-methyl-3-phenyl-1-oxa-4-azacyclododec-8-ene-5,12-dione: (9H-fluoren-9-yl)methyl ((3R,6R,11R,E)-11-methyl-5,12-dioxo-3-phenyl-1-oxa-4-azacyclododec-8-en-6-yl)carbamate (125 mg, 0.24 mmol), DMF (1.8 mL), and piperidine (0.6 mL) were added to a flask and stirred for 4 h. The mixture was then concentrated and purified by column chromatography (MeOH/DCM gradient), to give the desired product as a white solid (70 mg, 98%). LRMS (ESI+) (M+H): 303.14.

### Example N

### N-((3R,6R,11R,E)-11-methyl-5,12-dioxo-3-phenyl-1-oxa-4-azacyclododec-8-en-6-yl)acetamide

(3R,6R,11R,E)-6-amino-11-methyl-3-phenyl-1-oxa-4-azacyclododec-8-ene-5,12-dione (35 mg, 0.12 mmol) was added to a 4 mL vial with stir bar and sealed under nitrogen, followed by DMF (1 mL), NEt₃ (0.40 mL, 2.9 mmol) and acetic anhydride (0.27 mL, 2.9 mmol). The reaction was stirred for 48 h, then concentrated to dryness, re-dissolved with EtOAc, and washed successively with half-saturated aq. NaHCO₃, 1 M aq. HCl, and brine. The organic phase was dried over MgSO₄, filtered, and concentrated. The resulting crude material was purified by column chromatography (MeOH/DCM gradient), yielding the desired product as a white solid (27 mg, 68%). LRMS (ESI+) (M+H): 345.15.

### Example O

### (3R,E)-11-methyl-3-phenyl-1-oxa-4-azacyclododec-8-en-5-one

(R)—N-(2-hydroxy-1-phenylethyl)pent-4-enamide (760 mg, 3.47 mmol) was dissolved in THF (30 mL). (E)-diisopropyl diazene-1,2-dicarboxylate (1.05 g, 5.20 mmol) and triphenylphosphine (1.36 g, 5.20 mmol) were added. The reaction was stirred at room temperature for 1 hour. 2-methylpent-4-en-1-ol (350 mg, 3.47 mmol) (Ardisson, J. et al. Chem. Eur. J., 2008, 11092-11112.) was added and the reaction was stirred for 18 hours.

The reaction was quenched by addition of a 1N solution of hydrochloric acid, followed by dilution with ether and separation. The organic layer was washed with brine and dried with sodium sulfate. The organic layer was cooled to 0° C. and left not stirring for 1 hour. The precipitate was filtered off and washed with ether. The filtrate was concentrated under vacuum to yield N-((1R)-2-((2-methylpent-4-en-1-yl)oxy)-1-phenylethyl)pent-4-enamide. Using the general procedure for ring closing metathesis with toluene as solvent (3R,E)-11-methyl-3-phenyl-1-oxa-4-azacyclododec-8-en-5-one was prepared from N-((1R)-2-((2-methylpent-4-en-1-yl)oxy)-1-phenylethyl)pent-4-enamide (250 mg, 0.829 mmol). Product (3.5 mg) was obtained in 0.5% yield over two steps following purification by column chromatography (Hexane/Ethyl Acetate gradient). ¹H NMR (300 MHz, CDCl₃) δ 7.34 (d, J=46.6, 5H), 5.55 (s, 2H), 5.21 (s, 1H), 4.95 (s, 2H), 4.22 (s, 2H), 2.93-2.81 (m, 1H), 2.25 (d, J=46.4, 5H), 1.71 (s, 1H), 1.17 (d, J=56.6, 4H).

### Example P

### (R)-11,11-dimethyl-3-phenyl-1-oxa-4-azacyclododecane-5,8,12-trione

### (R)-2-(pent-4-ynamido)-2-phenylethyl 2,2-dimethylpent-4-enoate

Using general procedure A for amide bond formation with DCM as solvent (R)—N-(2-hydroxy-1-phenylethyl)pent-4-ynamide was prepared from (R)-2-amino-2-phenylethanol (2.1 g, 15.3 mmol) and pent-4-ynoic acid (1.0 g, 10 mmol). The product was obtained as a colorless oil and carried on to the next step without further purification. Using general procedure C for ester bond formation using DCM as solvent (R)-2-(pent-4-ynamido)-2-phenylethyl 2,2-dimethylpent-4-enoate was prepared from (R)—N-(2-hydroxy-1-phenylethyl)pent-4-ynamide (2.0 g, 9.21 mmol) and 2,2-dimethylpent-4-enoic acid (1.2 g, 9.21 mmol). The product (1.5 g) was obtained as a clear oil in 46% yield over two steps after column chromatography (Hexanes/Ethyl Acetate gradient). ¹H NMR (300 MHz, CDCl₃) δ 7.50-7.17 (m, 5H), 6.35 (s, 1H), 5.85 (s, 1H), 5.50 (t, J=14.4, 1H), 5.30 (s, 1H), 4.60-4.29 (m, 2H), 4.20 (t, J=9.1, 1H), 3.61 (t, J=8.0, 1H), 2.63-2.26 (m, 3H), 2.05 (s, 3H), 1.71 (dd, J=12.0, 20.2, 2H), 1.24 (d, J=6.9, 4H), 1.03-0.83 (m, 1H).

(R)-2-phenyl-2-(4-(triethoxysilyl)pent-4-enamido)ethyl 2,2-dimethylpent-4-enoate: To a solution of (R)-2-(pent-4-ynamido)-2-phenylethyl 2,2-dimethylpent-4-enoate (1.5 g, 4.58 mmol) in DCM (30 mL) was added triethoxysilane (1 mL, 5.5 mmol). The reaction was cooled to 0° C. and Tris(acetonitrile)cyclopentadienylruthenium (II) hexafluorophosphate (19 mg, 0.046 mmol) was added. The reaction was warmed to room temperature and stirred for 2 hours. The reaction was concentrated under vacuum. The product (1.0 g) was obtained as a clear oil in 44% yield over two steps after column chromatography (Hexanes/Ethyl Acetate gradient). ¹H NMR (300 MHz, CDCl₃) δ 7.40-7.19 (m, 5H), 6.38 (d, J=7.8, 1H), 5.60 (ddd, J=11.9, 20.6, 26.2, 3H), 5.29 (dd, J=6.6, 14.1, 1H), 5.05-4.86 (m, 2H), 4.30 (ddd, J=6.4, 11.3, 17.1, 3H), 3.94-3.73 (m, 6H), 2.59-2.31 (m, 4H), 2.18 (d, J=7.4, 2H), 1.47-0.96 (m, 15H).

(R)-11,11-dimethyl-3-phenyl-8-(triethoxysilyl)-1-oxa-4-azacyclododec-8-ene-5,12-dione: Using the general procedure for ring closing metathesis with toluene as solvent (R)-11,11-dimethyl-3-phenyl-8-(triethoxysilyl)-1-oxa-4-azacyclododec-8-ene-5,12-dione was prepared from (R)-2-phenyl-2-(4-(triethoxysilyl)pent-4-enamido)ethyl 2,2-dimethylpent-4-enoate (1.0 g, 3.04 mmol). The roduct (55 mg) was obtained in 6% yield following purification by column chromatography (Hexane/Ethyl Acetate gradient). ¹H NMR (300 MHz, CDCl₃) δ 7.28 (d, J=7.6, 5H), 6.59 (d, J=8.1, 1H), 5.65 (d, J=26.8, 2H), 5.38-5.11 (m, 2H), 5.04-4.89 (m, 1H), 4.39-4.16 (m, 2H), 3.82 (q, J=7.0, 3H), 2.45 (dd, J=6.3, 19.5, 3H), 2.07 (s, 2H), 1.94 (s, 1H), 1.35-0.93 (m, 15H).

(R)-11,11-dimethyl-3-phenyl-1-oxa-4-azacyclododecane-5,8,12-trione: To a solution of (R)-11,11-dimethyl-3-phenyl-8-(triethoxysilyl)-1-oxa-4-azacyclododec-8-ene-5,12-dione (55 mg, 0.122 mmol) in THF/MeOH (1 mL/2 mL) was added potassium fluoride (36 mg, 0.612 mmol), sodium bicarbonate (51 mg, 0.612 mmol) and 30% aqueous hydrogen peroxide (1.1 mL, 9.8 mmol). The reaction was stirred for 18 hours. The reaction was quenched by addition of a saturated solution of sodium thiosulfate, diluted with ethyl acetate and separated and the organic layer was washed with brine. The organic layer was then dried and concentrated under vacuum. The product (4.5 mg) was obtained as a white solid in 12% yield after column chromatography (Hexanes/Ethyl Acetate gradient). ¹H NMR (300 MHz, CDCl₃) δ 7.30 (m, 5H), 6.80 (d, J=8.2, 1H), 5.28 (dd, J=8.2, 13.5, 2H), 4.27 (ddd, J=6.4, 11.4, 16.5, 2H), 2.80 (td, J=3.2, 6.5, 2H), 2.50 (t, J=6.9, 2H), 2.28-2.01 (m, 2H), 1.75 (s, 1H), 1.37 (s, 1H), 1.30-0.97 (m, 5H).

### Example Q

### (3R,11S,E)-11-methyl-3-phenyl-1-oxa-4-azacyclododec-8-en-12-one

(R)-2-amino-2-phenylethanol (734 mg, 5.35 mmol) and pent-4-enal (300 mg, 3.57 mmol) were dissolved in THF (50 mL) and stirred for 1 hour. Sodium cyanoborohydride (3.4 g, 53.5 mmol) was added and the reaction was stirred for 18 hours. The reaction was quenched by addition of a 1N solution of hydrochloric acid and stirred for 1 hour. The reaction mixture was diluted with Ethyl Acetate and separated. The organic layer was washed three times with 1N hydrochloric acid followed by brine, then dried with sodium sulfate and concentrated under vacuum. (R)-2-(pent-4-en-1-ylamino)-2-phenylethanol was obtained as a colorless oil and carried on to the next step without further purification. Using general procedure C for ester bond formation using DCM as solvent (R)-2-(pent-4-en-1-ylamino)-2-phenylethyl 2-methylpent-4-enoate was prepared from (R)-2-(pent-4-en-1-ylamino)-2-phenylethanol (700 mg, 3.41 mmol) and (S)-2-methylpent-4-enoic acid (Chakraborty, T. K. et al. Synlett 2002, 2039-2040) (428 mg, 3.75 mmol). The product was obtained as a white solid and carried on to the next step without further purification. Using the general produce for ring closing metathesis with toluene as solvent (3R,11S,E)-11-methyl-3-phenyl-1-oxa-4-azacyclododec-8-en-12-one was prepared from (R)-2-(pent-4-en-1-ylamino)-2-phenylethyl 2-methylpent-4-enoate (900 mg, 2.99 mmol) to yield 400 mg of product as a white solid in 41% yield over three steps after column chromatography (Hexane/Ethyl Acetate gradient). ¹H NMR (300 MHz, CDCl₃) δ 7.31 (d, J=26.5, 5H), 6.28 (d, J=11.2, 1H), 5.58 (d, J=31.1, 2H), 5.26 (s, 1H), 4.44 (ddd, J=5.0, 12.7, 73.2, 2H), 2.81-1.98 (m, 8H), 1.34-1.05 (m, 4H).

### Example R

### (3R,11R,E)-3,11-dimethyl-1-oxa-4-azacyclododec-8-ene-5,12-dione

Using general procedure C for amide bond formation with DCM as solvent (R)—N-(1-hydroxypropan-2-yl)pent-4-enamide was prepared from (R)-2-aminopropan-1-ol (563 mg, 7.49 mmol) and pent-4-enoic acid (0.51 mL, 5 mmol). The product was obtained as a colorless oil and carried on to the next step without further purification. Using general procedure C for ester bond formation using DCM as solvent (R)—(R)-2-(pent-4-enamido)propyl 2-methylpent-4-enoate was prepared from (R)—N-(1-hydroxypropan-2-yl)pent-4-enamide (700 mg, 4.09 mmol) and (R)-2-methylpent-4-enoic acid (Chakraborty, T. K. et al. Synlett 2002, 2039-2040) (558 mg, 4.5 mmol). The product was obtained as a white solid and carried on to the next step without further purification. Using the general produce for ring closing metathesis with toluene as solvent (3R,11R,E)-3,11-dimethyl-1-oxa-4-azacyclododec-8-ene-5,12-dione was prepared from (R)—(R)-2-(pent-4-enamido)propyl 2-methylpent-4-enoate (1.0 g, 3.95 mmol) to yield 422 mg of product as a white solid in 38% yield over three steps after column chromatography (Hexane/Ethyl Acetate gradient). ¹H NMR (300 MHz, CDCl₃) δ 5.37 (ovrlp, 3H), 4.63 (t, J=11.5, 1H), 4.37 (m, 1H), 3.55 (dd, J=3.5, 11.6, 1H), 2.54 (m, 1H), 2.14-2.36 (ovrlp, 5H), 1.88 (m, 1H), 1.13 (dd, J=6.8, 13.2, 6H)

### Example S

### (3S,11R,E)-3,11-dimethyl-1-oxa-4-azacyclododec-8-ene-5,12-dione

Using general procedure C for amide bond formation with DCM as solvent (S)—N-(1-hydroxypropan-2-yl)pent-4-enamide was prepared from (S)-2-aminopropan-1-ol (113 mg, 1.5 mmol) and pent-4-enoic acid (0.1 mL, 1 mmol). The product was obtained as a colorless oil and carried on to the next step without further purification. Using general procedure C for ester bond formation using DCM as solvent (R)—(S)-2-(pent-4-enamido)propyl 2-methylpent-4-enoate was prepared from (S)—N-(1-hydroxypropan-2-yl)pent-4-enamide (157 mg, 0.92 mmol) and (R)-2-methylpent-4-enoic acid (Chakraborty, T. K. et al. Synlett 2002, 2039-2040) (125 mg, 1.0 mmol). The product was obtained as a white solid and carried on to the next step without further purification. Using the general produce for ring closing metathesis with toluene as solvent (3S,11R,E)-3,11-dimethyl-1-oxa-4-azacyclododec-8-ene-5,12-dione was prepared from (R)—(S)-2-(pent-4-enamido)propyl 2-methylpent-4-enoate (212 g, 0.84 mmol) to yield 79 mg of product as a white solid in 35% yield over three steps after column chromatography (Hexane/Ethyl Acetate gradient). ¹H NMR (300 MHz, CDCl₃) δ 5.46 (m, 3H), 4.22 (m, 3H), 2.71 (m, 1H), 2.28 (ovrlp, 5H), 1.19 (ovrlp, 7H).

### Example T

### (R,E)-3,11,11-trimethyl-1-oxa-4-azacyclododec-8-ene-5,12-dione

Using general procedure C for amide bond formation with DCM as solvent (R)—N-(1-hydroxypropan-2-yl)pent-4-enamide was prepared from (R)-2-aminopropan-1-ol (113 mg, 1.5 mmol) and pent-4-enoic acid (0.1 mL, 1 mmol). The product was obtained as a colorless oil and carried on to the next step without further purification. Using general procedure C for ester bond formation using DCM as solvent (R)-2-(pent-4-enamido)propyl 2,2-dimethylpent-4-enoate was prepared from (R)—N-(1-hydroxypropan-2-yl)pent-4-enamide (50 mg, 0.318 mmol) and 2,2-dimethylpent-4-enoic acid (45 mg, 0.35 mmol). The product was obtained as a white solid and carried on to the next step without further purification. Using the general produce for ring closing metathesis with toluene as solvent (R,E)-3,11,11-trimethyl-1-oxa-4-azacyclododec-8-ene-5,12-dione was prepared from (R)-2-(pent-4-enamido)propyl 2,2-dimethylpent-4-enoate (26 mg, 0.1 mmol) to yield 19 mg of product as a white solid in 8% yield over three steps after column chromatography (Hexane/Ethyl Acetate gradient).

### Example U

### (3R,11R)-3,11-dimethyl-1-oxa-4-azacyclododecane-5,12-dione

(3R,11R,E)-3,11-dimethyl-1-oxa-4-azacyclododec-8-ene-5,12-dione (82 mg, 0.36 mmol) in THF (3 mL) was stirred under hydrogen atmosphere in the presence of palladium 10 wt. % on activated carbon (77 mg). After 3 h at room temperature, the reaction mixture was filtered, washed with methanol, and evaporated. The product (81 mg) was obtained in 97% yield. ¹H NMR (300 MHz, CDCl₃) δ 5.28 (broad s, 1H), 4.68-4.39 (m, 2H), 3.73-3.57 (m, 1H), 2.61 (m, 1H), 2.30 (m, 1H), 2.01 (m, 2H), 1.75-1.19 (m, 9H), 1.14 (ovrlp, d, J=6.2, 4H).

### Example V

### (9R,14aR,E)-9-methyl-4,5,8,9,12,13,14,14a-octahydro-1H-pyrrolo[2,1-c][1,4]oxaazacyclododecine-3,10-dione

Using general procedure A for amide bond formation with DCM as solvent, (R)-1-((R)-2-(hydroxymethyl)pyrrolidin-1-yl)-2-methylpent-4-en-1-one was prepared from (R)-2-methylpent-4-enoic acid (113 mg, 0.99 mmol) and (S)-pyrrolidin-2-ylmethanol (100 mg, 0.99 mmol). The product was obtained and used directly in the following step without further purification. Using general procedure C for ester bond formation with DCM as solvent ((R)-1-((R)-2-methylpent-4-enoyl)pyrrolidin-2-yl)methyl pent-4-enoate was prepared from 4-pentenoic acid (0.11 mL, 1.01 mmol) and (R)-1-((R)-2-(hydroxymethyl)pyrrolidin-1-yl)-2-methylpent-4-en-1-one (195 mg, 0.98 mmol). The product was obtained and used directly in the following step without further purification. Using the general procedure for ring closing metathesis with toluene as solvent (9R,14aR,E)-9-methyl-4,5,8,9,12,13,14,14a-octahydro-1H-pyrrolo[2,1-c][1,4]oxaazacyclododecine-3,10-dione was prepared from ((R)-1-((R)-2-methylpent-4-enoyl)pyrrolidin-2-yl)methyl pent-4-enoate (156 mg, 0.56 mmol). The product (51 mg) was obtained in 20% yield over three steps following purification by column chromatography (Hex/EtOAc gradient).

### Example W

### (9S,14aS,E)-9-methyl-4,5,8,9,12,13,14,14a-octahydro-1H-pyrrolo[2,1-c][1,4]oxaazacyclododecine-3,10-dione

Using general procedure A for amide bond formation with DCM as solvent, (S)-1-((S)-2-(hydroxymethyl)pyrrolidin-1-yl)-2-methylpent-4-en-1-one was prepared from (S)-2-methylpent-4-enoic acid (56 mg, 0.49 mmol) and (S)-pyrrolidin-2-ylmethanol (50 mg, 0.49 mmol). The product was obtained and used directly in the following step without further purification. Using general procedure C for ester bond formation with DCM as solvent ((S)-1-((S)-2-methylpent-4-enoyl)pyrrolidin-2-yl)methyl pent-4-enoate was prepared from 4-pentenoic acid (56 μL, 0.55 mmol) and (S)-1-((S)-2-(hydroxymethyl)pyrrolidin-1-yl)-2-methylpent-4-en-1-one (98 mg, 0.49 mmol). The product was obtained and used directly in the following step without further purification. Using the general procedure for ring closing metathesis with toluene as solvent (9S,14aS,E)-9-methyl-4,5,8,9,12,13,14,14a-octahydro-1H-pyrrolo[2,1-c][1,4]oxaazacyclododecine-3,10-dione was prepared from ((S)-1-((S)-2-methylpent-4-enoyl)pyrrolidin-2-yl)methyl pent-4-enoate (105 mg, 0.38 mmol). The product (31 mg) was obtained in 25% yield over three steps following purification by column chromatography (Hex/EtOAc gradient). ¹H NMR (300 MHz, CDCl₃) δ 5.39 (m, 2H), 4.59 (m, 1H), 4.18 (ddd, J=5.9, 11.2, 15.3, 2H), 3.64-3.40 (m, 2H), 2.69 (ddd, J=5.5, 9.4, 13.7, 1H), 2.47-2.18 (m, 4H), 2.18-1.81 (m, 4H), 1.71 (d, J=11.8, 2H), 1.14 (d, J=6.8, 3H).

### Example X

### (3R,11R)-8,11-dimethyl-3-phenyl-1-oxa-4-azacyclododec-8-ene-5,12-dione

Using general procedure C for amide bond formation with DCM as solvent (R)—N-(2-hydroxy-1-phenylethyl)-4-methylpent-4-enamide was prepared from (R)-2-amino-2-phenylethanol (270 mg, 2.0 mmol) and 4-methylpent-4-enoic acid (150 mg, 1.3 mmol). The product was obtained as a colorless oil and carried on to the next step without further purification. Using general procedure C for ester bond formation using DCM as solvent (R)—(R)-2-(4-methylpent-4-enamido)-2-phenylethyl 2-methylpent-4-enoate was prepared from (R)—N-(2-hydroxy-1-phenylethyl)-4-methylpent-4-enamide (100 mg, 0.43 mmol) and (R)-2-methylpent-4-enoic acid (Chakraborty, T. K. et al. Synlett 2002, 2039-2040). The product was obtained as a white solid and carried on to the next step without further purification. Using the general produce for ring closing metathesis with toluene as solvent (3R,11R)-8,11-dimethyl-3-phenyl-1-oxa-4-azacyclododec-8-ene-5,12-dione was prepared from (R)—(R)-2-(4-methylpent-4-enamido)-2-phenylethyl 2-methylpent-4-enoate (141 g, 0.43 mmol) to yield 100 mg of product as a white solid in 26% yield over three steps after column chromatography (Hexane/Ethyl Acetate gradient). ¹H NMR (300 MHz, CDCl₃) δ 7.29 (m, 5H) 6.60 (d, J=7.9, 1H), 5.32 (dd, J=5.2, 10.1, 1H), 4.71 (d, J=16.8, 1H), 4.28 (ddd, J=8.1, 14.2, 16.0, 2H), 2.59-2.06 (ovrlp, m, 8H), 1.72 (ovrlp m, 4H), 1.08 (d, J=6.9, 3H).

### Example Y

### (3S,11R)-3,11-dimethyl-1-oxa-4-azacyclododecane-5,12-dione

(3S,11R,E)-3,11-dimethyl-1-oxa-4-azacyclododec-8-ene-5,12-dione (40 mg, 0.18 mmol) in THF (3 mL) was stirred under hydrogen atmosphere in the presence of palladium 10 wt. % on activated carbon (38 mg). After 3 h at room temperature, the reaction mixture was filtered, washed with methanol, and evaporated. The product (39 mg) was obtained in 97% yield. ¹H NMR (300 MHz, CDCl₃) δ 5.37 (broad s, 1H), 4.42 (m, 1H), 4.10 (ddd, J=7.1, 11.4, 15.5, 2H), 2.49 (m, 1H), 2.35 (dd, J=9.1, 11.8, 1H), 1.98-1.82 (m, 2H), 1.72-1.23 (m, 9H), 1.16 (dd, J=3.0, 6.8, 4H).

### Example Z

### (3R,11R)-8,11-dimethyl-3-phenyl-1-oxa-4-azacyclododecane-5,12-dione

(3R,11R)-8,11-dimethyl-3-phenyl-1-oxa-4-azacyclododec-8-ene-5,12-dione (25 mg, 0.08 mmol) in THF (3 mL) was stirred under hydrogen atmosphere in the presence of palladium 10 wt. % on activated carbon (18 mg). After 3 h at room temperature, the reaction mixture was filtered, washed with methanol, and evaporated. The product (24 mg) was obtained in 93% yield. ¹H NMR (300 MHz, CDCl₃) δ 7.30 (m, 11.9, 5H), 6.37 (d, J=8.0, 1H), 5.30 (dd, J=7.6, 12.5, 1H), 4.33 (ddd, J=6.2, 11.4, 16.2, 2H), 2.40 (dd, J=7.0, 13.7, 1H), 2.31-2.10 (m, 2H), 1.63-1.00 (m, 10H), 0.87 (d, J=1.6, 3H).

### Example AA

### (3R,11R)-8-amino-9-hydroxy-11-methyl-3-phenyl-1-oxa-4-azacyclododecane-5,12-dione

tert-butyl ((3R,11R)-9-hydroxy-11-methyl-5,12-dioxo-3-phenyl-1-oxa-4-azacyclododecan-8-yl)carbamate: To a solution of tert-butyl carbamate (130 mg, 1.11 mmol) in n-propanol (1 mL) was added a solution of sodium hydroxide in water (430 μL, 1.08 mmol) followed by tert-butyl hypochlorite (117 mg, 1.08 mmol). After fifteen minutes (3R,11R,E)-11-methyl-3-phenyl-1-oxa-4-azacyclododec-8-ene-5,12-dione was added (100 mg, 0.348 mmol) followed by a solution of potassium dioxidodioxoosmium (7 mg, 0.021 mmol) in water (100 mL). The reaction was stirred for 18 hours. The reaction was quenched by addition of sodium bissulfite (370 mg, 3.48 mmol), diluted with ethyl acetate and washed with a 1N solution of hydrochloric acid, then brine. The organic layer was dried, then concentrated under vacuum. The product (40 mg) was obtained as a white solid in 27% yield after column chromatography (MeOH/DCM gradient). ¹H NMR (300 MHz, CDCl₃) δ 7.33 (s, 5H), 6.56 (d, J=9.3, 1H), 5.68-5.43 (m, 1H), 5.05 (s, 2H), 4.11-3.33 (m, 5H), 2.66-1.70 (m, 7H), 1.44 (s, 8H), 1.22 (d, J=7.0, 3H).

(3R,11R)-8-amino-9-hydroxy-11-methyl-3-phenyl-1-oxa-4-azacyclododecane-5,12-dione: To a solution of tert-butyl ((3R,11R)-9-hydroxy-11-methyl-5,12-dioxo-3-phenyl-1-oxa-4-azacyclododecan-8-yl)carbamate (40 mg, 0.095 mmol) in DCM (2 mL) was added triethylsilane (111 mg, 0.95 mmol) followed by TFA (217 mg, 1.9 mmol). The reaction was stirred for 18 hours and then was concentrated under vacuum to provide product (30 mg) in 98% yield after column chromatography (MeOH/DCM gradient). ¹H NMR (300 MHz, MeOD) δ 7.36 (s, 5H), 5.43 (s, 1H), 4.45 (d, J=11.6, 1H), 4.25 (s, 1H), 3.67 (s, 1H), 3.36-3.20 (m, 4H), 2.81-1.72 (m, 8H), 1.20 (d, J=6.9, 4H).

### Example AB

### (8R,12R)-12-methyl-8-phenyloctahydro-2H-oxazolo[4,5-h][1,4]oxaazacyclododecine-2,6,11(7H,12H)-trione

(3R,11R)-8-amino-9-hydroxy-11-methyl-3-phenyl-1-oxa-4-azacyclododecane-5,12-dione was dissolved in DCM (1 mL) and di(1H-imidazol-1-yl)methanone (10 mg, 0.062 mmol) was added followed by i-Pr₂NEt (12 mg, 0.094 mmol). The reaction was stirred for 18 hours. The reaction was quenched by addition of a 1N solution of hydrochloric acid, diluted with ethyl acetate and separated and the organic layer was washed with brine. The organic layer was then dried and concentrated under vacuum. The product (8 mg) was obtained as a white solid in 37% yield after column chromatography (MeOH/DCM gradient). ¹H NMR (300 MHz, CDCl₃) δ 7.50-7.17 (m, 5H), 6.35 (s, 1H), 5.85 (s, 1H), 5.50 (t, J=14.4, 1H), 5.30 (s, 1H), 4.60-4.29 (m, 1H), 4.20 (t, J=9.1, 1H), 3.61 (t, J=8.0, 1H), 2.63-2.26 (m, 2H), 2.05 (s, 2H), 1.71 (dd, J=12.0, 20.2, 2H), 1.24 (d, J=6.9, 3H), 1.03-0.83 (m, 1H).

### Example AC

### (3R,11R,E)-11-amino-3-phenyl-1-oxa-4-azacyclododec-8-ene-5,12-dione

Triethylamine (6.58 ml, 47.2 mmol) was added to the TFA salt form of (3R,11R,E)-11-amino-3-phenyl-1-oxa-4-azacyclododec-8 ene-5,12-dione (95.0 mg, 0.236 mmol) and DCM (2.0 ml). After stirring for 2 hours, the reaction was diluted with DCM, washed with half-saturated Na₂CO₃ and brine. The organic layer collected was dried over Na₂SO₄ and dried under high vacuum to afford the product (30 mg) as a white solid in 99% yield. LRMS (ESI+) (M+H): 289.21

### Example AD

### (3S,11R,E)-11-methyl-3-phenyl-1-oxa-4-azacyclododec-8-ene-5,12-dione

(S)—N-(2-hydroxy-1-phenylethyl)pent-4-enamide: Using General Procedure B for Amide Bond Formation with DMF as solvent, (S)—N-(2-hydroxy-1-phenylethyl)pent-4-enamide was prepared from (S)-2-amino-2-phenylethanol (3.43 g, 24.97 mmol) and pent-4-enoic acid (2.52 mL, 24.97 mmol). The product (4.75 g) was obtained as a clear colorless oil in 87% yield after silica gel chromatography using an ISCO automated system (MeOH/DCM gradient). LRMS (ESI+) (M+H): 220.16

(R)—(S)-2-(pent-4-enamido)-2-phenylethyl 2-methylpent-4-enoate: Using General Procedure C for Ester Bond Formation with DCM as solvent, (R)—(S)-2-(pent-4-enamido)-2-phenylethyl 2-methylpent-4-enoate was prepared from (S)—N-(2-hydroxy-1-phenylethyl)pent-4-enamide (300.00 mg, 1.368 mmol) and (R)-2-methylpent-4-enoic acid (203 μl, 1.505 mmol). The product (320 mg) was isolated in 74% yield as a white solid after silica gel chromatography using an ISCO automated system (MeOH/DCM gradient). LRMS (ESI+) (M+H): 316.30

(3S,11R,E)-1-methyl-3-phenyl-1-oxa-4-azacyclododec-8-ene-5,12-dione: Using General Procedure for Ring-Closing Metathesis using toluene as solvent, (R)—(S)-2-(pent-4-enamido)-2-phenylethyl 2-methylpent-4-enoate (320 mg, 1.016 mmol) was used in the reaction and stirred overnight (16 hours) at room temperature. The product (210 mg) was isolated in 72% yield as a white solid after silica gel chromatography using an ISCO automated system (MeOH/DCM gradient). LRMS (ESI+) (M+H): 288.23

### Example AE

### (3S,11S,E)-11-methyl-3-phenyl-1-oxa-4-azacyclododec-8-ene-5,12-dione

(S)—(S)-2-(pent-4-enamido)-2-phenylethyl 2-methylpent-4-enoate: Using General Procedure C for Ester Bond Formation with DCM as solvent, (S)—(S)-2-(pent-4-enamido)-2-phenylethyl 2-methylpent-4-enoate was prepared from (S)—N-(2-hydroxy-1-phenylethyl)pent-4-enamide (300.00 mg, 1.368 mmol) and (S)-2-methylpent-4-enoic acid (203 μl, 1.505 mmol). The product (338 mg) was isolated in 78% yield as a white solid after silica gel chromatography using an ISCO automated system (MeOH/DCM gradient). LRMS (ESI+) (M+H): 316.29.

(3S,11S,E)-11-methyl-3-phenyl-1-oxa-4-azacyclododec-8-ene-5,12-dione: Using General Procedure for Ring-Closing Metathesis using toluene as solvent, (S)—(S)-2-(pent-4-enamido)-2-phenylethyl 2-methylpent-4-enoate (335 mg, 1.064 mmol) was used in the reaction and stirred overnight (16 hours) at room temperature. The product (225 mg) was isolated in 74% yield as a white solid after silica gel chromatography using an ISCO automated system (MeOH/DCM gradient). LRMS (ESI+) (M+H): 288.27

### Example AF

### (3R,11R,E)-1,11-dimethyl-3-phenyl-1,4-diazacyclododec-8-ene-5,12-dione

tert-butyl ((R)-2-((R)—N,2-dimethylpent-4-enamido)-1-phenylethyl)carbamate: Using General Procedure B for Amide Bond Formation with DMF as solvent, tert-butyl ((R)-2-((R)—N,2-dimethylpent-4-enamido)-1-phenylethyl)carbamate was prepared from (R)-tert-butyl (2-(methylamino)-1-phenylethyl)carbamate (200.0 mg, 0.799 mmol) and (R)-2-methylpent-4-enoic acid (130.0 μl, 0.666 mmol). The product (215 mg) was obtained as a white solid in 74% crude yield. LRMS (ESI+) (M+H): 247.28.

(R)—N—((R)-2-amino-2-phenylethyl)-N,2-dimethylpent-4-enamide: Using the N-Boc (N-tert-butyl-oxycarbonyl) removal step from General Procedure for Amino Macrocyles, (R)—N—((R)-2-amino-2-phenylethyl)-N,2-dimethylpent-4-enamide was prepared from tert-butyl ((R)-2-((R)—N,2-dimethylpent-4-enamido)-1-phenylethyl)carbamate (259.0 mg, 0.746 mmol). The product (175 mg) was isolated in 95% yield after silica chromatography using an ISCO automated system (MeOH/DCM gradient). LRMS (ESI+) (M+H): 247.28.

(R)—N,2-dimethyl-N—((R)-2-(pent-4-enamido)-2-phenylethyl)pent-4-enamide: Using General Procedure B for Amide Bond Formation with DMF as solvent, (R)—N,2-dimethyl-N—((R)-2-(pent-4-enamido)-2-phenylethyl)pent-4-enamide was prepared from (R)—N—((R)-2-amino-2-phenylethyl)-N,2-dimethylpent-4-enamide (385.8 mg, 1.566 mmol) and pent-4-enoic acid (134.0 μl, 1.305 mmol). The product (274 mg) was isolated in 64% yield after silica chromatography using an ISCO automated system (MeOH/DCM gradient). LRMS (ESI+) (M+H): 329.24.

(3R,11R,E)-1,11-dimethyl-3-phenyl-1,4-diazacyclododec-8-ene-5,12-dione: Using General Procedure for Ring-Closing Metathesis using toluene as solvent, (R)—N,2-dimethyl-N—((R)-2-(pent-4-enamido)-2-phenylethyl)pent-4-enamide (231.3 mg, 0.704 mmol) was used in the reaction and stirred overnight (16 hours) at room temperature. The product (145 mg) was isolated in 68% yield after trituration in EtOAc and filtered to obtain a white solid. LRMS (ESI+) (M+H): 301.21.

### Example AG

### N-((3R,11S,E)-5,12-dioxo-3-phenyl-1-oxa-4-azacyclododec-8-en-11-yl)acetamide

tert-butyl ((4R,8S)-5-oxo-8-(pent-4-enamido)-8-phenyloct-1-en-4-yl)carbamate: Using General Procedure C for Ester Bond Formation using DCM as solvent tert-butyl ((4R,8S)-5-oxo-8-(pent-4-enamido)-8-phenyloct-1-en-4-yl)carbamate was prepared from (R)—N-(2-hydroxy-1-phenylethyl)pent-4-enamide (5.00 g, 22.8 mmol) and dicyclohexylammonium (R)-2-((tert-butoxycarbonyl)amino)pent-4-enoate (9.04 g, 22.8 mmol). The product was obtained as a white solid in 82% isolated yield after silica chromatography using an ISCO automated system (MeOH/DCM gradient). LRMS (ESI+) (M+H): 317.20.

N-((3R,11S,E)-5,12-dioxo-3-phenyl-1-oxa-4-azacyclododec-8-en-11-yl)acetamide: Under nitrogen, triethylamine (6.58 ml, 47.2 mmol) was added to the TFA salt form of (3R,11S,E)-11-amino-3-phenyl-1-oxa-4-azacyclododec-8 ene-5,12-dione (95.0 mg, 0.236 mmol). After stirring for one minute, acetic anhydride (4.46 ml, 47.2 mmol) was added to the mixture, upon which the reaction turned slightly clear and colorless. The reaction was stopped after 8 hours. The crude reaction was dried under high vacuum and product was purified by trituration with EtOAc (×2), followed by filtration and washing with DCM. The isolated product (249 mg) is 99% yield in the form of a white solid. LRMS (ESI+) (M+H): 331.29.

### Example AH

### N-((3R,8S,9S,11R)-8,9-dihydroxy-5,12-dioxo-3-phenyl-1-oxa-4-azacyclododecan-11-yl)acetamide

To a solution of (3R,11R,E)-11-amino-3-phenyl-1-oxa-4-azacyclododec-8 ene-5,12-dione in tBuOH (3.4 ml), THF (908 μl), DI-water (227 μl) was added followed by N-methylmorpholine-N-oxide (50 wt % solution in H₂O) (128 μl, 0.545 mmol) and OsO4 (2.5 wt % solution in tBuOH) (596 μl, 0.045 mmol). The reaction mixture was stirred at room temperature. The reaction was complete after 6 hours. The crude reaction mixture was diluted with saturated sodium sulfite and stirred for 1 hour. The solution was further diluted with EtOAc and water. The aqueous layer was extracted with EtOAc (×2) and the combined organic extracts were washed with brine, dried over Na₂SO₄ and concentrated under high vacuum. The crude product was purified using silica gel chromatography using an ISCO automated system and a MeOH/DCM gradient. The product (48 mg) was isolated as a white solid in 29% yield. LRMS (ESI+) (M+H): 359.18

### Example AI

### N-((3R,11R,E)-5,12-dioxo-3-phenyl-1-oxa-4-azacyclododec-8-en-11-yl)-iso butyramide

DMF (15.0 ml) was added to a flask containing TFA salt form of (3R,11R,E)-11-amino-3-phenyl-1-oxa-4-azacyclododec-8 ene-5,12-dione (50.0 mg, 0.173 mmol). Triethylamine (2.417 ml, 17.34 mmol) and isobutyric anhydride (2.88 ml, 17.34 mmol) were added to the reaction under nitrogen. The reaction mixture was stirred overnight at room temp (15 hours). The crude reaction was concentrated under high vacuum and the resulting white solid was washed with EtOAc and 1M HCl. The EtOAc layer was separated, and washed again with 1M HCl, then brine. The organic layer was dried over Na₂SO₄, and concentrated under high vacuum. The product (40 mg) was isolated by trituration in EtOAc, followed by filtration to give a white solid in 64% yield. LRMS (ESI+) (M+H): 359.15.

### Example AJ

### N-((3R,11R)-5,12-dioxo-3-phenyl-1-oxa-4-azacyclododecan-11-yl)acetamide

In a flask, under nitrogen, 10% Pd (dry) on carbon (wet) (32.2 mg, 0.030 mmol) was added to N-((3R,11R,E)-5,12-dioxo-3-phenyl-1-oxa-4-azacyclododec-8-en-11-yl)acetamide (10.0 mg, 0.030 mmol) in MeOH (10.0 ml) and reaction was stirred 24 h under hydrogen. Upon completion of the reaction, the crude reaction was filtered through celite and concentrated under high vacuum to give isolated product (9.96 mg) in 99% yield as a white solid. LRMS (ESI+) (M+H): 333.15.

### Example AK

### N-((3R,11R,E)-5,12-dioxo-3-phenyl-1-oxa-4-azacyclododec-8-en-11-yl)-1-methyl-1H-imidazole-2-carboxamide

Under nitrogen, 1-Methyl-1H-imidazole-2-carboxylic acid (7.0 mg, 0.053 mmol) was dissolved in anhydrous DMF (6001) with (iPr)₂EtN (36.7 μL, 0.211 mmol). Next, the TFA salt form of (3R,11R,E)-11-amino-3-phenyl-1-oxa-4-azacyclododec-8 ene-5,12-dione, 1-hydroxybenzotriazole (12.11 mg, 0.079 mmol) and 1-ethyl-3-(3′-dimethylaminopropyl)carbodiimide (15.16 mg, 0.079 mmol) were added. The reaction mixture was stirred overnight (15 hours). Upon completion of the reaction, the reaction was diluted with EtOAc. The crude mixture was washed twice with saturated NH₄Cl (×2), the half-saturated NaHCO₃, and brine. The crude mixture was dried over Na₂SO₄ and concentrated under vacuum to yield a white wax. The product (20.1 mg) was isolated via silica chromatography using an ISCO automated system (MeOH/DCM gradient) in 95% yield in the form of a white solid. LRMS (ESI+) (M+H): 397.24.

### Example AL

### N-((3R,11R,E)-5,12-dioxo-3-phenyl-1-oxa-4-azacyclododec-8-en-11-yl)-1H-imidazole-2-carboxamide

Under nitrogen, 1H-imidazole-2-carboxylic acid (8.40 mg, 0.075 mmol) was dissolved in anhydrous DMF (1.2 ml) with (iPr)₂EtN (48.0 μl, 0.277 mmol). Next, the TFA salt form of (3R,11R,E)-11-amino-3-phenyl-1-oxa-4-azacyclododec-8 ene-5,12-dione (30.0 mg, 0.075 mmol), 1-hydroxybenzotriazole (15.93 mg, 0.104 mmol) and 1-ethyl-3-(3′-dimethylaminopropyl)carbodiimide (19.95 mg, 0.104 mmol) were added. The reaction mixture was stirred overnight (15 hours). Upon completion of the reaction, the reaction was diluted with EtOAc. The crude mixture was washed twice with saturated NH₄Cl (×2), the half-saturated NaHCO₃, and brine. The crude mixture was dried over Na₂SO₄ and concentrated under vacuum to yield a white wax. The product (23.8 mg) was isolated via silica chromatography using an ISCO automated system (MeOH/DCM gradient) in 90% yield in the form of a white solid. LRMS (ESI+) (M+H): 383.17

### Example AM

### 1-((3R,11R,E)-5,12-dioxo-3-phenyl-1-oxa-4-azacyclododec-8-en-11-yl)-3-ethylurea

Under nitrogen, the TFA salt form of (3R,11R,E)-11-amino-3-phenyl-1-oxa-4-azacyclododec-8 ene-5,12-dione (20.0 mg, 0.050 mmol) and triethylamine (21 μl, 0.149 mmol) were dissolved in 0.5 ml THF. Next, isocyanatoethane (3.94 μl, 0.050 mmol) dissolved in 0.5 ml THF was added to the solution and the reaction was stirred for 18 hours. Upon completion of the reaction, the crude mixture was dried under high vacuum. The product (14.2 mg) was isolated via silica chromatography using an ISCO automated system (MeOH/DCM gradient) in 79% yield in the form of a white solid. LRMS (ESI+) (M+H): 360.24.

### Example AN

### 1-benzyl-3-((3R,11R,E)-5,12-dioxo-3-phenyl-1-oxa-4-azacyclododec-8-en-11-yl)urea

Under nitrogen, the TFA salt form of (3R,11R,E)-11-amino-3-phenyl-1-oxa-4-azacyclododec-8 ene-5,12-dione (20.0 mg, 0.050 mmol) and triethylamine (21.0 μl, 0.149 mmol) were dissolved in 0.5 ml THF. Next, (isocyanatomethyl)benzene (6.06 μl, 0.050 mmol) dissolved in 0.5 ml THF was added to the solution and the reaction was stirred for 18 hours. Upon completion of the reaction, the crude mixture was dried under high vacuum. The product (10.9 mg) was isolated via silica chromatography using an ISCO automated system (MeOH/DCM gradient) in 52% yield in the form of a white solid. LRMS (ESI+) (M+H): 408.21

### Example AO

### N-((3R,11R,E)-5,12-dioxo-3-phenyl-1-oxa-4-azacyclododec-8-en-11-yl)-1H-pyrazole-3-carboxamide

Under nitrogen, 1H-pyrazole-5-carboxylic acid (9.03 mg, 0.081 mmol) was dissolved in anhydrous DMF (1.2 ml) with (iPr)₂EtN (52.0 μl, 0.298 mmol). Next, the TFA salt form of (3R,11R,E)-11-amino-3-phenyl-1-oxa-4-azacyclododec-8 ene-5,12-dione (30.0 mg, 0.075 mmol), 1-hydroxy-7-azabenzotriazole (17.24 mg, 0.112 mmol) and 1-ethyl-3-(3′-dimethylaminopropyl)carbodiimide (21.44 mg, 0.112 mmol) were added. The reaction mixture was stirred overnight (15 hours). Upon completion of the reaction, the reaction was diluted with EtOAc. The crude mixture was washed twice with saturated NH₄Cl (×2), the half-saturated NaHCO₃, and brine. The crude mixture was dried over Na₂SO₄ and concentrated under vacuum to yield a white wax. The product (23.8 mg) was isolated via silica chromatography using an ISCO automated system (MeOH/DCM gradient) in 83% yield in the form of a white solid. LRMS (ESI+) (M+H): 382.86

### Example AP

### (3R,11R,E)-11-methoxy-3-phenyl-1-oxa-4-azacyclododec-8-ene-5,12-dione

(3R,11R,E)-11-hydroxy-3-phenyl-1-oxa-4-azacyclododec-8-ene-5,12-dione (5 mg, 0.017 mmol) and silver oxide (40.0 mg, 0.173 mmol) were added to a vial with stir bar and sealed under nitrogen, then acetone (1 mL) and iodomethane (0.108 mL, 1.728 mmol) were added. The reaction was stirred for 26 h, then the reaction solution was removed (leaving the bulk of the silver oxide behind) and filtered through a syringe filter, washing the flask with additional acetone. The crude solution was concentrated and purified by column chromatography (Pasteur pipet column and EtOAc/hexanes gradient). The desired product was isolated as a white solid (3.9 mg, 74%). LRMS (ESI+) (M+H): 304.22.

### Example AQ

### (3R,12R,E)-12-methyl-3-phenyl-1-oxa-4-azacyclotridec-9-ene-5,13-dione

(R)—N-(2-hydroxy-1-phenylethyl)hex-5-enamide: Using general procedure A for amide bond formation with DCM as solvent, the desired product was prepared from (R)-2-amino-2-phenylethanol and hex-5-enoic acid (228 mg, 2.00 mmol). The product was obtained as a white solid (340 mg, 73%) after work-up using EtOAc as solvent, and concentrating the final product in the presence of hexanes. LRMS (ESI+) (M+H): 234.18.

(R)—(R)-2-(hex-5-enamido)-2-phenylethyl 2-methylpent-4-enoate: Using general procedure C for ester bond formation with DCM as solvent, the desired product was prepared from (R)—N-(2-hydroxy-1-phenylethyl)hex-5-enamide (170 mg, 0.73 mmol) and (R)-2-methylpent-4-enoic acid, prepared according to a literature procedure (Chakraborty, T. K. et al. Synlett 2002, 2039-2040). The product was obtained as a colorless oil (238 mg, 99%) after work-up using EtOAc as solvent. LRMS (ESI+) (M+H): 330.30.

(3R,12R,E)-12-methyl-3-phenyl-1-oxa-4-azacyclotridec-9-ene-5,13-dione: Using the general procedure for ring closing metathesis with toluene as solvent, the desired product was prepared from (R)—(R)-2-(hex-5-enamido)-2-phenylethyl 2-methylpent-4-enoate (100 mg, 0.30 mmol) to yield the product as a white solid (53 mg, 58%) after column chromatography (EtOAc/hexanes gradient). LRMS (ESI+) (M+H): 302.22.

### Example AR

### (3R,12R,Z)-12-methyl-3-phenyl-1-oxa-4-azacyclotridec-9-ene-5,13-dione

The title compound was isolated as the minor, more polar product in the above reaction, i.e. synthesis of compound AQ, as a white solid (8.0 mg, 9%). LRMS (ESI+) (M+H): 302.22.

### Example AS

### (3R,8S,9S,11R)-6,6-difluoro-8,9-dihydroxy-11-methyl-3-phenyl-1-oxa-4-azacyclododecane-5,12-dione

To a solution of (3R,11R,E)-6,6-difluoro-11-methyl-3-phenyl-1-oxa-4-azacyclododec-8-ene-5,12-dione (10 mg, 0.031 mmol) in 14:4:1 t-BuOH/THF/water (0.5 mL) in a 4 mL vial with stir bar was added NMO (50 wt % solution in water) (8.7 μL, 0.037 mmol) followed by OsO₄ (2.5 wt % solution in t-BuOH) (41 μL, 0.003 mmol). The mixture was stirred at room temperature for 20 h, then half-saturated aq. sodium sulfite was added (0.5 mL) to quench the reaction, and the mixture was stirred for 1 h. The reaction was then diluted with EtOAc and water and the layers separated. The aqueous layer was extracted twice with EtOAc, and the combined organic extracts were washed with brine, dried over MgSO₄, filtered, and concentrated. The crude product was purified by column chromatography (Pasteur pipet column with MeOH/DCM gradient), yielding the desired product as a white solid (4.6 mg, 42%). LRMS (ESI+) (M+H): 358.14.

### Example AT

### (3R,6R,11R,E)-6-hydroxy-11-methyl-3-phenyl-1-oxa-4-azacyclododec-8-ene-5,12-dione

(R)-2-((tert-butyldimethylsilyl)oxy)-N—((R)-2-hydroxy-1-phenylethyl)pent-4-enamide: Using general procedure A for amide bond formation with DCM as solvent, the desired product was prepared from (R)-2-amino-2-phenylethanol and (R)-2-((tert-butyldimethylsilyl)oxy)pent-4-enoic acid (115 mg, 0.50 mmol). The product was obtained as a colorless oil (84 mg, 48%) after work-up using EtOAc as solvent, and column chromatography (EtOAc/hexanes gradient). LRMS (ESI+) (M+H): 350.29.

(R)—(R)-2-((R)-2-((tert-butyldimethylsilyl)oxy)pent-4-enamido)-2-phenylethyl 2-methylpent-4-enoate: Using general procedure C for ester bond formation with DCM as solvent, the desired product was prepared from (R)-2-((tert-butyldimethylsilyl)oxy)-N—((R)-2-hydroxy-1-phenylethyl)pent-4-enamide (84 mg, 0.24 mmol) and (R)-2-methylpent-4-enoic acid, prepared according to a literature procedure (Chakraborty, T. K. et al. Synlett 2002, 2039-2040). The product was obtained as a colorless oil (102 mg, 95%) after work-up using EtOAc as solvent. LRMS (ESI+) (M+H): 446.26.

(3R,6R,11R,E)-6-((tert-butyldimethylsilyl)oxy)-1-methyl-3-phenyl-1-oxa-4-azacyclododec-8-ene-5,12-dione: Using the general procedure for ring closing metathesis with toluene as solvent, the desired product was prepared from (R)—(R)-2-((R)-2-((tert-butyldimethylsilyl)oxy)pent-4-enamido)-2-phenylethyl 2-methylpent-4-enoate (101 mg, 0.227 mmol). The product was obtained as a white solid (44 mg, 58%) after column chromatography (EtOAc/hexanes gradient). LRMS (ESI+) (M+H): 418.23.

(3R,6R,11R,E)-6-hydroxy-1-methyl-3-phenyl-1-oxa-4-azacyclododec-8-ene-5,12-dione: (3R,6R,11R,E)-6-((tert-butyldimethylsilyl)oxy)-11-methyl-3-phenyl-1-oxa-4-azacyclododec-8-ene-5,12-dione (21.5 mg, 0.051 mmol) was dissolved with THF (0.5 mL) and TBAF (1 M in THF, 0.103 mL, 0.103 mmol) was added. The reaction was stirred for 0.5 h, then it was diluted with aq. NH₄Cl and EtOAc, and the layers separated. The combined organics were washed with water, then brine, then dried over MgSO₄, filtered, and concentrated. The resulting solid was purified by column chromatography (EtOAc/hexanes gradient), yielding a white solid (15 mg, 97%). LRMS (ESI+) (M+H): 304.18.

### Example AU

### (3R,6R,11R,E)-6-methoxy-11-methyl-3-phenyl-1-oxa-4-azacyclododec-8-ene-5,12-dione

(3R,6R,11R,E)-6-hydroxy-11-methyl-3-phenyl-1-oxa-4-azacyclododec-8-ene-5,12-dione (12 mg, 0.040 mmol) and silver oxide (92 mg, 0.40 mmol) were added to a vial with stir bar and sealed under nitrogen, then acetone (1 mL) and iodomethane (0.25 mL, 4.0 mmol) were added. The reaction was stirred for 26 h, then the reaction solution was removed (leaving the bulk of the silver oxide behind) and filtered through a syringe filter, washing the flask with additional acetone. The solution was concentrated to give the desired product as a white solid (11.7 mg, 93%). LRMS (ESI+) (M+H): 318.20.

### Example AV

### (R,E)-3-methyl-11-phenyl-1-oxa-3,10-diazacyclododec-5-ene-2,9-dione

(R)—N-(2-hydroxy-1-phenylethyl)pent-4-enamide: Using general procedure D for amide bond formation, the desired product was prepared from pent-4-enoic acid (1.037 g, 10.36 mmol) and (R)-2-amino-2-phenylethanol. The product (1.76 g, 77%) was obtained as a white solid after the crude oil obtained after work-up was concentrated with hexanes. LRMS (ESI+) (M+H): 220.11.

(R)-2-(pent-4-enamido)-2-phenylethyl allyl(methyl)carbamate: 1,1′-Carbonyldiimidizole (81 mg, 0.50 mmol) was added to a flame-dried flask with stir bar and sealed under nitrogen. Dry CH₃CN (2 mL) was added to form a colorless solution, to which was added a solution of (R)—N-(2-hydroxy-1-phenylethyl)pent-4-enamide (100 mg, 0.46 mmol) in dry CH₃CN from a vial sealed under nitrogen (dropwise over 10 min. via syringe). The vial was rinsed with additional CH₃CN (2×0.25 mL). The reaction was stirred for 2 h, then N-methyl-N-allylamine (0.130 ml, 1.34 mmol) was added, and the reaction was stirred for another 4 h, then diluted with EtOAc and 1 M aq. HCl. The layers were separated, and the combined organics were washed again with aq. HCl, then brine. The organic layer was dried over MgSO₄, filtered, and concentrated, then the crude oil was purified via column chromatography (EtOAc/hexanes gradient), yielding the desired product as a colorless oil (115 mg, 80%). LRMS (ESI+) (M+H): 317.25.

(R,E)-3-methyl-11-phenyl-1-oxa-3,10-diazacyclododec-5-ene-2,9-dione: Using the general procedure for ring closing metathesis with DCM as solvent, (R,E)-3-methyl-11-phenyl-1-oxa-3,10-diazacyclododec-5-ene-2,9-dione was prepared from (R)-2-(pent-4-enamido)-2-phenylethyl allyl(methyl)carbamate (115 mg, 0.363 mmol). The crude reaction was diluted with EtOAc prior to work-up. The product (47 mg, 45%) was obtained as a white solid following purification by column chromatography (MeOH/DCM gradient). LRMS (ESI+) (M+H): 289.27.

### Example AW

### (5R)-9-methyl-5-phenyl-7-oxa-1,4,9,12,13-pentaazabicyclo[9.2.1]tetradeca-11(14),12-diene-3,8-dione

(R)-tert-butyl (2-hydroxy-1-phenylethyl)carbamate: Boc₂O (3.00 g, 13.75 mmol), THF (31.6 mL), TEA (2.11 mL, 15.1 mmol), and (R)-2-amino-2-phenylethanol (1.084 g, 7.90 mmol) were added to a flask and stirred for 17 h. The reaction was then diluted with EtOAc and 1 M aq. HCl, the layers were separated, and the organic phase was washed again with aq. HCl, then brine. The combined organics were dried over MgSO₄, filtered, and concentrated. The resulting crude oil was purified by column chromatography (EtOAc/hexanes gradient) to yield the desired product as a white solid (1.18 g, 63%). LRMS (ESI+) (M+H): 238.33.

(R)-2-((tert-butoxycarbonyl)amino)-2-phenylethyl methyl(prop-2-yn-1-yl)carbamate: 1,1′-Carbonyldiimidizole (301 mg, 1.85 mmol) was added to an oven-dried flask with stir bar and sealed under nitrogen, then dissolved with dry CH₃CN (11.2 mL). (R)-tert-Butyl 2-hydroxy-1-phenylethylcarbamate (400 mg, 1.69 mmol) was added to a separate oven-dried vial and sealed under nitrogen, then dissolved with dry 1:1 CH₃CN/THF (5 mL). The resulting solution was then added dropwise over 10 min. via syringe to the solution of CDI, and the vial was rinsed with additional CH₃CN (2×1 mL). The reaction was stirred for 2 h, then N-methylprop-2-yn-1-amine (0.43 mL, 5.06 mmol) was added, and the reaction was stirred for another 5 h. The mixture was then concentrated by rotary evaporator and diluted with EtOAc and 1 M aq. HCl. The layers were separated, and the organic phase was washed again with aq. HCl, then brine. The combined organics were dried over MgSO₄, filtered, and concentrated. The resulting crude oil was purified by column chromatography (EtOAc/hexanes gradient), yielding the desired product as a colorless oil (192 mg, 34%). LRMS (ESI+) (M+H): 333.27.

(R)-2-amino-2-phenylethyl methyl(prop-2-yn-1-yl)carbamate: (R)-2-((tert-Butoxycarbonyl)amino)-2-phenylethyl methyl(prop-2-yn-1-yl)carbamate (192 mg, 0.58 mmol) was dissolved with DCM (7 ml), then triethylsilane (0.46 ml, 2.89 mmol) and trifluoroacetic acid (0.89 ml, 11.6 mmol) was added. The reaction was stirred for 20 h, then it was quenched by pipetting slowly into aq. NaHCO₃ to neutralize. The mixture was diluted with DCM and water, the layers were separated, and the organic phase was washed with brine, dried over Na₂SO₄, filtered, and concentrated to give the desired product as a pale yellow oil (118 mg, 88%). LRMS (ESI+) (M+H): 233.25.

(R)-2-(2-chloroacetamido)-2-phenylethyl methyl(prop-2-yn-1-yl)carbamate: (R)-2-Amino-2-phenylethyl methyl(prop-2-ynyl)carbamate (118 mg, 0.51 mmol) was added to a flask with stir bar and sealed under nitrogen, followed by DCM (5 mL). The solution was cooled on ice, then NEt₃ (0.092 mL, 0.66 mmol) was added, followed by 2-chloroacetyl chloride (0.049 mL, 0.61 mmol), added dropwise. Note: 2-chloroacetychloride is volatile and very toxic. The dark purple reaction was quenched after 40 min. by adding aq. NaHCO₃. The mixture was diluted with water and DCM, the phases were separated, and the organic phase was washed with brine, dried over MgSO₄, filtered, and concentrated. The resulting oil was purified by column chromatography (EtOAc/hexanes gradient) to give the desired product as a pale yellow oil (151 mg, 96%). LRMS (ESI+) (M+H): 309.13.

(R)-2-(2-azidoacetamido)-2-phenylethyl methyl(prop-2-yn-1-yl)carbamate: (R)-2-(2-chloroacetamido)-2-phenylethyl methyl(prop-2-yn-1-yl)carbamate (151 mg, 0.49 mmol) was dissolved with acetone (5 mL), and sodium azide (318 mg, 4.89 mmol) was added. The reaction was refluxed for 8 h, and the conversion of chloride to azide was followed by LC-MS. After 8 h, the reaction was deemed complete. The precipitated salts were removed by filtration through Celite, and the filter cake was washed with EtOAc. The mother liquor was diluted with water to dissolve any remaining sodium azide, partially concentrated to remove most of the acetone, and re-diluted with EtOAc. The layers were separated after the addition of some brine to facilitate separation, then the organic phase was dried over MgSO₄, filtered, and concentrated to provide the desired product as a pale yellow oil (129 mg, 84%). LRMS (ESI+) (M+H): 316.18.

(5R)-9-methyl-5-phenyl-7-oxa-1,4,9,12,13-pentaazabicyclo[9.2.1]tetradeca-11(14),12-diene-3,8-dione: (R)-2-(2-Azidoacetamido)-2-phenylethyl methyl(prop-2-yn-1-yl)carbamate (129 mg, 0.41 mmol) and toluene (45 mL) were added to a flask with stir bar. The flask was sealed and flushed well with nitrogen, then heated in an oil bath at 55° C. Amberlyst-21 resin loaded with CuPF₆ (4.53 g, 0.091 mmol/g, 0.41 mmol) was quickly added, and the reaction was stirred vigorously for 16 h. The Cu resin was synthesized according to a literature procedure (Kelly, A. R. et al. Org. Lett. 11, 2257-2260, 2009). The mixture was then filtered through Celite and washed with 1:1 MeOH/DCM (250 mL). The crude solution was concentrated then redissolved with 1:1 MeOH/DMSO, before undergoing purification by preparative LC-MS (acetonitrile/water gradient). The fractions containing the desired product were combined and concentrated to remove most of the CH₃CN, then extracted with EtOAc (3×30 mL), dried over MgSO₄, filtered, and concentrated to give the desired product as a white solid (3.3 mg, 2.6%). LRMS (ESI+) (M+H): 316.23.

### Example AX

### (R,E)-3-(4-chlorobenzyl)-11-phenyl-1-oxa-3,10-diazacyclododec-5-ene-2,9-dione

N-(4-chlorobenzyl)prop-2-en-1-amine: 4-Chlorobenzaldehyde (2.00 g, 14.2 mmol), 3 Å molecular sieves (4.27 g), MeOH (71 mL), and allylamine (1.28 ml, 17.1 mmol) were added to a flask and stirred for 2 h. The mixture was then cooled on ice and NaBH₄ (0.81 g, 21.3 mmol) was added in portions over 5 min. The reaction was removed from the ice bath and stirred for 2 h, then quenched by slowly adding water (100 mL). The crude mixture was filtered through Celite and washed with ether (300 mL total), then the layers were separated, and the aqueous layer was re-extracted with 3×50 mL ether. The combined extracts were washed with brine, dried over Na₂SO₄, filtered, and concentrated to provide the desired product as a colorless liquid (2.23 g, 86%). LRMS (ESI+) (M+H): 182.08.

(R)-2-(pent-4-enamido)-2-phenylethyl allyl(4-chlorobenzyl)carbamate: 4-Nitrophenylchloroformate (193 mg, 0.96 mmol) was sealed in a flask under nitrogen, then dry DCM (4 mL) and triethylamine (0.280 mL, 2.01 mmol) were added. (R)—N-(2-hydroxy-1-phenylethyl)pent-4-enamide (200 mg, 0.91 mmol) was added to a separate vial and sealed under nitrogen, then dissolved with dry DCM (1 mL). The resulting solution was added via syringe to the flask over 1 min., and the vial was rinsed with additional DCM (2×0.25 mL). The reaction was stirred for 0.5 h, then N-(4-chlorobenzyl)prop-2-en-1-amine (199 mg, 1.09 mmol) in DCM (1 mL) was added. The reaction was stirred for 16 h, then diluted with DCM, washed twice w with 1 M aq. HCl, then three times with half-saturated aq. NaHCO₃, followed by brine. The organic phase was dried over MgSO₄, filtered, and concentrated, then the crude oil was purified by column chromatography (EtOAc/hexanes gradient) to yield 71 mg (18%) of the desired product as a white solid. LRMS (ESI+) (M+H): 427.23

(R,E)-3-(4-chlorobenzyl)-1-phenyl-1-oxa-3,10-diazacyclododec-5-ene-2,9-dione: Using the general procedure for ring closing metathesis with toluene as solvent, the desired product was prepared from (R)-2-(pent-4-enamido)-2-phenylethyl allyl(4-chlorobenzyl)carbamate (35 mg, 0.082 mmol). The crude reaction was diluted with EtOAc prior to work-up. Product (8.1 mg, 25%) was obtained as a white solid following purification by column chromatography (MeOH/DCM gradient). LRMS (ESI+) (M+H): 399.11.

### Example AY

### (3R,11R,E)-6,6,11-trimethyl-3-phenyl-1-oxa-4-azacyclododec-8-ene-5,12-dione

(R)—N-(2-hydroxy-1-phenylethyl)-2,2-dimethylpent-4-enamide: 2,2-Dimethyl-4-pentenoic acid (128 mg, 1.0 mmol) was added to a flame-dried flask with stir bar and sealed under nitrogen. Dry DCM (5.0 mL) was added, then the flask was cooled on ice and oxalyl chloride (0.092 ml, 1.05 mmol) was added, followed by DMF (1 drop from a 22-gauge needle). The reaction was removed from the ice bath and stirred for 20 h, then (i-Pr)₂NEt (0.437 ml, 2.50 mmol) was added, followed by (R)-2-phenylglycinol (206 mg, 1.50 mmol). The reaction was stirred for another 20 h, then it was diluted with EtOAc and 1 M aq. HCl. The layers were separated, and the combined organics were washed again with aq. HCl, then twice with half-saturated aq. NaHCO₃, then brine. The organic phase was dried over MgSO₄, filtered, and concentrated to give the desired product as a white solid (214 mg, 87%). LRMS (ESI+) (M+H): 248.20.

(R)—(R)-2-(2,2-dimethylpent-4-enamido)-2-phenylethyl 2-methylpent-4-enoate: Using general procedure C for ester bond formation with DCM as solvent, the desired product was prepared from (R)—N-(2-hydroxy-1-phenylethyl)-2,2-dimethylpent-4-enamide (50 mg, 0.20 mmol) and (R)-2-methylpent-4-enoic acid, prepared according to a literature procedure (Chakraborty, T. K. et al. Synlett 2002, 2039-2040). The product was obtained as a white solid (68 mg, 99%) after work-up using EtOAc as solvent. LRMS (ESI+) (M+H): 344.31.

(3R,11R,E)-6,6,11-trimethyl-3-phenyl-1-oxa-4-azacyclododec-8-ene-5,12-dione: Using the general procedure for ring closing metathesis using toluene as solvent, the desired product was prepared from (R)—(R)-2-(2,2-dimethylpent-4-enamido)-2-phenylethyl 2-methylpent-4-enoate (69 mg, 0.20 mmol), yielding a colorless oil (51 mg, 81%) after column chromatography (EtOAc/hexanes gradient). LRMS (ESI+) (M+H): 316.22.

### Compound AZ

### (R)-2-(pent-4-enamido)-2-phenylethyl 2,2-dimethylpent-4-enoate

2,2-Dimethyl-4-pentenoic acid (154 mg, 1.20 mmol) was added to an oven-dried flask with stir bar and sealed under nitrogen. Dry DCE (5.0 mL) was added and the flask was cooled on ice, then oxalyl chloride (0.105 mL, 1.20 mmol) and DMF (1 drop from a 22-gauge needle) were added. The reaction was removed from the ice and stirred for 17 h, then cooled on ice again before (i-Pr)₂NEt (0.52 mL, 3.00 mmol), (R)—N-(2-hydroxy-1-phenylethyl)pent-4-enamide (219 mg, 1.00 mmol) and DMAP (12.2 mg, 0.10 mmol) were added. The reaction was stirred for 5 h, then it was diluted with EtOAc and 2 M aq. HCl. The layers were separated, and the combined organics were washed again with aq. HCl, then twice with half-saturated aq. NaHCO₃, then brine. The organic phase was dried over MgSO₄, filtered, and concentrated, and the resulting crude solid was purified by column chromatography (EtOAc/hexanes gradient), yielding the desired product as a colorless oil (69 mg, 21%). LRMS (ESI+) (M+H): 330.28.

### Example BB

### N-(4-chlorobenzyl)-2-((2R,3S,6S,8S,9S)-8,9-dihydroxy-3,4-dimethyl-5,12-dioxo-2-phenyl-1-oxa-4-azacyclododecan-6-yl)acetamide

N-(4-chlorobenzyl)-2-((2R,3S,6S,E)-3,4-dimethyl-5,12-dioxo-2-phenyl-1-oxa-4-azacyclododec-8-en-6-yl)acetamide (38 mg, 0.079 mmol) was dissolved in a mixture of t-BuOH/THF/H₂O 15/4/1 (786 μL). To this solution were added successively osmium tetroxide (2.5 wt. % solution in t-BuOH, 103 μL, 7.9 mol) and N-methylmorpholine-N-oxide (50 wt. % solution in water, 22 μL, 0.094 mmol). The mixture was stirred for 3 h, after which a solution of saturated sodium sulfite was added and stirred for 1 h. After extraction with ethyl acetate, the organic phase was dried and concentrated. The product (24 mg) was obtained in 59% yield after column chromatography (MeOH/DCM gradient). LRMS (ESI+) (M+H): 518.23.

### Example BC

### (2R,3S,6R,E)-3,4,6-trimethyl-2-phenyl-1-oxa-4-azacyclododec-8-ene-5,12-dione

(R)—N-((1R,2S)-1-hydroxy-1-phenylpropan-2-yl)-N,2-dimethylpent-4-enamide: Using general procedure A for amide bond formation with DMF as solvent, the title compound was prepared from (R)-2-methylpent-4-enoic acid (Chakraborty, T. K. et al. Synlett 2039-2040, 2002] (76 mg, 0.666 mmol) and (1R,2S)-2-(methylamino)-1-phenylpropan-1-ol (100 mg, 0.605 mmol). The product (99 mg) was obtained in 63% yield after column chromatography (Hex/EA gradient). LRMS (ESI+) (M+Na): 284.22.

(1R,2S)-2-((R)—N,2-dimethylpent-4-enamido)-1-phenylpropyl pent-4-enoate: Using general procedure C for ester bond formation with DCM as solvent the title compound was prepared from 4-pentenoic acid (43 μL, 0.417 mmol) and (R)—N-((1R,2S)-1-hydroxy-1-phenylpropan-2-yl)-N,2-dimethylpent-4-enamide (99 mg, 0.379 mmol). The product (93 mg) was obtained in 71% yield after column chromatography (Hex/EA gradient). LRMS (ESI+) (M+Na): 366.20.

(2R,3S,6R,E)-3,4,6-trimethyl-2-phenyl-1-oxa-4-azacyclododec-8-ene-5,12-dione: Using the general procedure for ring closing metathesis with toluene as solvent the title compound was prepared from (1R,2S)-2-((R)—N,2-dimethylpent-4-enamido)-1-phenylpropyl pent-4-enoate (93 mg, 0.27 mmol). The product (8 mg) was obtained in 10% yield following purification by column chromatography (Hex/EA gradient). LRMS (ESI+) (M+Na): 338.27.

### Example BD

### (9R,14aS,E)-9-methyl-4,5,8,9,12,13,14,14a-octahydro-1H-pyrrolo[2,1-c][1,4]oxaazacyclododecine-3,10-dione

(R)-1-((S)-2-(hydroxymethyl)pyrrolidin-1-yl)-2-methylpent-4-en-1-one: Using general procedure A for amide bond formation with DCM as solvent, the title compound was prepared from (R)-2-methylpent-4-enoic acid (Chakraborty, T. K. et al. Synlett 2039-2040, 2002] (99 mg, 0.87 mmol) and (S)-pyrrolidin-2-ylmethanol (80 mg, 0.791 mmol). The product (106 mg) was obtained in 68% yield after column chromatography (Hex/EA gradient). LRMS (ESI+) (M+H): 198.16.

((S)-1-((R)-2-methylpent-4-enoyl)pyrrolidin-2-yl)methyl pent-4-enoate: Using general procedure C for ester bond formation with DCM as solvent the title compound was prepared from 4-pentenoic acid (60 μL, 0.591 mmol) and (R)-1-((S)-2-(hydroxymethyl)pyrrolidin-1-yl)-2-methylpent-4-en-1-one (106 mg, 0.537 mmol). The product (121 mg) was obtained in 81% yield after column chromatography (Hex/EA gradient). LRMS (ESI+) (M+H): 280.23.

(9R,14aS,E)-9-methyl-4,5,8,9,12,13,14,14a-octahydro-1H-pyrrolo[2,1-c][1,4]oxaazacyclododecine-3,10-dione: Using the general procedure for ring closing metathesis with toluene as solvent the title compound was prepared from ((S)-1-((R)-2-methylpent-4-enoyl)pyrrolidin-2-yl)methyl pent-4-enoate (121 mg, 0.433 mmol). The product (46 mg) was obtained in 42% yield following purification by column chromatography (Hex/EA gradient). LRMS (ESI+) (M+H): 252.16.

### Example BE

### (9R,14aS)-9-methyldecahydro-1H-pyrrolo[2,1-c][1,4]oxaazacyclododecine-3,10-dione

(9R,14aS,E)-9-methyl-4,5,8,9,12,13,14,14a-octahydro-1H-pyrrolo[2,1-c][1,4]oxaazacyclododecine-3,10-dione (15 mg, 0.06 mmol) in methanol (597 μL) was stirred under hydrogen atmosphere in the presence of palladium 10 wt. % on activated carbon (7 mg). After 3 h at room temperature, the reaction mixture was filtered, washed with methanol, and evaporated. The product (15 mg) was obtained in 99% yield. LRMS (ESI+) (M+H): 254.15.

### Example BF

### 2-((2R,6S,8S,9S)-8,9-dihydroxy-5,12-dioxo-2-phenyl-1-oxa-4-azacyclododecan-6-yl)-N-(thiophen-2-ylmethyl)acetamide

2-((2R,6S,E)-5,12-dioxo-2-phenyl-1-oxa-4-azacyclododec-8-en-6-yl)-N-(thiophen-2-ylmethyl)acetamide (10 mg, 0.023 mmol) was dissolved in a mixture of t-BuOH/THF/H₂O 15/4/1 (235 μL). To this solution were added successively osmium tetroxide (2.5 wt. % solution in t-BuOH, 31 μL, 2.35 μmol) and N-methylmorpholine-N-oxide (50 wt. % solution in water, 6.6 μL, 0.028 mmol). The mixture was stirred for 12 h, after which a solution of saturated sodium sulfite is added and stirred for 1 h. After extraction with ethyl acetate, the organic phase was dried and concentrated. The product (4 mg) was obtained in 19% yield after column chromatography (MeOH/DCM gradient). LRMS (ESI+) (M+H): 461.36.

### Example BG

### N-((2R,3S,6R,E)-3,4-dimethyl-5,12-dioxo-2-phenyl-1-oxa-4-azacyclododec-8-en-6-yl)acetamide

(9H-fluoren-9-yl)methyl (R)-1-(((1R,2S)-1-hydroxy-1-phenylpropan-2-yl)(methyl)amino)-1-oxopent-4-en-2-ylcarbamate: Using general procedure A for amide bond formation with DCM as solvent, the title compound was prepared from (R)-2-(((9H-fluoren-9-yl)methoxy)carbonylamino)pent-4-enoic acid [Hruby, V. J. et al. Org. Lett. 6, 3285-3288, 2004](408 mg, 1.21 mmol) and (1R,2S)-2-(methylamino)-1-phenylpropan-1-ol (200 mg, 1.21 mmol). The product (587 mg) was obtained and used in the next step without purification. LRMS (ESI+) (M+H): 485.34.

(1R,2S)-2-((R)-2-(((9H-fluoren-9-yl)methoxy)carbonylamino)-N-methylpent-4-enamido)-1-phenylpropyl pent-4-enoate: Using general procedure C for ester bond formation with DCM as solvent the title compound was prepared from 4-pentenoic acid (136 μL, 1.332 mmol) and (9H-fluoren-9-yl)methyl (R)-1-(((1R,2S)-1-hydroxy-1-phenylpropan-2-yl)(methyl)amino)-1-oxopent-4-en-2-ylcarbamate (587 mg, 1.211 mmol). The product (485 mg) was obtained in 71% yield over 2 steps after column chromatography (Hex/EA gradient). LRMS (ESI+) (M+H): 567.41.

(9H-fluoren-9-yl)methyl (2R,3S,6R,E)-3,4-dimethyl-5,12-dioxo-2-phenyl-1-oxa-4-azacyclododec-8-en-6-ylcarbamate: Using the general procedure for ring closing metathesis with toluene as solvent the title compound was prepared from (1R,2S)-2-((R)-2-(((9H-fluoren-9-yl)methoxy)carbonylamino)-N-methylpent-4-enamido)-1-phenylpropyl pent-4-enoate (485 mg, 0.856 mmol). The product (75 mg) was obtained in 16% yield following purification by column chromatography (Hex/EA gradient). LRMS (ESI+) (M+H): 539.35.

(2R,3S,6R,E)-6-amino-3,4-dimethyl-2-phenyl-1-oxa-4-azacyclododec-8-ene-5,12-dione: (9H-fluoren-9-yl)methyl (2R,3S,6R,E)-3,4-dimethyl-5,12-dioxo-2-phenyl-1-oxa-4-azacyclododec-8-en-6-ylcarbamate (75 mg, 0.139 mmol) was dissolved in DMF (5.1 mL) and piperidine (1.03 mL, 10.44 mmol) was added dropwise. After 30 minutes, the reaction is complete as shown by LCMS. Solvents are evaporated and coevaporated with toluene 3 times. The product (44 mg) was obtained and used in the next step without further purification. LRMS (ESI+) (M+H): 317.15.

N-((2R,3S,6R,E)-3,4-dimethyl-5,12-dioxo-2-phenyl-1-oxa-4-azacyclododec-8-en-6-yl)acetamide: (2R,3S,6R,E)-6-amino-3,4-dimethyl-2-phenyl-1-oxa-4-azacyclododec-8-ene-5,12-dione (44 mg, 0.139 mmol) was dissolved in DMF (2.8 mL) under argon atmosphere. Triethylamine (586 μL, 4.17 mmol) and acetic anhydride (394 μL, 4.17 mmol) were successively added in one portion and the reaction mixture was stirred at room temperature for 6 h. Solvents were evaporated before the mixture was dissolved with ethyl acetate and successively washed with NH₄Cl solution and NaHCO₃ solution. The product (18 mg) was obtained in 36% yield after purification by column chromatography (DCM/MeOH gradient). LRMS (ESI+) (M+H): 359.49.

### Example BH

### N-((9R,14aS,E)-3,10-dioxo-3,4,5,8,9,10,12,13,14,14a-decahydro-1H-pyrrolo[2,1-c][1,4]oxaazacyclododecin-9-yl)acetamide

tert-butyl (R)-1-((S)-2-(hydroxymethyl)pyrrolidin-1-yl)-1-oxopent-4-en-2-ylcarbamate: Using general procedure A for amide bond formation with DCM as solvent, the title compound was prepared from (R)-2-(tert-butoxycarbonylamino)pent-4-enoic acid dicyclohexylamine salt (484 mg, 2.247 mmol) and (S)-pyrrolidin-2-ylmethanol (250 mg, 2.472 mmol). The product (670 mg) was obtained and used in the next step without purification. LRMS (ESI+) (M+H): 299.18.

((S)-1-((R)-2-(tert-butoxycarbonylamino)pent-4-enoyl)pyrrolidin-2-yl)methyl pent-4-enoate: Using general procedure C for ester bond formation with DCM as solvent the title compound was prepared from 4-pentenoic acid (252 μL, 2.47 mmol) and tert-butyl (R)-1-((S)-2-(hydroxymethyl)pyrrolidin-1-yl)-1-oxopent-4-en-2-ylcarbamate (670 mg, 2.245 mmol). The product (721 mg) was obtained in 84% yield over 2 steps after column chromatography (Hex/EA gradient). LRMS (ESI+) (M+H): 381.22.

tert-butyl (9R,14aS,E)-3,10-dioxo-3,4,5,8,9,10,12,13,14,14a-decahydro-1H-pyrrolo[2,1-c][1,4]oxaazacyclododecin-9-ylcarbamate: Using the general procedure for ring closing metathesis with toluene as solvent the title compound was prepared from ((S)-1-((R)-2-(tert-butoxycarbonylamino)pent-4-enoyl)pyrrolidin-2-yl)methyl pent-4-enoate (721 mg, 1.895 mmol). The product (124 mg) was obtained in 19% yield following purification by column chromatography (Hex/EA gradient). LRMS (ESI+) (M+H): 353.18.

(9R,14aS,E)-9-amino-4,5,8,9,14,14a-hexahydro-1H-pyrrolo[2,1-c][1,4]oxaazacyclododecine-3,10(12H,13H)-dione: tert-butyl (9R,14aS,E)-3,10-dioxo-3,4,5,8,9,10,12,13,14,14a-decahydro-1H-pyrrolo[2,1-c][1,4]oxaazacyclododecin-9-ylcarbamate (124 mg, 0.352 mmol) was dissolved in DCM (352 μL) and trifluoroacetic acid (136 μL, 1.759 mmol) was added dropwise. After 2 h, the reaction was complete as shown by LCMS. Solvents were evaporated and coevaporated with toluene 3 times. The product (89 mg) was obtained and used in the next step without further purification. LRMS (ESI+) (M+H): 253.09.

N-((9R,14aS,E)-3,10-dioxo-3,4,5,8,9,10,12,13,14,14a-decahydro-1H-pyrrolo[2,1-c][1,4]oxaazacyclododecin-9-yl)acetamide: (9R,14aS,E)-9-amino-4,5,8,9,14,14a-hexahydro-1H-pyrrolo[2,1-c][1,4]oxaazacyclododecine-3,10(12H, 13H)-dione (89 mg, 0.353 mmol) was dissolved in DMF (7 mL) under argon atmosphere. Triethylamine (2.9 mL, 21.16 mmol) and acetic anhydride (2 mL, 21.16 mmol) were successively added in one portion and the reaction mixture was stirred at room temperature for 15 h. Solvents were evaporated before the mixture was dissolved with ethyl acetate and successively washed with NH₄Cl solution and NaHCO₃ solution. The product (77 mg) was obtained in 74% yield after purification by column chromatography (DCM/MeOH gradient). LRMS (ESI+) (M+H): 295.26.

### Example BI

### N-((2R,3R,6R,E)-3,4-dimethyl-5,12-dioxo-2-phenyl-1-oxa-4-azacyclododec-8-en-6-yl)acetamide

tert-butyl (R)-1-(((1R,2R)-1-hydroxy-1-phenylpropan-2-yl)(methyl)amino)-1-oxopent-4-en-2-ylcarbamate: Using general procedure A for amide bond formation with DCM as solvent, the title compound was prepared from (R)-2-(tert-butoxycarbonylamino)pent-4-enoic acid dicyclohexylamine salt (167 mg, 0.776 mmol) and (1R,2R)-2-(methylamino)-1-phenylpropan-1-ol (70 mg, 0.424 mmol). The product (154 mg) was obtained and used in the next step without purification. LRMS (ESI+) (M+H): 363.18.

(1R,2R)-2-((R)-2-(tert-butoxycarbonylamino)-N-methylpent-4-enamido)-1-phenylpropyl pent-4-enoate: Using general procedure C for ester bond formation with DCM as solvent the title compound was prepared from 4-pentenoic acid (48 μL, 0.467 mmol) and tert-butyl (R)-1-(((1R,2R)-1-hydroxy-1-phenylpropan-2-yl)(methyl)amino)-1-oxopent-4-en-2-ylcarbamate (154 mg, 0.425 mmol). The product (158 mg) was obtained in 84% yield over 2 steps after column chromatography (Hex/EA gradient). LRMS (ESI+) (M+H): 445.21.

tert-butyl (2R,3R,6R,E)-3,4-dimethyl-5,12-dioxo-2-phenyl-1-oxa-4-azacyclododec-8-en-6-ylcarbamate: Using the general procedure for ring closing metathesis with toluene as solvent the title compound was prepared from (1R,2R)-2-((R)-2-(tert-butoxycarbonylamino)-N-methylpent-4-enamido)-1-phenylpropyl pent-4-enoate (158 mg, 0.355 mmol). The product (89 mg) was obtained in 60% yield following purification by column chromatography (Hex/EA gradient). LRMS (ESI+) (M+H): 417.22.

(2R,3R,6R,E)-6-amino-3,4-dimethyl-2-phenyl-1-oxa-4-azacyclododec-8-ene-5,12-dione: tert-butyl (2R,3R,6R,E)-3,4-dimethyl-5,12-dioxo-2-phenyl-1-oxa-4-azacyclododec-8-en-6-ylcarbamate (89 mg, 0.214 mmol) was dissolved in DCM (427 μL) and trifluoroacetic acid (82 μL, 1.068 mmol) was added dropwise. After 2 h, the reaction was complete as shown by LCMS. Solvents were evaporated and coevaporated with toluene 3 times. Product (67 mg) was obtained and used in the next step without further purification. LRMS (ESI+) (M+H): 317.24.

N-((2R,3R,6R,E)-3,4-dimethyl-5,12-dioxo-2-phenyl-1-oxa-4-azacyclododec-8-en-6-yl)acetamide: (2R,3R,6R,E)-6-amino-3,4-dimethyl-2-phenyl-1-oxa-4-azacyclododec-8-ene-5,12-dione (67 mg, 0.212 mmol) was dissolved in DMF (2.6 mL) under argon atmosphere. Triethylamine (1786 μL, 12.71 mmol) and acetic anhydride (1201 μL, 12.71 mmol) were successively added in one portion and the reaction mixture was stirred at room temperature for 15 h. Solvents were evaporated before the mixture was dissolved with ethyl acetate and successively washed with NH₄Cl solution and NaHCO₃ solution. The product (49 mg) was obtained in 65% yield after purification by column chromatography (DCM/MeOH gradient). LRMS (ESI+) (M+H): 359.16.

### Example BJ

### N-(4-chlorobenzyl)-2-((3S,10aR,15aS,E)-2,9-dioxo-2,3,4,7,8,9,10,10a,15,15a-decahydroindeno[2,1-b][1,4]oxaazacyclododecin-3-yl)acetamide

To a solution of tert-butyl 2-((3S,10aR,15aS,E)-2,9-dioxo-2,3,4,7,8,9,10,10a,15,15a-decahydroindeno[2,1-b][1,4]oxaazacyclododecin-3-yl)acetate (100 mg, 0.25 mmol) in CH₂Cl₂ (2.8 mL) cooled in an ice/water bath was added trifluoroacetic acid (288 μL, 3.75 mmol) and the reaction mixture was stirred 4 h at which point TLC analysis indicated complete consumption of starting material. The reaction mixture was concentrated and coevaporated 3 times with toluene. Then, using general procedure A for amide bond formation with DMF as solvent, the title compound was prepared from 2-((3S,10aR,15aS,E)-2,9-dioxo-2,3,4,7,8,9,10,10a,15,15a-decahydroindeno[2,1-b][1,4]oxaazacyclododecin-3-yl)acetic acid (86 mg, 0.25 mmol) and (4-chlorophenyl)methanamine (34 μl, 0.28 mmol). The product (72 mg) was obtained in 62% yield over 2 steps after column chromatography (MeOH/DCM gradient). LRMS (ESI+) (M+H): 467.26.

### Compound BK

### (3R,10aR,15aR,E)-3-methyl-3,4,7,8,10,10a,15,15a-octahydroindeno[2,1-b][1,4]oxaazacyclododecine-2,9-dione

N-((1R,2R)-2-hydroxy-2,3-dihydro-1H-inden-1-yl)pent-4-enamide: Using general procedure A for amide bond formation with DMF as solvent, the desired product was prepared from (1R,2R)-1-amino-2,3-dihydro-1H-inden-2-ol (118.0 mg, 0.769 mmol) and pent-4-enoic acid (70.0 mg, 0.07 mL, 0.699 mmol). The product was obtained as a white solid (120.0 mg, 74.2% yield) after column chromatography (EtOAc/hexane gradient). LRMS (ESI+) (M+H): 232.16.

(R)-(1R,2R)-1-(pent-4-enamido)-2,3-dihydro-1H-inden-2-yl 2-methylpent-4-enoate: Using general procedure C for ester bond formation with DMF as solvent, the desired product was prepared from N-((1R,2R)-2-hydroxy-2,3-dihydro-1H-inden-1-yl)pent-4-enamide (120.0 mg, 0.52 mmol) and (R)-2-methylpent-4-enoic acid (77.0 mg, 0.57 mmol), prepared according to a literature procedure (Chakraborty, T. K. et al. Synlett 2002, 2039-2040). The product was obtained as a white solid (80.0 mg, 47.1% yield) after column chromatography (EtOAc/hexane gradient). LRMS (ESI+) (M+H): 328.60.

(3R,10aR,15aR,E)-3-methyl-3,4,7,8,10,10a,15,15a-octahydroindeno[2,1-b][1,4]oxaazacyclododecine-2,9-dione: Using the general procedure for ring closing metathesis using DCM as solvent, the desired product was prepared from (R)-(1R,2R)-1-(pent-4-enamido)-2,3-dihydro-1H-inden-2-yl 2-methylpent-4-enoate (80.0 mg, 0.244 mmol), yielding a white solid (45.2 mg, 61.8% yield). LRMS (ESI+) (M+H): 300.50.

### Example BL

### (3R,10aS,13aS,E)-3-methyl-3,4,7,8,10,10a,11,12,13,13a-decahydrocyclopenta[b][1,4]oxaazacyclododecine-2,9-dione

N-((1S,2S)-2-hydroxycyclopentyl)pent-4-enamide: Using general procedure A for amide bond formation with DCM as solvent, the desired product was prepared from (1S,2S)-2-aminocyclopentanol (100.0 mg, 0.99 mmol) and pent-4-enoic acid (109.0 mg, 0.11 mL, 1.09 mmol). The product was obtained as a white solid (70 mg, 38.6%) after column chromatography (EtOAc/hexane gradient). LRMS (ESI+) (M+H): 184.16.

(R)-(1S,2S)-2-(pent-4-enamido)cyclopentyl 2-methylpent-4-enoate: Using general procedure C for ester bond formation with DCM as solvent, the desired product was prepared from N-((1S,2S)-2-hydroxycyclopentyl)pent-4-enamide (70.0 mg, 0.38 mmol) and (R)-2-methylpent-4-enoic acid (48.0 mg, 0.42 mmol), prepared according to a literature procedure (Chakraborty, T. K. et al. Synlett 2002, 2039-2040). The product was obtained as a white solid (62.0 mg, 58.1% yield) after column chromatography (EtOAc/hexane gradient). LRMS (ESI+) (M+H): 280.25.

(3R,10aS,13aS,E)-3-methyl-3,4,7,8,10,10a,11,12,13,13a-decahydrocyclopenta[b][1,4]oxaazacyclododecine-2,9-dione: Using the general procedure for ring closing metathesis using toluene as solvent, the desired product was prepared from (R)-(1S,2S)-2-(pent-4-enamido)cyclopentyl 2-methylpent-4-enoate (62.0 mg, 0.22 mmol), yielding a white solid (14.2 mg, 25.5% yield). LRMS (ESI+) (M): 251.76.

### Example BM

### (3R,10aS,13aR,E)-3-methyl-3,4,7,8,10,10a,11,12,13,13a-decahydrocyclopenta[b][1,4]oxaazacyclododecine-2,9-dione

(R)-(1R,2S)-2-(pent-4-enamido)cyclopentyl 2-methylpent-4-enoate: Using general procedure A for amide bond formation with DCM as solvent, the desired product was prepared from (1R,2S)-2-aminocyclopentanol (158.0 mg, 1.56 mmol) and pent-4-enoic acid (130.0 mg, 0.13 mL, 1.29 mmol). The product was obtained as a white solid (40.0 mg, 16.8% yield) after column chromatography (EtOAc/hexane gradient). Using general procedure C for ester bond formation with DCM as solvent, the desired product was prepared from N-((1S,2R)-2-hydroxycyclopentyl)pent-4-enamide (40.0 mg, 0.22 mmol) and (R)-2-methylpent-4-enoic acid (27.4 mg, 0.24 mmol), prepared according to a literature procedure (Chakraborty, T. K. et al. Synlett 2002, 2039-2040). The product was obtained as a white solid (61.0 mg, 100.0% yield) after column chromatography (EtOAc/hexane gradient). LRMS (ESI+) (M+H): 280.31.

(3R,10aS,13aR,E)-3-methyl-3,4,7,8,10,10a,11,12,13,13a-decahydrocyclopenta[b][1,4]oxaazacyclododecine-2,9-dione: Using the general procedure for ring closing metathesis using toluene as solvent, the desired product was prepared from (R)-(1R,2S)-2-(pent-4-enamido)cyclopentyl 2-methylpent-4-enoate (63.0 mg, 0.23 mmol), yielding a white solid (18.3 mg, 32.4% yield). LRMS (ESI+) (M): 251.76.

### Example BN

### (3S,6S,11R,E)-6,11-dimethyl-3-(thiophen-2-yl)-1-oxa-4-azacyclododec-8-ene-5,12-dione

(S)—N—((S)-2-hydroxy-1-(thiophen-2-yl)ethyl)-2-methylpent-4-enamide: Using general procedure A for amide bond formation with DCM as solvent, the desired product was prepared from (S)-2-amino-2-(thiophen-2-yl)ethanol-HCl salt (61.4 mg, 0.342 mmol) and (S)-2-methylpent-4-enoic acid (65.0 mg, 0.342 mmol), prepared according to a literature procedure (Chakraborty, T. K. et al. Synlett 2002, 2039-2040). The product was obtained as a white solid (58.3 mg, 71.3%) after column chromatography (EtOAc/hexane gradient). LRMS (ESI+) (M+H): 240.14.

(R)—(S)-2-((S)-2-methylpent-4-enamido)-2-(thiophen-2-yl)ethyl 2-methylpent-4-enoate: Using general procedure C for ester bond formation with DCM as solvent, the desired product was prepared from (S)—N—((S)-2-hydroxy-1-(thiophen-2-yl)ethyl)-2-methylpent-4-enamide (58.3 mg, 0.24 mmol) and (R)-2-methylpent-4-enoic acid (30.6 mg, 0.27 mmol), prepared according to a literature procedure (Chakraborty, T. K. et al. Synlett 2002, 2039-2040). The product was obtained as a white solid (51.6 mg, 63.1% yield) after column chromatography (EtOAc/hexane gradient). LRMS (ESI+) (M+H): 336.27.

(3S,6S,11R,E)-6,11-dimethyl-3-(thiophen-2-yl)-1-oxa-4-azacyclododec-8-ene-5,12-dione: Using the general procedure for ring closing metathesis using toluene as solvent, the desired product was prepared from (R)—(S)-2-((S)-2-methylpent-4-enamido)-2-(thiophen-2-yl)ethyl 2-methylpent-4-enoate (51.6 mg, 0.154 mmol), yielding a white solid (35.3 mg, 74.7% yield) after column chromatography (EtOAc/hexanes gradient). LRMS (ESI+) (M+H): 308.54.

### Example BO

### (3R,6S,11R,E)-6,11-dimethyl-3-(pyridin-2-yl)-1-oxa-4-azacyclododec-8-ene-5,12-dione

(S)—N—((R)-2-hydroxy-1-(pyridin-2-yl)ethyl)-2-methylpent-4-enamide: Using general procedure A for amide bond formation with DCM as solvent, the desired product was prepared from (R)-2-amino-2-(pyridin-2-yl)ethanol-HCl salt (101.0 mg, 0.578 mmol) and (S)-2-methylpent-4-enoic acid (100.0 mg, 0.526 mmol), prepared according to a literature procedure (Chakraborty, T. K. et al. Synlett 2002, 2039-2040). The product was obtained as a white solid (31.1 mg, 25.3%) after column chromatography (EtOAc/hexane gradient). LRMS (ESI+) (M+H): 235.28.

(R)—(R)-2-((S)-2-methylpent-4-enamido)-2-(pyridin-2-yl)ethyl 2-methylpent-4-enoate: Using general procedure C for ester bond formation with DCM as solvent, the desired product was prepared from (S)—N—((R)-2-hydroxy-1-(pyridin-2-yl)ethyl)-2-methylpent-4-enamide (31.1 mg, 0.133 mmol) and (R)-2-methylpent-4-enoic acid (16.67 mg, 0.146 mmol), prepared according to a literature procedure (Chakraborty, T. K. et al. Synlett 2002, 2039-2040). The product was obtained as a white solid (14.8 mg, 33.7% yield) after column chromatography (EtOAc/hexane gradient). LRMS (ESI+) (M+H): 331.32

(3R,6S,11R,E)-6,11-dimethyl-3-(pyridin-2-yl)-1-oxa-4-azacyclododec-8-ene-5,12-dione: Using the general procedure for ring closing metathesis using toluene as solvent, the desired product was prepared from (R)—(R)-2-((S)-2-methylpent-4-enamido)-2-(pyridin-2-yl)ethyl 2-methylpent-4-enoate (14.8 mg, 0.045 mmol), yielding a white solid (12.0 mg, 89% yield) after column chromatography (EtOAc/hexanes gradient). LRMS (ESI+) (M+2H): 304.10.

### Compound BP

(2R,6R,E)-6-methyl-2-phenyl-1-oxa-4-azacyclododec-8-ene-5,12-dione:

(R)—N—((R)-2-hydroxy-2-phenylethyl)-2-methylpent-4-enamide: Using general procedure A for amide bond formation with DCM as solvent, the desired product was prepared from (R)-2-amino-phenylethanol (166.0 mg, 1.21 mmol) and (R)-2-methylpent-4-enoic acid (100.0 mg, 0.81 mmol), prepared according to a literature procedure (Chakraborty, T. K. et al. Synlett 2002, 2039-2040). The product was obtained as a white solid (185.0 mg, 99.0% yield) after column chromatography (EtOAc/hexane gradient). LRMS (ESI+) (M): 232.63.

(R)-2-((R)-2-methylpent-4-enamido)-1-phenylethyl pent-4-enoate: Using general procedure C for ester bond formation with DCM as solvent, the desired product was prepared from (R)—N—((R)-2-hydroxy-2-phenylethyl)-2-methylpent-4-enamide (188.0 mg, 0.81 mmol) and 4-pentenoic acid (89.0 mg, 0.89 mmol). The product was obtained as a white solid (209.0 mg, 82.0% yield) after column chromatography (EtOAc/hexane gradient). LRMS (ESI+) (M+Na): 338.30.

(2R,6R,E)-6-methyl-2-phenyl-1-oxa-4-azacyclododec-8-ene-5,12-dione: Using the general procedure for ring closing metathesis using toluene as solvent, the desired product was prepared from (R)-2-((R)-2-methylpent-4-enamido)-1-phenylethyl pent-4-enoate (209.0 mg, 0.66 mmol), yielding a white solid (150.0 mg, 79.0% yield). LRMS (ESI+) (M+H): 288.24.

### Example BQ

### (2R,6S,E)-6-(4-chlorobenzyl)-2-phenyl-1-oxa-4-azacyclododec-8-ene-5,12-dione

(S)-2-(4-chlorobenzyl)-N—((R)-2-hydroxy-2-phenylethyl)pent-4-enamide: Using general procedure B for amide bond formation with DMF as solvent (S)-2-(4-chlorobenzyl)-N—((R)-2-hydroxy-2-phenylethyl)pent-4-enamide was prepared from (R)-2-amino-2-phenylethanol (270 mg, 2.0 mmol) and (S)-2-(4-chlorobenzyl)pent-4-enoic acid (400 mg, 1.8 mmol). The product (600 mg) was obtained in 98% yield after column chromatography (MeOH/DCM gradient). LRMS (ESI+) (M+H): 344.30.

(R)-2-((S)-2-(4-chlorobenzyl)pent-4-enamido)-1-phenylethyl pent-4-enoate: Using general procedure C for ester bond formation using DCM as solvent (R)-2-((S)-2-(4-chlorobenzyl)pent-4-enamido)-1-phenylethyl pent-4-enoate was prepared from (S)-2-(4-chlorobenzyl)-N—((R)-2-hydroxy-2-phenylethyl)pent-4-enamide (600 mg, 1.7 mmol) and pent-4-enoic acid (210 μl, 2.1 mmol). The product (360 mg) was obtained as a colorless oil in 49% yield after column chromatography (MeOH/DCM gradient). LRMS (ESI+) (M+Na): 448.39.

(2R,6S,E)-6-(4-chlorobenzyl)-2-phenyl-1-oxa-4-azacyclododec-8-ene-5,12-dione: Using the general produce for ring closing metathesis with toluene as solvent (2R,6S,E)-6-(4-chlorobenzyl)-2-phenyl-1-oxa-4-azacyclododec-8-ene-5,12-dione was prepared from (R)-2-((S)-2-(4-chlorobenzyl)pent-4-enamido)-1-phenylethyl pent-4-enoate (360 mg, 0.85 mmol) to yield 170 mg of product as a white solid in 51% yield after column chromatography (MeOH/DCM gradient). LRMS (ESI+) (M+H): 398.23.

### Example BR

### (2R,6S,E)-6-(4-chlorobenzyl)-4-methyl-2-phenyl-1-oxa-4-azacyclododec-8-ene-5,12-dione

To a solution of (2R,6S,E)-6-(4-chlorobenzyl)-2-phenyl-1-oxa-4-azacyclododec-8-ene-5,12-dione (64 mg, 0.16 mmol) and iodomethane (30 μL, 0.48 mmol) in DMF (800 μL) cooled in an ice/water bath was added sodium hydride (6.4 mg, 0.16 mmol). The reaction mixture was slowly warmed to rt. After 4 h, TLC analysis shows no starting material remains. The reaction was diluted with H₂O and EtOAC and the layers separated. The aqueous is extracted with EtOAc twice then the combined organic extracts are washed with brine, dried over Na₂SO₄, filtered, and concentrated. The product (35 mg) was obtained as a white solid in 53% yield after column chromatography (MeOH/DCM gradient). LRMS (ESI+) (M+H): 412.26.

### Example BS

### (3R,11S,E)-11-methyl-3-phenyl-1-oxa-4-azacyclododec-8-ene-5,12-dione

Using general procedure C for ester bond formation using DCM as solvent (S)—((R)-2-pent-4-enamido-2-phenylethyl) 2-methylpent-4-enoate was prepared from (R)—N-(2-hydroxy-1-phenylethyl)pent-4-enamide (89 mg, 0.41 mmol) and (S)-2-methylpent-4-enoic acid (51 mg, 0.45 mmol), prepared according to a literature procedure (Chakraborty, T. K. et al. Synlett 2002, 2039-2040). The product (120 mg) was obtained as a white solid in 94% yield after column chromatography (MeOH/DCM gradient). Using the general produce for ring closing metathesis with toluene as solvent (3R,11S,E)-11-methyl-3-phenyl-1-oxa-4-azacyclododec-8-ene-5,12-dione was prepared from (S)—((R)-2-pent-4-enamido-2-phenylethyl) 2-methylpent-4-enoate (120 mg, 0.38 mmol) to yield 48 mg of product as a white solid in 44% yield after column chromatography (MeOH/DCM gradient). LRMS (ESI+) (M+H): 288.19.

### Example BU

### (3R,8S,9S,11R)-8,9-dihydroxy-11-methyl-3-phenyl-1-oxa-4-azacyclododecane-5,12-dione

To a solution of (3R,11R,E)-11-methyl-3-phenyl-1-oxa-4-azacyclododec-8-ene-5,12-dione (140 mg, 0.49 mmol) in tBuOH (3.7 mL), THF (970 μL), and water (110 μL) was added N-methyl morpholine N-oxide (50 wt % solution in H₂O) (140 μL, 0.59 mmol) followed by OsO₄ (2.5 wt % solution in tBuOH) (640 μL, 0.05 mmol). The reaction mixture was stirred 3 h at rt at which point TLC analysis indicated complete consumption of starting material. The reaction mixture was diluted with sat. Na₂SO₃ and stirred 1 h. The solution was subsequently further diluted with EtOAc and water and the layers separated. The aqueous was extracted 2× with EtOAc and the combined organic extracts were washed with brine, dried over Na₂SO₄, filtered, and concentrated. The crude product was purified using column chromatography (MeOH/DCM gradient) to yield 106 mg of product as a colorless oil in 68% yield. LRMS (ESI+) (M+H): 322.31.

### Example BV

### (3R,10aR,15aS,E)-3-methyl-3,4,7,8,10,10a,15,15a-octahydroindeno[2,1-b][1,4]oxaazacyclododecine-2,9-dione

Using general procedure B for amide bond formation with DMF as solvent N-((1R,2S)-2-hydroxy-2,3-dihydro-1H-inden-1-yl)pent-4-enamide was prepared from (1R,2S)-1-amino-2,3-dihydro-1H-inden-2-ol (300 mg, 2.0 mmol) and pent-4-enoic acid (205 μL, 2.0 mmol). The product (5.3 g) was obtained as a colorless oil in 74% yield after column chromatography (MeOH/DCM gradient). Using general procedure B for amide bond formation with DMF as solvent N-((1R,2S)-2-hydroxy-2,3-dihydro-1H-inden-1-yl)pent-4-enamide was prepared from (1R,2S)-1-amino-2,3-dihydro-1H-inden-2-ol (300 mg, 2.0 mmol) and pent-4-enoic acid (205 μL, 2.0 mmol). This compound (5.3 g) was obtained as a colorless oil in 74% yield after column chromatography (MeOH/DCM gradient). N-((1R,2S)-2-hydroxy-2,3-dihydro-1H-inden-1-yl)pent-4-enamide was then combined with (R)-2-methylpent-4-enoic acid (196 mg, 1.7 mmol), prepared according to a literature procedure (Chakraborty, T. K. et al. Synlett 2002, 2039-2040), according to general procedure C for ester bond formation using DCM as solvent to yield the title compound. The product (232 mg) was obtained as a white solid in 47% yield after column chromatography (MeOH/DCM gradient). Using the general produce for ring closing metathesis with toluene as solvent (3R,10aR,15aS,E)-3-methyl-3,4,7,8,10,10a,15,15a-octahydroindeno[2,1-b][1,4]oxaazacyclododecine-2,9-dione was prepared from (R)-((1R,2S)-1-pent-4-enamido-2,3-dihydro-1H-inden-2-yl) 2-methylpent-4-enoate (232 mg, 0.71 mmol) to yield 114 mg of product as a white solid in 54% yield after column chromatography (MeOH/DCM gradient). LRMS (ESI+) (M+H): 300.26.

### Compound BW

### 2-((2R,6S,8S,9S)-8,9-dihydroxy-5,12-dioxo-2-phenyl-1-oxa-4-azacyclododecan-6-yl)-N—((R)-1,2,3,4-tetrahydronaphthalen-1-yl)acetamide

To a solution of 2-((2R,6S,E)-5,12-dioxo-2-phenyl-1-oxa-4-azacyclododec-8-en-6-yl)-N—((R)-1,2,3,4-tetrahydronaphthalen-1-yl)acetamide (68 mg, 0.15 mmol) in tBuOH (1.1 mL), THF (0.30 mL), and water (0.07 mL) was added NMO (50 wt % solution in H₂O) (42 μl, 0.12 mmol) followed by OsO4 (2.5 wt % solution in tBuOH) (150 μl, 0.015 mmol). The reaction mixture was stirred at room temperature for 6 h at which point no starting material remained. The reaction mixture was diluted with sat. sodium sulfite and stirred 1 h. The solution was further diluted with EtOAc and water and the layers separated. The aqueous was extracted 2× with EtOAc and the combined organic extracts were washed with brine, dried over Na₂SO₄, filtered, and concentrated. The crude product was purified using silica gel chromatography using an ISCO automated system and a MeOH/DCM gradient to yield 41 mg of desired product as a white solid in 56% yield. LRMS (ESI+) (M+H): 495.29.

### Example BX

### N-((2R,3R,11R,E)-2-methyl-5,12-dioxo-3-phenyl-1-oxa-4-azacyclododec-8-en-11-yl)acetamide

N-((1R,2R)-2-hydroxy-1-phenylpropyl)pent-4-enamide: Using general procedure B for amide bond formation with DMF as solvent the title compound was prepared from (1R,2R)-1-amino-1-phenylpropan-2-ol (140 mg, 0.74 mmol), prepared according to literature procedure (Evan J. W. and Ellman J. A., J. Org. Chem., 2003, 68, 9948-9957), and pent-4-enoic acid (82 μl, 0.81 mmol). The product (172) was obtained as a colorless oil in quantitative yield following purification by column chromatography (MeOH/DCM gradient). LRMS (ESI+) (M+H): 234.09.

(R)-((1R,2R)-1-pent-4-enamido-1-phenylpropan-2-yl) 2-(tert-butoxycarbonylamino)pent-4-enoate: Using general procedure C for ester bond formation the title compound was prepared from N-((1R,2R)-2-hydroxy-1-phenylpropyl)pent-4-enamide (172 mg, 0.74 mmol) and (R)-2-(tert-butoxycarbonylamino)pent-4-enoic acid (190 mg, 0.89 mmol). The product (83 mg) was obtained in 25% yield following column chromatography (MeOH/DCM gradient). LRMS (ESI+) (M+Na): 431.37.

N-((2R,3R,11R,E)-2-methyl-5,12-dioxo-3-phenyl-1-oxa-4-azacyclododec-8-en-11-yl)acetamide: Using the general procedure for ring closing metathesis with toluene as solvent 73 mg tert-butyl (2R,3R,11R,E)-2-methyl-5,12-dioxo-3-phenyl-1-oxa-4-azacyclododec-8-en-11-ylcarbamate was prepared from (R)-((1R,2R)-1-pent-4-enamido-1-phenylpropan-2-yl) 2-(tert-butoxycarbonylamino)pent-4-enoate (83 mg, 0.19 mmol). To this product was added trifluoroacetic acid (0.34 ml, 4.5 mmol) and triethylsilane (0.72 ml, 4.5 mmol) and the solution was stirred 90 min then concentrated. The crude product was dissolved in 0.38 mL DMF and triethylamine (1.1 ml, 11.2 mmol) and acetic anhydride (1.6 ml, 4.5 mmol) were added and the reaction was stirred 16 h at which point LC-MS analysis indicated complete consumption of starting material. The reaction mixture was concentrated and the crude product was purified by column chromatography (MeOH/DCM gradient) to yield 25 mg of product as a white solid in 37% yield. LRMS (ESI+) (M+H): 345.28.

### Example BY

### N-(4-chlorobenzyl)-2-((1S,6R,10S,12S)-4,9-dioxo-6-phenyl-5,13-dioxa-8-azabicyclo[10.1.0]tridecan-10-yl)acetamide

To a solution of N-(4-chlorobenzyl)-2-((2R,6S,E)-5,12-dioxo-2-phenyl-1-oxa-4-azacyclododec-8-en-6-yl)acetamide (150 mg, 0.33 mmol) in DCM (2.1 mL) cooled in an ice-water bath was added mCPBA (16 mg, 0.69 mmol). The reaction mixture was slowly warmed to room temperature and stirred 6 h at which point no starting material remained. The reaction mixture was concentrated and purified using silica gel chromatography (MeOH/DCM gradient) to yield 72 mg of product as a white solid in 46% yield. LRMS (ESI+) (M+H): 471.06.

### Example BZ

### N-(4-chlorobenzyl)-2-((2R,6S,8S,9S)-8,9-dihydroxy-5,12-dioxo-2-phenyl-1-oxa-4-azacyclododecan-6-yl)acetamide

To a solution of N-(4-chlorobenzyl)-2-((2R,6S,E)-5,12-dioxo-2-phenyl-1-oxa-4-azacyclododec-8-en-6-yl)acetamide (150 mg, 0.33 mmol) in tBuOH (2.5 mL), THF (0.66 mL), and water (0.07 mL) was added NMO (50 wt % solution in H₂O) (93 μl, 0.40 mmol) followed by OsO4 (2.5 wt % solution in tBuOH) (430 μl, 0.033 mmol). The reaction mixture was stirred at room temperature for 6 h at which point no starting material remained. The reaction mixture was diluted with sat. sodium sulfite and stirred 1 h. The solution was further diluted with EtOAc and water and the layers separated. The aqueous was extracted 2× with EtOAc and the combined organic extracts were washed with brine, dried over Na₂SO₄, filtered, and concentrated. The crude product was purified using silica gel chromatography using an ISCO automated system and a MeOH/DCM gradient to yield 57 mg of desired product as a white solid in 35% yield. LRMS (ESI+) (M+H): 489.12.

### Example CB

### (R,E)-2-phenyl-1-oxa-4-azacyclododec-8-ene-5,12-dione

(R)-2-pent-4-enamido-1-phenylethyl pent-4-enoate: To a solution of pent-4-enoic acid (740 μL, 7.3 mmol) in DMF (4.5 mL) was added HOBt (990 mg, 7.3 mmol) and EDC (1.4 g, 7.3 mmol) and the resulting solution was stirred 30 min. (R)-2-amino-phenylethanol (500 mg, 3.64 mmol), iPr₂NEt (1.3 mL, 7.3 mmol), and DMAP (89 mg, 0.73 mmol) were subsequently added and the reaction mixture was stirred 16 h. The reaction mixture was then diluted with EtOAc and sat. NH₄Cl and the layers separated. The aqueous was extracted 2× with EtOAc and the combined organic extracts were washed with 1N HCl and brine, dried over Na₂SO₄, filtered, and concentrated. The crude product was purified using silica gel chromatography (MeOH/DCM gradient) to yield 160 mg of product as a white solid in 15% yield. LRMS (ESI+) (M+Na): 324.30.

(R,E)-2-phenyl-1-oxa-4-azacyclododec-8-ene-5,12-dione: Using the general produce for ring closing metathesis with toluene as solvent (R,E)-2-phenyl-1-oxa-4-azacyclododec-8-ene-5,12-dione was prepared from (R)-2-pent-4-enamido-1-phenylethyl pent-4-enoate (160 mg, 0.53 mmol) to yield 95 mg of product as a white solid in 66% yield after column chromatography (MeOH/DCM gradient). LRMS (ESI+) (M+H): 274.25.

### Example CC

### 3-(4-chlorophenyl)-N-((3R,11R,E)-5,12-dioxo-3-phenyl-1-oxa-4-azacyclododec-8-en-11-yl)propanamide

Using general procedure B for amide bond formation with DCM as solvent (3R,11R,E)-11-amino-3-phenyl-1-oxa-4-azacyclododec-8-ene-5,12-dione (178 mg, 0.62 mmol) was combined with 2-(4-chlorophenyl)ethanamine (171 mg, 0.93 mmol) to yield the title compound as a white solid following purification of the crude product by column chromatography (MeOH/DCM). LRMS (ESI−) (M−H): 453.04.

### Example CD

### N-(4-chlorobenzyl)-2-((1S,3S,7R,12S)-4,9-dioxo-7-phenyl-5,13-dioxa-8-azabicyclo[10.1.0]tridecan-3-yl)acetamide

To a solution of N-(4-chlorobenzyl)-2-((3R,11S,E)-5,12-dioxo-3-phenyl-1-oxa-4-azacyclododec-8-en-11-yl)acetamide (48 mg, 0.11 mmol) in DCM (660 μl) cooled in an ice-water bath was added mCPBA (50 mg, 0.22 mmol). The reaction mixture was slowly warmed to room temperature and stirred 6 h at which point an additional equivalent of mCPBA was added. After stirring an additional 3 h the reaction mixture was diluted with sat. Na₂SO₃ and stirred 1 h then further diluted with DCM and the layers separated. The aqueous was extracted 2× with DCM and the combined organic extracts were washed with sat. NaHCO₃ and brine, dried over Na₂SO₄, filtered, and concentrated. The crude product was purified using column chromatography (MeOH/DCM gradient) to yield 6 mg of product as a white solid in 12% yield. LRMS (ESI−) (M−H): 469.00.

### Example CE

### (3R,11R,E)-11-azido-3-phenyl-1-oxa-4-azacyclododec-8-ene-5,12-dione

A solution of sodium azide (595 mg, 9.15 mmol) in DI-water (1.495 ml, 8.30E+04 mol) was cooled in an ice bath and treated with DCM (2.503 ml, 38.9 mmol). The resulting biphasic mixture was stirred vigorously and treated with trifluoromethanesulfonic anhydride (308 μl, 1.85 mmol) over a period of 5 min. The mixture was stirred in an ice bath for 2 hours. Subsequently, the organic phase was separated and the aqueous phase extracted 2× with DCM. The total volume of the TfN₃ solution was approximately 5 ml. The TFA salt form of (3R,11R,E)-11-amino-3-phenyl-1-oxa-4-azacyclododec-8 ene-5,12-dione (374 mg, 0.93 mmol) was dissolved in 3 ml of DI-water and treated with potassium carbonate (192 mg, 1.39 mmol) and copper (II) sulfate pentahydrate (2.197 mg, 8.8 mol). To this solution, MeOH (6 ml) was added, followed by the TfN₃ solution (5 ml). More MeOH was added to aid homogeneity. The reaction was stirred overnight at room temperature (14 hours). Upon completion of the reaction, the crude mixture was diluted with EtOAc, and washed 3× with half-saturated NaHCO₃. The organic layers were combined and dried to afford an off-white solid. This crude solid was triturated from a solution of 80% EtOAc in hexanes and filtered to afford a clean white solid (286 mg) in 99% yield. LRMS (ESI+) (M+H): 316.25.

### Example CF

### (3R,11R,E)-11-(4-(4-chlorophenyl)-1H-1,2,3-triazol-1-yl)-3-phenyl-1-oxa-4-azacyclododec-8-ene-5,12-dione

To a suspension of the TFA salt form of (3R,11R,E)-11-amino-3-phenyl-1-oxa-4-azacyclododec-8 ene-5,12-dione (10 mg, 0.032 mmol) and 1-chloro-4-ethynylbenzene (6.52 mg, 0.048 mmol) in 1:1 mixture of tert-butanol and water (1.0 ml), sodium ascorbate (63.0 mg, 0.318 mmol) in 100 μl water was added, followed by copper (II) sulfate pentahydrate (7.94 mg, 0.032 mmol) in 100 μl. The reaction mixture remained a cloudy white suspension, upon which 200 μl MeOH was added to aid solubility. The reaction was stirred overnight for 14 hours. After completion of the reaction, the crude reaction was diluted with DCM, and washed with water. The crude product was purified using silica gel chromatography using an ISCO automated system using a MeOH/DCM gradient. The isolated product (12.2 mg) was obtained in 85% yield as a white solid. LRMS (ESI+) (M+H): 451.31

### Example CG

### (3R,11R,E)-3-phenyl-11-(1H-1,2,3-triazol-1-yl)-1-oxa-4-azacyclododec-8-ene-5,12-dione

A solution of the TFA salt form of (3R,11R,E)-11-amino-3-phenyl-1-oxa-4-azacyclododec-8 ene-5,12-dione (10 mg, 0.032 mmol) in toluene (4.0 ml) was treated with ethynyltrimethylsilane (88 μl, 0.635 mmol). The reaction was heated to 80° C. for 10 hours. The crude reaction mixture was purified using silica gel chromatography using an ISCO automated system using a MeOH/DCM gradient. The isolated product (9.64 mg) was obtained in 89% yield as a white solid. LRMS (ESI+) (M+H): 341.34.

### Example CH

### (3R,11R,E)-11-morpholino-3-phenyl-1-oxa-4-azacyclododec-8-ene-5,12-dione

(3R,11R,E)-11-(2H-indazol-2-yl)-3-phenyl-1-oxa-4-azacyclododec-8-ene-5,12-dione:

In a flask, 2-nitrobenzaldehyde (5.63 mg, 0.037 mmol), the TFA salt form of (3R,11R,E)-11-amino-3-phenyl-1-oxa-4-azacyclododec-8 ene-5,12-dione (10 mg, 0.025 mmol), triethylamine (10.39 μl, 0.075 mmol), and 4 Å molecular sieves were heated in DCM (10.0 ml) for 4 hours at 50° C. The reaction was cooled and triethylphosphite (21.31 μl, 0.124 mmol) was added. The reaction mixture was heated to 110° C. overnight (14 hours) in toluene. The crude reaction mixture was concentrated under high vacuum. The product (0.6 mg) was isolated via silica chromatography using an ISCO automated system (MeOH/DCM gradient) in 0.6% yield in the form of a white solid. LRMS (ESI+) (M+H): 390.15.

(3R,11R,E)-11-morpholino-3-phenyl-1-oxa-4-azacyclododec-8-ene-5,12-dione: (3R,11R,E)-11-amino-3-phenyl-1-oxa-4-azacyclododec-8-ene-5,12-dione (0.029 g, 0.10 mmol) was dissolved in N,N-dimethylformamide (0.5 mL) to give a clear colorless solution. 2-Bromoethyl ether (15 μL, 0.12 mmol) was added followed by the addition of solid sodium carbonate (0.054 g, 0.51 mmol) and sodium iodide (1.5 mg, 10 mol). The flask was sealed under argon and heated to 60° C. for 20 hours to eventually give a cloudy orange solution with a fine white suspension. After cooling to room temperature, the reaction was filtered through celite with acetonitrile. The filtrate was concentrated under reduced pressure to give orange solid. This material was partitioned between chloroform (50 mL) and water (20 mL). The aqueous layer was further extracted with additional chloroform (3×50 mL). Afterwards, the combined chloroform layers were washed with brine (50 mL) then dried over magnesium sulfate, filtered, and concentrated under reduced pressure to give a yellow solid. The crude material was purified by column chromatography over silica gel (20% v/v methanol in dichloromethane/dichloromethane:0/100 to 20/80) to give a white solid (30.2 mg, 83%). Alternatively, the crude material may be recrystallized from boiling toluene. LRMS (ESI+) (M+H): 359.35.

### Example CI

### N-((3R,11R,E)-3-(4-fluorophenyl)-5,12-dioxo-1-oxa-4-azacyclododec-8-en-11-yl)acetamide

(R)—N-(1-(4-fluorophenyl)-2-hydroxyethyl)pent-4-enamide: Using General Procedure C for Amide Bond Formation with DCM (3.2 mL), the title compound was prepared from (R)-2-amino-2-(4-fluorophenyl)ethanol (0.0500 g, 0.322 mmol), 4-pentenoic acid (0.0323 mg, 0.322 mmol), DMAP (3.9 mg, 0.032 mmol) and EDCI (0.0930 g, 0.483 mmol). The crude product was purified by column chromatography over silica gel (20% v/v methanol in dichloromethane/dichloromethane:0/100 to 50/50) to afford the title compound as a white solid (68.6 mg, 90%). LRMS (ESI+) (M+): 237.98.

(R,R)-2-(4-fluorophenyl)-2-(pent-4-enamido)ethyl 2-((tert-butoxycarbonyl)amino)pent-4-enoate: Using General Procedure C for Amide Bond Formation with DCM (2.9 mL), the title compound was prepared from (R)—N-(1-(4-fluorophenyl)-2-hydroxyethyl)pent-4-enamide (68.6 mg, 0.289 mmol), N-Boc-allylglycine dicyclohexylamine salt (120.0 mg, 0.304 mmol), DMAP (3.5 mg, 0.029 mmol) and EDCI (83.0 mg, 0.434 mmol). The crude product was purified by column chromatography over silica gel (20% v/v methanol in dichloromethane/dichloromethane: 0/100 to 25/75) to afford the title compound as a white solid (98.5 mg, 78%). LRMS (ESI+) (M+): 435.35.

tert-butyl ((3R,11R,E)-3-(4-fluorophenyl)-5,12-dioxo-1-oxa-4-azacyclododec-8-en-11-yl)carbamate: Using the General Procedure for Ring Closing Metathesis with dichloromethane (23.0 mL) as solvent, the title compound was prepared from (R,R)-2-(4-fluorophenyl)-2-(pent-4-enamido)ethyl 2-((tert-butoxycarbonyl)amino)pent-4-enoate (98.5 mg, 0.227 mmol), Grubbs' second generation catalyst (19.3 mg, 0.023 mmol). The crude product was purified by column chromatography over silica gel (20% v/v methanol in dichloromethane/dichloromethane:0/100 to 20/80) to afford the title compound as a white solid (78.6 mg, 85%). LRMS (ESI−) [M+HC(O)O⁻]: 451.23.

N-((3R,11R,E)-3-(4-fluorophenyl)-5,12-dioxo-1-oxa-4-azacyclododec-8-en-11-yl)acetamide: tert-butyl ((3R,11R,E)-3-(4-fluorophenyl)-5,12-dioxo-1-oxa-4-azacyclododec-8-en-11-yl)carbamate (78.6 mg, 0.193 mmol) was suspended in dichloromethane (0.35 mL) and then treated with triethylsilane (0.31 mL, 1.9 mmol). Trifluoroacetic acid (0.30 mL, 3.9 mmol) was slowly added dropwise to give a clear solution that bubbled violently. The reaction was stirred 2.5 hours at room temperature until the bubbling ceased. The reaction was then concentrated under reduced pressure to give a white solid. This crude material was suspended in triethylamine (2.70 mL, 19.3 mmol) before adding acetic anhydride (1.80 mL, 19.3 mmol) in a dropwise fashion. The resulting mixture was stirred at room temperature for 16 hours before being concentrated under reduced pressure. The solid residue was partitioned between chloroform (50 mL) and water (20 mL). The aqueous layer was further extracted with chloroform (3×50 mL), and the combined organic layers were washed with brine (50 mL), then dried over magnesium sulfate, filtered, and concentrated under reduced pressure to give a white solid. This material was purified by column chromatography over silica gel (20% v/v methanol in dichloromethane/dichloromethane:0/100 to 40/60) to afford the title compound as a white solid (30.5 mg, 45% over 2 steps). LRMS (ESI+) [M+]: 348.95.

### Example CJ

### (4S,12R,E)-12-methyl-4-phenyl-1-oxa-5-azacyclododec-9-ene-2,6-dione

(S)-3-((tert-butoxycarbonyl)amino)-3-phenylpropanoic acid: (S)-3-amino-3-phenylpropanoic acid (0.200 g, 1.21 mmol) was dissolved in THF (3.0 ml) and Water (3.0 ml) and the resulting white mixture was cooled to 0° C. Finely powdered NaHCO₃ (0.305 g, 3.63 mmol) was added in one portion, followed by Boc₂O (0.317 g, 1.45 mmol). The reaction was stirred overnight for 10 hours while it gradually warmed to room temperature. The reaction was washed with diethyl ether (2×10 mL). The remaining aqueous layer was carefully acidified to pH 4 with saturated citric acid solution (aqueous). The solution turned clear before a white solid eventually precipitated. After extraction with dichloromethane (4×10 mL), the extracts were dried over magnesium sulfate, filtered, and concentrated under reduced pressure to give the product as a white solid (0.2877 g). This material was used without further purification.

(S)—(R)-pent-4-en-2-yl 3-((tert-butoxycarbonyl)amino)-3-phenylpropanoate: Using General Procedure C for Amide Bond Formation with DCM (5.5 mL), the title compound was prepared from (S)-3-((tert-butoxycarbonyl)amino)-3-phenylpropanoic acid (0.2877 g), (R)-pent-4-en-2-ol (0.17 mL, 1.6 mmol), DMAP (0.013 g, 0.11 mmol) and EDCI (0.312 g, 1.63 mmol). The crude product was obtained as a yellow oil (0.3632 g) that was used immediately without further purification.

(S)—(R)-pent-4-en-2-yl 3-amino-3-phenylpropanoate: (S)—(R)-pent-4-en-2-yl 3-((tert-butoxycarbonyl)amino)-3-phenylpropanoate (0.3632 g) was treated with tri-isopropylsilane (46.6 μL, 0.227 mmol) and H₂O (4.08 μL, 0.227 mmol) to give a clear pale yellow solution. Trifluoroacetic acid (642 μL, 8.62 mmol) was slowly added dropwise to give a red-brown solution that bubbled violently. The reaction was stirred 15 minutes at room temperature until the bubbling ceased. The reaction was then diluted with dichloromethane (5 mL) before carefully and slowly adding saturated NaHCO₃ solution (aqueous, 10 mL). This mixture was stirred vigorously until bubbling ceased. The aqueous layer was removed and the remaining organics were washed with another 10 mL of saturated NaHCO₃ solution. The combined aqueous washings were back-extracted with dichloromethane (2×10 mL) before all of the combined organic layers were dried over magnesium sulfate, filtered, and concentrated under reduced pressure to give the crude product (0.2622 g) as a clear, dark yellow oil that was used without further purification.

(S)—(R)-pent-4-en-2-yl 3-(pent-4-enamido)-3-phenylpropanoate: Using General Procedure C for Ester Bond Formation with DCM (4.4 mL) as solvent, the title compound was prepared from (S)—(R)-pent-4-en-2-yl 3-amino-3-phenylpropanoate (0.2622 g), 4-pentenoic acid (0.23 mL, 2.2 mmol), DMAP (0.014 g, 0.11 mmol) and EDCI (0.323 g, 1.67 mmol) in DCM (4.4 mL). After work-up, the crude material was purified by column chromatography over silica gel (hexanes/ethyl acetate: 100/0 to 50/50) to provide the product as a cloudy oil (0.2462 g, 0.781 mmol, 65% yield over 4 steps). ¹H NMR (300 MHz, CDCl₃): δ 7.39-7.16 (m, 6H), 6.62 (d, J=8.0 Hz, 1H), 5.83 (ddd, J=6.4, 12.6, 16.8 Hz, 1H), 5.62-5.38 (m, 2H), 5.03 (ddd, J=6.5, 16.5, 21.5 Hz, 3H), 4.88 (dd, J=6.2, 12.6 Hz, 1H), 2.92 (dd, J=5.7, 15.6 Hz, 1H), 2.80 (dd, J=5.8, 15.6 Hz, 1H), 2.47-2.28 (m, 3H), 2.16 (dd, J=6.2, 11.9 Hz, 1H), 1.14 (d, J=6.3 Hz, 2H).

(4S,12R,E)-12-methyl-4-phenyl-1-oxa-5-azacyclododec-9-ene-2,6-dione: Using the General Procedure for Ring Closing Metathesis with toluene (78 mL) as solvent, the title compound was prepared from (S)—(R)-pent-4-en-2-yl 3-(pent-4-enamido)-3-phenylpropanoate (0.2462 g, 0.781 mmol), Grubbs' second generation catalyst (0.066 g, 0.078 mmol). After work-up, the crude material was purified by column chromatography over silica gel (hexanes/ethyl acetate: 90/10 to 0/100) to provide the product as a white solid (0.1757 g, 0.614 mmol, 79% yield). LRMS (ESI+) (M+H): 287.93.

### Example CK

### (3R,11R,E)-11-methyl-3-phenyl-1-oxa-5-azacyclododec-8-ene-4,12-dione

(S)-4-benzyl-3-((R)-3-(benzyloxy)-2-phenylpropanoyl)oxazolidin-2-one: The title compound was prepared as described in the literature (J. Am. Chem. Soc. 2003, 125, 7914-7922).

(R)-3-(benzyloxy)-2-phenylpropanoic acid: A solution of (S)-4-benzyl-3-((R)-3-(benzyloxy)-2-phenylpropanoyl)oxazolidin-2-one (0.6485 g, 1.561 mmol) in THF (25 ml) and H₂O (6.25 ml) was cooled to 0° C. before adding hydrogen peroxide (30% weight in H₂O, 1.59 ml, 15.6 mmol) and powdered lithium hydroxide (0.131 g, 3.12 mmol). The cloudy reaction was stirred for 30 minutes at 0° C. then warmed to room temperature and stirred for another hour. The reaction was cooled to 0° C. again then quenched with saturated sodium sulfite solution (aqueous, 4 mL). The mixture was warmed to room temperature before the organic layer was removed. The remaining aqueous layer was washed with DCM (2×25 mL), then the combined organic layers were back-extracted with saturated NaHCO₃ solution (aqueous, 3×25 mL). The combined aqueous layers were carefully acidified to pH 4 with solid citric acid (˜7 g) and extracted with ethyl acetate (4×50 mL). The combined extracts were washed with brine then dried over magnesium sulfate, filtered, and concentrated under reduced pressure to give the crude product as a cloudy oil (0.3275 g).

(R)-3-(benzyloxy)-N-(but-3-en-1-yl)-2-phenylpropanamide: Using General Procedure C for Amide Bond Formation with DCM (6.2 mL) as solvent, the title compound was prepared from (R)-3-(benzyloxy)-2-phenylpropanoic acid (0.3275 g), but-3-en-1-amine (0.18 ml, 1.9 mmol), DMAP (0.016 g, 0.128 mmol), and EDCI (0.367 g, 1.92 mmol). After work-up, the crude material was purified by column chromatography over silica gel (hexanes/ethyl acetate: 100/0 to 60/40) to provide the product as a colorless oil that solidifies upon standing (0.2684 g, 0.867 mmol, 56% yield over 2 steps). LRMS (ESI+) (M+): 309.97.

(R)—N-(but-3-en-1-yl)-3-hydroxy-2-phenylpropanamide: To a solution of naphthalene (137.0 mg, 1.067 mmol) in THF (3.6 mL) was added lithium wire (6.2 mg, 0.89 mmol). This mixture was stirred at room temperature for 2 hours until all of the metal dissolved and a dark green color persisted. The resulting solution was then cooled to −25° C. before the dropwise addition of (R)-3-(benzyloxy)-N-(but-3-en-1-yl)-2-phenylpropanamide (55.0 mg, 0.178 mmol) as a THF solution (1.2 mL). The reaction was stirred at −25° C. for 1 hour. While still cold, the reaction was diluted with saturated ammonium chloride solution (aqueous, 2.5 mL) and H₂O (2.5 mL). The mixture was warmed to room temperature before it was extracted with Et₂O (3×5 mL). The combined organic layers were dried over magnesium sulfate, filtered, and concentrated under reduced pressure to give colorless crystals. The crude material was purified by column chromatography over silica gel (hexanes/ethyl acetate: 100/0 to 0/100) to provide the product as a white powder (34.6 mg, 0.158 mmol, 89% yield). LRMS (ESI+) (M+H): 220.40.

(R)—(R)-3-(but-3-en-1-ylamino)-3-oxo-2-phenylpropyl 2-methylpent-4-enoate: Using General Procedure C for Ester Bond Formation with DCM (2.0 mL) as solvent, the title compound was prepared from (R)—N-(but-3-en-1-yl)-3-hydroxy-2-phenylpropanamide (0.0451 g, 0.206 mmol), (R)-2-methylpent-4-enoic acid (6.8 M solution in Et₂O, 0.0360 ml, 0.247 mmol), DMAP (0.002 g, 0.02 mmol), and EDCI (0.0591 g, 0.309 mmol). After work-up, the crude material was obtained as a clear oil (67.1 mg) that was used without further purification. LRMS (ESI+) (M+): 316.02.

(3R,11R,E)-11-methyl-3-phenyl-1-oxa-5-azacyclododec-8-ene-4,12-dione: Using the General Procedure for Ring Closing Metathesis with toluene (20.5 mL) as solvent, the title compound was prepared from (R)—(R)-3-(but-3-en-1-ylamino)-3-oxo-2-phenylpropyl 2-methylpent-4-enoate (0.0649 g, 0.206 mmol), Grubbs' second generation catalyst (0.0175 g, 0.021 mmol). After work-up, the crude material was purified by column chromatography over silica gel (hexanes/ethyl acetate: 100/0 to 30/70) to provide the product as a white solid (0.0374 g, 0.130 mmol, 63% yield over 2 steps). LRMS (ESI+H) (M+): 288.26.

### Example CL

### N-((2S,3S,6R,E)-3,4-dimethyl-5,12-dioxo-2-phenyl-1-oxa-4-azacyclododec-8-en-6-yl)acetamide

tert-butyl ((R)-1-(((1S,2S)-1-hydroxy-1-phenylpropan-2-yl)(methyl)amino)-1-oxopent-4-en-2-yl)carbamate: Using general procedure A for amide bond formation with DCM as solvent, the title compound was prepared from (R)-2-(tert-butoxycarbonylamino)pent-4-enoic acid dicyclohexylamine salt (240 mg, 0.61 mmol) and (1S,2S)-2-(methylamino)-1-phenylpropan-1-ol (100 mg, 0.61 mmol). Product (219 mg) was obtained and used in the next step without purification. LRMS (ESI+) (M+H): 363.37.

(1S,2S)-2-((R)-2-((tert-butoxycarbonyl)amino)-N-methylpent-4-enamido)-1-phenylpropyl pent-4-enoate: Using general procedure C for ester bond formation with DCM as solvent the title compound was prepared from 4-pentenoic acid (68 μL, 0.67 mmol) and tert-butyl ((R)-1-(((1S,2S)-1-hydroxy-1-phenylpropan-2-yl)(methyl)amino)-1-oxopent-4-en-2-yl)carbamate (219 mg, 0.60 mmol). The product (183 mg) was obtained in 68% yield over 2 steps after column chromatography (Hex/EA gradient). LRMS (ESI+) (M+H): 445.42.

tert-butyl ((2S,3S,6R,E)-3,4-dimethyl-5,12-dioxo-2-phenyl-1-oxa-4-azacyclododec-8-en-6-yl)carbamate: Using the general procedure for ring closing metathesis with toluene as solvent the title compound was prepared from (1S,2S)-2-((R)-2-((tert-butoxycarbonyl)amino)-N-methylpent-4-enamido)-1-phenylpropyl pent-4-enoate (183 mg, 0.41 mmol). The product (26 mg) was obtained in 15% yield following purification by column chromatography (Hex/EA gradient). LRMS (ESI+) (M+H): 417.37.

(2S,3S,6R,E)-6-amino-3,4-dimethyl-2-phenyl-1-oxa-4-azacyclododec-8-ene-5,12-dione: tert-butyl ((2S,3S,6R,E)-3,4-dimethyl-5,12-dioxo-2-phenyl-1-oxa-4-azacyclododec-8-en-6-yl)carbamate (25 mg, 0.06 mmol) was dissolved in DCM (600 μL) and trifluoroacetic acid (23 μL, 0.30 mmol) was added dropwise. After 4 h, the reaction is complete as shown by LCMS. Solvents are evaporated and coevaporated with toluene 3 times. The product (19 mg) was obtained and used in the next step without further purification. LRMS (ESI+) (M+H): 317.33.

N-((2S,3S,6R,E)-3,4-dimethyl-5,12-dioxo-2-phenyl-1-oxa-4-azacyclododec-8-en-6-yl)acetamide: (2S,3S,6R,E)-6-amino-3,4-dimethyl-2-phenyl-1-oxa-4-azacyclododec-8-ene-5,12-dione (19 mg, 0.06 mmol) was dissolved in DMF (1.2 mL) under argon atmosphere. Triethylamine (506 μL, 3.60 mmol) and acetic anhydride (341 μL, 3.60 mmol) were successively added in one portion and the reaction mixture was stirred at room temperature for 15 h. Solvents were evaporated before the mixture was dissolved with ethyl acetate and successively washed with NH₄Cl solution and NaHCO₃ solution. The product (5 mg) was obtained in 23% yield after purification by column chromatography (DCM/MeOH gradient). LRMS (ESI+) (M+H): 359.35.

### Example CM

### N-((9R,14aS,E)-1,10-dioxo-3,4,5,8,9,10,12,13,14,14a-decahydro-1H-pyrrolo[2,1-c][1,4]oxaazacyclododecin-9-yl)acetamide

(S)-1-tert-butyl 2-pent-4-enyl pyrrolidine-1,2-dicarboxylate: Using general procedure C for ester bond formation with DCM as solvent the title compound was prepared from pent-4-en-1-ol (53 μL, 0.51 mmol) and (S)-1-(tert-butoxycarbonyl)pyrrolidine-2-carboxylic acid (100 mg, 0.465 mmol). The product (118 mg) was obtained in 90% yield and used in the next step without purification. LRMS (ESI+) (M+Na): 306.30.

(S)-pent-4-enyl pyrrolidine-2-carboxylate: (S)-1-tert-butyl 2-pent-4-enyl pyrrolidine-1,2-dicarboxylate (118 mg, 0.42 mmol) was dissolved in DCM (833 μL) and trifluoroacetic acid (160 μL, 2.08 mmol) was added dropwise. The pAfter 3 h, the reaction is complete as shown by LCMS. Solvents are evaporated and coevaporated with toluene 3 times. The product (76 mg) was obtained and used in the next step without further purification. LRMS (ESI+) (M+H): 184.20.

(S)-pent-4-enyl 1-((R)-2-(tert-butoxycarbonylamino)pent-4-enoyl)pyrrolidine-2-carboxylate: Using general procedure A for amide bond formation with DCM as solvent, the title compound was prepared from (R)-2-(tert-butoxycarbonylamino)pent-4-enoic acid dicyclohexylamine salt (164 mg, 0.415 mmol) and (S)-pent-4-enyl pyrrolidine-2-carboxylate (76 mg, 0.415 mmol). Product (81 mg) was obtained in 51% yield over 2 steps following purification by column chromatography (Hex/EA gradient). LRMS (ESI+) (M+H): 381.38.

tert-butyl (9R,14aS,E)-1,10-dioxo-3,4,5,8,9,10,12,13,14,14a-decahydro-1H-pyrrolo[2,1-c][1,4]oxaazacyclododecin-9-ylcarbamate: Using the general procedure for ring closing metathesis with toluene as solvent the title compound was prepared from (S)-pent-4-enyl 1-((R)-2-(tert-butoxycarbonylamino)pent-4-enoyl)pyrrolidine-2-carboxylate (81 mg, 0.213 mmol). The product (31 mg) was obtained in 41% yield following purification by column chromatography (Hex/EA gradient). LRMS (ESI+) (M+H): 353.32.

(9R,14aS,E)-9-amino-4,5,8,9,12,13,14,14a-octahydro-1H-pyrrolo[2,1-c][1,4]oxaazacyclododecine-1,10(3H)-dione: tert-butyl (9R,14aS,E)-1,10-dioxo-3,4,5,8,9,10,12,13,14,14a-decahydro-1H-pyrrolo[2,1-c][1,4]oxaazacyclododecin-9-ylcarbamate (31 mg, 0.088 mmol) was dissolved in DCM (880 μL) and trifluoroacetic acid (34 μL, 0.44 mmol) was added dropwise. After 5 h, the reaction is complete as shown by LCMS. Solvents are evaporated and coevaporated with toluene 3 times. The product (22 mg) was obtained and used in the next step without further purification. LRMS (ESI+) (M+H): 253.26.

N-((9R,14aS,E)-1,10-dioxo-3,4,5,8,9,10,12,13,14,14a-decahydro-1H-pyrrolo[2,1-c][1,4]oxaazacyclododecin-9-yl)acetamide: (9R,14aS,E)-9-amino-4,5,8,9,12,13,14,14a-octahydro-1H-pyrrolo[2,1-c][1,4]oxaazacyclododecine-1,10(3H)-dione (22 mg, 0.087 mmol) was dissolved in DMF (1.7 mL) under argon atmosphere. Triethylamine (735 μL, 5.23 mmol) and acetic anhydride (495 μL, 5.23 mmol) were successively added in one portion and the reaction mixture was stirred at room temperature for 15 h. Solvents were evaporated under vacuum. The product (19 mg) was obtained in 74% yield after purification by column chromatography (DCM/MeOH gradient). LRMS (ESI+) (M+H): 295.28.

### Example CN

### N-((9R,14aS,Z)-3,10-dioxo-3,4,5,8,9,10,12,13,14,14a-decahydro-1H-pyrrolo[2,1-c][1,4]oxaazacyclododecin-9-yl)acetamide

tert-butyl ((9R,14aS,Z)-3,10-dioxo-3,4,5,8,9,10,12,13,14,14a-decahydro-1H-pyrrolo[2,1-c][1,4]oxaazacyclododecin-9-yl)carbamate: Using the general procedure for ring closing metathesis with toluene as solvent the title compound was prepared from ((S)-1-((R)-2-(tert-butoxycarbonylamino)pent-4-enoyl)pyrrolidin-2-yl)methyl pent-4-enoate (721 mg, 1.895 mmol). The product (130 mg) was obtained in 20% yield following purification by column chromatography (Hex/EA gradient). LRMS (ESI+) (M+H): 353.18.

(9R,14aS,Z)-9-amino-4,5,8,9,14,14a-hexahydro-1H-pyrrolo[2,1-c][1,4]oxaazacyclododecine-3,10(12H,13H)-dione: tert-butyl ((9R,14aS,Z)-3,10-dioxo-3,4,5,8,9,10,12,13,14,14a-decahydro-1H-pyrrolo[2,1-c][1,4]oxaazacyclododecin-9-yl)carbamate (130 mg, 0.369 mmol) was dissolved in DCM (369 μL) and trifluoroacetic acid (142 μL, 1.844 mmol) was added dropwise. After 2 h, the reaction is complete as shown by LCMS. Solvents are evaporated and coevaporated with toluene 3 times. The product (93 mg) was obtained and used in the next step without further purification. LRMS (ESI+) (M+H): 253.09.

N-((9R,14aS,Z)-3,10-dioxo-3,4,5,8,9,10,12,13,14,14a-decahydro-1H-pyrrolo[2,1-c][1,4]oxaazacyclododecin-9-yl)acetamide: (9R,14aS,Z)-9-amino-4,5,8,9,14,14a-hexahydro-1H-pyrrolo[2,1-c][1,4]oxaazacyclododecine-3,10(12H, 13H)-dione (93 mg, 0.369 mmol) was dissolved in DMF (7.3 mL) under argon atmosphere. Triethylamine (3.1 mL, 22.12 mmol) and acetic anhydride (2.1 mL, 22.12 mmol) were successively added in one portion and the reaction mixture was stirred at room temperature for 15 h. Solvents were evaporated before the mixture was dissolved with ethyl acetate and successively washed with NH₄Cl solution and NaHCO₃ solution. The product (98 mg) was obtained in 90% yield after purification by column chromatography (DCM/MeOH gradient). LRMS (ESI+) (M+H): 295.26.

### Example CO

### (3R,11R,E)-3-phenyl-11-(1H-pyrazol-1-yl)-1-oxa-4-azacyclododec-8-ene-5,12-dione

To a solution of (3R,11R,E)-11-amino-3-phenyl-1-oxa-4-azacyclododec-8 ene-5,12-dione (100.0 mg, 0.25 mmol) and sodium hypochlorite (0.309 μl, 5.00 mol) in water, added potassium cyanate (40.6 mg, 0.500 mmol). The mixture was heated to 60° C. under nitrogen for 1 hour. Another portion of potassium cyanate (40.6 mg, 0.500 mmol) was added and heating continued for 2 hours. The mixture was then cooled and acidified to pH 1 with concentrated HCl. The precipitate was filtered, washed with water, and dried to afford the hydantoic acid intermediate. To an ice-cold solution of the hydantoic acid intermediate in 2.5N KCl (11.0 mg, 0.200 mmol), 6% sodium hypochlorite (12.0 μl, 0.200 mmol) was slowly added. The reaction mixture was heated to 80° C. for 1.5 hours. Subsequently, toluene and hydrazine monohydrate (10.01 mg, 0.200 mmol) were added and the mixture vigorously stirred while HCl (6.08 μl, 0.200 mmol) was added. The crude mixture was heated to 80° C. for 30 min.; then the phases separated and the aqueous layer extracted with toluene. The aqueous layer was dried under vacuum and the salt product digested with ethanol. The ethanol solution was neutralized to pH 6.4 using diethylamine. Next, 90% ethanol (1.5 ml) was added and the mixture was cooled to 0° C. Sulfuric acid (42.5 μl, 42.5 mmol) was added dropwise. After the resulting solution was warmed to room temp, 1,1,3,3-tetraethoxypropane (32.7 μl, 0.135 mmol) was added dropwise and mixture was refluxed for 3 h, allowed to cool to room temperature and neutralized to pH7 with Na₂CO₃. The resulting solution was extracted with DCM, dried over Na₂SO and concentrated under high vacuum. The product (1.83 mg) was isolated via silica chromatography using an ISCO automated system (MeOH/DCM gradient) in 8% yield in the form of a white solid. LRMS (ESI+) (M+H): 343.22.

### Example CP

### (3R,11R,E)-11-(2H-indazol-2-yl)-3-phenyl-1-oxa-4-azacyclododec-8-ene-5,12-dione

In a flask, 2-nitrobenzaldehyde (5.63 mg, 0.037 mmol), TFA salt form of (3R,11R,E)-11-amino-3-phenyl-1-oxa-4-azacyclododec-8 ene-5,12-dione (10 mg, 0.025 mmol), triethylamine (10.39 μl, 0.075 mmol), and 4 Å molecular sieves were heated in DCM (10.0 ml) for 4 hours at 50° C. The reaction was cooled and triethylphosphite (21.31 μl, 0.124 mmol) was added. The reaction mixture was heated to 110° C. overnight (14 hours) in toluene. The crude reaction mixture was concentrated under high vacuum The product (0.6 mg) was isolated via silica chromatography using an ISCO automated system (MeOH/DCM gradient) in 0.6% yield in the form of a white solid. LRMS (ESI+) (M+H): 390.15.

### Example CQ

### (2R,6S,E)-6-(2-oxo-2-(piperidin-1-yl)ethyl)-2-phenyl-1-oxa-4-azacyclododec-8-ene-5,12-dione

26 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using piperidine (6.4 mg, 0.08 mmol, 1.0 eq) in the final coupling step. LRMS (ESI+) (M+H): 399.30.

### Example CR

### 2-((2R,6S,E)-5,12-dioxo-2-phenyl-1-oxa-4-azacyclododec-8-en-6-yl)-N—((R)-1,2,3,4-tetrahydronaphthalen-1-yl)acetamide

41 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using (R)-1,2,3,4-tetrahydronaphthalen-1-amine (17 mg, 0.12 mmol, 1.0 eq) in the final coupling step. LRMS (ESI+) (M+H): 461.45.

### Example CS

### N-(4-chlorobenzyl)-2-((3R,11S,E)-3-(4-fluorophenyl)-5,12-dioxo-1-oxa-4-azacyclododec-8-en-11-yl)acetamide

(R)-2-amino-2-(4-fluorophenyl)ethanol: Lithium aluminum hydride (348 mg, 9.16 mmol) was suspended in anhydrous THF (5 ml) under nitrogen atmosphere and cooled to 0° C. (R)-2-amino-2-(4-fluorophenyl)acetic acid (500 mg, 2.96 mmol) dissolved in THF (11 ml) was then added dropwise. After the addition was complete, the reaction mixture was refluxed overnight. After cooling the mixture to 0° C., water (0.3 ml) was added slowly, followed by sat. K₂CO₃aq. (0.3 ml). Addition of an excess of K₂CO₃ powder was followed by filtration, washing with THF and CHCl₃ and evaporation of the solvents. The product (258 mg) was obtained in 57% yield after column chromatography (DCM/MeOH gradient). LRMS (ESI+) (M+H): 156.08.

(R)—N-(1-(4-fluorophenyl)-2-hydroxyethyl)pent-4-enamide: Using general procedure A for amide bond formation with DMF as solvent, the title compound was prepared from pent-4-enoic acid (72 μl, 0.71 mmol) and (R)-2-amino-2-(4-fluorophenyl)ethanol (100 mg, 0.64 mmol). The product (153 mg) was obtained and used in the next step without purification. LRMS (ESI+) (M+H): 238.14.

(S)-4-tert-butyl 1-((R)-2-(4-fluorophenyl)-2-(pent-4-enamido)ethyl) 2-allylsuccinate: Using general procedure C for ester bond formation with DCM as solvent the title compound was prepared from (S)-2-(2-tert-butoxy-2-oxoethyl)pent-4-enoic acid (152 mg, 0.71 mmol) and (R)—N-(1-(4-fluorophenyl)-2-hydroxyethyl)pent-4-enamide (153 mg, 0.64 mmol). The product (166 mg) was obtained in 60% yield over 2 steps after column chromatography (Hex/EA gradient). LRMS (ESI+) (M+Na): 456.32.

tert-butyl 2-((3R,11S,E)-3-(4-fluorophenyl)-5,12-dioxo-1-oxa-4-azacyclododec-8-en-11-yl)acetate: Using the general procedure for ring closing metathesis with toluene as solvent the title compound was prepared from (S)-4-tert-butyl 1-((R)-2-(4-fluorophenyl)-2-(pent-4-enamido)ethyl) 2-allylsuccinate (166 mg, 0.38 mmol). The product (111 mg) was obtained as a white solid in 72% yield following purification by column chromatography (Hex/EA gradient). LRMS (ESI−) (M−H): 404.17.

N-(4-chlorobenzyl)-2-((3R,11S,E)-3-(4-fluorophenyl)-5,12-dioxo-1-oxa-4-azacyclododec-8-en-11-yl)acetamide: To a solution of tert-butyl 2-((3R,11S,E)-3-(4-fluorophenyl)-5,12-dioxo-1-oxa-4-azacyclododec-8-en-11-yl)acetate (100 mg, 0.25 mmol) in CH₂Cl₂ (2.7 mL) cooled in an ice/water bath was added trifluoroacetic acid (283 μL, 3.70 mmol) and the reaction mixture was stirred 4 h at which point TLC analysis indicated complete consumption of starting material. The reaction mixture was concentrated and coevaporated 3 times with toluene. Then, using general procedure A for amide bond formation with DMF as solvent, the title compound was prepared from 2-((3R,11S,E)-3-(4-fluorophenyl)-5,12-dioxo-1-oxa-4-azacyclododec-8-en-11-yl)acetic acid (86 mg, 0.25 mmol) and (4-chlorophenyl)methanamine (33 μl, 0.27 mmol). The product (98 mg) was obtained in 84% yield over 2 steps after column chromatography (MeOH/DCM gradient). LRMS (ESI+) (M+H): 473.25.

### Example CT

### 2-((2R,6S,E)-5,12-dioxo-2-phenyl-1-oxa-4-azacyclododec-8-en-6-yl)-N-(4,4,4-trifluorobutyl)acetamide

31 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using 4,4,4-trifluorobutan-1-amine (15 mg, 0.12 mmol, 1.0 eq) in the final coupling step. LRMS (ESI+) (M+H): 441.39.

### Example CU

### N-(4-chlorobenzyl)-2-((2R,6S,E)-4-methyl-5,12-dioxo-2-phenyl-1-oxa-4-azacyclododec-8-en-6-yl)acetamide

tert-butyl 2-((2R,6S,E)-4-methyl-5,12-dioxo-2-phenyl-1-oxa-4-azacyclododec-8-en-6-yl)acetate: To a solution of tert-butyl 2-((2R,6S,E)-5,12-dioxo-2-phenyl-1-oxa-4-azacyclododec-8-en-6-yl)acetate (200 mg, 0.516 mmol) and iodomethane (96 μL, 1.55 mmol) in DMF (2.58 mL) cooled in an ice/water bath was added sodium hydride (20.6 mg, 0.516 mmol). The reaction mixture was slowly warmed to rt. Upon stirring 4 h, TLC analysis indicated complete consumption of starting material. The reaction was diluted with H₂O and EtOAC and the layers separated. The aqueous was extracted 2× with EtOAc then the combined organic extracts were washed with brine, dried over Na₂SO₄, filtered, and concentrated. The crude product was purified using silica gel chromatography (MeOH/DCM gradient) to yield 207 mg of desired product as a colorless oil in quantitative yield. LRMS (M+H)⁺: 402.12.

N-(4-chlorobenzyl)-2-((2R,6S,E)-4-methyl-5,12-dioxo-2-phenyl-1-oxa-4-azacyclododec-8-en-6-yl)acetamide: To a solution of tert-butyl 2-((2R,6S,E)-4-methyl-5,12-dioxo-2-phenyl-1-oxa-4-azacyclododec-8-en-6-yl)acetate (130 mg, 0.32 mmol) in DCM (250 μL) cooled in an ice/water bath was added trifluoroacetic acid (112 μL, 1.5 mmol). The reaction mixture was stirred 1 h at which point no starting material remained and the solution was concentrated and dried under high vacuum to yield 2-((2R,6S,E)-4-methyl-5,12-dioxo-2-phenyl-1-oxa-4-azacyclododec-8-en-6-yl)acetic acid in quantitative yield. This intermediate was combined with (4-chlorophenyl)methanamine (40 μL, 0.32 mmol) using general procedure B for amide bond formation to yield the title compound. The product (54 mg) was obtained as a colorless oil in 36% yield. LRMS (ESI+) (M+H): 469.10.

### Example CV

### 2-((2R,6S,11R,E)-11-amino-5,12-dioxo-2-phenyl-1-oxa-4-azacyclododec-8-en-6-yl)-N-(4-chlorobenzyl)acetamide

(S)-tert-butyl 3-(((R)-2-(((R)-2-((((9H-fluoren-9-yl)methoxy)carbonyl)amino)pent-4-enoyl)oxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (140 mg, 0.39 mmol) and (R)—N-Fmoc-allylglycine, synthesized according to a literature procedure (Hruby, V. J. et al. Org. Lett. 6, 3285-3288, 2004). The product was obtained as a white solid after work-up with EtOAc as solvent (247 mg, 97%). LRMS (ESI+) (M+H): 675.72.

tert-butyl 2-((2R,6S,11R,E)-11-((((9H-fluoren-9-yl)methoxy)carbonyl)amino)-5,12-dioxo-2-phenyl-1-oxa-4-azacyclododec-8-en-6-yl)acetate: Using the general procedure for ring closing metathesis using DCM as solvent, the desired product was prepared from (S)-tert-butyl 3-(((R)-2-(((R)-2-((((9H-fluoren-9-yl)methoxy)carbonyl)amino)pent-4-enoyl)oxy)-2-phenylethyl)carbamoyl)hex-5-enoate (255 mg, 0.39 mmol). The product was obtained as a white solid (200 mg, 82%) after column chromatography (MeOH/DCM gradient). LRMS (ESI+) (M+H): 625.20.

(9H-fluoren-9-yl)methyl ((2R,6S,11R,E)-6-(2-((4-chlorobenzyl)amino)-2-oxoethyl)-5,12-dioxo-2-phenyl-1-oxa-4-azacyclododec-8-en-11-yl)carbamate: tert-Butyl 2-((2R,6S,11R,E)-11-((((9H-fluoren-9-yl)methoxy)carbonyl)amino)-5,12-dioxo-2-phenyl-1-oxa-4-azacyclododec-8-en-6-yl)acetate (200 mg, 0.32 mmol) was added to a flask, followed by DCM (6.4 mL), triethylsilane (0.51 mL, 3.2 mmol), and trifluoroacetic acid (0.49 mL, 6.4 mmol). The reaction was stirred for 4 h, then concentrated and dried well under high vacuum. The resulting crude carboxylic acid was reacted with 4-chlorobenzylamine according to general procedure A for amide bond formation, using DMF as solvent. The desired product was isolated as a precipitate from the reaction using a Hirsch funnel and washed with MeOH (137 mg, 62% overall). LRMS (ESI+) (M+H): 692.67.

2-((2R,6S,11R,E)-11-amino-5,12-dioxo-2-phenyl-1-oxa-4-azacyclododec-8-en-6-yl)-N-(4-chlorobenzyl)acetamide: (9H-fluoren-9-yl)methyl ((2R,6S,11R,E)-6-(2-((4-chlorobenzyl)amino)-2-oxoethyl)-5,12-dioxo-2-phenyl-1-oxa-4-azacyclododec-8-en-11-yl)carbamate (137 mg, 0.20 mmol), DMF (3 mL), and piperidine (0.5 mL) were added to a flask and stirred for 3 h. The resulting suspension was concentrated and purified by column chromatography (MeOH/chloroform gradient), to give the desired product as a white solid (68 mg, 73%). LRMS (ESI+) (M+H): 469.96.

### Example CW

### 2-((2R,6S,E)-5,12-dioxo-2-phenyl-1-oxa-4-azacyclododec-8-en-6-yl)-N-isobutylacetamide

2-((2R,6S,E)-5,12-dioxo-2-phenyl-1-oxa-4-azacyclododec-8-en-6-yl)acetic acid (86 mg, 0.26 mmol) was combined with 2-methylpropan-1-amine (29 μl, 0.29 mmol) using general procedure B for amide bond formation with DCM as solvent to yield the title compound. The product (45 mg) was obtained as a white solid following purification of the crude product by column chromatography (MeOH/DCM gradient). LRMS (ESI+) (M+H): 387.15.

### Example CX

### N-(4-chlorobenzyl)-2-((3R,11S,E)-3-(4-chlorophenyl)-5,12-dioxo-1-oxa-4-azacyclododec-8-en-11-yl)acetamide

(R)-2-amino-2-(4-chlorophenyl)ethanol: Lithium aluminum hydride (317 mg, 8.35 mmol) was suspended in anhydrous THF (5 ml) under nitrogen atmosphere and cooled to 0° C. (R)-2-amino-2-(4-chlorophenyl)acetic acid (500 mg, 2.69 mmol) dissolved in THF (10 ml) was then added dropwise. After the addition was complete, the reaction mixture was refluxed overnight. After cooling the mixture to 0° C., water (0.3 ml) was added slowly, followed by sat. K₂CO₃aq. (0.3 ml). Addition of an excess of K₂CO₃ powder was followed by filtration and washing with THF and CHCl₃. Evaporation of the solvents gave 510 mg of a yellow solid. Product (312 mg) was obtained in 68% yield after column chromatography (DCM/MeOH gradient). LRMS (ESI+) (M+H): 172.05.

(R)—N-(1-(4-chlorophenyl)-2-hydroxyethyl)pent-4-enamide: Using general procedure A for amide bond formation with DMF as solvent, the title compound was prepared from pent-4-enoic acid (65 μl, 0.64 mmol) and (R)-2-amino-2-(4-chlorophenyl)ethanol (100 mg, 0.58 mmol). The product (148 mg) was obtained and used in the next step without purification. LRMS (ESI+) (M+H): 254.12.

(S)-4-tert-butyl 1-((R)-2-(4-chlorophenyl)-2-(pent-4-enamido)ethyl) 2-allylsuccinate: Using general procedure C for ester bond formation with DCM as solvent the title compound was prepared from (S)-2-(2-tert-butoxy-2-oxoethyl)pent-4-enoic acid (137 mg, 0.64 mmol) and (R)—N-(1-(4-chlorophenyl)-2-hydroxyethyl)pent-4-enamide (148 mg, 0.58 mmol). The product (128 mg) was obtained in 49% yield over 2 steps after column chromatography (Hex/EA gradient). LRMS (ESI+) (M+Na): 472.31.

tert-butyl 2-((3R,11S,E)-3-(4-chlorophenyl)-5,12-dioxo-1-oxa-4-azacyclododec-8-en-11-yl)acetate: Using the general procedure for ring closing metathesis with toluene as solvent the title compound was prepared from (S)-4-tert-butyl 1-((R)-2-(4-chlorophenyl)-2-(pent-4-enamido)ethyl) 2-allylsuccinate (128 mg, 0.28 mmol). The product (83 mg) was obtained as a white solid in 69% yield following purification by column chromatography (Hex/EA gradient). LRMS (ESI−) (M−H): 420.14.

N-(4-chlorobenzyl)-2-((3R,11S,E)-3-(4-chlorophenyl)-5,12-dioxo-1-oxa-4-azacyclododec-8-en-11-yl)acetamide: To a solution of tert-butyl 2-((3R,11S,E)-3-(4-chlorophenyl)-5,12-dioxo-1-oxa-4-azacyclododec-8-en-11-yl)acetate (70 mg, 0.17 mmol) in CH₂Cl₂ (1.8 mL) cooled in an ice/water bath was added trifluoroacetic acid (191 μL, 2.49 mmol) and the reaction mixture was stirred 4 h at which point TLC analysis indicated complete consumption of starting material. The reaction mixture was concentrated and coevaporated 3 times with toluene. Then, using general procedure A for amide bond formation with DMF as solvent, the title compound was prepared from 2-((3R,11S,E)-3-(4-chlorophenyl)-5,12-dioxo-1-oxa-4-azacyclododec-8-en-11-yl)acetic acid (61 mg, 0.17 mmol) and (4-chlorophenyl methanamine (22 μl, 0.18 mmol). The product (37 mg) was obtained in 46% yield over 2 steps after column chromatography (MeOH/DCM gradient). LRMS (ESI+) (M+H): 491.16.

### Example CY

### N-(4-chlorobenzyl)-2-((3R,11S,E)-5,12-dioxo-3-phenyl-1-oxa-4-azacyclododec-8-en-11-yl)acetamide

2-((3R,11S,E)-5,12-dioxo-3-phenyl-1-oxa-4-azacyclododec-8-en-11-yl)acetic acid: To a solution of tert-butyl 2-((3R,11S,E)-5,12-dioxo-3-phenyl-1-oxa-4-azacyclododec-8-en-11-yl)acetate (130 mg, 0.34 mmol) in DCM (260 μL) cooled in an ice/water bath was added trifluoroacetic acid (120 μL, 1.5 mmol). The reaction mixture was stirred 1 h at which point no starting material remained and the solution was concentrated and dried under high vacuum to yield the title compound in quantitative yield. LRMS (ESI+) (M+H): 332.21.

N-(4-chlorobenzyl)-2-((3R,11S,E)-5,12-dioxo-3-phenyl-1-oxa-4-azacyclododec-8-en-11-yl)acetamide: 2-((3R,11S,E)-5,12-dioxo-3-phenyl-1-oxa-4-azacyclododec-8-en-11-yl)acetic acid (110 mg, 0.34 mmol) was combined with (4-chlorophenyl)methanamine (53 μL, 0.44 mmol) using general procedure B for amide bond formation to yield the title compound. The product (75 mg) was obtained as a white solid in 49% yield. LRMS (ESI+) (M+H): 455.10.

### Example CZ

### N-(4-chlorobenzyl)-2-((2R,3S,6S,E)-3,4-dimethyl-5,12-dioxo-2-phenyl-1-oxa-4-azacyclododec-8-en-6-yl)acetamide

tert-butyl 2-((2R,3S,6S,E)-3,4-dimethyl-5,12-dioxo-2-phenyl-1-oxa-4-azacyclododec-8-en-6-yl)acetate: To a solution of tert-butyl 2-((2R,3S,6S,E)-3-methyl-5,12-dioxo-2-phenyl-1-oxa-4-azacyclododec-8-en-6-yl)acetate (100 mg, 0.25 mmol) and iodomethane (47 μl, 0.747 mmol) in DMF (1.2 mL) cooled in an ice/water bath was added sodium hydride (60% in mineral oil) (6 mg, 0.25 mmol). After 2 h, the reaction was complete and the mixture was diluted with ethyl acetate and washed twice with aqueous HCl 0.1N, then brine. The resulting solution was dried over sodium sulfate, filtered, and concentrated. The product (60 mg) was obtained in 58% yield after column chromatography (Hex/EA gradient). LRMS (ESI+) (M+Na): 438.45.

2-((2R,3S,6S,E)-3,4-dimethyl-5,12-dioxo-2-phenyl-1-oxa-4-azacyclododec-8-en-6-yl)acetic acid: To a solution of tert-butyl 2-((2R,3S,6S,E)-3,4-dimethyl-5,12-dioxo-2-phenyl-1-oxa-4-azacyclododec-8-en-6-yl)acetate (59 mg, 0.142 mmol) in DCM (1 mL) cooled in an ice/water bath was added trifluoroacetic acid (141 μL, 1.85 mmol) and the reaction mixture was stirred for 2 h. The reaction mixture was concentrated, coevaporated 3 times with toluene and the product was utilized in the next step without further purification. LRMS (ESI−) (M−H): 358.31.

N-(4-chlorobenzyl)-2-((2R,3S,6S,E)-3,4-dimethyl-5,12-dioxo-2-phenyl-1-oxa-4-azacyclododec-8-en-6-yl)acetamide: Using general procedure A for amide bond formation with DCM as solvent, the title compound was prepared from 2-((2R,3S,6S,E)-3,4-dimethyl-5,12-dioxo-2-phenyl-1-oxa-4-azacyclododec-8-en-6-yl)acetic acid (51 mg, 0.142 mmol) and (4-chlorophenyl)methanamine (19 μL, 0.156 mmol). The product (42 mg) was obtained as a white solid in 61% yield after column chromatography (MeOH/DCM gradient). LRMS (ESI−) (M−H): 481.92.

### Example DA

### N-(4-chlorobenzyl)-2-((3S,11S,E)-5,12-dioxo-3-phenyl-1-oxa-4-azacyclododec-8-en-11-yl)acetamide

(S)—N-(2-hydroxy-1-phenylethyl)pent-4-enamide: Using general procedure A for amide bond formation with DMF as solvent, the title compound was prepared from pent-4-enoic acid (164 μl, 1.60 mmol) and (S)-2-amino-2-phenylethanol (200 mg, 1.46 mmol). The product (173 mg) was obtained and used in the next step without purification. LRMS (ESI+) (M+H): 220.18.

(S)-4-tert-butyl 1-((S)-2-pent-4-enamido-2-phenylethyl) 2-allylsuccinate: Using general procedure C for ester bond formation with DCM as solvent the title compound was prepared from (S)-2-(2-tert-butoxy-2-oxoethyl)pent-4-enoic acid (344 mg, 1.41 mmol) and (S)—N-(2-hydroxy-1-phenylethyl)pent-4-enamide (320 mg, 1.46 mmol). Product (302 mg) was obtained in 50% yield over 2 steps after column chromatography (Hex/EA gradient). LRMS (ESI+) (M+H): 416.17.

tert-butyl 2-((3S,11S,E)-5,12-dioxo-3-phenyl-1-oxa-4-azacyclododec-8-en-11-yl)acetate: Using the general procedure for ring closing metathesis with toluene as solvent the title compound was prepared from (S)-4-tert-butyl 1-((S)-2-pent-4-enamido-2-phenylethyl) 2-allylsuccinate (302 mg, 0.73 mmol). The product (234 mg) was obtained as a white solid in 83% yield following purification by column chromatography (Hex/EA gradient). LRMS (ESI+) (M+Na): 410.40.

N-(4-chlorobenzyl)-2-((3S,11S,E)-5,12-dioxo-3-phenyl-1-oxa-4-azacyclododec-8-en-11-yl)acetamide: To a solution of tert-butyl 2-((3S,11S,E)-5,12-dioxo-3-phenyl-1-oxa-4-azacyclododec-8-en-11-yl)acetate (232 mg, 0.60 mmol) in CH₂Cl₂ (8.5 mL) cooled in an ice/water bath was added trifluoroacetic acid (688 μL, 8.98 mmol) and the reaction mixture was stirred 4 h at which point TLC analysis indicated complete consumption of starting material. The reaction mixture was concentrated and coevaporated 3 times with toluene. Then, using general procedure A for amide bond formation with DMF as solvent, the title compound was prepared from 2-((3S,11S,E)-5,12-dioxo-3-phenyl-1-oxa-4-azacyclododec-8-en-11-yl)acetic acid (198 mg, 0.60 mmol) and (4-chlorophenyl)methanamine (80 μl, 0.66 mmol). The product (184 mg) was obtained in 68% yield over 2 steps after column chromatography (MeOH/DCM gradient). LRMS (ESI+) (M+H): 455.39.

### Example DB

### N-(4-chlorobenzyl)-2-((2R,6S,11S)-11-methyl-5,12-dioxo-2-phenyl-1-oxa-4-azacyclododecan-6-yl)acetamide

N-(4-Chlorobenzyl)-2-((2R,6S,11S,E)-11-methyl-5,12-dioxo-2-phenyl-1-oxa-4-azacyclododec-8-en-6-yl)acetamide (22 mg, 0.047 mmol), 4-methylbenzenesulfonohydrazide (437 mg, 2.35 mmol), and DME (2.8 mL) were added to a 15 mL flask. The mixture was heated to 80° C., and a solution of sodium acetate (192 mg, 2.35 mmol) in water (2.8 mL) was added over 2 h via syringe pump. The reaction was then stirred for an additional 2 h, before it was removed from the heat and diluted with EtOAc and water. The phases were separated, and the organic phase was washed twice with 1 M aq. HCl, then brine. The organic phase was dried over MgSO₄, filtered, and concentrated. The resulting crude material was purified by column chromatography (MeOH/DCM gradient), giving the desired product as a white solid (13 mg, 59%). LRMS (ESI+) (M+H): 471.33.

### Example DC

### N-(4-chlorobenzyl)-2-((2R,3S,6S)-3,4-dimethyl-5,12-dioxo-2-phenyl-1-oxa-4-azacyclododecan-6-yl)acetamide

tert-butyl 2-((2R,3S,6S)-3,4-dimethyl-5,12-dioxo-2-phenyl-1-oxa-4-azacyclododecan-6-yl)acetate: tert-butyl 2-((2R,3S,6S,E)-3,4-dimethyl-5,12-dioxo-2-phenyl-1-oxa-4-azacyclododec-8-en-6-yl)acetate (35 mg, 0.084 mmol) in methanol (421 μL) was stirred under hydrogen atmosphere in the presence of palladium 10 wt. % on activated carbon (10 mg). After 2 h at room temperature, the reaction mixture was filtered, washed with methanol, and evaporated. The product (34 mg) was obtained in 97% yield and used in the next step without further purification. LRMS (ESI+) (M+Na): 440.38.

N-(4-chlorobenzyl)-2-((2R,3S,6S)-3,4-dimethyl-5,12-dioxo-2-phenyl-1-oxa-4-azacyclododecan-6-yl)acetamide: To a solution of tert-butyl 2-((2R,3S,6S)-3,4-dimethyl-5,12-dioxo-2-phenyl-1-oxa-4-azacyclododecan-6-yl)acetate (34 mg, 0.081 mmol) in DCM (582 μL) cooled in an ice/water bath was added trifluoroacetic acid (94 μL, 1.22 mmol) and the reaction mixture was stirred 4 h at which point TLC analysis indicated complete consumption of starting material. The reaction mixture was concentrated, coevaporated 3 times with toluene. Using general procedure A for amide bond formation with DMF as solvent, the title compound was prepared from 2-((2R,3S,6S)-3,4-dimethyl-5,12-dioxo-2-phenyl-1-oxa-4-azacyclododecan-6-yl)acetic acid (29 mg, 0.0.081 mmol) and (4-chlorophenyl)methanamine (11 μL, 0.089 mmol). The product (27 mg) was obtained as a white solid in 69% yield after column chromatography (MeOH/DCM gradient). LRMS (ESI+) (M+H): 485.20.

### Example DD

### N-((6-chloropyridin-3-yl)methyl)-2-((3R,11S,E)-5,12-dioxo-3-phenyl-1-oxa-4-azacyclododec-8-en-11-yl)acetamide

2-((3R,11S,E)-5,12-dioxo-3-phenyl-1-oxa-4-azacyclododec-8-en-11-yl)acetic acid (86 mg, 0.26 mmol) was combined with (6-chloropyridin-3-yl)methanamine (37 mg, 0.26 mmol) using general procedure B for amide bond formation to yield the title compound. The product (62 mg) was obtained as a white solid in 52% yield. LRMS (ESI−) (M−H): 454.02.

### Example DE

### N-(4-chlorobenzyl)-2-((2R,6S,11S,E)-11-(isopropylamino)-5,12-dioxo-2-phenyl-1-oxa-4-azacyclododec-8-en-6-yl)acetamide

2-((2R,6S,11S,E)-11-amino-5,12-dioxo-2-phenyl-1-oxa-4-azacyclododec-8-en-6-yl)-N-(4-chlorobenzyl)acetamide (55 mg, 0.12 mmol) was suspended in a flask with MeOH (5 mL) and DCM (0.5 mL). Acetone (1 mL, 14 mmol) was added, followed by sodium cyanoborohydride (110 mg, 1.76 mmol), and 1 drop of acetic acid (Pasteur pipet). The flask was sealed and the reaction was stirred for 17 h, before concentrating to remove the acetone. The mixture was diluted with EtOAc and aq. NaHCO₃, then the phases were separated and the organics were washed with brine and dried over Na₂SO₄, filtered, and concentrated to provide the desired product as a white solid (57 mg, 95%). LRMS (ESI+) (M+H): 512.15.

### Example DF

### N-(4-chlorobenzyl)-2-((2R,6S,11S,E)-11-methyl-5,12-dioxo-2-phenyl-1-oxa-4-azacyclododec-8-en-6-yl)acetamide

(S)-tert-butyl 3-(((R)-2-(((S)-2-methylpent-4-enoyl)oxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (180 mg, 0.50 mmol) and (S)-2-methylpent-4-enoic acid, prepared according to a literature procedure (Chakraborty, T. K. et al. Synlett 2002, 2039-2040). The product was obtained as a white solid after work-up with EtOAc as solvent (211 mg, 98%). LRMS (ESI+) (M+Na): 452.53.

tert-butyl 2-((2R,6S,11S,E)-11-methyl-5,12-dioxo-2-phenyl-1-oxa-4-azacyclododec-8-en-6-yl)acetate: Using the general procedure for ring closing metathesis using DCM as solvent, the desired product was prepared from (S)-tert-butyl 3-(((R)-2-(((S)-2-methylpent-4-enoyl)oxy)-2-phenylethyl)carbamoyl)hex-5-enoate (216 mg, 0.50 mmol), yielding the product as a white solid (176 mg, 87%) after column chromatography (EtOAc/hexanes gradient). LRMS (ESI+) (M+Na): 424.42.

N-(4-chlorobenzyl)-2-((2R,6S,11S,E)-11-methyl-5,12-dioxo-2-phenyl-1-oxa-4-azacyclododec-8-en-6-yl)acetamide: tert-Butyl 2-((2R,6S,11S,E)-11-methyl-5,12-dioxo-2-phenyl-1-oxa-4-azacyclododec-8-en-6-yl)acetate (76 mg, 0.19 mmol) was added to a flask, followed by DCM (3.8 mL), triethylsilane (0.30 mL, 1.9 mmol), and trifluoroacetic acid (0.29 mL, 3.8 mmol). The reaction was stirred for 16 h, then concentrated and dried well under high vacuum. The resulting crude carboxylic acid was reacted with 4-chlorobenzylamine according to general procedure A for amide bond formation, using DMF as solvent. The desired product was isolated as a white solid (75 mg, 84%) after work-up using EtOAc as solvent and purification by column chromatography (MeOH/DCM gradient). LRMS (ESI+) (M+H): 469.13.

### Example DH

### N-(4-chlorobenzyl)-2-((2R,6S,E)-5,12-dioxo-2-phenyl-1-oxa-4-azacyclododec-8-en-6-yl)acetamide

2-((2R,6S,E)-5,12-dioxo-2-phenyl-1-oxa-4-azacyclododec-8-en-6-yl)acetic acid (120 mg, 0.36 mmol) was combined with (4-chlorophenyl)methanamine (50 μL, 0.40 mmol) using general procedure B for amide bond formation with DCM as solvent to yield the title compound. Product (41 mg) was obtained as a white solid in 25% yield. LRMS (ESI+) (M+H): 455.22.

### Example DI

### N-(4-chlorobenzyl)-2-((3R,6S,11S,E)-6-methyl-5,12-dioxo-3-phenyl-1-oxa-4-azacyclododec-8-en-11-yl)acetamide

(S)—N—((R)-2-hydroxy-1-phenylethyl)-2-methylpent-4-enamide: Using general procedure A for amide bond formation with DCM as solvent, the desired product was prepared from (R)-2-amino-2-phenylethanol and (S)-2-methylpent-4-enoic acid (92 wt % in Et₂O, 186 mg, 1.50 mmol), prepared according to a literature procedure (Chakraborty, T. K. et al. Synlett 2002, 2039-2040). The product was obtained as a colorless oil (173 mg, 49%) after work-up using DCM as solvent, and column chromatography (EtOAc/hexanes gradient). LRMS (ESI+) (M+H): 234.16.

(S)-4-tert-butyl 1-((R)-2-((S)-2-methylpent-4-enamido)-2-phenylethyl) 2-allylsuccinate: Using general procedure C for ester bond formation with DCM as solvent, the desired product was prepared from (S)—N—((R)-2-hydroxy-1-phenylethyl)-2-methylpent-4-enamide (50 mg, 0.15 mmol) and (S)-2-(2-(tert-butoxy)-2-oxoethyl)pent-4-enoic acid (199 mg, 0.93 mmol). The colorless oil thus obtained after work-up with DCM as solvent was used immediately in the ring closing metathesis reaction. LRMS (ESI+) (M−t-Bu): 374.27.

tert-butyl 2-((3R,6S,11S,E)-6-methyl-5,12-dioxo-3-phenyl-1-oxa-4-azacyclododec-8-en-11-yl)acetate: Using the general procedure for ring closing metathesis with toluene as solvent, the desired product was prepared from (S)-4-tert-butyl 1-((R)-2-((S)-2-methylpent-4-enamido)-2-phenylethyl) 2-allylsuccinate (301 mg, 0.70 mmol), yielding a white solid (175 mg, 62%) after column chromatography (EtOAc/hexanes gradient). LRMS (ESI+) (M−t-Bu): 346.22.

tert-butyl 2-((3R,6S,11S,Z)-6-methyl-5,12-dioxo-3-phenyl-1-oxa-4-azacyclododec-8-en-11-yl)acetate: The title compound was isolated as the minor, less polar product in the above reaction, as a white solid (16 mg, 6%). LRMS (ESI+) (M−t-Bu): 346.22.

N-(4-chlorobenzyl)-2-((3R,6S,11S,E)-6-methyl-5,12-dioxo-3-phenyl-1-oxa-4-azacyclododec-8-en-11-yl)acetamide: tert-Butyl 2-((3R,6S,11S,E)-6-methyl-5,12-dioxo-3-phenyl-1-oxa-4-azacyclododec-8-en-11-yl)acetate (135 mg, 0.336 mmol) was added to a flask, followed by DCM (10 mL), Et₃SiH (0.54 mL, 3.36 mmol) and trifluoroacetic acid (1.0 mL, 13.0 mmol). The reaction was stirred for 17 h, then concentrated and dried well under high vacuum. 31 mg (0.090 mmol) of the resulting crude carboxylic acid was reacted with 4-chlorobenzylamine according to general procedure A for amide bond formation, using DMF as solvent. A portion of the desired product was filtered off with a Hirsch funnel, and the remainder was isolated after work-up according to the general procedure using EtOAc as solvent, yielding a white solid (34 mg, 80%). LRMS (ESI+) (M+H): 469.28.

### Example DJ

### 2-((2R,6S,11S,E)-11-acetamido-5,12-dioxo-2-phenyl-1-oxa-4-azacyclododec-8-en-6-yl)-N-(4-chlorobenzyl)acetamide

2-((2R,6S,11S,E)-11-amino-5,12-dioxo-2-phenyl-1-oxa-4-azacyclododec-8-en-6-yl)-N-(4-chlorobenzyl)acetamide (30 mg, 0.064 mmol) was added to a 4 mL vial with stir bar and sealed under nitrogen, followed by DMF (1 mL), NEt₃ (0.22 mL, 1.60 mmol) and acetic anhydride (0.15 mL, 1.60 mmol). The reaction was stirred for 48 h, then concentrated to dryness, re-dissolved with EtOAc, and washed successively with half-saturated aq. NaHCO₃, 1 M aq. HCl, and brine. The organic phase was dried over MgSO₄, filtered, and concentrated. The resulting crude material was purified by column chromatography (MeOH/DCM gradient), yielding the desired product as a white solid (32 mg, 97%). LRMS (ESI+) (M+H): 512.11.

### Example DK

### (2R,6S,E)-6-(2-morpholino-2-oxoethyl)-2-phenyl-1-oxa-4-azacyclododec-8-ene-5,12-dione

23 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using morpholine (6.6 mg, 0.08 mmol, 1.0 eq) in the final coupling step. LRMS (ESI+) (M+H): 401.28.

### Example DL

### N-(4-chlorobenzyl)-2-((2R,6S)-5,12-dioxo-2-phenyl-1-oxa-4-azacyclododecan-6-yl)acetamide

To a solution of tert-butyl 2-((2R,6S)-5,12-dioxo-2-phenyl-1-oxa-4-azacyclododecan-6-yl)acetate (68 mg, 0.18 mmol) in DCM (130 μL) cooled in an ice/water bath was added trifluoroacetic acid (61 μL, 0.79 mmol). The reaction mixture was stirred 30 min at which point no starting material remained and the solution was concentrated and dried under high vacuum to yield 2-((2R,6S)-5,12-dioxo-2-phenyl-1-oxa-4-azacyclododecan-6-yl)acetic acid in quantitative yield. This intermediate was combined with (4-chlorophenyl)methanamine (21 μL, 0.18 mmol) using general procedure B for amide bond formation with DCM as solvent to yield the title compound. The product (15 mg) was obtained as a white solid in 19% yield over the two steps. LRMS (ESI+) (M+H): 457.30.

### Example DM

### 2-((2R,3S,6S,11S,E)-11-acetamido-3,4-dimethyl-5,12-dioxo-2-phenyl-1-oxa-4-azacyclododec-8-en-6-yl)-N-(4-chlorobenzyl)acetamide

(S)-tert-butyl 3-(((1R,2S)-1-((S)-2-(((9H-fluoren-9-yl)methoxy)carbonylamino)pent-4-enoyloxy)-1-phenylpropan-2-yl)(methyl)carbamoyl)hex-5-enoate: Using general procedure C for ester bond formation with DCM as solvent the title compound was prepared from (S)-2-(((9H-fluoren-9-yl)methoxy)carbonylamino)pent-4-enoic acid (84 mg, 0.249 mmol) and (S)-tert-butyl 3-(((1R,2S)-1-hydroxy-1-phenylpropan-2-yl)(methyl)carbamoyl)hex-5-enoate (90 mg, 0.249 mmol). The product (170 mg) was obtained and used in the next step without purification. LRMS (ESI+) (M+H): 681.62.

tert-butyl 2-((2R,3S,6S,11S,E)-11-(((9H-fluoren-9-yl)methoxy)carbonylamino)-3,4-dimethyl-5,12-dioxo-2-phenyl-1-oxa-4-azacyclododec-8-en-6-yl)acetate: Using the general procedure for ring closing metathesis with toluene as solvent the title compound was prepared from (S)-tert-butyl 3-(((1R,2S)-1-((S)-2-(((9H-fluoren-9-yl)methoxy)carbonylamino)pent-4-enoyloxy)-1-phenylpropan-2-yl)(methyl)carbamoyl)hex-5-enoate (170 mg, 0.25 mmol). The product (82 mg) was obtained in 50% yield following purification by column chromatography (Hex/EA gradient). LRMS (ESI+) (M+H): 653.19.

2-((2R,3S,6S,11S,E)-11-(((9H-fluoren-9-yl)methoxy)carbonylamino)-3,4-dimethyl-5,12-dioxo-2-phenyl-1-oxa-4-azacyclododec-8-en-6-yl)acetic acid: To a solution of tert-butyl 2-((2R,3S,6S,11S,E)-11-(((9H-fluoren-9-yl)methoxy)carbonylamino)-3,4-dimethyl-5,12-dioxo-2-phenyl-1-oxa-4-azacyclododec-8-en-6-yl)acetate (82 mg, 0.126 mmol) in CH₂Cl₂ (1.2 mL) cooled in an ice/water bath was added trifluoroacetic acid (144 μL, 1.884 mmol) and the reaction mixture was stirred 4 h at which point LCMS analysis indicated complete consumption of starting material. The reaction mixture was concentrated, coevaporated 3 times with toluene. The product (75 mg) was obtained and used in the next step without purification. LRMS (ESI+) (M+Na): 597.00.

(9H-fluoren-9-yl)methyl (2R,3S,6S,11S,E)-6-(2-(4-chlorobenzylamino)-2-oxoethyl)-3,4-dimethyl-5,12-dioxo-2-phenyl-1-oxa-4-azacyclododec-8-en-11-ylcarbamate: Using general procedure A for amide bond formation with DMF as solvent, the title compound was prepared from 2-((2R,3S,6S,11S,E)-11-(((9H-fluoren-9-yl)methoxy)carbonylamino)-3,4-dimethyl-5,2-dioxo-2-phenyl-1-oxa-4-azacyclododec-8-en-6-yl)acetic acid (75 mg, 0.126 mmol) and (4-chlorophenyl)methanamine (17 μL, 0.138 mmol). The product (75 mg) was obtained in 83% yield after column chromatography (MeOH/DCM gradient). LRMS (ESI+) (M+H): 720.37.

2-((2R,3S,6S,11S,E)-11-acetamido-3,4-dimethyl-5,12-dioxo-2-phenyl-1-oxa-4-azacyclododec-8-en-6-yl)-N-(4-chlorobenzyl)acetamide: (9H-fluoren-9-yl)methyl (2R,3S,6S,11S,E)-6-(2-(4-chlorobenzylamino)-2-oxoethyl)-3,4-dimethyl-5,12-dioxo-2-phenyl-1-oxa-4-azacyclododec-8-en-11-ylcarbamate (75 mg, 0.104 mmol) was dissolved in DMF (2.3 mL) and piperidine (771 μL, 7.81 mmol) was added dropwise. After 30 minutes, the reaction is complete as shown by TLC. Solvents are evaporated and coevaporated with toluene 3 times. The product (29 mg) was obtained and used in the next step without further purification. 2-((2R,3S,6S,11S,E)-11-amino-3,4-dimethyl-5,12-dioxo-2-phenyl-1-oxa-4-azacyclododec-8-en-6-yl)-N-(4-chlorobenzyl)acetamide (29 mg, 0.058 mmol) was dissolved in DMF (3.0 mL) under argon atmosphere. Triethylamine (818 μL, 5.82 mmol) and acetic anhydride (550 μL, 5.82 mmol) were successively added in one portion and the reaction mixture was stirred at room temperature for 6 h. Solvents were evaporated before the mixture was dissolved with ethyl acetate and successively washed with NH₄Cl solution and NaHCO₃ solution. The product (29 mg) was obtained in 52% yield over 2 steps after purification by column chromatography (DCM/MeOH gradient). LRMS (ESI+) (M+Na): 562.97.

### Example DN

### 2-((2R,6S,E)-5,12-dioxo-2-phenyl-1-oxa-4-azacyclododec-8-en-6-yl)-N-((1-methylpiperidin-4-yl)methyl)acetamide

tert-Butyl 2-((2R,6S,E)-5,12-dioxo-2-phenyl-1-oxa-4-azacyclododec-8-en-6-yl)acetate (305 mg, 0.79 mmol) was added to a flask, followed by DCM (5 mL), triethylsilane (1.3 mL, 7.9 mmol), and trifluoroacetic acid (1.2 mL, 15.7 mmol). The reaction was stirred for 4 h, then concentrated and dried well under high vacuum. 80 mg (0.24 mmol) of this material was reacted with (1-methylpiperidin-4-yl)methanamine according to general procedure C for amide bond formation with DCM as solvent. The desired product was isolated as a precipitate from the reaction using a Hirsch funnel and washed with DCM (70 mg, 66%). LRMS (ESI+) (M+H): 442.05

### Example DO

### 2-((2R,6S,E)-5,12-dioxo-2-phenyl-1-oxa-4-azacyclododec-8-en-6-yl)-N,N-diethylacetamide

tert-Butyl 2-((2R,6S,E)-5,12-dioxo-2-phenyl-1-oxa-4-azacyclododec-8-en-6-yl)acetate (305 mg, 0.79 mmol) was added to a flask, followed by DCM (5 mL), triethylsilane (1.3 mL, 7.9 mmol), and trifluoroacetic acid (1.2 mL, 15.7 mmol). The reaction was stirred for 4 h, then concentrated and dried well under high vacuum. 50 mg (0.15 mmol) of the resulting crude carboxylic acid was reacted with diethylamine according to general procedure C for amide bond formation (with the addition of 2 eq. of (i-Pr)₂NEt), using DCM as solvent. The desired product was isolated from the reaction according to the general procedure using EtOAc as solvent for the work-up (21 mg, 36%). LRMS (ESI+) (M+H): 387.05.

### Eample DP

### N-(4-chlorobenzyl)-2-((9S,14aS,E)-3,10-dioxo-3,4,5,8,9,10,12,13,14,14a-decahydro-1H-pyrrolo[2,1-c][1,4]oxaazacyclododecin-9-yl)acetamide

(S)-tert-butyl 3-((S)-2-(hydroxymethyl)pyrrolidine-1-carbonyl)hex-5-enoate: Using general procedure A for amide bond formation with DCM as solvent, the title compound was prepared from (S)-2-(2-tert-butoxy-2-oxoethyl)pent-4-enoic acid (481 mg, 2.25 mmol) and (S)-pyrrolidin-2-ylmethanol (250 mg, 2.47 mmol). The product (638 mg) was obtained in 95% yield after column chromatography (MeOH/DCM gradient). LRMS (ESI+) (M+Na): 320.31.

(S)-tert-butyl 3-((S)-2-((pent-4-enoyloxy)methyl)pyrrolidine-1-carbonyl)hex-5-enoate: Using general procedure C for ester bond formation with DCM as solvent the title compound was prepared from 4-pentenoic acid (238 μL, 2.334 mmol) and (S)-tert-butyl 3-((S)-2-(hydroxymethyl)pyrrolidine-1-carbonyl)hex-5-enoate (631 mg, 2.12 mmol). The product (542 mg) was obtained in 68% yield after column chromatography (Hex/EtOAc gradient). LRMS (ESI+) (M+Na): 402.40.

tert-butyl 2-((9S,14aS,E)-3,10-dioxo-3,4,5,8,9,10,12,13,14,14a-decahydro-1H-pyrrolo[2,1-c][1,4]oxaazacyclododecin-9-yl)acetate: Using the general procedure for ring closing metathesis with toluene as solvent the title compound was prepared from (S)-tert-butyl 3-((S)-2-((pent-4-enoyloxy)methyl)pyrrolidine-1-carbonyl)hex-5-enoate (542 mg, 1.428 mmol). The product (420 mg) was obtained as a white solid in 84% yield following purification by column chromatography (Hex/EtOAc gradient). LRMS (ESI+) (M+Na): 374.36.

N-(4-chlorobenzyl)-2-((9S,14aS,E)-3,10-dioxo-3,4,5,8,9,10,12,13,14,14a-decahydro-1H-pyrrolo[2,1-c][1,4]oxaazacyclododecin-9-yl)acetamide: To a solution of tert-butyl 2-((9S,14aS,E)-3,10-dioxo-3,4,5,8,9,10,12,13,14,14a-decahydro-1H-pyrrolo[2,1-c][1,4]oxaazacyclododecin-9-yl)acetate (420 mg, 1.195 mmol) in DCM (17 mL) cooled in an ice/water bath was added trifluoroacetic acid (1373 μL, 17.93 mmol) and the reaction mixture was stirred 3 h at which point TLC analysis indicated complete consumption of starting material. The reaction mixture was concentrated, coevaporated 3 times with toluene. Then, using general procedure A for amide bond formation with DMF as solvent, the title compound was prepared from 2-((9S,14aS,E)-3,10-dioxo-3,4,5,8,9,10,12,13,14,14a-decahydro-1H-pyrrolo[2,1-c][1,4]oxaazacyclododecin-9-yl)acetic acid (353 mg, 1.195 mmol) and (4-chlorophenyl)methanamine (161 μL, 1.315 mmol). The product (431 mg) was obtained as a white solid in 86% yield after column chromatography (MeOH/DCM gradient). LRMS (ESI+) (M+H): 419.30.

### Example DQ

### 2-((2R,6S,E)-5,12-dioxo-2-phenyl-1-oxa-4-azacyclododec-8-en-6-yl)-N-(4-nitrobenzyl)acetamide

82 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using (4-nitrophenyl)methanamine (50 mg, 0.33 mmol, 1.1 eq) in the final coupling step. LRMS (ESI+) (M+H): 466.28.

### Example DR

### 2-((2R,3S,6S,E)-3,4-dimethyl-5,12-dioxo-2-phenyl-1-oxa-4-azacyclododec-8-en-6-yl)-N-(2-ethoxyethyl)acetamide

Using general procedure A for amide bond formation with DMF as solvent, the title compound was prepared from 2-((2R,3S,6S,E)-3,4-dimethyl-5,12-dioxo-2-phenyl-1-oxa-4-azacyclododec-8-en-6-yl)acetic acid (28 mg, 0.078 mmol) and 2-ethoxyethanamine (7.64 mg, 0.086 mmol). The product (22 mg) was obtained in 66% yield after column chromatography (MeOH/DCM gradient). LRMS (ESI+) (M+H): 431.24.

### Example DS

### N-(4-chlorobenzyl)-2-((4S,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetamide

tert-butyl 2-((4S,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetate: Using general procedure C for ester bond formation with DCM as solvent (S)-tert-butyl 3-((S)-2-((((S)-2-methylpent-4-enoyl)oxy)methyl)pyrrolidine-1-carbonyl)hex-5-enoate was prepared from (R)-2-methylpent-4-enoic acid (69 mg, 0.61 mmol) and ((S)-tert-butyl 3-((S)-2-(hydroxymethyl)pyrrolidine-1-carbonyl)hex-5-enoate (164 mg, 0.55 mmol). The product was obtained as a clear oil and used directly in the next step. Using the general procedure for ring closing metathesis with toluene as solvent tert-butyl 2-((4S,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetate was prepared from (S)-tert-butyl 3-((S)-2-((pent-4-enoyloxy)methyl)pyrrolidine-1-carbonyl)hex-5-enoate (217 mg, 0.55 mmol). The product (52 mg) was obtained as a clear oil in 26% yield over two steps following purification by column chromatography (Hex/EtOAc gradient). ¹H NMR (300 MHz, CDCl₃) δ 5.63-5.32 (m, 2H), 4.75 (m, 1H), 4.52 (t, J=11.3, 1H), 4.03 (dt, J=7.9, 12.6, 1H), 3.58 (dt, J=3.3, 6.5, 1H), 3.34-3.20 (m, 1H), 2.99 (t, J=6.6, 1H), 2.25 (ovrlp m, 10H), 1.71-1.59 (m, 1H), 1.40 (s, 9H), 1.15 (d, J=6.8, 3H).

N-(4-chlorobenzyl)-2-((4S,9S,14aS,E)-4-methyl-3,10-dioxo-3,4,5,8,9,12,12,13,14,14a-decahydro-1H-pyrrolo[2,1-c][1,4]oxaazacyclododecin-9-yl)acetamide: To a solution of tert-butyl 2-((4S,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetate (52 mg, 0.142 mmol) in CH₂Cl₂ (2 mL) cooled in an ice/water bath was added trifluoroacetic acid (216 μL, 2.85 mmol) and the reaction mixture was stirred 3 h at which point TLC analysis indicated complete consumption of starting material. The reaction mixture was concentrated, coevaporated 3 times with toluene. Then, using general procedure A for amide bond formation with DMF as solvent, the title compound was prepared from 2-((4S,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetic acid (44 mg, 0.142 mmol) and (4-chlorophenyl)methanamine (35 μL, 0.284 mmol). The product (34 mg) was obtained as a white solid in 54% yield after column chromatography (MeOH/DCM gradient). ¹H NMR (300 MHz, CDCl₃) δ 7.38-7.05 (m, 4H), 5.60-5.28 (m, 2H), 4.65 (s, 1H), 4.49 (t, J=10.0, 1H), 4.41-4.22 (m, 2H), 3.75-3.58 (m, 1H), 3.50 (dd, J=2.6, 11.7, 1H), 3.25-3.08 (m, 2H), 2.63-2.44 (m, 2H), 2.44-2.28 (m, 2H), 2.23-1.94 (m, 4H), 1.82 (dd, J=9.2, 17.3, 1H), 1.72-1.50 (m, 3H), 1.13 (d, J=6.8, 3H).

### Example DT

### N-(4-chlorobenzyl)-2-((4S,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetamide

tert-butyl 2-((4S,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetate: Using general procedure C for ester bond formation with DCM as solvent (S)-tert-butyl 3-((S)-2-((((S)-2-methylpent-4-enoyl)oxy)methyl)pyrrolidine-1-carbonyl)hex-5-enoate was prepared from (R)-2-methylpent-4-enoic acid (69 mg, 0.61 mmol) and ((S)-tert-butyl 3-((S)-2-(hydroxymethyl)pyrrolidine-1-carbonyl)hex-5-enoate (164 mg, 0.55 mmol). Product was obtained as a clear oil and used directly in the next step. Using the general procedure for ring closing metathesis with toluene as solvent tert-butyl 2-((4S,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetate was prepared from (S)-tert-butyl 3-((S)-2-((pent-4-enoyloxy)methyl)pyrrolidine-1-carbonyl)hex-5-enoate (217 mg, 0.55 mmol). The product (40 mg) was obtained as a clear oil in 20% yield over two steps following purification by column chromatography (Hex/EtOAc gradient). ¹H NMR (300 MHz, CDCl₃) δ 5.90-5.60 (m, 2H), 5.07 (m, J=15.6, 4H), 4.36 (m, 1H), 4.13 (dd, J=6.0, 17.1, 2H), 3.73 (broad s, 1H), 3.52 (broad s, 1H), 2.96 (s, 1H), 2.79-1.64 (m, 11H), 1.41 (s, 9H), 1.17 (s, 3H).

N-(4-chlorobenzyl)-2-((4S,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetamide: To a solution of tert-butyl 2-((4S,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetate (40 mg, 0.109 mmol) in CH₂Cl₂ (2 mL) cooled in an ice/water bath was added trifluoroacetic acid (166 μL, 2.19 mmol) and the reaction mixture was stirred 3 h at which point TLC analysis indicated complete consumption of starting material. The reaction mixture was concentrated, coevaporated 3 times with toluene. Then, using general procedure A for amide bond formation with DMF as solvent, the title compound was prepared from 2-((4S,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetic acid (34 mg, 0.109 mmol) and (4-chlorophenyl)methanamine (27 μL, 0.22 mmol). The product (8 mg) was obtained as a white solid in 17% yield after column chromatography (MeOH/DCM gradient). ¹H NMR (300 MHz, CDCl₃) δ 7.23 (m, 4H), 6.49 (s, 1H), 5.52 (ddd, J=6.7, 16.9, 25.8, 2H), 4.65 (s, 1H), 4.34 (d, J=5.3, 2H), 3.96 (d, J=16.6, 2H), 3.59 (dt, J=6.4, 9.0, 1H), 3.44-3.15 (m, 2H), 2.77-2.63 (m, 1H), 2.60-2.01 (overlp m, 5H), 1.90-1.54 (ovrlp m, 5H), 1.25 (d, J=7.1, 3H).

### Example DU

### N-(4-chlorobenzyl)-2-((4R,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetamide

tert-butyl 2-((4R,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetate: Using general procedure C for ester bond formation with DCM as solvent (S)-tert-butyl 3-((S)-2-((((R)-2-methylpent-4-enoyl)oxy)methyl)pyrrolidine-1-carbonyl)hex-5-enoate was prepared from (S)-2-methylpent-4-enoic acid (64 mg, 0.56 mmol) and ((S)-tert-butyl 3-((S)-2-(hydroxymethyl)pyrrolidine-1-carbonyl)hex-5-enoate (152 mg, 0.51 mmol). The product was obtained as a clear oil and used directly in the next step. Using the general procedure for ring closing metathesis with toluene as solvent tert-butyl 2-((4R,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetate was prepared from (S)-tert-butyl 3-((S)-2-((((R)-2-methylpent-4-enoyl)oxy)methyl)pyrrolidine-1-carbonyl)hex-5-enoate (201 mg, 0.51 mmol). The product was obtained as a clear oil and used directly in the next step without further purification.

N-(4-chlorobenzyl)-2-((4R,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetamide: To a solution of tert-butyl 2-((4R,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetate (31 mg, 0.084 mmol) in CH₂Cl₂ (2 mL) cooled in an ice/water bath was added trifluoroacetic acid (129 μL, 1.70 mmol) and the reaction mixture was stirred 3 h at which point TLC analysis indicated complete consumption of starting material. The reaction mixture was concentrated, coevaporated 3 times with toluene. Then, using general procedure A for amide bond formation with DMF as solvent, the title compound was prepared from 2-((4R,9S,14aS,E)-4-methyl-3,10-dioxo-3,4,5,8,9,10,12,13,14,14a-decahydro-1H-pyrrolo[2,1-c][1,4]oxaazacyclododecin-9-yl)acetic acid (26 mg, 0.084 mmol) and (4-chlorophenyl)methanamine (21 μL, 0.168 mmol). The product (16 mg) was obtained as a white solid in 44% yield after column chromatography (MeOH/DCM gradient). ¹H NMR (300 MHz, CDCl₃) δ 7.47-7.02 (m, 4H), 6.50 (s, 1H), 5.66-5.27 (m, 2H), 4.49 (d, J=7.9, 1H), 4.32 (q, J=6.5, 2H), 4.17-3.92 (m, 2H), 3.63 (s, 1H), 3.28 (s, 1H), 3.07 (s, 1H), 2.65-1.56 (m, 11H), 1.15 (d, J=6.9, 3H).

### Example DV

### N-(4-chlorobenzyl)-2-((4R,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetamide

tert-butyl 2-((4R,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetate: Using general procedure C for ester bond formation with DCM as solvent (S)-tert-butyl 3-((S)-2-((((R)-2-methylpent-4-enoyl)oxy)methyl)pyrrolidine-1-carbonyl)hex-5-enoate was prepared from (S)-2-methylpent-4-enoic acid (64 mg, 0.56 mmol) and ((S)-tert-butyl 3-((S)-2-(hydroxymethyl)pyrrolidine-1-carbonyl)hex-5-enoate (152 mg, 0.51 mmol). The product was obtained as a clear oil and used directly in the next step. Using the general procedure for ring closing metathesis with toluene as solvent tert-butyl 2-((4R,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetate was prepared from (S)-tert-butyl 3-((S)-2-((((R)-2-methylpent-4-enoyl)oxy)methyl)pyrrolidine-1-carbonyl)hex-5-enoate (201 mg, 0.51 mmol). The product was obtained as a clear oil and used directly in the next step without further purification.

N-(4-chlorobenzyl)-2-((4R,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetamide: To a solution of tert-butyl 2-((4R,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetate (25 mg, 0.068 mmol) in CH₂Cl₂ (2 mL) cooled in an ice/water bath was added trifluoroacetic acid (104 μL, 1.37 mmol) and the reaction mixture was stirred 3 h at which point TLC analysis indicated complete consumption of starting material. The reaction mixture was concentrated, coevaporated 3 times with toluene. Then, using general procedure A for amide bond formation with DMF as solvent, the title compound was prepared from 2-((4R,9S,14aS,Z)-4-methyl-3,10-dioxo-3,4,5,8,9,10,12,13,14,14a-decahydro-1H-pyrrolo[2,1-c][1,4]oxaazacyclododecin-9-yl)acetic acid (21 mg, 0.068 mmol) and (4-chlorophenyl)methanamine (17 μL, 0.136 mmol). The product (4 mg) was obtained as a white solid in 15% yield after column chromatography (MeOH/DCM gradient). ¹H NMR (300 MHz, CDCl₃) δ 7.40-7.11 (m, 4H), 6.31 (s, 1H), 5.61-5.27 (m, 2H), 4.68 (s, 1H), 4.36 (d, J=11.9, 1H), 4.11-3.74 (m, 2H), 3.59 (s, 2H), 3.30 (s, 1H), 2.73-2.01 (m, 5H), 1.70 (d, J=42.5, 6H), 1.28 (d, J=7.1, 4H).

### Example DW

### (3R,11S,E)-11-(3-(4-chlorophenyl)-2-oxopropyl)-1-methyl-3-phenyl-1,4-diazacyclododec-8-ene-5,12-dione

(R)-tert-butyl (2-amino-1-phenylethyl)carbamate): A solution of diethyl azodicarboxylate (40% weight in toluene) (2.88 ml, 7.27 mmol) was added drop-wise to a suspension of dicyclohexylammonium (R)-2-((tert-butoxycarbonyl)amino)pent-4-enoate (3.00 g, 7.17 mmol), phthalimide (1.05 g, 7.17 mmol), and triphenylphosphine (1.88 g, 7.17 mmol) in THF (74.3 ml). The mixture was stirred at room temperature overnight (15 hours) and concentrated under vacuum to afford the crude phthalimide derivative, (R)-tert-butyl (2-(1,3-dioxoisoindolin-2-yl)-1-phenylethyl)carbamate as a cloudy yellow oil. The phthalimide derivative was dissolved in EtOH (55.8 ml). Hydrazine hydrate (2.10 ml, 43 mmol) was added, upon which the mixture turned cloudy. The crude reaction was heated to reflux at 80° C. for 5 hours. The crude reaction was then cooled, diluted with DI-water (70.0 mL), and extracted with DCM (4×40 ml). The organic extracts were combined, dried over Na₂SO₄, and dried under high vacuum to afford the diamine derivative, (R)-tert-butyl (2-amino-1-phenylethyl)carbamate (1.146 g), in 67% yield as a clear, light yellow oil after silica chromatography using an ISCO automated system using MeOH/DCM gradient. LRMS (ESI+) (M+H): 237.22

(R)-tert-butyl (2-(benzylamino)-1-phenylethyl)carbamate: To a solution of the (R)-tert-butyl (2-amino-1-phenylethyl)carbamate (1.21 g, 5.13 mmol) in MeOH (15.0 ml), benzaldehyde (1.04 ml, 10.25 mmol), molecular sieves 3A^{°}, and triethylamine (2.14 ml, 15.38 mmol) were added. The reaction mixture was stirred at room temperature for 1 hour, and then cooled to 0° C. Sodium borohydride (582.0 mg, 15.38 mmol) was added in small portions, and the reaction mixture was stirred for an additional hour. Upon completion of the reaction, the solvent was removed under vacuum and residue was partitioned between with EtOAc and DI-water. The organic solution was extracted with 0.5N HCl (3×25 ml). The aqueous solution was neutralized with NH₄OH (25%) and extracted with DCM, (3×25 ml). The combined organic layers were dried over Na₂SO₄, concentrated under vacuum, and purified over silica chromatography using an ISCO automated system (MeOH/DCM gradient) to give (R)-tert-butyl (2-(benzylamino)-1-phenylethyl)carbamate (1.501 g) in 90% yield as a clear oil. LRMS (ESI+) (M+H): 327.27.

(R)-tert-butyl (2-(benzyl(methyl)amino)-1-phenylethyl)carbamate: (R)-tert-butyl (2-(benzylamino)-1-phenylethyl)carbamate (1.198 g, 3.67 mmol) was dissolved in a solution of triethylamine (1.56 ml, 11.20 mmol) and MeOH (15.0 ml). To the mixture, formaldehyde (0.83 ml, 11.20 mmol) was added, and the reaction was stirred for 45 minutes. The reaction was cooled down to 0° C. to which sodium borohydride (0.424 g, 11.20 mmol) was added and mixture was stirred for an additional 45 min. The addition of formaldehyde and NaBH₄ was repeated twice more after every 45 min. Upon completion of the reaction, the solvent was evaporated and residue dissolved in water, acidified to pH 6 with 1N HCl, and extracted with DCM. The organic layer was dried over Na₂SO₄ to afford (R)-tert-butyl (2-(benzyl(methyl)amino)-1-phenylethyl)carbamate (1.225 g) as a light yellow oil in 98% crude yield. LRMS (ESI+) (M+H): 341.19.

(R)-tert-butyl (2-(methylamino)-1-phenylethyl)carbamate: Crude (R)-tert-butyl (2-(benzyl(methyl)amino)-1-phenylethyl)carbamate (1.225 g, 3.60 mmol) was dissolved in MeOH (40.0 ml), upon which 10% Pd (dry) on carbon (wet) (586 mg, 5.51 mmol) was added and the reaction was stirred under hydrogen overnight (16 hours). Upon completion of the reaction, the crude mixture was filtered over celite and purified over silica chromatography (basic column, MeOH/DCM gradient) to afford (R)-tert-butyl (2-(methylamino)-1-phenylethyl)carbamate (903 mg), in 99% yield as a light yellow oil. LRMS (ESI+) (M+H): 341.29.

(S)-tert-butyl-3-(((R)-2-((tert-butoxycarbonyl)amino)-2-phenylethyl)(methyl)-carbamoyl)hex-5-enoate: Using General Procedure B for Amide Bond Formation with DMF as solvent (S)-tert-butyl-3-(((R)-2-((tert-butoxycarbonyl)amino)-2-phenylethyl)(methyl)-carbamoyl)hex-5-enoate was prepared from (S)-2-(2-(tert-butoxy)-2-oxoethyl)pent-4-enoic acid (130.0 μl, 0.666 mmol) was dissolved in anhydrous DMF (10 ml) and (R)-tert-butyl 2-(methylamino)-1-phenylethylcarbamate (200.0 mg, 0.799 mmol). The product (220 mg) was obtained as a white solid in 74% crude yield. LRMS (ESI+) (M+H): 447.28.

(S)-3-(((R)-2-amino-2-phenylethyl)(methyl)carbamoyl)hex-5-enoic acid: Using the N-Boc (N-tert-butyl-oxycarbonyl) removal step from General Procedure for Amino Macrocyles, ((S)-3-(((R)-2-amino-2-phenylethyl)(methyl)carbamoyl)hex-5-enoic acid was prepared from tert-butyl (S)-tert-butyl-3-(((R)-2-((tert-butoxycarbonyl)amino)-2-phenylethyl)(methyl)-carbamoyl)hex-5-enoate (223.0 mg, 0.499 mmol). The product (141.0 mg) was isolated in 97% yield after silica chromatography using an ISCO automated system (MeOH/DCM gradient). LRMS (ESI+) (M+H): 291.19.

(S)—N—((R)-2-amino-2-phenylethyl)-2-(3-(4-chlorophenyl)-2-oxopropyl)-N-methylpent-4-enamide: Using General Procedure B for Amide Bond Formation with DMF as solvent, (S)—N—((R)-2-amino-2-phenylethyl)-2-(3-(4-chlorophenyl)-2-oxopropyl)-N-methylpent-4-enamide was prepared from 4-chlorobenzylamine (303 μl, 2.480 mmol) and (S)-3-(((R)-2-amino-2-phenylethyl)(methyl)carbamoyl)hex-5-enoic acid (144 mg, 0.496 mmol) in DMF. The crude product (178 mg) was isolated in 87% yield. LRMS (ESI+) (M+H): 414.15.

(S)-2-(3-(4-chlorophenyl)-2-oxopropyl)-N-methyl-N—((R)-2-(pent-4-enamido)-2-phenylethyl)pent-4-enamide: Using General Procedure B for Amide Bond Formation with DMF as solvent, (S)-2-(3-(4-chlorophenyl)-2-oxopropyl)-N-methyl-N—((R)-2-(pent-4-enamido)-2-phenylethyl)pent-4-enamide was prepared from pent-4-enoic acid (39.0 μl, 0.380 mmol) and (S)—N—((R)-2-amino-2-phenylethyl)-2-(3-(4-chlorophenyl)-2-oxopropyl)-N-methylpent-4-enamide (188.8 mg, 0.456 mmol) in DMF. The product (145 mg) was isolated in 77% yield. LRMS (ESI+) (M+H): 496.28.

(3R,11S,E)-1-(3-(4-chlorophenyl)-2-oxopropyl)-1-methyl-3-phenyl-1,4-diazacyclododec-8-ene-5,12-dione: Using General Procedure for Ring-Closing Metathesis using toluene as solvent, (S)-2-(3-(4-chlorophenyl)-2-oxopropyl)-N-methyl-N—((R)-2-(pent-4-enamido)-2-phenylethyl)pent-4-enamide (29.3 mg, 0.059 mmol) was used in the reaction and stirred overnight (16 hours) at room temperature. The product (15 mg) was isolated in 54% yield after trituration in EtOAc and filtered to obtain a white solid. LRMS (ESI+) (M+Na): 490.73.

### Example DX

### 2-((3R,11S,E)-5,12-dioxo-3-phenyl-1-oxa-4-azacyclododec-8-en-11-yl)-N-(5-methyl-1H-imidazol-2-yl)acetamide

Under nitrogen, 5-methyl-1H-imidazol-2-amine (9.50 mg, 0.098 mmol) was dissolved in anhydrous DMF (1.5 ml) to which diisopropylethylamine (60 μl, 0.362 mmol) was added. Next, 2-((3R,11S,E)-5,12-dioxo-3-phenyl-1-oxa-4-azacyclododec-8-en-11-yl)acetic acid (30 mg, 0.091 mmol), 1-hydroxy-7-azabenzotriazole (20.93 mg, 0.136 mmol), and (1-ethyl-3-(3′-dimethylaminopropyl)carbodiimide) (26.0 mg, 0.136 mmol) were added. The reaction was stirred overnight (15 hours). Upon completion of the reaction, the crude mixture was diluted with EtOAc, washed (×2) with saturated NH₄Cl, half-saturated NaHCO₃, and brine. The combined organic layers were dried over Na₂SO₄ and concentrated under high vacuum. The product was purified via silica gel chromatography using an ISCO automated system (MeOH/DCM gradient). The isolated product (29.7 mg) was obtained in 80% yield as a white solid. LRMS (ESI+) (M+H): 411.23.

### Example DY

### 2-((3R,11S,E)-5,12-dioxo-3-phenyl-1-oxa-4-azacyclododec-8-en-11-yl)-N-(1H-pyrazol-3-yl)acetamide

Under nitrogen, 1H-pyrazol-3-amine (3.76 mg, 0.045 mmol) was dissolved in anhydrous DMF (7001) to which diisopropylethylamine (30 μl, 0.362 mmol) was added. Next, 2-((3R,11S,E)-5,12-dioxo-3-phenyl-1-oxa-4-azacyclododec-8-en-11-yl)acetic acid (15 mg, 0.045 mmol), 1-hydroxy-7-azabenzotriazole (10.47 mg, 0.068 mmol), and (1-ethyl-3-(3′-dimethylaminopropyl)carbodiimide) (13.2 mg, 0.068 mmol) were added. The reaction was stirred overnight (15 hours). Upon completion of the reaction, the crude mixture was diluted with EtOAc, washed (×2) with saturated NH₄Cl, half-saturated NaHCO₃, and brine. The combined organic layers were dried over Na₂SO₄ and concentrated under high vacuum. The product was purified via silica gel chromatography using an ISCO automated system (MeOH/DCM gradient). The isolated product (13.9 mg) was obtained in 77% yield as a white solid. LRMS (ESI+) (M+H): 397.19.

### Example DZ

### N-(4-chlorobenzyl)-2-((2R,6S,E)-5,13-dioxo-2-phenyl-1-oxa-4-azacyclotridec-8-en-6-yl)acetamide

(R)—N-(2-hydroxy-1-phenylethyl)hex-5-enamide: Using general procedure A for amide bond formation with DCM as solvent, the desired product was prepared from (R)-2-amino-2-phenylethanol and hex-5-enoic acid (228 mg, 2.00 mmol). The product was obtained as a white solid (340 mg, 73%) after work-up using EtOAc as solvent, and concentrating the final product in the presence of hexanes. LRMS (ESI+) (M+H): 234.18.

(R)—(R)-2-(hex-5-enamido)-2-phenylethyl 2-methylpent-4-enoate: Using general procedure C for ester bond formation with DCM as solvent, the desired product was prepared from (R)—N-(2-hydroxy-1-phenylethyl)hex-5-enamide (170 mg, 0.73 mmol) and (R)-2-methylpent-4-enoic acid, prepared according to a literature procedure (Chakraborty, T. K. et al. Synlett 2002, 2039-2040). The product was obtained as a colorless oil (238 mg, 99%) after work-up using EtOAc as solvent. LRMS (ESI+) (M+H): 330.30.

(S)-tert-butyl 3-(((R)-2-hydroxy-2-phenylethyl)carbamoyl)hex-5-enoate: Using general procedure A for amide bond formation with DCM as solvent, the desired product was prepared from (S)-2-(2-tert-butoxy-2-oxoethyl)pent-4-enoic acid (4.29 g, 20.0 mmol) and (R)-2-amino-1-phenylethanol. The product was obtained as a white solid after work-up according to the general procedure using EtOAc as solvent (5.16 g, 77%). LRMS (ESI−) (M+HCO₂): 378.28.

(S)-tert-butyl 3-(((R)-2-(hex-5-enoyloxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (157 mg, 0.44 mmol) and hex-5-enoic acid. The product was obtained as a white solid after work-up with EtOAc as solvent (188 mg, 100%). LRMS (ESI+) (M+H): 430.08.

tert-butyl 2-((2R,6S,E)-5,13-dioxo-2-phenyl-1-oxa-4-azacyclotridec-8-en-6-yl)acetate: Using the general procedure for ring closing metathesis with toluene as solvent, the desired product was prepared from (S)-tert-butyl 3-(((R)-2-(hex-5-enoyloxy)-2-phenylethyl)carbamoyl)hex-5-enoate (191 mg, 0.45 mmol). The product was obtained as a white solid (134 mg, 75%) after column chromatography (EtOAc/hexanes gradient). LRMS (ESI+) (M+H): 402.17.

N-(4-chlorobenzyl)-2-((2R,6S,E)-5,13-dioxo-2-phenyl-1-oxa-4-azacyclotridec-8-en-6-yl)acetamide: tert-Butyl 2-((2R,6S,E)-5,13-dioxo-2-phenyl-1-oxa-4-azacyclotridec-8-en-6-yl)acetate (103 mg, 0.26 mmol) was added to a flask, followed by DCM (5 mL), triethylsilane (0.41 mL, 2.57 mmol), and trifluoroacetic acid (0.40 mL, 5.1 mmol). The reaction was stirred for 15 h, then concentrated and dried well under high vacuum, yielding 88.5 mg (100%) of the crude carboxylic acid as a beige solid. 28 mg (0.081 mmol) of this material was reacted with 4-chlorobenzylamine according to general procedure A for amide bond formation with DMF as solvent. The desired product was isolated as a precipitate from the reaction using a Hirsch funnel and washed with DCM (26 mg, 69%). LRMS (ESI+) (M+H): 469.27.

### Example EA

### N-(4-chlorobenzyl)-2-((2R,6S,11R,E)-11-methyl-5,12-dioxo-2-phenyl-1-oxa-4-azacyclododec-8-en-6-yl)acetamide

(S)-tert-butyl 3-(((R)-2-(((S)-2-methylpent-4-enoyl)oxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (180 mg, 0.50 mmol) and (S)-2-methylpent-4-enoic acid, prepared according to a literature procedure (Chakraborty, T. K. et al. Synlett 2002, 2039-2040). The product was obtained as a white solid after work-up with EtOAc as solvent (211 mg, 98%). LRMS (ESI+) (M+Na): 452.53.

tert-butyl 2-((2R,6S,11S,E)-11-methyl-5,12-dioxo-2-phenyl-1-oxa-4-azacyclododec-8-en-6-yl)acetate: Using the general procedure for ring closing metathesis using DCM as solvent, the desired product was prepared from (S)-tert-butyl 3-(((R)-2-(((S)-2-methylpent-4-enoyl)oxy)-2-phenylethyl)carbamoyl)hex-5-enoate (216 mg, 0.50 mmol), yielding the product as a white solid (176 mg, 87%) after column chromatography (EtOAc/hexanes gradient). LRMS (ESI+) (M+Na): 424.42.

(S)-tert-butyl 3-(((R)-2-(((R)-2-methylpent-4-enoyl)oxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (50 mg, 0.15 mmol) and (R)-2-methylpent-4-enoic acid, prepared according to a literature procedure (Chakraborty, T. K. et al. Synlett 2002, 2039-2040). The product was obtained as a pale yellow oil after work-up with EtOAc as solvent (54 mg, 83%). LRMS (ESI+) (M+Na): 452.53.

tert-butyl 2-((2R,6S,11R,E)-11-methyl-5,12-dioxo-2-phenyl-1-oxa-4-azacyclododec-8-en-6-yl)acetate: Using the general procedure for ring closing metathesis with toluene as solvent, the desired product was prepared from (S)-tert-butyl 3-(((R)-2-(((R)-2-methylpent-4-enoyl)oxy)-2-phenylethyl)carbamoyl)hex-5-enoate (53 mg, 0.12 mmol), yielding a white solid (38 mg, 76%) after column chromatography (EtOAc/hexanes gradient). LRMS (ESI+) (M+Na): 424.48.

N-(4-chlorobenzyl)-2-((2R,6S,11R,E)-11-methyl-5,12-dioxo-2-phenyl-1-oxa-4-azacyclododec-8-en-6-yl)acetamide: tert-Butyl 2-((2R,6S,11R,E)-11-methyl-5,12-dioxo-2-phenyl-1-oxa-4-azacyclododec-8-en-6-yl)acetate (37 mg, 0.092 mmol) was added to a flask, followed by DCM (1 mL) and trifluoroacetic acid (0.071 mL, 0.92 mmol). The reaction was stirred for 4 h, then concentrated and dried well under high vacuum. 15.9 mg (0.046 mmol) of the resulting crude carboxylic acid was reacted with 4-chlorobenzylamine according to general procedure A for amide bond formation, using DMF as solvent. The desired product was isolated as a white solid (10.8 mg, 50%) after work-up using EtOAc as solvent and purification by column chromatography (MeOH/DCM gradient). LRMS (ESI+) (M+H): 469.13.

### Example EB

### 2-((2R,6S,11S,E)-11-amino-5,12-dioxo-2-phenyl-1-oxa-4-azacyclododec-8-en-6-yl)-N-(4-chlorobenzyl)acetamide

(S)-tert-butyl 3-(((R)-2-(((S)-2-((((9H-fluoren-9-yl)methoxy)carbonyl)amino)pent-4-enoyl)oxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (333 mg, 0.93 mmol) and L-N-Fmoc-allylglycine. The product was obtained as a white solid (566 mg, 93%) after work-up using EtOAc as solvent and purification by column chromatography (EtOAc/hexanes gradient). LRMS (ESI+) (M+Na): 675.72.

tert-butyl 2-((2R,6S,11S,E)-11-((((9H-fluoren-9-yl)methoxy)carbonyl)amino)-5,12-dioxo-2-phenyl-1-oxa-4-azacyclododec-8-en-6-yl)acetate: Using the general procedure for ring closing metathesis using DCM as solvent, the desired product was prepared from (S)-tert-butyl 3-(((R)-2-(((S)-2-((((9H-fluoren-9-yl)methoxy)carbonyl)amino)pent-4-enoyl)oxy)-2-phenylethyl)carbamoyl)hex-5-enoate (566 mg, 0.87 mmol), yielding the product as a white solid (429 mg, 79%). LRMS (ESI+) (M+H): 625.20.

(9H-fluoren-9-yl)methyl ((2R,6S,11S,E)-6-(2-((4-chlorobenzyl)amino)-2-oxoethyl)-5,12-dioxo-2-phenyl-1-oxa-4-azacyclododec-8-en-11-yl)carbamate: tert-Butyl 2-((2R,6S,11S,E)-11-((((9H-fluoren-9-yl)methoxy)carbonyl)amino)-5,12-dioxo-2-phenyl-1-oxa-4-azacyclododec-8-en-6-yl)acetate (566 mg, 0.91 mmol) was added to a flask, followed by DCM (18.1 mL), triethylsilane (1.45 mL, 9.1 mmol), and trifluoroacetic acid (1.40 mL, 18.1 mmol). The reaction was stirred for 15 h, then concentrated and dried well under high vacuum. 340 mg (0.60 mmol) of the resulting crude carboxylic acid was reacted with 4-chlorobenzylamine according to general procedure A for amide bond formation, using DMF as solvent. The desired product was isolated as a precipitate from the reaction using a Hirsch funnel and washed with MeOH (157 mg, 38%). LRMS (ESI+) (M+H): 692.67.

2-((2R,6S,11S,E)-11-amino-5,12-dioxo-2-phenyl-1-oxa-4-azacyclododec-8-en-6-yl)-N-(4-chlorobenzyl)acetamide: (9H-fluoren-9-yl)methyl ((2R,6S,11S,E)-6-(2-((4-chlorobenzyl)amino)-2-oxoethyl)-5,12-dioxo-2-phenyl-1-oxa-4-azacyclododec-8-en-11-yl)carbamate (157 mg, 0.23 mmol), DMF (1.7 mL), and piperidine (0.6 mL) were added to a flask and stirred for 6 h. The resulting suspension was concentrated, pulverized, and washed well with DCM (filtering with a Hirsch funnel) to remove the Fmoc byproduct, yielding the desired product as a white solid (90 mg, 84%). LRMS (ESI+) (M+H): 470.13.

### Example EC

### N-(4-chlorobenzyl)-2-((2R,6S,11S,E)-11-hydroxy-5,12-dioxo-2-phenyl-1-oxa-4-azacyclododec-8-en-6-yl)acetamide

(S)-tert-butyl 3-(((R)-2-(((R)-2-((((9H-fluoren-9-yl)methoxy)carbonyl)amino)pent-4-enoyl)oxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (140 mg, 0.39 mmol) and (R)—N-Fmoc-allylglycine, synthesized according to a literature procedure (Hruby, V. J. et al. Org. Lett. 6, 3285-3288, 2004). The product was obtained as a white solid after work-up with EtOAc as solvent (247 mg, 97%). LRMS (ESI+) (M+H): 675.72.

tert-butyl 2-((2R,6S,11R,E)-11-((((9H-fluoren-9-yl)methoxy)carbonyl)amino)-5,12-dioxo-2-phenyl-1-oxa-4-azacyclododec-8-en-6-yl)acetate: Using the general procedure for ring closing metathesis using DCM as solvent, the desired product was prepared from (S)-tert-butyl 3-(((R)-2-(((R)-2-((((9H-fluoren-9-yl)methoxy)carbonyl)amino)pent-4-enoyl)oxy)-2-phenylethyl)carbamoyl)hex-5-enoate (255 mg, 0.39 mmol). The product was obtained as a white solid (200 mg, 82%) after column chromatography (MeOH/DCM gradient). LRMS (ESI+) (M+H): 625.20.

(9H-fluoren-9-yl)methyl ((2R,6S,11R,E)-6-(2-((4-chlorobenzyl)amino)-2-oxoethyl)-5,12-dioxo-2-phenyl-1-oxa-4-azacyclododec-8-en-11-yl)carbamate: tert-Butyl 2-((2R,6S,11R,E)-11-((((9H-fluoren-9-yl)methoxy)carbonyl)amino)-5,12-dioxo-2-phenyl-1-oxa-4-azacyclododec-8-en-6-yl)acetate (200 mg, 0.32 mmol) was added to a flask, followed by DCM (6.4 mL), triethylsilane (0.51 mL, 3.2 mmol), and trifluoroacetic acid (0.49 mL, 6.4 mmol). The reaction was stirred for 4 h, then concentrated and dried well under high vacuum. The resulting crude carboxylic acid was reacted with 4-chlorobenzylamine according to general procedure A for amide bond formation, using DMF as solvent. The desired product was isolated as a precipitate from the reaction using a Hirsch funnel and washed with MeOH (137 mg, 62% overall). LRMS (ESI+) (M+H): 692.67.

(S)-tert-butyl 3-(((R)-2-(((S)-2-((tert-butyldimethylsilyl)oxy)pent-4-enoyl)oxy)-2-phenylethyl)carbamoyl)hex-5-enoate: Using general procedure C for ester bond formation with DCM as solvent, the desired product was prepared from (S)-tert-butyl 3-(((R)-2-hydroxy-2-phenylethyl)carbamoyl)hex-5-enoate (71 mg, 0.21 mmol) and (S)-2-((tert-butyldimethylsilyl)oxy)pent-4-enoic acid. The product was obtained as a colorless oil (75 mg, 65%) after work-up with EtOAc as solvent and purification by column chromatography (EtOAc/hexanes gradient). LRMS (ESI+) (M+Na): 568.62.

tert-butyl 2-((2R,6S,11S,E)-11-((tert-butyldimethylsilyl)oxy)-5,12-dioxo-2-phenyl-1-oxa-4-azacyclododec-8-en-6-yl)acetate: Using the general procedure for ring closing metathesis using toluene as solvent, the desired product was prepared from (S)-tert-butyl 3-(((R)-2-(((S)-2-((tert-butyldimethylsilyl)oxy)pent-4-enoyl)oxy)-2-phenylethyl)carbamoyl)hex-5-enoate (75 mg, 0.14 mmol), yielding a colorless oil (66 mg, 93%) after column chromatography (EtOAc/hexanes gradient). LRMS (ESI+) (M+Na): 540.58.

2-((2R,6S,11S,E)-11-((tert-butyldimethylsilyl)oxy)-5,12-dioxo-2-phenyl-1-oxa-4-azacyclododec-8-en-6-yl)-N-(4-chlorobenzyl)acetamide: tert-Butyl 2-((2R,6S,11S,E)-11-((tert-butyldimethylsilyl)oxy)-5,12-dioxo-2-phenyl-1-oxa-4-azacyclododec-8-en-6-yl)acetate (66 mg, 0.127 mmol) was added to a flask with stir bar, followed by DCM (2 mL) and trifluoroacetic acid (1 mL). The reaction was stirred for 15 h, then concentrated. The crude solid was pulverized and extracted with DCM, leaving behind a mostly insoluble byproduct, which was consistent with compound without the TBS protecting group. The solid was filtered off, and the mother liquor was concentrated to a beige solid (45 mg, 61%). The resulting crude carboxylic acid was reacted with 4-chlorobenzylamine according to general procedure A for amide bond formation, using DMF as solvent. The reaction was worked up according to the procedure using EtOAc as solvent, with MeOH and DCM added before filtration to keep the product in solution. The crude product was purified by column chromatography (MeOH/DCM gradient), then preparative HPLC-MS (CH₃CN/water gradient). The pure fractions were concentrated in a high-vacuum evaporator, yielding the desired product as a white solid (11.7 mg, 21%). LRMS (ESI+) (M+H): 585.20.

N-(4-chlorobenzyl)-2-((2R,6S,11S,E)-11-hydroxy-5,12-dioxo-2-phenyl-1-oxa-4-azacyclododec-8-en-6-yl)acetamide: 2-((2R,6S,11S,E)-11-((tert-butyldimethylsilyl)oxy)-5,12-dioxo-2-phenyl-1-oxa-4-azacyclododec-8-en-6-yl)-N-(4-chlorobenzyl)acetamide (4.7 mg, 0.0080 mmol) was added to a plastic vial w/stir bar, along with THF (2 mL) to facilitate the transfer. HF-pyridine (1 drop) was added via a 1 mL plastic syringe. The reaction was stirred for 24 h, after which time the resulting white precipitate was filtered with a Hirsch funnel and washed with THF, giving a white solid (3.0 mg, 79%). LRMS (ESI+) (M+H): 471.40.

### Example ED

### 2-((3R,6R,11S,E)-6-benzyl-5,12-dioxo-3-phenyl-1-oxa-4-azacyclododec-8-en-11-yl)-N-(4-chlorobenzyl)acetamide

R)-2-benzylpent-4-enoic acid: (S)-4-Benzyl-3-pent-4-enoyloxazolidin-2-one (1.30 g, 5.00 mmol), was added to a flask with stir bar and sealed under nitrogen. Dry THF (21 mL) was added, and the solution was cooled to −78° C. in a dry ice/acetone bath. Sodium hexamethyldisilazide solution (6.25 mL of a 1M solution in THF, 6.25 mmol) was added dropwise by syringe. The reaction was removed from the ice bath for 10 min. and then recooled to −78° C. before benzyl bromide (0.95 mL, 8.00 mmol) was added dropwise. The reaction was then stirred at −78° C. for 7 h, and then quenched by adding acetic acid (1 mL). The mixture was diluted with half-saturated aq. NaHCO₃ (70 mL) and EtOAc (70 mL), and the layers were separated and the aqueous layer re-extracted with EtOAc (50 mL). The combined organics were washed with brine and dried over Na₂SO₄, filtered, and concentrated to a yellow oil. This was purified by column chromatography (EtOAc/hexanes gradient), yielding (S)-4-benzyl-3-((R)-2-benzylpent-4-enoyl)oxazolidin-2-one as a white solid (1.15 g, 66%). 1.10 g (3.15 mmol) of this material was subjected to the hydrolysis conditions reported in the literature (Evans, D. A. et al. Org. Syn. 68, 83-87, 1990), providing (R)-2-benzylpent-4-enoic acid (581 mg, 96%) as a colorless liquid, consistent with literature characterization data (Gouverneur, V. et al. Angew. Chem. Int. Ed. 47, 357-360, 2008).

(R)-2-benzyl-N—((R)-2-hydroxy-1-phenylethyl)pent-4-enamide: Using general procedure A for amide bond formation with DCM as solvent, the desired product was prepared from (R)-2-amino-2-phenylethanol and (R)-2-benzylpent-4-enoic acid (199 mg, 1.00 mmol), yielding a white solid (269 mg, 87%) after work-up using DCM as solvent. LRMS (ESI+) (M+H): 310.20.

(S)-1-((R)-2-((R)-2-benzylpent-4-enamido)-2-phenylethyl) 4-tert-butyl 2-allylsuccinate: Using general procedure C for ester bond formation with DCM as solvent, the desired product was prepared from (R)-2-benzyl-N—((R)-2-hydroxy-1-phenylethyl)pent-4-enamide (259 mg, 0.15 mmol) and (S)-2-(2-(tert-butoxy)-2-oxoethyl)pent-4-enoic acid. The desired product was obtained as a colorless oil (425 mg, 100%) after work-up with DCM as solvent. LRMS (ESI+) (M−t-Bu): 450.32.

tert-butyl 2-((3R,6R,11S,E)-6-benzyl-5,12-dioxo-3-phenyl-1-oxa-4-azacyclododec-8-en-11-yl)acetate: Using the general procedure for ring closing metathesis with toluene as solvent, the desired product was prepared from (S)-1-((R)-2-((R)-2-benzylpent-4-enamido)-2-phenylethyl) 4-tert-butyl 2-allylsuccinate (423 mg, 0.84 mmol), yielding a white solid (220 mg, 55%) after column chromatography (EtOAc/hexanes gradient). LRMS (ESI+) (M−t-Bu): 422.28.

2-((3R,6R,11S,E)-6-benzyl-5,12-dioxo-3-phenyl-1-oxa-4-azacyclododec-8-en-11-yl)-N-(4-chlorobenzyl)acetamide: tert-Butyl 2-((3R,6R,11S,E)-6-benzyl-5,12-dioxo-3-phenyl-1-oxa-4-azacyclododec-8-en-11-yl)acetate (186 mg, 0.389 mmol) was added to flask with stir bar, followed by DCM (10 mL), Et₃SiH (0.5 mL) and trifluoroacetic acid (1 mL). The reaction was stirred for 16 h, then concentrated and dried well under high vacuum. 40 mg (0.095 mmol) of the resulting crude carboxylic acid was reacted with 4-chlorobenzylamine according to general procedure A for amide bond formation, using DMF as solvent. The desired product was isolated as a white solid (48 mg, 93%) after work-up with EtOAc as solvent. LRMS (ESI+) (M+H): 545.20.

### Example EE

### N-(4-chlorobenzyl)-2-((3R,6S,11S,Z)-6-methyl-5,12-dioxo-3-phenyl-1-oxa-4-azacyclododec-8-en-11-yl)acetamide

(S)—N—((R)-2-hydroxy-1-phenylethyl)-2-methylpent-4-enamide: Using general procedure A for amide bond formation with DCM as solvent, the desired product was prepared from (R)-2-amino-2-phenylethanol and (S)-2-methylpent-4-enoic acid (92 wt % in Et₂O, 186 mg, 1.50 mmol), prepared according to a literature procedure (Chakraborty, T. K. et al. Synlett 2002, 2039-2040). The product was obtained as a colorless oil (173 mg, 49%) after work-up using DCM as solvent, and column chromatography (EtOAc/hexanes gradient). LRMS (ESI+) (M+H): 234.16.

(S)-4-tert-butyl 1-((R)-2-((S)-2-methylpent-4-enamido)-2-phenylethyl) 2-allylsuccinate: Using general procedure C for ester bond formation with DCM as solvent, the desired product was prepared from (S)—N—((R)-2-hydroxy-1-phenylethyl)-2-methylpent-4-enamide (50 mg, 0.15 mmol) and (S)-2-(2-(tert-butoxy)-2-oxoethyl)pent-4-enoic acid (199 mg, 0.93 mmol). The colorless oil thus obtained after work-up with DCM as solvent was used immediately in the ring closing metathesis reaction. LRMS (ESI+) (M−t-Bu): 374.27.

tert-butyl 2-((3R,6S,11S,E)-6-methyl-5,12-dioxo-3-phenyl-1-oxa-4-azacyclododec-8-en-11-yl)acetate: Using the general procedure for ring closing metathesis with toluene as solvent, the desired product was prepared from (S)-4-tert-butyl 1-((R)-2-((S)-2-methylpent-4-enamido)-2-phenylethyl) 2-allylsuccinate (301 mg, 0.70 mmol), yielding a white solid (175 mg, 62%) after column chromatography (EtOAc/hexanes gradient). LRMS (ESI+) (M−t-Bu): 346.22.

tert-butyl 2-((3R,6S,11S,Z)-6-methyl-5,12-dioxo-3-phenyl-1-oxa-4-azacyclododec-8-en-11-yl)acetate: The title compound was isolated as the minor, less polar product in the above reaction, as a white solid (16 mg, 6%). LRMS (ESI+) (M−t-Bu): 346.22.

N-(4-chlorobenzyl)-2-((3R,6S,11S,Z)-6-methyl-5,12-dioxo-3-phenyl-1-oxa-4-azacyclododec-8-en-11-yl)acetamide: tert-Butyl 2-((3R,6S,11S,Z)-6-methyl-5,12-dioxo-3-phenyl-1-oxa-4-azacyclododec-8-en-11-yl)acetate (15.8 mg, 0.039 mmol) was added to a vial, followed by DCM (0.5 mL), Et₃SiH (0.050 mL, 0.31 mmol) and trifluoroacetic acid (0.1 mL, 1.30 mmol). The reaction was stirred for 17 h, then concentrated and dried well under high vacuum. The resulting crude carboxylic acid was reacted with 4-chlorobenzylamine according to general procedure A for amide bond formation, using DMF as solvent. The desired product was filtered off with a Hirsch funnel, yielding a white solid (6.1 mg, 33%). LRMS (ESI+) (M+H): 469.03.

### Example EF

### 2-((2R,6S,E)-5,12-dioxo-2-phenyl-1-oxa-4-azacyclododec-8-en-6-yl)-N-((tetrahydro-2H-pyran-4-yl)methyl)acetamide

16 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using (tetrahydro-2H-pyran-4-yl)methanamine (18 mg, 0.11 mmol) in the final coupling step. LRMS (ESI+) (M+H): 429.42.

### Example ES

### (2R,6S,E)-6-(2-oxo-2-(4-phenylpiperazin-1-yl)ethyl)-2-phenyl-1-oxa-4-azacyclododec-8-ene-5,12-dione

29 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using 1-phenylpiperazine (15 mg, 0.09 mmol, 1.0 eq) in the final coupling step. LRMS (ESI+) (M+H): 476.38.

### Example EX

### 2-((2R,6S,E)-4-methyl-5,12-dioxo-2-phenyl-1-oxa-4-azacyclododec-8-en-6-yl)-N-((tetrahydro-2H-pyran-4-yl)methyl)acetamide

To a solution of tert-butyl 2-((2R,6S,E)-4-methyl-5,12-dioxo-2-phenyl-1-oxa-4-azacyclododec-8-en-6-yl)acetate (50 mg, 0.13 mmol) in DCM (1 mL) cooled in an ice/water bath was added trifluoroacetic acid (96 μL, 1.3 mmol). The reaction mixture was stirred 3 h at which point no starting material remained and the solution was concentrated and dried under high vacuum to yield 2-((2R,6S,E)-4-methyl-5,12-dioxo-2-phenyl-1-oxa-4-azacyclododec-8-en-6-yl)acetic acid in quantitative yield. This intermediate was combined with (tetrahydro-2H-pyran-4-yl)methanamine (21 mg, 0.19 mmol) using general procedure B for amide bond formation with DCM as solvent to yield the title compound. The product (26 mg) was obtained as a white solid in 87% yield following purification of the crude product by column chromatography (MeOH/DCM gradient). LRMS (ESI+) (M+H): 443.35.

### Example EY

### N-(4-chlorobenzyl)-2-((3R,11S,E)-3-(2-methoxyphenyl)-5,12-dioxo-1-oxa-4-azacyclododec-8-en-11-yl)acetamide

(R)-2-amino-2-(2-methoxyphenyl)ethanol: Lithium aluminum hydride (227 mg, 5.99 mmol) was suspended in anhydrous THF (5 ml) under nitrogen atmosphere and cooled to 0° C. (R)-2-amino-2-(2-methoxyphenyl)acetic acid (350 mg, 1.93 mmol) dissolved in THF (15 ml) was then added dropwise. After the addition was complete, the reaction mixture was refluxed overnight. After cooling the mixture to 0° C., water (0.3 ml) was added slowly, followed by sat. K₂CO₃aq. (0.3 ml). Addition of an excess of K₂CO₃ powder was followed by filtration, washing with THF and CHCl₃ and evaporation of the solvents. The product (192 mg) was obtained in 59% yield after column chromatography (DCM/MeOH gradient). LRMS (ESI+) (M+H): 168.10.

(R)—N-(2-hydroxy-1-(2-methoxyphenyl)ethyl)pent-4-enamide: Using general procedure A for amide bond formation with DMF as solvent, the title compound was prepared from pent-4-enoic acid (67 μl, 0.66 mmol) and (R)-2-amino-2-(2-methoxyphenyl)ethanol (100 mg, 0.60 mmol). The product (149 mg) was obtained and used in the next step without purification. LRMS (ESI+) (M+H): 250.16.

(S)-4-tert-butyl 1-((R)-2-(2-methoxyphenyl)-2-(pent-4-enamido)ethyl) 2-allylsuccinate: Using general procedure C for ester bond formation with DCM as solvent the title compound was prepared from (S)-2-(2-tert-butoxy-2-oxoethyl)pent-4-enoic acid (141 mg, 0.66 mmol) and (R)—N-(2-hydroxy-1-(2-methoxyphenyl)ethyl)pent-4-enamide (149 mg, 0.60 mmol). The product (188 mg) was obtained in 71% yield over 2 steps after column chromatography (Hex/EA gradient). LRMS (ESI+) (M+Na): 468.34.

tert-butyl 2-((3R,11S,E)-3-(2-methoxyphenyl)-5,12-dioxo-1-oxa-4-azacyclododec-8-en-11-yl)acetate: Using the general procedure for ring closing metathesis with toluene as solvent the title compound was prepared from (S)-4-tert-butyl 1-((R)-2-(2-methoxyphenyl)-2-(pent-4-enamido)ethyl) 2-allylsuccinate (188 mg, 0.42 mmol). The product (128 mg) was obtained as a white solid in 73% yield following purification by column chromatography (Hex/EA gradient). LRMS (ESI−) (M−H): 416.20.

N-(4-chlorobenzyl)-2-((3R,11S,E)-3-(2-methoxyphenyl)-5,12-dioxo-1-oxa-4-azacyclododec-8-en-11-yl)acetamide: To a solution of tert-butyl 2-((3R,11S,E)-3-(2-methoxyphenyl)-5,12-dioxo-1-oxa-4-azacyclododec-8-en-11-yl)acetate (100 mg, 0.24 mmol) in CH₂Cl₂ (2.7 mL) cooled in an ice/water bath was added trifluoroacetic acid (275 μL, 3.59 mmol) and the reaction mixture was stirred 4 h at which point TLC analysis indicated complete consumption of starting material. The reaction mixture was concentrated and coevaporated 3 times with toluene. Then, using general procedure A for amide bond formation with DMF as solvent, the title compound was prepared from 2-((3R,11S,E)-3-(2-methoxyphenyl)-5,12-dioxo-1-oxa-4-azacyclododec-8-en-11-yl)acetic acid (87 mg, 0.24 mmol) and (4-chlorophenyl)methanamine (32 μl, 0.26 mmol). The product (40 mg) was obtained in 34% yield over 2 steps after column chromatography (MeOH/DCM gradient). LRMS (ESI+) (M+H): 485.25.

### Example EZ

### N-(4-chlorobenzyl)-2-((2R,3S,6S,Z)-3,4-dimethyl-5,12-dioxo-2-phenyl-1-oxa-4-azacyclododec-8-en-6-yl)acetamide

(S)-tert-butyl 3-(((1R,2S)-1-hydroxy-1-phenylpropan-2-yl)(methyl)carbamoyl)hex-5-enoate: Using general procedure A for amide bond formation with DCM as solvent, the title compound was prepared from (S)-2-(2-tert-butoxy-2-oxoethyl)pent-4-enoic acid (970 mg, 4.53 mmol) and (1R,2S)-2-(methylamino)-1-phenylpropan-1-ol (823 mg, 4.98 mmol). The product (1.5 g) was obtained in 92% yield after column chromatography (MeOH/DCM gradient). LRMS (ESI+) (M+H): 362.28.

(S)-tert-butyl 3-(methyl((1R,2S)-1-(pent-4-enoyloxy)-1-phenylpropan-2-yl)carbamoyl)hex-5-enoate: Using general procedure C for ester bond formation with DCM as solvent the title compound was prepared from 4-pentenoic acid (0.47 mL, 4.56 mmol) and (S)-tert-butyl 3-(((1R,2S)-1-hydroxy-1-phenylpropan-2-yl)(methyl)carbamoyl)hex-5-enoate (1.5 g, 4.15 mmol). The product (1.45 g) was obtained in 79% yield after column chromatography (Hex/EA gradient). LRMS (ESI+) (M+Na): 466.36.

tert-butyl 2-((2R,3S,6S,Z)-3,4-dimethyl-5,12-dioxo-2-phenyl-1-oxa-4-azacyclododec-8-en-6-yl)acetate: Using the general procedure for ring closing metathesis with toluene as solvent the title compound was prepared from (S)-tert-butyl 3-(methyl((1R,2S)-1-(pent-4-enoyloxy)-1-phenylpropan-2-yl)carbamoyl)hex-5-enoate (1.43 g, 3.22 mmol). The product (156 mg) was obtained in 12% yield following purification by column chromatography (Hex/EA gradient). LRMS (ESI+) (M+Na): 438.37.

N-(4-chlorobenzyl)-2-((2R,3S,6S,Z)-3,4-dimethyl-5,12-dioxo-2-phenyl-1-oxa-4-azacyclododec-8-en-6-yl)acetamide: To a solution of tert-butyl 2-((2R,3S,6S,Z)-3,4-dimethyl-5,12-dioxo-2-phenyl-1-oxa-4-azacyclododec-8-en-6-yl)acetate (33 mg, 0.079 mmol) in DCM (567 μL) cooled in an ice/water bath was added trifluoroacetic acid (91 μL, 1.19 mmol) and the reaction mixture was stirred 4 h at which point TLC analysis indicated complete consumption of starting material. The reaction mixture was concentrated, coevaporated 3 times with toluene. Using general procedure A for amide bond formation with DMF as solvent, the title compound was prepared from 2-((2R,3S,6S,Z)-3,4-dimethyl-5,12-dioxo-2-phenyl-1-oxa-4-azacyclododec-8-en-6-yl)acetic acid (28 mg, 0.079 mmol) and (4-chlorophenyl)methanamine (11 μL, 0.087 mmol). The product (28 mg) was obtained in 73% yield after column chromatography (MeOH/DCM gradient). LRMS (ESI+) (M+Na): 483.36.

### Example FA

### 2-((2R,3S,6S,E)-3,4-dimethyl-5,12-dioxo-2-phenyl-1-oxa-4-azacyclododec-8-en-6-yl)-N-(4,4,4-trifluorobutyl)acetamide

Using general procedure A for amide bond formation with DMF as solvent, the title compound was prepared from 2-((2R,3S,6S,E)-3,4-dimethyl-5,12-dioxo-2-phenyl-1-oxa-4-azacyclododec-8-en-6-yl)acetic acid (30 mg, 0.083 mmol) and 4,4,4-trifluorobutan-1-amine (12 mg, 0.092 mmol). The product (36 mg) was obtained in 92% yield after column chromatography (MeOH/DCM gradient). LRMS (ESI+) (M+H): 469.24.

### Example FB

### N-(4-chlorobenzyl)-2-((9S,14aS)-3,10-dioxododecahydro-1H-pyrrolo[2,1-c][1,4]oxaazacyclododecin-9-yl)acetamide

N-(4-chlorobenzyl)-2-((9S,14aS,E)-3,10-dioxo-3,4,5,8,9,10,12,13,14,14a-decahydro-1H-pyrrolo[2,1-c][1,4]oxaazacyclododecin-9-yl)acetamide (30 mg, 0.072 mmol), 4-methylbenzenesulfonohydrazide (667 mg, 3.58 mmol) and sodium acetate (294 mg, 3.58 mmol) in DME/water 1/1 (8.6 mL) were stirred at 80° C. for 4 h. The mixture was extracted with ethyl acetate followed by successive wash using NH₄Cl solution and NaHCO₃ solution. The organic phase was dried over sodium sulfate, filtered and concentrated. The product (17 mg) was obtained in 56% yield after column chromatography (MeOH/DCM gradient). LRMS (ESI+) (M+H): 421.15.

### Example FC

### 2-((2R,3S,6S,E)-3,4-dimethyl-5,12-dioxo-2-phenyl-1-oxa-4-azacyclododec-8-en-6-yl)-N-((1-methylpiperidin-4-yl)methyl)acetamide

tert-butyl 2-((2R,3S,6S,E)-3,4-dimethyl-5,12-dioxo-2-phenyl-1-oxa-4-azacyclododec-8-en-6-yl)acetate: To a solution of tert-butyl 2-((2R,3S,6S,E)-3-methyl-5,12-dioxo-2-phenyl-1-oxa-4-azacyclododec-8-en-6-yl)acetate (100 mg, 0.25 mmol) and iodomethane (47 μl, 0.747 mmol) in DMF (1.2 mL) cooled in an ice/water bath was added sodium hydride (60% in mineral oil) (6 mg, 0.25 mmol). After 2 h, the reaction was complete and the mixture was diluted with ethyl acetate and washed twice with aqueous HCl 0.1N, then brine. The resulting solution was dried over sodium sulfate, filtered, and concentrated. The product (60 mg) was obtained in 58% yield after column chromatography (Hex/EA gradient). LRMS (ESI+) (M+Na): 438.45.

2-((2R,3S,6S,E)-3,4-dimethyl-5,12-dioxo-2-phenyl-1-oxa-4-azacyclododec-8-en-6-yl)acetic acid: To a solution of tert-butyl 2-((2R,3S,6S,E)-3,4-dimethyl-5,12-dioxo-2-phenyl-1-oxa-4-azacyclododec-8-en-6-yl)acetate (59 mg, 0.142 mmol) in DCM (1 mL) cooled in an ice/water bath was added trifluoroacetic acid (141 μL, 1.85 mmol) and the reaction mixture was stirred for 2 h. The reaction mixture was concentrated, coevaporated 3 times with toluene and the product was utilized in the next step without further purification. LRMS (ESI−) (M−H): 358.31.

2-((2R,3S,6S,E)-3,4-dimethyl-5,12-dioxo-2-phenyl-1-oxa-4-azacyclododec-8-en-6-yl)-N-((1-methylpiperidin-4-yl)methyl)acetamide: Using general procedure A for amide bond formation with DMF as solvent, the title compound was prepared from 2-((2R,3S,6S,E)-3,4-dimethyl-5,12-dioxo-2-phenyl-1-oxa-4-azacyclododec-8-en-6-yl)acetic acid (28 mg, 0.078 mmol) and (1-methylpiperidin-4-yl)methanamine (11 mg, 0.086 mmol). The product (11 mg) was obtained in 31% yield after column chromatography (MeOH/DCM gradient). LRMS (ESI+) (M+H): 470.37.

### Example FD

### N-(cyclohexylmethyl)-2-((2R,6S,E)-5,12-dioxo-2-phenyl-1-oxa-4-azacyclododec-8-en-6-yl)acetamide

56 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using cyclohexylmethanamine (43 μl, 0.33 mmol, 1.1 eq) in the final coupling step. LRMS (ESI+) (M+H): 427.38.

### Example FE

### N-((1-benzylpiperidin-4-yl)methyl)-2-((2R,6S,E)-5,12-dioxo-2-phenyl-1-oxa-4-azacyclododec-8-en-6-yl)acetamide

56 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using (1-benzylpiperidin-4-yl)methanamine (68 mg, 0.33 mmol, 1.1 eq) in the final coupling step. LRMS (ESI+) (M+H): 518.49.

### Example FF

### N-(4-chlorobenzyl)-2-((9S,14aS,E)-3,10-dioxo-1,2,3,4,5,8,9,10,12,13,14,14a-dodecahydropyrrolo[1,2-a][1,4]diazacyclododecin-9-yl)acetamide

(S)-tert-butyl 3-((S)-2-(azidomethyl)pyrrolidine-1-carbonyl)hex-5-enoate: A mixture of (S)-tert-butyl 3-((S)-2-(hydroxymethyl)pyrrolidine-1-carbonyl)hex-5-enoate (100 mg, 0.336 mmol) and diphenyl phosphorazidate (87 μL, 0.404 mmol) was dissolved in dry DMF (580 μL) under nitrogen atmosphere and DBU (61 μL, 0.404 mmol) is added dropwise. The reaction mixture was warmed to 75° C. for 7 h, after which LCMS showed the reaction was complete. The mixture was dissolved in ethyl acetate and washed successively with NH₄Cl solution then brine. The organic phase was dried over sodium sulfate, filtered and concentrated. The product (70 mg) was obtained in 65% yield after column chromatography (Hex/EtOAc gradient). LRMS (ESI+) (M+Na): 345.23.

(S)-tert-butyl 3-((S)-2-(aminomethyl)pyrrolidine-1-carbonyl)hex-5-enoate: (S)-tert-butyl 3-((S)-2-(azidomethyl)pyrrolidine-1-carbonyl)hex-5-enoate (95 mg, 0.295 mmol) and triphenylphosphine (155 mg, 0.59 mmol) in THF/water 9/1 (4 mL) under nitrogen atmosphere were stirred for 12 h, after which time TLC analysis showed no azide remaining. Water (0.2 mL) was added and the reaction mixture was stirred for 2 h to hydrolyze the phosphoimine intermediate. The mixture was then concentrated and coevaporated with toluene. The product was obtained and used in the next step without further purification. LRMS (ESI+) (M+H): 297.28.

(S)-tert-butyl 3-((S)-2-(pent-4-enamidomethyl)pyrrolidine-1-carbonyl)hex-5-enoate: Using general procedure A for amide bond formation with DCM as solvent, the title compound was prepared from pent-4-enoic acid (13 μL, 0.123 mmol) and (S)-tert-butyl 3-((S)-2-(aminomethyl)pyrrolidine-1-carbonyl)hex-5-enoate (33 mg, 0.112 mmol). The product (40 mg) was obtained in 94% yield after column chromatography (MeOH/DCM gradient). LRMS (ESI+) (M+Na): 401.38.

tert-butyl 2-((9S,14aS,E)-3,10-dioxo-1,2,3,4,5,8,9,10,12,13,14,14a-dodecahydropyrrolo[1,2-a][1,4]diazacyclododecin-9-yl)acetate: Using the general procedure for ring closing metathesis with toluene as solvent the title compound was prepared from (S)-tert-butyl 3-((S)-2-(pent-4-enamidomethyl)pyrrolidine-1-carbonyl)hex-5-enoate (84 mg, 0.22 mmol). The product (22 mg) was obtained in 28% yield following purification by column chromatography (Hex/EtOAc gradient). LRMS (ESI+) (M+Na): 373.33.

N-(4-chlorobenzyl)-2-((9S,14aS,E)-3,10-dioxo-1,2,3,4,5,8,9,10,12,13,14,14a-dodecahydropyrrolo[1,2-a][1,4]diazacyclododecin-9-yl)acetamide: To a solution of tert-butyl 2-((9S,14aS,E)-3,10-dioxo-1,2,3,4,5,8,9,10,12,13,14,14a-dodecahydropyrrolo[1,2-a][1,4]diazacyclododecin-9-yl)acetate (17 mg, 0.049 mmol) in CH₂Cl₂ (346 μL) cooled in an ice/water bath was added trifluoroacetic acid (74 μL, 0.97 mmol) and the reaction mixture was stirred for 4 h. The reaction mixture was concentrated, coevaporated 3 times with toluene and the product was utilized in the next step without further purification. Then, using general procedure A for amide bond formation with DMF as solvent, the title compound was prepared from 2-((9S,14aS,E)-3,10-dioxo-1,2,3,4,5,8,9,10,12,13,14,14a-dodecahydropyrrolo[1,2-a][1,4]diazacyclododecin-9-yl)acetic acid (14 mg, 0.049 mmol) and (4-chlorophenyl)methanamine (7 μL, 0.057 mmol). The product (11 mg) was obtained in 52% yield over 2 steps after column chromatography (MeOH/DCM gradient). LRMS (ESI+) (M+H): 418.16.

### Example FG

### N-(4-chlorobenzyl)-2-((4S,9S,14aS,E)-4-methyl-3,10-dioxo-1,2,3,4,5,8,9,10,12,13,14,14a-dodecahydropyrrolo[1,2-a][1,4]diazacyclododecin-9-yl)acetamide

(S)-tert-butyl 3-((S)-2-(((S)-2-methylpent-4-enamido)methyl)pyrrolidine-1-carbonyl)hex-5-enoate: Using general procedure A for amide bond formation with DCM as solvent, the title compound was prepared from (S)-2-methylpent-4-enoic acid (24 mg, 0.208 mmol) and (S)-tert-butyl 3-((S)-2-(aminomethyl)pyrrolidine-1-carbonyl)hex-5-enoate (56 mg, 0.189 mmol). The product was utilized in the next step without further purification. LRMS (ESI+) (M+Na): 415.40.

tert-butyl 2-((4S,9S,14aS,E)-4-methyl-3,10-dioxo-1,2,3,4,5,8,9,10,12,13,14,14a-dodecahydropyrrolo[1,2-a][1,4]diazacyclododecin-9-yl)acetate: Using the general procedure for ring closing metathesis with toluene as solvent the title compound was prepared from (S)-tert-butyl 3-((S)-2-(((S)-2-methylpent-4-enamido)methyl)pyrrolidine-1-carbonyl)hex-5-enoate (74 mg, 0.19 mmol). Product (17 mg) was obtained in 25% yield following purification by column chromatography (MeOH/DCM gradient). LRMS (ESI+) (M+Na): 387.36.

N-(4-chlorobenzyl)-2-((4S,9S,14aS,E)-4-methyl-3,10-dioxo-1,2,3,4,5,8,9,10,12,13,14,14a-dodecahydropyrrolo[1,2-a][1,4]diazacyclododecin-9-yl)acetamide: To a solution of tert-butyl 2-((4S,9S,14aS,E)-4-methyl-3,10-dioxo-1,2,3,4,5,8,9,10,12,13,14,14a-dodecahydropyrrolo[1,2-a][1,4]diazacyclododecin-9-yl)acetate (17 mg, 0.047 mmol) in CH₂Cl₂ (333 μL) cooled in an ice/water bath was added trifluoroacetic acid (71 μL, 0.93 mmol) and the reaction mixture was stirred for 4 h. The reaction mixture was concentrated, coevaporated 3 times with toluene and the product was utilized in the next step without further purification. Then, using general procedure A for amide bond formation with DMF as solvent, the title compound was prepared from 2-((4S,9S,14aS,E)-4-methyl-3,10-dioxo-1,2,3,4,5,8,9,10,12,13,14,14a-dodecahydropyrrolo[1,2-a][1,4]diazacyclododecin-9-yl)acetic acid (14 mg, 0.047 mmol) and (4-chlorophenyl)methanamine (7 μL, 0.057 mmol). Product (6 mg) was obtained in 29% yield over 2 steps after column chromatography (MeOH/DCM gradient). LRMS (ESI+) (M+H): 432.17.

### Example FH

### 2-((4S,9S,14aS,E)-4-acetamido-3,10-dioxo-3,4,5,8,9,10,12,13,14,14a-decahydro-1H-pyrrolo[2,1-c][1,4]oxaazacyclododecin-9-yl)-N-(4-chlorobenzyl)acetamide

(S)-tert-butyl 3-((S)-2-(((S)-2-(((9H-fluoren-9-yl)methoxy)carbonylamino)pent-4-enoyloxy)methyl)pyrrolidine-1-carbonyl)hex-5-enoate: Using general procedure C for ester bond formation with DCM as solvent the title compound was prepared from (S)-2-(((9H-fluoren-9-yl)methoxy)carbonylamino)pent-4-enoic acid (82 mg, 0.244 mmol) and (S)-tert-butyl 3-((S)-2-(hydroxymethyl)pyrrolidine-1-carbonyl)hex-5-enoate (66 mg, 0.222 mmol). The product (123 mg) was obtained in 90% yield after column chromatography (Hex/EtOAc gradient). LRMS (ESI+) (M+Na): 639.57.

tert-butyl 2-((4S,9S,14aS,E)-4-(((9H-fluoren-9-yl)methoxy)carbonylamino)-3,10-dioxo-3,4,5,8,9,10,12,13,14,14a-decahydro-1H-pyrrolo[2,1-c][1,4]oxaazacyclododecin-9-yl)acetate: Using the general procedure for ring closing metathesis with toluene as solvent the title compound was prepared from (S)-tert-butyl 3-((S)-2-(((S)-2-(((9H-fluoren-9-yl)methoxy)carbonylamino)pent-4-enoyloxy)methyl)pyrrolidine-1-carbonyl)hex-5-enoate (123 mg, 0.199 mmol). Product (79 mg) was obtained in 67% yield following purification by column chromatography (MeOH/DCM gradient). LRMS (ESI+) (M+Na): 611.53.

(9H-fluoren-9-yl)methyl (4S,9S,14aS,E)-9-(2-(4-chlorobenzylamino)-2-oxoethyl)-3,10-dioxo-3,4,5,8,9,10,12,13,14,14a-decahydro-1H-pyrrolo[2,1-c][1,4]oxaazacyclododecin-4-ylcarbamate: To a solution of tert-butyl 2-((4S,9S,14aS,E)-4-(((9H-fluoren-9-yl)methoxy)carbonylamino)-3,10-dioxo-3,4,5,8,9,10,12,13,14,14a-decahydro-1H-pyrrolo[2,1-c][1,4]oxaazacyclododecin-9-yl)acetate (79 mg, 0.134 mmol) in CH₂Cl₂ (1 mL) cooled in an ice/water bath was added trifluoroacetic acid (206 μL, 2.68 mmol) and the reaction mixture was stirred 5 h at which point TLC analysis indicated complete consumption of starting material. The reaction mixture was concentrated, coevaporated 3 times with toluene. Then, using general procedure A for amide bond formation with DMF as solvent, the title compound was prepared from 2-((4S,9S,14aS,E)-4-(((9H-fluoren-9-yl)methoxy)carbonylamino)-3,10-dioxo-3,4,5,8,9,10,12,13,14,14a-decahydro-1H-pyrrolo[2,1-c][1,4]oxaazacyclododecin-9-yl)acetic acid (71 mg, 0.134 mmol) and (4-chlorophenyl)methanamine (18 μL, 0.149 mmol). The product (44 mg) was obtained in 50% yield over 2 steps after column chromatography (MeOH/DCM gradient). LRMS (ESI+) (M+H): 656.20.

2-((4S,9S,14aS,E)-4-acetamido-3,10-dioxo-3,4,5,8,9,10,12,13,14,14a-decahydro-1H-pyrrolo[2,1-c][1,4]oxaazacyclododecin-9-yl)-N-(4-chlorobenzyl)acetamide: (9H-fluoren-9-yl)methyl (4S,9S,14aS,E)-9-(2-(4-chlorobenzylamino)-2-oxoethyl)-3,10-dioxo-3,4,5,8,9,10,12,13,14,14a-decahydro-1H-pyrrolo[2,1-c][1,4]oxaazacyclododecin-4-ylcarbamate (44 mg, 0.067 mmol) was dissolved in DMF (2 mL) and piperidine (497 μL, 5.03 mmol) was added dropwise. After 30 minutes, the reaction is complete as shown by LCMS. Solvents are evaporated and coevaporated with toluene 3 times. Then, the resulting amine, 2-((4S,9S,14aS,E)-4-amino-3,10-dioxo-3,4,5,8,9,10,12,13,14,14a-decahydro-1H-pyrrolo[2,1-c][1,4]oxaazacyclododecin-9-yl)-N-(4-chlorobenzyl)acetamide (15 mg, 0.035 mmol) was dissolved in DMF (1.7 mL) under argon atmosphere. Triethylamine (486 μL, 3.46 mmol) and acetic anhydride (327 μL, 3.46 mmol) were successively added in one portion and the reaction mixture was stirred at room temperature for 15 h. Solvents were evaporated before the mixture was dissolved with ethyl acetate and successively washed with NH₄Cl solution and NaHCO₃ solution. The product (7 mg) was obtained in 43% yield over 2 steps after purification by column chromatography (DCM/MeOH gradient). LRMS (ESI+) (M+H): 476.15.

### Example FI

### (S,E)-N-(4-chlorobenzyl)-2-(5,12-dioxo-1-oxa-4-azacyclododec-8-en-6-yl)acetamide

(S)-tert-butyl 3-((2-(pent-4-enoyloxy)ethyl)carbamoyl)hex-5-enoate: Using general procedure A for amide bond formation with DMF as solvent, the title compound was prepared from 2-aminoethanol (62 μl, 1.03 mmol) and (S)-2-(2-tert-butoxy-2-oxoethyl)pent-4-enoic acid (200 mg, 0.93 mmol). The product was obtained and used in the next step without purification. Using general procedure C for ester bond formation with DCM as solvent the title compound was prepared from pent-4-enoic acid (96 μl, 0.94 mmol) and (S)-tert-butyl 3-(2-hydroxyethylcarbamoyl)hex-5-enoate (220 mg, 0.85 mmol). The product (220 mg) was obtained in 76% yield over 2 steps after column chromatography (Hex/EA gradient). LRMS (ESI+) (M+Na): 362.19.

(S,E)-N-(4-chlorobenzyl)-2-(5,12-dioxo-1-oxa-4-azacyclododec-8-en-6-yl)acetamide: Using the general procedure for ring closing metathesis with toluene as solvent the title compound was prepared from (S)-tert-butyl 3-((2-(pent-4-enoyloxy)ethyl)carbamoyl)hex-5-enoate (220 mg, 0.65 mmol). The product was obtained and used in the next step without purification. Then, to a solution of (S,E)-tert-butyl 2-(5,12-dioxo-1-oxa-4-azacyclododec-8-en-6-yl)acetate (99 mg, 0.32 mmol) in CH₂Cl₂ (2.3 mL) cooled in an ice/water bath was added trifluoroacetic acid (487 μL, 6.36 mmol) and the reaction mixture was stirred 4 h at which point TLC analysis indicated complete consumption of starting material. The reaction mixture was concentrated and coevaporated 3 times with toluene. Then, using general procedure A for amide bond formation with DCM as solvent, the title compound was prepared from (S,E)-2-(5,12-dioxo-1-oxa-4-azacyclododec-8-en-6-yl)acetic acid (81 mg, 0.32 mmol) and (4-chlorophenyl)methanamine (43 μl, 0.35 mmol). The product (70 mg) was obtained as a white solid in 58% yield over 3 steps after column chromatography (MeOH/DCM gradient). LRMS (ESI+) (M+H): 379.15.

### Example FJ

### N-(4-chlorobenzyl)-2-((2R,6S,E)-2-methyl-5,12-dioxo-1-oxa-4-azacyclododec-8-en-6-yl)acetamide

(S)-tert-butyl 3-(((R)-2-(pent-4-enoyloxy)propyl)carbamoyl)hex-5-enoate: Using general procedure A for amide bond formation with DMF as solvent, the title compound was prepared from (R)-1-aminopropan-2-ol (58 mg, 0.77 mmol) and (S)-2-(2-tert-butoxy-2-oxoethyl)pent-4-enoic acid (150 mg, 0.70 mmol). The product was obtained and used in the next step without purification. Using general procedure C for ester bond formation with DCM as solvent the title compound was prepared from pent-4-enoic acid (72 μl, 0.71 mmol) and (S)-tert-butyl 3-((R)-2-hydroxypropylcarbamoyl)hex-5-enoate (220 mg, 0.85 mmol). The product (200 mg) was obtained and used in the next step without purification. LRMS (ESI+) (M+H): 354.22.

N-(4-chlorobenzyl)-2-((2R,6S,E)-2-methyl-5,12-dioxo-1-oxa-4-azacyclododec-8-en-6-yl)acetamide: Using the general procedure for ring closing metathesis with toluene as solvent the title compound was prepared from (S)-tert-butyl 3-(((R)-2-(pent-4-enoyloxy)propyl)carbamoyl)hex-5-enoate (200 mg, 0.57 mmol). Product was obtained and used in the next step without purification. Then, to a solution of tert-butyl 2-((2R,6S,E)-2-methyl-5,12-dioxo-1-oxa-4-azacyclododec-8-en-6-yl)acetate (82 mg, 0.25 mmol) in CH₂Cl₂ (1.8 mL) cooled in an ice/water bath was added trifluoroacetic acid (386 μL, 5.04 mmol) and the reaction mixture was stirred 4 h at which point TLC analysis indicated complete consumption of starting material. The reaction mixture was concentrated and coevaporated 3 times with toluene. Then, using general procedure A for amide bond formation with DMF as solvent, the title compound was prepared from 2-((2R,6S,E)-2-methyl-5,12-dioxo-1-oxa-4-azacyclododec-8-en-6-yl)acetic acid (68 mg, 0.25 mmol) and (4-chlorophenyl)methanamine (34 μl, 0.28 mmol). Product (75 mg) was obtained as a white solid in 76% yield over 3 steps after column chromatography (MeOH/DCM gradient). LRMS (ESI+) (M+H): 392.99.

### Example FK

### N-(4-chlorobenzyl)-2-((2R,6S,E)-2-methyl-5,12-dioxo-1-oxa-4-azacyclododec-8-en-6-yl)acetamide

(S)-tert-butyl 3-(((R)-2-(pent-4-enoyloxy)propyl)carbamoyl)hex-5-enoate: Using general procedure A for amide bond formation with DMF as solvent, the title compound was prepared from (R)-1-aminopropan-2-ol (58 mg, 0.77 mmol) and (S)-2-(2-tert-butoxy-2-oxoethyl)pent-4-enoic acid (150 mg, 0.70 mmol). The product was obtained and used in the next step without purification. Using general procedure C for ester bond formation with DCM as solvent the title compound was prepared from pent-4-enoic acid (72 μl, 0.71 mmol) and (S)-tert-butyl 3-((R)-2-hydroxypropylcarbamoyl)hex-5-enoate (220 mg, 0.85 mmol). Product (200 mg) was obtained and used in the next step without purification. LRMS (ESI+) (M+H): 354.22.

N-(4-chlorobenzyl)-2-((2R,6S,E)-2-methyl-5,12-dioxo-1-oxa-4-azacyclododec-8-en-6-yl)acetamide: Using the general procedure for ring closing metathesis with toluene as solvent the title compound was prepared from (S)-tert-butyl 3-(((R)-2-(pent-4-enoyloxy)propyl)carbamoyl)hex-5-enoate (200 mg, 0.57 mmol). The product was obtained and used in the next step without purification. Then, to a solution of tert-butyl 2-((2R,6S,E)-2-methyl-5,12-dioxo-1-oxa-4-azacyclododec-8-en-6-yl)acetate (82 mg, 0.25 mmol) in CH₂Cl₂ (1.8 mL) cooled in an ice/water bath was added trifluoroacetic acid (386 μL, 5.04 mmol) and the reaction mixture was stirred 4 h at which point TLC analysis indicated complete consumption of starting material. The reaction mixture was concentrated and coevaporated 3 times with toluene. Then, using general procedure A for amide bond formation with DMF as solvent, the title compound was prepared from 2-((2R,6S,E)-2-methyl-5,12-dioxo-1-oxa-4-azacyclododec-8-en-6-yl)acetic acid (68 mg, 0.25 mmol) and (4-chlorophenyl)methanamine (34 μl, 0.28 mmol). The product (75 mg) was obtained as a white solid in 76% yield over 3 steps after column chromatography (MeOH/DCM gradient). LRMS (ESI+) (M+H): 392.99.

### Example FL

### 2-((2R,6S,E)-5,12-dioxo-2-phenyl-1-oxa-4-azacyclododec-8-en-6-yl)-N-(thiophen-2-ylmethyl)acetamide

56 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using thiophen-2-ylmethanamine (34 μl, 0.33 mmol, 1.1 eq) in the final coupling step. LRMS (ESI+) (M+H): 427.24.

### Example FM

### N-(4-chlorobenzyl)-2-((1S,9S,14aS,E)-1-methyl-3,10-dioxo-3,4,5,8,9,10,12,13,14,14a-decahydro-1H-pyrrolo[2,1-c][1,4]oxaazacyclododecin-9-yl)acetamide

(S)-tert-butyl 3-((S)-2-formylpyrrolidine-1-carbonyl)hex-5-enoate: DMSO (287 μL, 4.04 mmol) was dissolved in DCM (2.7 mL) under argon atmosphere and cooled to −78° C. Oxalyl chloride 2M in DCM (1.009 mL, 2.018 mmol) was slowly added dropwise and the mixture was stirred at −78° C. for 1 h. A solution of (S)-tert-butyl 3-((S)-2-(hydroxymethyl)pyrrolidine-1-carbonyl)hex-5-enoate (400 mg, 1.345 mmol) in DCM (2.2 ml) was then added dropwise and the mixture was stirred for 3 h at −78° C. Triethylamine (750 μL, 5.38 mmol) was added and the solution was stirred at room temperature for 3 h. The mixture was then dissolved in DCM and washed with NH₄Cl solution then brine, dried over sodium sulfate, filtered and concentrated. The product (334 mg) was obtained in 84% yield after column chromatography (Hex/EtOAc gradient). LRMS (ESI+) (M+H): 296.31.

(S)-tert-butyl 3-((S)-2-((S)-1-hydroxyethyl)pyrrolidine-1-carbonyl)hex-5-enoate: (S)-tert-butyl 3-((S)-2-formylpyrrolidine-1-carbonyl)hex-5-enoate (160 mg, 0.542 mmol) was dissolved in diethyl ether (25 mL) under nitrogen atmosphere and cooled to −78° C. Methyl magnesium bromide, 3M in diethyl ether (217 μL, 0.650 mmol) was slowly added dropwise and the mixture was stirred at −78° C. for 2 h. The reaction mixture was then warmed up to room temperature, dissolved in ethyl acetate and washed with NH₄Cl solution then brine. The organic phase was then dried over sodium sulfate, filtered and concentrated. The product (92 mg) was obtained in 55% yield after column chromatography (Hex/EtOAc gradient). LRMS (ESI+) (M+H): 312.29.

(S)-tert-butyl 3-((S)-2-((S)-1-(pent-4-enoyloxy)ethyl)pyrrolidine-1-carbonyl)hex-5-enoate: Using general procedure C for ester bond formation with DCM as solvent the title compound was prepared from 4-pentenoic acid (22 μL, 0.21 mmol) and (S)-tert-butyl 3-((S)-2-((S)-1-hydroxyethyl)pyrrolidine-1-carbonyl)hex-5-enoate (59 mg, 0.189 mmol). The product (54 mg) was obtained in 72% yield after column chromatography (Hex/EtOAc gradient). LRMS (ESI+) (M+H): 394.30.

tert-butyl 2-((1S,9S,14aS,E)-1-methyl-3,10-dioxo-3,4,5,8,9,10,12,13,14,14a-decahydro-1H-pyrrolo[2,1-c][1,4]oxaazacyclododecin-9-yl)acetate: Using the general procedure for ring closing metathesis with toluene as solvent the title compound was prepared from (S)-tert-butyl 3-((S)-2-((S)-1-(pent-4-enoyloxy)ethyl)pyrrolidine-1-carbonyl)hex-5-enoate (54 mg, 0.137 mmol). The product (22 mg) was obtained in 44% yield following purification by column chromatography (Hex/EtOAc gradient). LRMS (ESI+) (M+H): 366.36.

N-(4-chlorobenzyl)-2-((1S,9S,14aS,E)-1-methyl-3,10-dioxo-3,4,5,8,9,10,12,13,14,14a-decahydro-1H-pyrrolo[2,1-c][1,4]oxaazacyclododecin-9-yl)acetamide: To a solution of tert-butyl 2-((1S,9S,14aS,E)-1-methyl-3,10-dioxo-3,4,5,8,9,10,12,13,14,14a-decahydro-1H-pyrrolo[2,1-c][1,4]oxaazacyclododecin-9-yl)acetate (22 mg, 0.06 mmol) in CH₂Cl₂ (430 μL) cooled in an ice/water bath was added trifluoroacetic acid (92 μL, 1.20 mmol) and the reaction mixture was stirred 3 h at which point TLC analysis indicated complete consumption of starting material. The reaction mixture was concentrated, coevaporated 3 times with toluene. Then, using general procedure A for amide bond formation with DMF as solvent, the title compound was prepared from 2-((1S,9S,14aS,E)-1-methyl-3,10-dioxo-3,4,5,8,9,10,12,13,14,14a-decahydro-1H-pyrrolo[2,1-c][1,4]oxaazacyclododecin-9-yl)acetic acid (18 mg, 0.06 mmol) and (4-chlorophenyl)methanamine (9 μL, 0.066 mmol). The product (21 mg) was obtained in 81% yield over 2 steps after column chromatography (MeOH/DCM gradient). LRMS (ESI+) (M+H): 433.21.

### Example FN

### N-(4-chlorobenzyl)-2-((1S,9S,14aS,Z)-1-methyl-3,10-dioxo-3,4,5,8,9,10,12,13,14,14a-decahydro-1H-pyrrolo[2,1-c][1,4]oxaazacyclododecin-9-yl)acetamide

tert-butyl 2-((1S,9S,14aS,Z)-1-methyl-3,10-dioxo-3,4,5,8,9,10,12,13,14,14a-decahydro-1H-pyrrolo[2,1-c][1,4]oxaazacyclododecin-9-yl)acetate: Using the general procedure for ring closing metathesis with toluene as solvent the title compound was prepared from (S)-tert-butyl 3-((S)-2-((S)-1-(pent-4-enoyloxy)ethyl)pyrrolidine-1-carbonyl)hex-5-enoate (54 mg, 0.137 mmol). Product (12 mg) was obtained in 24% yield following purification by column chromatography (Hex/EtOAc gradient). LRMS (ESI+) (M+H): 366.34.

N-(4-chlorobenzyl)-2-((1S,9S,14aS,Z)-1-methyl-3,10-dioxo-3,4,5,8,9,10,12,13,14,14a-decahydro-1H-pyrrolo[2,1-c][1,4]oxaazacyclododecin-9-yl)acetamide: To a solution of tert-butyl 2-((1S,9S,14aS,Z)-1-methyl-3,10-dioxo-3,4,5,8,9,10,12,13,14,14a-decahydro-1H-pyrrolo[2,1-c][1,4]oxaazacyclododecin-9-yl)acetate (12 mg, 0.033 mmol) in CH₂Cl₂ (235 μL) cooled in an ice/water bath was added trifluoroacetic acid (50 μL, 0.66 mmol) and the reaction mixture was stirred 3 h at which point TLC analysis indicated complete consumption of starting material. The reaction mixture was concentrated, coevaporated 3 times with toluene. Then, using general procedure A for amide bond formation with DMF as solvent, the title compound was prepared from 2-((1S,9S,14aS,Z)-1-methyl-3,10-dioxo-3,4,5,8,9,10,12,13,14,14a-decahydro-1H-pyrrolo[2,1-c][1,4]oxaazacyclododecin-9-yl)acetic acid (10 mg, 0.033 mmol) and (4-chlorophenyl)methanamine (5 μL, 0.04 mmol). The product (7 mg) was obtained in 50% yield over 2 steps after column chromatography (MeOH/DCM gradient). LRMS (ESI+) (M+H): 433.21.

### Example FO

### N-(4-chlorobenzyl)-2-((2R,3R,6S,E)-3,4-dimethyl-5,12-dioxo-2-phenyl-1-oxa-4-azacyclododec-8-en-6-yl)acetamide

(S)-tert-butyl 3-(((1R,2R)-1-hydroxy-1-phenylpropan-2-yl)(methyl)carbamoyl)hex-5-enoate: Using general procedure A for amide bond formation with DCM as solvent, the title compound was prepared from (S)-2-(2-tert-butoxy-2-oxoethyl)pent-4-enoic acid (130 mg, 0.605 mmol) and (1R,2R)-2-(methylamino)-1-phenylpropan-1-ol (100 mg, 0.605 mmol). The product (219 mg) was obtained and used in the next step without further purification. LRMS (ESI+) (M+Na): 384.28.

(S)-tert-butyl 3-(methyl((1R,2R)-1-(pent-4-enoyloxy)-1-phenylpropan-2-yl)carbamoyl)hex-5-enoate: Using general procedure C for ester bond formation with DCM as solvent the title compound was prepared from 4-pentenoic acid (68 μL, 0.666 mmol) and (S)-tert-butyl 3-(((1R,2R)-1-hydroxy-1-phenylpropan-2-yl)(methyl)carbamoyl)hex-5-enoate (219 mg, 0.605 mmol). The product (210 mg) was obtained in 78% yield after column chromatography (Hex/EA gradient). LRMS (ESI+) (M+H): 444.23.

tert-butyl 2-((2R,3R,6S,E)-3,4-dimethyl-5,12-dioxo-2-phenyl-1-oxa-4-azacyclododec-8-en-6-yl)acetate: Using the general procedure for ring closing metathesis with toluene as solvent the title compound was prepared from (S)-tert-butyl 3-(methyl((1R,2R)-1-(pent-4-enoyloxy)-1-phenylpropan-2-yl)carbamoyl)hex-5-enoate (210 mg, 0.473 mmol). The product (151 mg) was obtained in 77% yield following purification by column chromatography (Hex/EA gradient). LRMS (ESI+) (M+Na): 438.19.

2-((2R,3R,6S,E)-3,4-dimethyl-5,12-dioxo-2-phenyl-1-oxa-4-azacyclododec-8-en-6-yl)acetic acid: To a solution of tert-butyl 2-((2R,3R,6S,E)-3,4-dimethyl-5,12-dioxo-2-phenyl-1-oxa-4-azacyclododec-8-en-6-yl)acetate (151 mg, 0.363 mmol) in DCM (2.6 mL) cooled in an ice/water bath was added trifluoroacetic acid (557 μL, 7.279 mmol) and the reaction mixture was stirred 4 h at which point LCMS analysis indicated complete consumption of starting material. The reaction mixture was concentrated, coevaporated 3 times with toluene. The product (131 mg) was obtained and used in the next step without purification. LRMS (ESI−) (M−H): 358.24.

N-(4-chlorobenzyl)-2-((2R,3R,6S,E)-3,4-dimethyl-5,12-dioxo-2-phenyl-1-oxa-4-azacyclododec-8-en-6-yl)acetamide: Using general procedure A for amide bond formation with DCM as solvent, the title compound was prepared from 2-((2R,3R,6S,E)-3,4-dimethyl-5,12-dioxo-2-phenyl-1-oxa-4-azacyclododec-8-en-6-yl)acetic acid (65 mg, 0.181 mmol) and (4-chlorophenyl)methanamine (24 μL, 0.199 mmol). The product (60 mg) was obtained in 69% yield after column chromatography (MeOH/DCM gradient). LRMS (ESI+) (M+H): 483.15.

### Example FP

### N-(4-chlorobenzyl)-2-((2R,3S,6S,E)-3-methyl-5,12-dioxo-2-phenyl-1-oxa-4-azacyclododec-8-en-6-yl)acetamide

(S)-tert-butyl 3-((1R,2S)-1-hydroxy-1-phenylpropan-2-ylcarbamoyl)hex-5-enoate: Using general procedure A for amide bond formation with DMF as solvent, the title compound was prepared from (S)-2-(2-tert-butoxy-2-oxoethyl)pent-4-enoic acid (2.14 g, 10 mmol) and (1R,2S)-2-amino-1-phenylpropan-1-ol (1.66 g, 11 mmol). The product (2.9 g) was obtained in 83% yield after column chromatography (MeOH/DCM gradient). LRMS (ESI−) (M−H): 346.66.

(S)-tert-butyl 3-((1R,2S)-1-(pent-4-enoyloxy)-1-phenylpropan-2-ylcarbamoyl)hex-5-enoate: Using general procedure C for ester bond formation with DCM as solvent the title compound was prepared from 4-pentenoic acid (0.49 mL, 4.75 mmol) and (S)-tert-butyl 3-((1R,2S)-1-hydroxy-1-phenylpropan-2-ylcarbamoyl)hex-5-enoate (1.5 g, 4.32 mmol). The product (1.5 g) was obtained in 81% yield after column chromatography (Hex/EA gradient). LRMS (ESI+) (M+Na): 452.49.

tert-butyl 2-((2R,3S,6S,E)-3-methyl-5,12-dioxo-2-phenyl-1-oxa-4-azacyclododec-8-en-6-yl)acetate: Using the general procedure for ring closing metathesis with toluene as solvent the title compound was prepared from (S)-tert-butyl 3-((1R,2S)-1-(pent-4-enoyloxy)-1-phenylpropan-2-ylcarbamoyl)hex-5-enoate (1.7 g, 3.98 mmol). The product (850 mg) was obtained as a white solid in 53% yield following purification by column chromatography (Hex/EA gradient). LRMS (ESI+) (M+Na): 424.45.

N-(4-chlorobenzyl)-2-((2R,3S,6S,E)-3-methyl-5,12-dioxo-2-phenyl-1-oxa-4-azacyclododec-8-en-6-yl)acetamide: To a solution of tert-butyl 2-((2R,3S,6S,E)-3-methyl-5,12-dioxo-2-phenyl-1-oxa-4-azacyclododec-8-en-6-yl)acetate (121 mg, 0.3 mmol) in CH₂Cl₂ (2 mL) cooled in an ice/water bath was added trifluoroacetic acid (324 μL, 4.24 mmol) and the reaction mixture was stirred 2 h at which point TLC analysis indicated complete consumption of starting material. The reaction mixture was concentrated, coevaporated 3 times with toluene, and a portion of the crude carboxylic acid was utilized immediately. Using general procedure A for amide bond formation with DMF as solvent, the title compound was prepared from 2-((2R,3S,6S,E)-3-methyl-5,12-dioxo-2-phenyl-1-oxa-4-azacyclododec-8-en-6-yl)acetic acid and (4-chlorophenyl)methanamine (45 mg, 0.32 mmol). The product (118 mg) was obtained as a white solid in 87% yield after column chromatography (MeOH/DCM gradient). LRMS (ESI−) (M−H): 467.79.

### Example FQ

### N-(4-chlorobenzyl)-2-((3R,11S,E)-3-isopropyl-5,12-dioxo-1-oxa-4-azacyclododec-8-en-11-yl)acetamide

(S)-4-tert-butyl 1-((R)-3-methyl-2-pent-4-enamidobutyl) 2-allylsuccinate: Using general procedure A for amide bond formation with DMF as solvent, (R)—N-(1-hydroxy-3-methylbutan-2-yl)pent-4-enamide was prepared from pent-4-enoic acid (272 μl, 2.67 mmol) and (R)-2-amino-3-methylbutan-1-ol (250 mg, 2.42 mmol). The product was obtained and used in the next step without purification. Using general procedure C for ester bond formation with DCM as solvent the title compound was prepared from (S)-2-(2-tert-butoxy-2-oxoethyl)pent-4-enoic acid (571 mg, 2.67 mmol) and (R)—N-(1-hydroxy-3-methylbutan-2-yl)pent-4-enamide (449 mg, 2.42 mmol). The product (439 mg) was obtained in 48% yield over 2 steps after column chromatography (Hex/EA gradient). LRMS (ESI+) (M+Na): 404.41.

tert-butyl 2-((3R,11S,E)-3-isopropyl-5,12-dioxo-1-oxa-4-azacyclododec-8-en-11-yl)acetate: Using the general procedure for ring closing metathesis with toluene as solvent the title compound was prepared from (S)-4-tert-butyl 1-((R)-3-methyl-2-pent-4-enamidobutyl) 2-allylsuccinate (439 mg, 1.15 mmol). The product (309 mg) was obtained in 76% yield following purification by column chromatography (Hex/EA gradient). LRMS (ESI+) (M+Na): 376.39.

N-(4-chlorobenzyl)-2-((3R,11S,E)-3-isopropyl-5,12-dioxo-1-oxa-4-azacyclododec-8-en-11-yl)acetamide: To a solution of tert-butyl 2-((3R,11S,E)-3-isopropyl-5,12-dioxo-1-oxa-4-azacyclododec-8-en-11-yl)acetate (309 mg, 0.87 mmol) in CH₂Cl₂ (6.5 mL) cooled in an ice/water bath was added trifluoroacetic acid (1004 μL, 13.11 mmol) and the reaction mixture was stirred 4 h at which point TLC analysis indicated complete consumption of starting material. The reaction mixture was concentrated and coevaporated 3 times with toluene. Using general procedure A for amide bond formation with DMF as solvent, the title compound was prepared from 2-((3R,11S,E)-3-isopropyl-5,12-dioxo-1-oxa-4-azacyclododec-8-en-11-yl)acetic acid (260 mg, 0.87 mmol) and (4-chlorophenyl)methanamine (118 μl, 0.96 mmol). The product (268 mg) was obtained as a white solid in 73% yield over 2 steps after column chromatography (MeOH/DCM gradient). LRMS (ESI+) (M+H): 421.38.

### Example FR

### 2-((3R,11S,E)-3-butyl-5,12-dioxo-1-oxa-4-azacyclododec-8-en-11-yl)-N-(4-chlorobenzyl)acetamide

(S)-4-tert-butyl 1-((R)-2-pent-4-enamidohexyl) 2-allylsuccinate: Using general procedure A for amide bond formation with DMF as solvent, (R)—N-(1-hydroxyhexan-2-yl)pent-4-enamide was prepared from pent-4-enoic acid (239 μl, 2.35 mmol) and (R)-2-aminohexan-1-ol (250 mg, 2.13 mmol). The product was obtained and used in the next step without purification. Using general procedure C for ester bond formation with DCM as solvent the title compound was prepared from (S)-2-(2-tert-butoxy-2-oxoethyl)pent-4-enoic acid (503 mg, 2.35 mmol) and (R)—N-(1-hydroxyhexan-2-yl)pent-4-enamide (425 mg, 2.13 mmol). The product (588 mg) was obtained in 70% yield over 2 steps after column chromatography (Hex/EA gradient). LRMS (ESI+) (M+Na): 418.44.

tert-butyl 2-((3R,11S,E)-3-butyl-5,12-dioxo-1-oxa-4-azacyclododec-8-en-11-yl)acetate: Using the general procedure for ring closing metathesis with toluene as solvent the title compound was prepared from (S)-4-tert-butyl 1-((R)-2-pent-4-enamidohexyl) 2-allylsuccinate (580 mg, 1.47 mmol). The product (348 mg) was obtained in 65% yield following purification by column chromatography (Hex/EA gradient). LRMS (ESI+) (M+Na): 390.42.

2-((3R,11S,E)-3-butyl-5,12-dioxo-1-oxa-4-azacyclododec-8-en-11-yl)-N-(4-chlorobenzyl)acetamide: To a solution of tert-butyl 2-((3R,11S,E)-3-butyl-5,12-dioxo-1-oxa-4-azacyclododec-8-en-11-yl)acetate (345 mg, 0.94 mmol) in CH₂Cl₂ (7 mL) cooled in an ice/water bath was added trifluoroacetic acid (1078 μL, 14.08 mmol) and the reaction mixture was stirred 4 h at which point TLC analysis indicated complete consumption of starting material. The reaction mixture was concentrated and coevaporated 3 times with toluene. Using general procedure A for amide bond formation with DMF as solvent, the title compound was prepared from 2-((3R,11S,E)-3-butyl-5,12-dioxo-1-oxa-4-azacyclododec-8-en-11-yl)acetic acid (292 mg, 0.94 mmol) and (4-chlorophenyl)methanamine (126 μl, 1.03 mmol). The product (293 mg) was obtained as a white solid in 72% yield over 2 steps after column chromatography (MeOH/DCM gradient). LRMS (ESI+) (M+H): 435.40.

### Example FS

### 2-((3R,11S,E)-3-benzyl-5,12-dioxo-1-oxa-4-azacyclododec-8-en-11-yl)-N-(4-chlorobenzyl)acetamide

(S)-4-tert-butyl 1-((R)-2-(pent-4-enamido)-3-phenylpropyl) 2-allylsuccinate: Using general procedure A for amide bond formation with DMF as solvent, (R)—N-(1-hydroxy-3-phenylpropan-2-yl)pent-4-enamide was prepared from pent-4-enoic acid (186 μl, 1.82 mmol) and (R)-2-amino-3-phenylpropan-1-ol (250 mg, 1.65 mmol). The product was obtained and used in the next step without purification. Using general procedure C for ester bond formation with DCM as solvent the title compound was prepared from (S)-2-(2-tert-butoxy-2-oxoethyl)pent-4-enoic acid (390 mg, 1.82 mmol) and (R)—N-(1-hydroxy-3-phenylpropan-2-yl)pent-4-enamide (386 mg, 1.65 mmol). The product (351 mg) was obtained in 50% yield over 2 steps after column chromatography (Hex/EA gradient). LRMS (ESI+) (M+Na): 452.45.

tert-butyl 2-((3R,11S,E)-3-benzyl-5,12-dioxo-1-oxa-4-azacyclododec-8-en-11-yl)acetate: Using the general procedure for ring closing metathesis with toluene as solvent the title compound was prepared from (S)-4-tert-butyl 1-((R)-2-(pent-4-enamido)-3-phenylpropyl) 2-allylsuccinate (350 mg, 0.82 mmol). The product (213 mg) was obtained in 65% yield following purification by column chromatography (Hex/EA gradient). LRMS (ESI+) (M+Na): 424.44.

2-((3R,11S,E)-3-benzyl-5,12-dioxo-1-oxa-4-azacyclododec-8-en-11-yl)-N-(4-chlorobenzyl)acetamide: To a solution of tert-butyl 2-((3R,11S,E)-3-benzyl-5,12-dioxo-1-oxa-4-azacyclododec-8-en-11-yl)acetate (210 mg, 0.52 mmol) in CH₂Cl₂ (4 mL) cooled in an ice/water bath was added trifluoroacetic acid (601 μL, 7.85 mmol) and the reaction mixture was stirred 4 h at which point TLC analysis indicated complete consumption of starting material. The reaction mixture was concentrated and coevaporated 3 times with toluene. Using general procedure A for amide bond formation with DMF as solvent, the title compound was prepared from 2-((3R,11S,E)-3-benzyl-5,12-dioxo-1-oxa-4-azacyclododec-8-en-11-yl)acetic acid (181 mg, 0.52 mmol) and (4-chlorophenyl)methanamine (70 μl, 0.58 mmol). The product (193 mg) was obtained as a white solid in 79% yield over 2 steps after column chromatography (MeOH/DCM gradient). LRMS (ESI+) (M+H): 469.42.

### Example FT

### N-(4-chlorobenzyl)-2-((4S,14aS,E)-3,10-dioxo-3,4,5,8,9,10,12,13,14,14a-decahydro-1H-pyrrolo[2,1-c][1,4]oxaazacyclododecin-4-yl)acetamide

(S)-4-tert-butyl 1-(((S)-1-(pent-4-enoyl)pyrrolidin-2-yl)methyl) 2-allylsuccinate: Using general procedure A for amide bond formation with DMF as solvent, (S)-1-(2-(hydroxymethyl)pyrrolidin-1-yl)pent-4-en-1-one was prepared from pent-4-enoic acid (277 μl, 2.72 mmol) and (S)-pyrrolidin-2-ylmethanol (250 mg, 2.47 mmol). The product was obtained and used in the next step without purification. Using general procedure C for ester bond formation with DCM as solvent the title compound was prepared from (S)-2-(2-tert-butoxy-2-oxoethyl)pent-4-enoic acid (583 mg, 2.72 mmol) and (S)-1-(2-(hydroxymethyl)pyrrolidin-1-yl)pent-4-en-1-one (453 mg, 2.47 mmol). The product (392 mg) was obtained in 42% yield over 2 steps after column chromatography (Hex/EA gradient). LRMS (ESI+) (M+Na): 402.40.

tert-butyl 2-((4S,14aS,E)-3,10-dioxo-3,4,5,8,9,10,12,13,14,14a-decahydro-1H-pyrrolo[2,1-c][1,4]oxaazacyclododecin-4-yl)acetate: Using the general procedure for ring closing metathesis with toluene as solvent the title compound was prepared from (S)-4-tert-butyl 1-(((S)-1-(pent-4-enoyl)pyrrolidin-2-yl)methyl) 2-allylsuccinate (390 mg, 1.03 mmol). The product (62 mg) was obtained in 18% yield following purification by column chromatography (Hex/EA gradient). LRMS (ESI+) (M+Na): 374.37.

N-(4-chlorobenzyl)-2-((4S,14aS,E)-3,10-dioxo-3,4,5,8,9,10,12,13,14,14a-decahydro-1H-pyrrolo[2,1-c][1,4]oxaazacyclododecin-4-yl)acetamide: To a solution of tert-butyl 2-((4S,14aS,E)-3,10-dioxo-3,4,5,8,9,10,12,13,14,14a-decahydro-1H-pyrrolo[2,1-c][1,4]oxaazacyclododecin-4-yl)acetate (62 mg, 0.18 mmol) in CH₂Cl₂ (2 mL) cooled in an ice/water bath was added trifluoroacetic acid (203 μL, 2.65 mmol) and the reaction mixture was stirred 4 h at which point TLC analysis indicated complete consumption of starting material. The reaction mixture was concentrated and coevaporated 3 times with toluene. Using general procedure A for amide bond formation with DMF as solvent, the title compound was prepared from 2-((4S,14aS,E)-3,10-dioxo-3,4,5,8,9,10,12,13,14,14a-decahydro-1H-pyrrolo[2,1-c][1,4]oxaazacyclododecin-4-yl)acetic acid (52 mg, 0.18 mmol) and (4-chlorophenyl)methanamine (24 μl, 0.19 mmol). The product (39 mg) was obtained in 53% yield over 2 steps after column chromatography (MeOH/DCM gradient). LRMS (ESI+) (M+H): 419.37.

### Example FU

### N-(4-chlorobenzyl)-2-((3R,11S,E)-3-cyclohexyl-5,12-dioxo-1-oxa-4-azacyclododec-8-en-11-yl)acetamide

(S)-4-tert-butyl 1-((R)-2-cyclohexyl-2-pent-4-enamidoethyl) 2-allylsuccinate: Using general procedure A for amide bond formation with DMF as solvent, (R)—N-(1-cyclohexyl-2-hydroxyethyl)pent-4-enamide was prepared from pent-4-enoic acid (225 μl, 2.20 mmol) and (R)-2-amino-2-cyclohexylethanol (286 mg, 2.00 mmol). The product was obtained and used in the next step without purification. Using general procedure C for ester bond formation with DCM as solvent the title compound was prepared from (S)-2-(2-tert-butoxy-2-oxoethyl)pent-4-enoic acid (471 mg, 2.20 mmol) and (R)—N-(1-cyclohexyl-2-hydroxyethyl)pent-4-enamide (451 mg, 2.00 mmol). Product (173 mg) was obtained in 21% yield over 2 steps after column chromatography (Hex/EA gradient). LRMS (ESI+) (M+Na): 444.49.

tert-butyl 2-((3R,11S,E)-3-cyclohexyl-5,12-dioxo-1-oxa-4-azacyclododec-8-en-11-yl)acetate: Using the general procedure for ring closing metathesis with toluene as solvent the title compound was prepared from (S)-4-tert-butyl 1-((R)-2-cyclohexyl-2-pent-4-enamidoethyl) 2-allylsuccinate (142 mg, 0.34 mmol). The product (58 mg) was obtained as a white solid in 44% yield following purification by column chromatography (Hex/EA gradient). LRMS (ESI+) (M+Na): 416.44.

N-(4-chlorobenzyl)-2-((3R,11S,E)-3-cyclohexyl-5,12-dioxo-1-oxa-4-azacyclododec-8-en-11-yl)acetamide: To a solution of tert-butyl 2-((3R,11S,E)-3-cyclohexyl-5,12-dioxo-1-oxa-4-azacyclododec-8-en-11-yl)acetate (58 mg, 0.15 mmol) in CH₂Cl₂ (2 mL) cooled in an ice/water bath was added trifluoroacetic acid (169 μL, 2.21 mmol) and the reaction mixture was stirred 4 h at which point TLC analysis indicated complete consumption of starting material. The reaction mixture was concentrated and coevaporated 3 times with toluene. Using general procedure A for amide bond formation with DMF as solvent, the title compound was prepared from 2-((3R,11S,E)-3-cyclohexyl-5,12-dioxo-1-oxa-4-azacyclododec-8-en-11-yl)acetic acid (50 mg, 0.15 mmol) and (4-chlorophenyl)methanamine (20 μl, 0.16 mmol). The product (42 mg) was obtained in 62% yield over 2 steps after column chromatography (MeOH/DCM gradient). LRMS (ESI+) (M+H): 461.44.

### Example FW

### tert-butyl 2-((3S,10aR,15aS,E)-2,9-dioxo-2,3,4,7,8,9,10,10a,15,15a-decahydroindeno[2,1-b][1,4]oxaazacyclododecin-3-yl)acetate

N-((1R,2S)-2-hydroxy-2,3-dihydro-1H-inden-1-yl)pent-4-enamide: Using general procedure A for amide bond formation with DMF as solvent, the title compound was prepared from pent-4-enoic acid (75 μl, 0.74 mmol) and (1R,2S)-1-amino-2,3-dihydro-1H-inden-2-ol (100 mg, 0.67 mmol). The product (155 mg) was obtained and used in the next step without purification. LRMS (ESI+) (M+H): 232.14.

(S)-4-tert-butyl 1-((1R,2S)-1-(pent-4-enamido)-2,3-dihydro-1H-inden-2-yl) 2-allylsuccinate: Using general procedure C for ester bond formation with DCM as solvent the title compound was prepared from (S)-2-(2-tert-butoxy-2-oxoethyl)pent-4-enoic acid (158 mg, 0.74 mmol) and N-((1R,2S)-2-hydroxy-2,3-dihydro-1H-inden-1-yl)pent-4-enamide (155 mg, 0.67 mmol). The product (202 mg) was obtained in 71% yield over 2 steps after column chromatography (Hex/EA gradient). LRMS (ESI+) (M+Na): 450.33.

tert-butyl 2-((3S,10aR,15aS,E)-2,9-dioxo-2,3,4,7,8,9,10,10a,15,15a-decahydroindeno[2,1-b][1,4]oxaazacyclododecin-3-yl)acetate: Using the general procedure for ring closing metathesis with toluene as solvent the title compound was prepared from (S)-4-tert-butyl 1-((1R,2S)-1-(pent-4-enamido)-2,3-dihydro-1H-inden-2-yl) 2-allylsuccinate (200 mg, 0.47 mmol). The product (113 mg) was obtained as a white solid in 61% yield following purification by column chromatography (Hex/EA gradient). LRMS (ESI−) (M−H): 398.19.

### Example FZ

### 2-((3R,11S,E)-5,12-dioxo-3-phenyl-1-oxa-4-azacyclododec-8-en-11-yl)-N-((tetrahydro-2H-pyran-4-yl)methyl)acetamide

To a solution of tert-butyl 2-((3R,11S,E)-5,12-dioxo-3-phenyl-1-oxa-4-azacyclododec-8-en-11-yl)acetate (100 mg, 0.26 mmol) in DCM (200 μL) cooled in an ice/water bath was added trifluoroacetic acid (89 μL, 1.2 mmol). The reaction mixture was stirred 30 min at which point no starting material remained and the solution was concentrated. The product obtained, 2-((3R,11S,E)-5,12-dioxo-3-phenyl-1-oxa-4-azacyclododec-8-en-11-yl)acetic acid, was combined with (6-chloropyridin-3-yl)methanamine (37 mg, 0.26 mmol) using general procedure B for amide bond formation to yield the title compound. The product (62 mg) was obtained as a white solid in 52% yield over the two steps. LRMS (ESI+) (M+H): 455.77.

### Example GA

### tert-butyl 2-((2R,6S,E)-5,12-dioxo-2-phenyl-1-oxa-4-azacyclododec-8-en-6-yl)acetate

Using the general procedure for ring closing metathesis with toluene as solvent tert-butyl 2-((2R,6S,E)-5,12-dioxo-2-phenyl-1-oxa-4-azacyclododec-8-en-6-yl)acetate was prepared from (S)-tert-butyl 3-((R)-2-(pent-4-enoyloxy)-2-phenylethylcarbamoyl)hex-5-enoate (4.4 g, 11 mmol). The product (2.6 g) was obtained as a white solid in 73% yield following purification by column chromatography (MeOH/DCM gradient). LRMS (ESI+) (M+Na): 410.38.

### Example GB

### (R)-2-((S)-2-(2-(4-chlorobenzylamino)-2-oxoethyl)pent-4-enamido)-1-phenylethyl pent-4-enoate

To a solution of (S)-tert-butyl 3-((R)-2-(pent-4-enoyloxy)-2-phenylethylcarbamoyl)hex-5-enoate (100 mg, 0.24 mmol) in DCM (190 μL) cooled in an ice/water bath was added trifluoroacetic acid (84 μL, 1.1 mmol). The reaction mixture was stirred 30 min at which point no starting material remained and the solution was concentrated and dried under high vacuum to yield (S)-3-(((R)-2-pent-4-enamido-2-phenylethoxy)carbonyl)hex-5-enoic acid in quantitative yield. This intermediate was combined with (4-chlorophenyl)methanamine (33 μL, 0.27 mmol) using general procedure B for amide bond formation to yield the title compound. The product (15 mg) was obtained as a white solid in 13% yield. LRMS (ESI+) (M+H): 483.25.

### Example GC

### 2-((2R,6S,E)-5,12-dioxo-2-phenyl-1-oxa-4-azacyclododec-8-en-6-yl)acetic acid

To a solution of tert-butyl 2-((2R,6S,E)-5,12-dioxo-2-phenyl-1-oxa-4-azacyclododec-8-en-6-yl)acetate (140 mg, 0.36 mmol) in DCM (280 μL) cooled in an ice/water bath was added trifluoroacetic acid (125 μL, 1.6 mmol). The reaction mixture was stirred 30 min at which point no starting material remained and the solution was concentrated and dried under high vacuum to yield the title compound in quantitative yield. LRMS (ESI+) (M+H): 332.18.

### Example GD

### N-((6-chloropyridin-3-yl)methyl)-2-((2R,6S,E)-5,12-dioxo-2-phenyl-1-oxa-4-azacyclododec-8-en-6-yl)acetamide

2-((2R,6S,E)-5,12-dioxo-2-phenyl-1-oxa-4-azacyclododec-8-en-6-yl)acetic acid (86 mg, 0.26 mmol) was combined with 2-chloro-5-aminomethylpyridine (40.7 mg, 0.29 mmol) using general procedure B for amide bond formation with DCM as solvent to yield the title compound. The product (50 mg) was obtained as a white solid following purification of the crude product by column chromatography (MeOH/DCM gradient). LRMS (ESI+) (M+H): 456.08.

### Example GE

### N-(biphenyl-4-ylmethyl)-2-((2R,6S,E)-5,12-dioxo-2-phenyl-1-oxa-4-azacyclododec-8-en-6-yl)acetamide

114 mg of this analog was prepared similarly to N-((6-chloropyridin-3-yl)methyl)-2-((2R,6S,E)-5,12-dioxo-2-phenyl-1-oxa-4-azacyclododec-8-en-6-yl)acetamide except using biphenyl-4-ylmethanamine (520 mg, 2.9 mmol, 1.0 eq) in the final coupling step. LRMS (ESI+) (M+H): 497.17.

### Example GF

### tert-butyl 2-((3R,11S,E)-5,12-dioxo-3-phenyl-1-oxa-4-azacyclododec-8-en-11-yl)acetate

Using the general procedure for ring closing metathesis with toluene as solvent tert-butyl 2-((3R,11S,E)-5,12-dioxo-3-phenyl-1-oxa-4-azacyclododec-8-en-11-yl)acetate was prepared from (S)-4-tert-butyl 1-((R)-2-pent-4-enamido-2-phenylethyl) 2-allylsuccinate (1.7 g, 4.1 mmol). The product (1.5 g) was obtained as a white solid in 94% yield following purification by column chromatography (MeOH/DCM gradient). LRMS (ESI+) (M+Na): 410.42.

### Example GG

### N-((6-chloropyridin-3-yl)methyl)-2-((2R,6S,E)-4-methyl-5,12-dioxo-2-phenyl-1-oxa-4-azacyclododec-8-en-6-yl)acetamide

To a solution of tert-butyl 2-((2R,6S,E)-4-methyl-5,12-dioxo-2-phenyl-1-oxa-4-azacyclododec-8-en-6-yl)acetate (34 mg, 0.08 mmol) in DCM (65 μL) cooled in an ice/water bath was added trifluoroacetic acid (30 μL, 0.38 mmol). The reaction mixture was stirred 1 h at which point no starting material remained and the solution was concentrated and dried under high vacuum to yield 2-((2R,6S,E)-4-methyl-5,12-dioxo-2-phenyl-1-oxa-4-azacyclododec-8-en-6-yl)acetic acid in quantitative yield. This intermediate was combined with (6-chloropyridin-3-yl)methanamine (12 mg, 0.08 mmol) using general procedure B for amide bond formation to yield the title compound. The product (19 mg) was obtained as a white solid in 48% yield. LRMS (ESI+) (M+H): 469.84.

### Example GH

### N-(4-chlorobenzyl)-2-((3R,11S,E)-4-methyl-5,12-dioxo-3-phenyl-1-oxa-4-azacyclododec-8-en-11-yl)acetamide

tert-butyl 2-((3R,11S,E)-4-methyl-5,12-dioxo-3-phenyl-1-oxa-4-azacyclododec-8-en-11-yl)acetate: To a solution of tert-butyl 2-((3R,11S,E)-5,12-dioxo-3-phenyl-1-oxa-4-azacyclododec-8-en-11-yl)acetate (100 mg, 0.26 mmol) and iodomethane (48 μL, 0.77 mmol) in DMF (1.3 mL) cooled in an ice/water bath was added sodium hydride (6 mg, 0.26 mmol). The reaction mixture was slowly warmed to rt and stirred 8 h at which point 0.5 equivalents of sodium hydride was added. The reaction mixture was stirred an additional 12 h then diluted with sat. NH₄Cl and EtOAc and the layers separated. The aqueous was extracted 2× with EtOAc and the combined organic extracts were washed with brine, dried over Na₂SO₄, filtered, and concentrated. The crude product was purified using silica gel chromatography (MeOH/DCM gradient) to yield 100 mg of slightly impure product that was carried on to the subsequent step. LRMS (ESI+) (M+H): 424.39.

N-(4-chlorobenzyl)-2-((3R,11S,E)-4-methyl-5,12-dioxo-3-phenyl-1-oxa-4-azacyclododec-8-en-11-yl)acetamide: To a solution of tert-butyl 2-((3R,11S,E)-4-methyl-5,12-dioxo-3-phenyl-1-oxa-4-azacyclododec-8-en-11-yl)acetate (100 mg, 0.25 mmol) in DCM (190 μL) cooled in an ice/water bath was added trifluoroacetic acid (120 μL, 1.5 mmol). The reaction mixture was stirred 30 min at which point no starting material remained and the solution was concentrated and dried under high vacuum to yield 2-((3R,11S,E)-4-methyl-5,12-dioxo-3-phenyl-1-oxa-4-azacyclododec-8-en-11-yl)acetic acid in quantitative yield. This product was combined with (4-chlorophenyl)methanamine (30 μL, 0.25 mmol) using general procedure B for amide bond formation to yield the title compound. The product (10 mg) was obtained as a white solid in 9% yield. LRMS (ESI−) (M−H): 467.11.

### Example GI

### 2-((2R,3R,6S,E)-3,4-dimethyl-5,12-dioxo-2-phenyl-1-oxa-4-azacyclododec-8-en-6-yl)-N-(2-ethoxyethyl)acetamide

Using general procedure A for amide bond formation with DCM as solvent, the title compound was prepared from 2-((2R,3R,6S,E)-3,4-dimethyl-5,12-dioxo-2-phenyl-1-oxa-4-azacyclododec-8-en-6-yl)acetic acid (65 mg, 0.181 mmol) and 2-ethoxyethanamine (18 mg, 0.199 mmol). The product (41 mg) was obtained in 53% yield after column chromatography (MeOH/DCM gradient). LRMS (ESI+) (M+H): 431.24.

### Example GJ

### N-((4S,12R,E)-6,13-dioxo-4-phenyl-1-oxa-5-azacyclotridec-9-en-12-yl)acetamide

(S)—N-(3-hydroxy-1-phenylpropyl)pent-4-enamide: Using general procedure A for amide bond formation with DCM as solvent, the title compound was prepared from pent-4-enoic acid (74 μl, 0.73 mmol) and (S)-3-amino-3-phenylpropan-1-ol (100 mg, 0.66 mmol). The product (154 mg) was obtained and used in the next step without purification. LRMS (ESI+) (M+H): 234.26.

(R)—((S)-3-pent-4-enamido-3-phenylpropyl) 2-(tert-butoxycarbonylamino)pent-4-enoate: Using general procedure C for ester bond formation with DCM as solvent the title compound was prepared from (R)-2-(tert-butoxycarbonylamino)pent-4-enoic acid (288 mg, 1.34 mmol) and (S)—N-(3-hydroxy-1-phenylpropyl)pent-4-enamide (154 mg, 0.66 mmol). The product (183 mg) was obtained in 64% yield over 2 steps after column chromatography (Hex/EA gradient). LRMS (ESI+) (M+H): 431.43.

tert-butyl (4S,12R,E)-6,13-dioxo-4-phenyl-1-oxa-5-azacyclotridec-9-en-12-ylcarbamate: Using the general procedure for ring closing metathesis with toluene as solvent the title compound was prepared from (R)—((S)-3-pent-4-enamido-3-phenylpropyl) 2-(tert-butoxycarbonylamino)pent-4-enoate (103 mg, 0.24 mmol). The product (44 mg) was obtained in 46% yield following purification by column chromatography (Hex/EA gradient). LRMS (ESI+) (M+H): 403.41.

(4S,12R,E)-12-amino-4-phenyl-1-oxa-5-azacyclotridec-9-ene-6,13-dione: tert-butyl (4S,12R,E)-6,13-dioxo-4-phenyl-1-oxa-5-azacyclotridec-9-en-12-ylcarbamate (44 mg, 0.11 mmol) was dissolved in DCM (1093 μL) and trifluoroacetic acid (168 μL, 2.19 mmol) and triethylsilane (175 μL, 1.09 mmol) were added dropwise. After 7 h, the reaction is complete as shown by LCMS. Solvents are evaporated and coevaporated with toluene 3 times. The product (33 mg) was obtained and used in the next step without further purification. LRMS (ESI+) (M+H): 303.31.

N-((4S,12R,E)-6,13-dioxo-4-phenyl-1-oxa-5-azacyclotridec-9-en-12-yl)acetamide: (4S,12R,E)-12-amino-4-phenyl-1-oxa-5-azacyclotridec-9-ene-6,13-dione (33 mg, 0.11 mmol) was dissolved in DMF (2.2 mL) under argon atmosphere. Triethylamine (920 μL, 6.55 mmol) and acetic anhydride (619 μL, 6.55 mmol) were successively added in one portion and the reaction mixture was stirred at room temperature for 15 h. Solvents were evaporated before the mixture was dissolved with ethyl acetate and successively washed with NH₄Cl solution and NaHCO₃ solution. The product (18 mg) was obtained in 48% yield over 2 steps after purification by column chromatography (DCM/MeOH gradient). LRMS (ESI+) (M+H): 345.32.

### Compound GO

### N-((3R,11R,E)-5,12-dioxo-3-phenyl-1-oxa-4-azacyclododec-8-en-11-yl)-N-methylacetamide

A mixture of N-((3R,11R,E)-5,12-dioxo-3-phenyl-1-oxa-4-azacyclododec-8-en-11-yl)acetamide (20.00 mg, 0.061 mmol) and iodomethane (11.3 μl, 0.182 mmol) in DMF (500 μl) was cooled in an ice bath. Sodium hydride, 60% weight in oil dispersion (1.453 mg, 0.061 mmol) was added to the reaction. The reaction mixture was slowly warmed to room temperature and monitored by LCMS. After 12 h the reaction was stopped and quenched with water and extracted with DCM×3. The crude product was purified using silica gel chromatography using an ISCO automated system and a MeOH/DCM gradient. The product (8.34 mg) was isolated as a white solid in 40% yield. LRMS (ESI+) (M+H): 346.27.

### Compound GU

### (R,E)-3-(2-oxo-2-(piperidin-1-yl)ethyl)-11-phenyl-1-oxa-3,10-diazacyclododec-5-ene-2,9-dione tert-butyl 2-(4-nitrophenylsulfonamido)acetate

Glycine tert-butyl ester hydrochloride (2.5 g, 14.9 mmol) was sealed in a flask with stir bar under nitrogen and dissolved with DCM (60 mL) before triethylamine (4.26 mL, 30.6 mmol) was added. 4-Nitrobenzene-1-sulfonyl chloride (3.64 g, 16.4 mmol) was sealed under nitrogen in a separate flask and dissolved with DCM (16.4 mL, 14.9 mmol). The resulting solution was added by syringe over 5 min. to the solution of glycine tert-butyl ester hydrochloride, and the reaction was stirred for 18 h. The reaction was quenched by pouring it onto half-saturated aq. NaHCO₃ (100 mL). It was then diluted with more DCM and water, the layers were separated, and the organic layer was washed again with aq. NaHCO₃, then brine. The organics were dried over MgSO₄, filtered, and concentrated to give the desired product as a white solid (4.73 g, 100%). LRMS (ESI−) (M−H): 315.24.

tert-butyl 2-(allylamino)acetate hydrochloride: tert-Butyl 2-(4-nitrophenylsulfonamido)acetate (2.00 g, 6.32 mmol) and finely-ground potassium carbonate (1.75 g, 12.7 mmol) were added to a flask with stir bar and sealed under nitrogen, then dry DMF (32 mL) was added. Allyl bromide (0.59 mL, 7.0 mmol) was added dropwise, then the reaction was stirred vigorously for 18 h. The reaction was then diluted with EtOAc (150 mL) and dilute aq. NaHCO₃ (150 mL), the layers were separated, and the organic phase was washed with water×3, then brine. The organic phase was dried over MgSO₄, filtered, and concentrated to a yellow oil. The crude material was purified by column chromatography (EtOAc/hexanes gradient) to yield a yellow solid (1.92 g, 85%). The nosyl protecting group was removed by mixing the intermediate tert-butyl 2-(N-allyl-4-nitrophenylsulfonamido)acetate (1.92 g, 5.39 mmol) with finely-ground potassium carbonate (2.23 g, 16.2 mmol) in a flask with stir bar and sealing under nitrogen. DMF (36 mL) was added and a vent line to an oil bubbler was attached, then benzenethiol (0.66 ml, 6.5 mmol) was added via syringe. The suspension was stirred vigorously for 6 h, then diluted with water (150 mL) and Et₂O (150 mL). The layers were separated, then the organic phase was washed with water (75 mL×3). 1 M aq. HCl (50 mL) was added, then the layers were separated and the aqueous layer was washed with Et₂O (40 mL), then basified with 10% aq. NaOH to pH>10, causing the free base to precipitate. The solid was extracted with Et₂O (2×40 mL), then the combined organics were washed with brine, dried over Na₂SO₄, and filtered. The desired hydrochloride salt was formed by adding 4 M HCl in dioxane (˜2 mL) slowly. The resulting white solid was filtered with a Buchner funnel and washed with Et₂O (660 mg, 59%). LRMS (ESI+) (M+H): 172.21.

(R)-4-nitrophenyl (2-(pent-4-enamido)-2-phenylethyl)carbonate: 4-Nitrophenylchloroformate (956 mg, 4.74 mmol) was added to a flask with stir bar and sealed under nitrogen, then dissolved with dry DCM (18 mL). The flask was cooled on ice, then pyridine (1.48 mL, 18.2 mmol) was added, followed by (R)—N-(2-hydroxy-1-phenylethyl)pent-4-enamide (800 mg, 3.65 mmol). The reaction was removed from the ice bath and stirred for 18 h, then concentrated. The resulting waxy solid was redissolved with DCM and purified by column chromatography (EtOAc/hexanes gradient), yielding the desired product as a waxy off-white solid (1.155 g, 82%). LRMS (ESI+) (M+H): 385.29.

(R)-tert-butyl 2-(allyl((2-(pent-4-enamido)-2-phenylethoxy)carbonyl)amino)acetate: (R)-4-Nitrophenyl 2-pent-4-enamido-2-phenylethyl carbonate (507 mg, 1.32 mmol) was added to a flask with stir bar and sealed under nitrogen. Dry DCM (6.5 mL) was added, then the reaction was cooled on ice and (i-Pr)₂NEt (0.58 mL, 3.30 mmol) was added, followed by tert-butyl 2-(allylamino)acetate hydrochloride (288 mg, 1.39 mmol). The reaction was found to be sluggish, so DMAP (10 mg, 0.08 mmol) was added, the flask was sealed with a plastic cap (and tape to secure it), then heated at 40° C. for 6 h. The reaction was quenched with half-saturated aq. NaHCO₃ solution and diluted with DCM. The phases were separated and the organic layer was washed with aq. NaHCO₃×4 to remove 4-nitrophenol, then brine. The crude was dried over MgSO₄, concentrated to a colorless oil, and purified by column chromatography (EtOAc/hexanes gradient), yielding the desired product as a colorless oil (324 mg, 59%). LRMS (ESI+) (M+H): 417.38.

(R,E)-tert-butyl 2-(2,9-dioxo-11-phenyl-1-oxa-3,10-diazacyclododec-5-en-3-yl)acetate: Using the general procedure for ring closing metathesis using toluene as solvent, the desired product was prepared from (R)-tert-butyl 2-(allyl((2-(pent-4-enamido)-2-phenylethoxy)carbonyl)amino)acetate (324 mg, 0.78 mmol), yielding a colorless oil (138 mg, 48%) after column chromatography (EtOAc/hexanes gradient). LRMS (ESI−) (M+formate): 433.12.

(R,E)-3-(2-oxo-2-(piperidin-1-yl)ethyl)-11-phenyl-1-oxa-3,10-diazacyclododec-5-ene-2,9-dione: (R,E)-tert-butyl 2-(2,9-dioxo-11-phenyl-1-oxa-3,10-diazacyclododec-5-en-3-yl)acetate (138 mg, 0.36 mmol) was added to a flask, followed by DCM (3.5 mL), triethylsilane (0.57 mL, 3.6 mmol), and trifluoroacetic acid (0.55 mL, 7.1 mmol). The reaction was stirred for 15 h, then concentrated and dried well under high vacuum, pulverizing several times to ensure that the silanes were removed. 58 mg (0.18 mmol) of the crude intermediate carboxylic acid was reacted with piperidine according to general procedure A for amide bond formation with DCM as solvent. The desired product was isolated from the reaction according to the general procedure using EtOAc as solvent for the work-up, followed by column chromatography (MeOH/DCM gradient), yielding a white solid (31 mg, 44%). LRMS (ESI+) (M+H): 400.39.

### Example 2

### In Vitro Activity

The activity of compounds of the invention is tested in Shh-LIGHT2 cells (ATCC, Manassas Va.) (Tapaile, J., et al., 2000 supra), which is an NIH3T3 cell line with a Gli-dependent firefly luciferase reporter. These cells can be used to demonstrate the efficacy of Shh pathway inhibitors (cyclopamine) and activators (purmorphamine and SAG) (Chen et al. and Tapaile et al., supra; Surajit, S., Chen, J. K. Nat. Chem. Biol. 2, 29-30, 2006). Shh pathway activity is inferred by measuring firefly luciferase levels after a 30 h incubation with compound in the presence of HCM.

To explore further the potential mechanism of Shh pathway inhibition involving direct perturbation of the ShhN protein complex, compounds are tested in a Ptc1^{-/-} cell line derived from mouse embryos lacking Ptc1 function. The cell line has both Ptc1 alleles replaced with a β-galoctosidase (β-gal) reporter (Surajit et al., supra). Because Ptc1 inhibits Hh pathway activation by repressing Smo function, removing both Ptc1 alleles results in constitutive pathway activation. Small-molecule pathway inhibitors that act downstream of Ptc1 remain active in this cell line. In the Ptc1^{-/-} cell line, Shh pathway activity is proportional to the 3-gal levels observed after 30 h of incubation with compound. If no significant difference is observed when the Ptc1^{-/-} cell line is treated with HCM or low serum-containing culture medium (LCCM), this confirms that the Ptc1 receptor is absent, the Shh pathway is constitutively activated and Shh does not increase Shh pathway activation. Previous studies have demonstrated that cyclopamine, whose target (Smo) is downstream of Ptc1 (Chen, J. K., Taipale J., Cooper, M. K.; Beachy, P. A. Genes Dev. 16, 2743-2748, 2002), is effective at ablating 3-gal reporter activity in this cell line.

### Example 3

### Surface Plasmon Resonance (SPR) Protein Binding Assays

Biacore™ T100 (GE Healthcare, Uppsala, Sweden) was used to perform the experiments described herein. Sensor surface preparation and interaction analyses experiments were performed at 25° C. Prior to surface preparation, lyophilized ShhN protein (R&D Systems) was dissolved in either water or PBS buffer, at pH 7.4 and protein purity was determined by Nu-Page 4-12% Bis-Tris gel in MOPS buffer with a silver stain sensor preparation.

ShhN was immobilized onto series S sensor chip CM4 via a standard N-ethyl-N′-(dimethylaminopropyl)carbodiimide/N-hydroxysuccinimide (EDC/NHS) amine coupling procedures (Biacore Sensor Surface Handbook, 2003, version AA, Biacore, Uppsala, Sweden). Shh was diluted to 10 g/mL in 10 mM sodium acetate pH 5.5 for these procedures and resultant immobilization levels were 1000-1200 R.U.s. Control surfaces were prepared similarly without protein derivatization and were utilized as a reference surfaces for compound binding experiments.

For compound interaction analyses, 0.01 M Hepes, pH 7.4, 0.15 M NaCl, 0.05% Surfactant P20 and 5% DMSO were used for both running and sample buffers. Compound samples were prepared by serial dilution in the range 0.78 μM-25 μM and flow over control and derivatized surfaces for two minutes at a flow-rate of 80 μL/min. Zero concentration blank buffer cycles were included as negative control samples. Solvent correction procedures were also included to compensate for any DMSO related bulk refractive index variations and were performed as described previously (Karlsson, R. et al. Anal. Biochem. 278, 1-13, 2000).

Data analysis was carried out using Biacore T100 evaluation software v1.1.1. Data were prepared by subtraction of reference surface data and blank buffer sample data, a procedure commonly referred to as ‘double referencing’ (Myszka, D. G. J. Mol. Recognit. 12, 1-6, 1999). Solvent correction was then applied as described previously.

The results of this assay are found in Table 2. Compounds with excellent binding (K_{D} of less than 100 nM) are represented by “***,” compounds with very good binding (K_{D} of 100 nM or greater and less than 1 μM) are represented by “**,” compounds with good binding (K_{D} of 1 μM or greater and less than 100 μM) are represented by “*,” and compounds with to no binding (K_{D} of 100 μM or greater) are represented by “—.”

### Example 4

### Primary Keratinocyte Cell Culture and Artificial Skin Equivalents

To study the potency of the compounds of the invention in cells, select compounds of the invention were conducted with keratinocytes and the reduction in Gli mRNA was measured. Culturing of primary human keratinocytes was previously described (Nguyen, B. C., et al., Genes & Dev. 20:1028-1042 (2006)). The full thickness skin model, EpiDermFT System (MatTek, Ashland), consisted of normal, human-derived epidermal keratinocytes and normal, human-derived dermal fibroblasts which have been cultured to form a multilayered, highly differentiated model of the human dermis and epidermis. The results of this assay are found in Table 2. Compounds with excellent inhibition (IC₅₀ of less than 100 nM) are represented by “***,” compounds with very good inhibition (IC₅₀ of 100 nM or greater and less than 1 μM) are represented by “**,” compounds with good inhibition (IC₅₀ of 1 M or greater and less than 100 μM) are represented by “*,” and compounds with to no binding (K_{D} of 100 M or greater) are represented by “—.”

### Example 5

### Analysis of Gene Expression by Real Time RT-PCR

Gene expression is compared by quantifying mRNA levels by real time RT-PCR. For this, total RNA preparations (1 g) are used in a reverse transcriptase reaction with a mix of oligonucleotide dT and random hexamer primers, followed by real time PCR with gene-specific primers (Gli1: Qiagen Quantitect PrimerAssay QT00060501; Gli2: Qiagen Quantitect PrimerAssay QT00018648), using an Icycler IQ™ real time detection system (Bio-Rad) according to the manufacturer's recommendation, with SYBR Green (Bio-Rad) for detection. Each sample is tested in triplicate, and the results are normalized by real time PCR of the same cDNA with 36B4 primers (36B4 forward primer: 5′-GCA ATG TTG CCA GTG TCT GT 3 (SEQ ID: 1); reverse primer, 5′-GCC TTG ACC TTT TCA GCA AG-3′ (SEQ ID: 2)).

### Example 6

### Induction of Osteogenic Differentiation

Using assays previously described in Dwyer et al., J. Biol. Chem. 282, (2007), 8959-68 and Nakamura et al., Biochm. Biophys. Res. Comm., 237 (1997) 465-9, select compounds of the invention were screened for their ability to modulate osteoblast differentiation. The results of this assay are found in Table 2. Compounds with excellent inhibition (IC₅₀ of less than 100 nM) are represented by “***,” compounds with very good inhibition (IC₅₀ of 100 nM or greater and less than 1 μM) are represented by “**,” compounds with good inhibition (IC₅₀ of 1 μM or greater and less than 100 μM) are represented by “*,” and compounds with to no binding (K_{D} of 100 μM or greater) are represented by “—.”

TABLE 2

| | | | IC₅₀ (nM) | IC₅₀ (Gli1) |
|---|---|---|---|---|
| | Compound | K_{D}(nM) | C3H10T1/2 | (nM) |
| A | 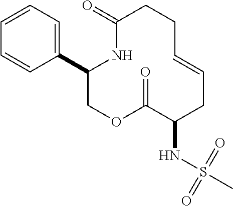 | | *** | |
| B | 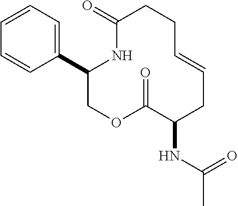 | *** | *** | |
| C | 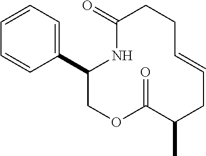 | *** | ** | *** |
| D | 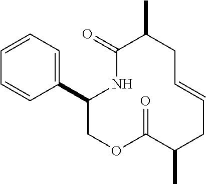 | *** | ** | |
| E | 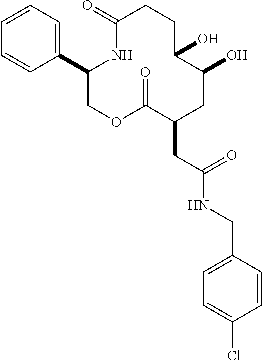 | *** | * | *** |
| F | 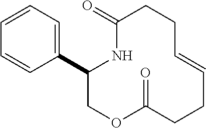 | *** | ** | |
| G | 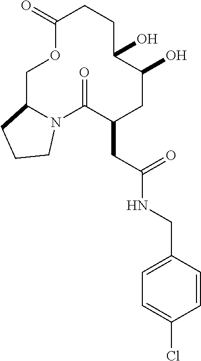 [SMILES: O=C(C[C@@H]1C[C@H](O)[C@@H](O)CCC(=O)OC[C@@H]2CCCN2C1=O)NCc1ccc(Cl)cc1] | ** | * | |
| H | 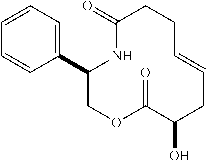 | *** | * | |
| I | 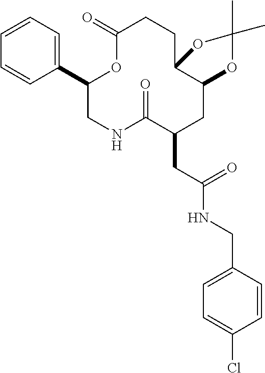 | *** | * | *** |
| J | 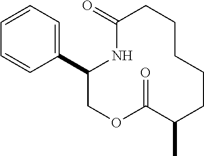 | *** | * | |
| K | 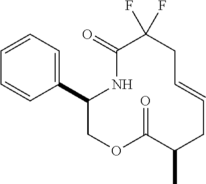 | ** | — | |
| L | 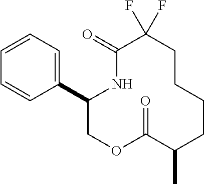 | | — | |
| M | 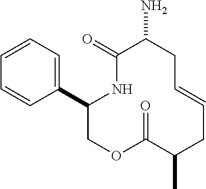 | ** | — | |
| N | 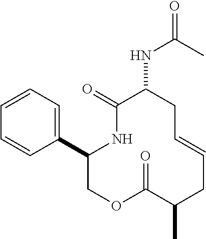 | ** | — | |
| O | 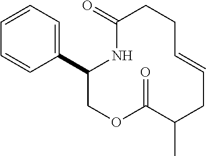 | | | |
| P | 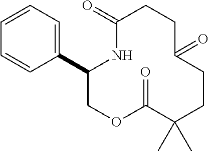 | | | |
| Q | 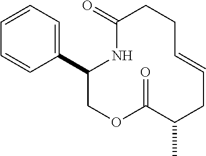 | | | |
| R | 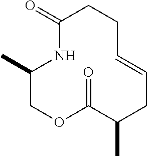 | | | |
| S | 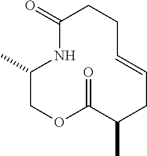 | | | |
| T | 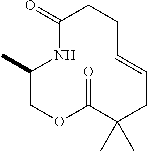 | | | |
| U | 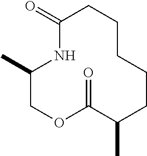 | | | |
| V | 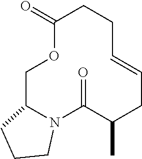 [SMILES: C[C@@H]1C/C=C/CCC(=O)OC[C@H]2CCCN2C1=O] | | | |
| W | 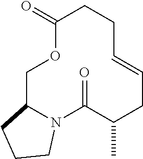 [SMILES: C[C@H]1C/C=C/CCC(=O)OC[C@@H]2CCCN2C1=O] | | | |
| X | 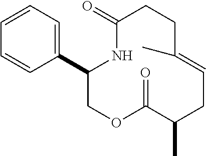 | | | |
| Y | 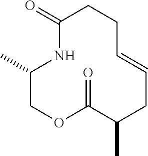 | | | |
| Z | 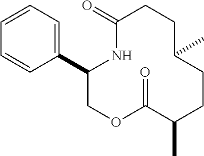 | | | |
| AA | 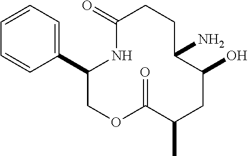 | | | |
| AB | 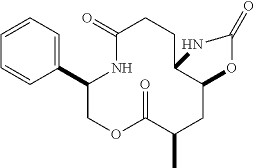 | | | |
| AC | 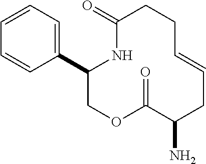 | * | | |
| AD | 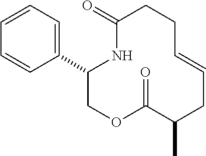 | | | |
| AE | 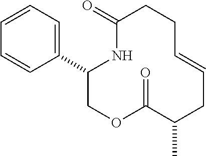 | | | |
| AF | 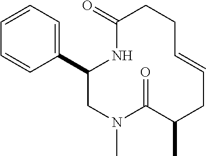 | | | |
| AG | 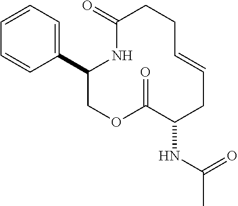 | | | |
| AH | 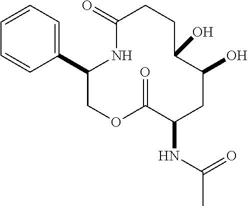 | | | |
| AI | 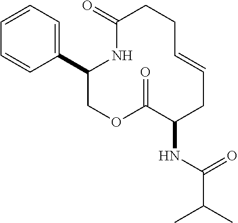 | | | |
| AJ | 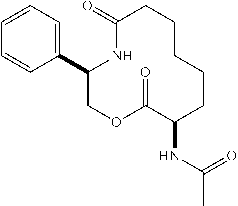 | | | |
| AK | 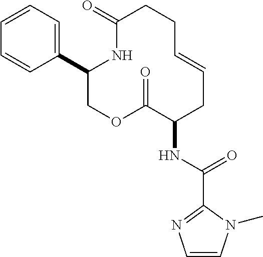 | | | |
| AL | 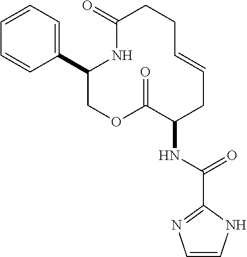 | | | |
| AM | 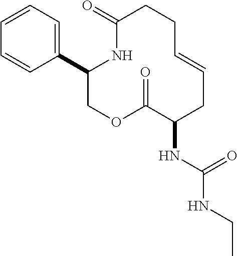 | | | |
| AN | 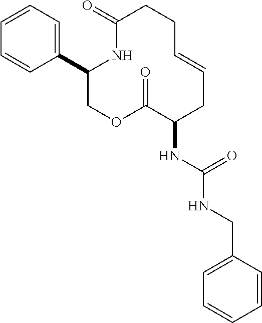 | | | |
| AO | 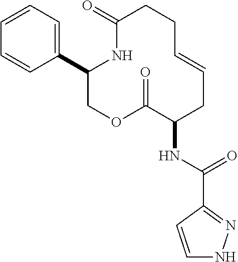 | | | |
| AP | 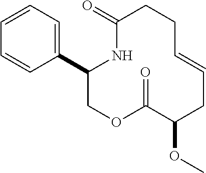 | | | |
| AQ | 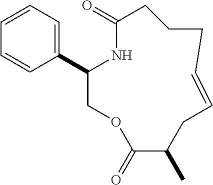 | | | |
| AR | 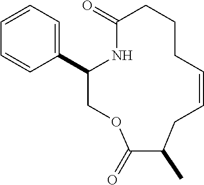 | | | |
| AS | 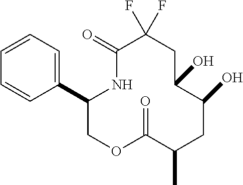 | | | |
| AT | 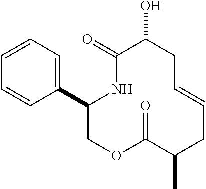 | | | |
| AU | 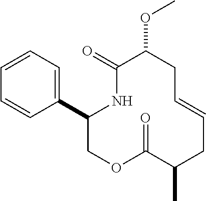 | | | |
| AV | 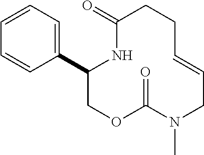 | | | |
| AW | 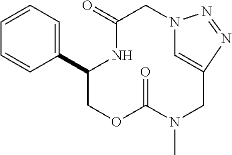 | | | |
| AX | 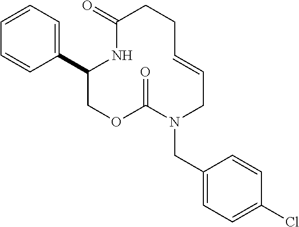 | | | |
| AY | 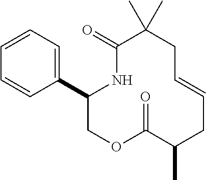 | * | | |
| AZ | 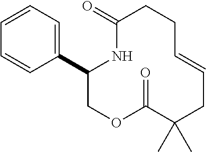 | — | | |
| BA | 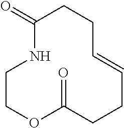 [SMILES: O=C1CC/C=C/CCC(=O)OCCN1] | | | |
| BB | 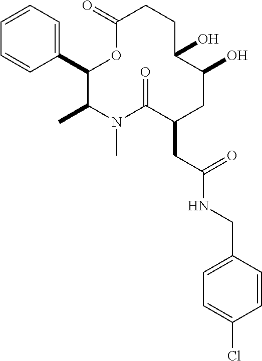 | * | | |
| BC | 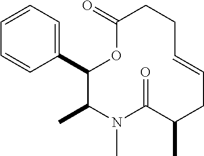 | * | | |
| BD | 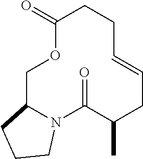 [SMILES: C[C@@H]1C/C=C/CCC(=O)OC[C@@H]2CCCN2C1=O] | * | | |
| BE | 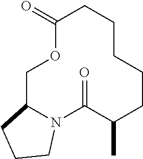 [SMILES: C[C@@H]1CCCCCC(=O)OC[C@@H]2CCCN2C1=O] | * | | |
| BF | 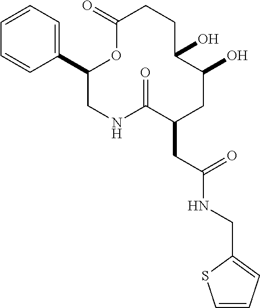 | | | |
| BG | 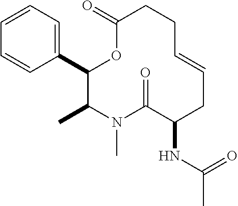 | | | |
| BH | 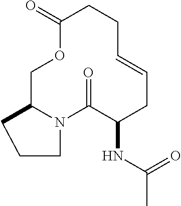 [SMILES: CC(=O)N[C@@H]1C/C=C/CCC(=O)OC[C@@H]2CCCN2C1=O] | | | |
| BI | 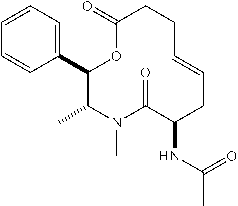 | | | |
| BJ | 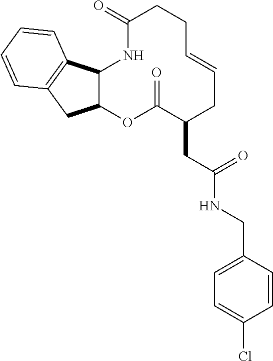 | * | | |
| BK | 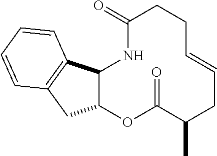 | | | |
| BL | 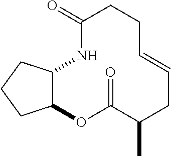 | | | |
| BM | 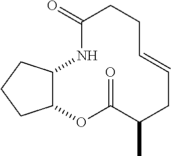 | | | |
| BN | 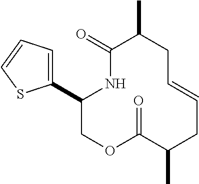 [SMILES: C[C@@H]1C/C=C/C[C@H](C)C(=O)N[C@H](c2cccs2)COC1=O] | | | |
| BO | 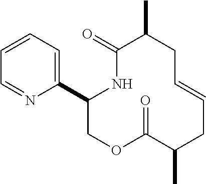 [SMILES: C[C@@H]1C/C=C/C[C@H](C)C(=O)N[C@H](c2ccccn2)COC1=O] | | | |
| BP | 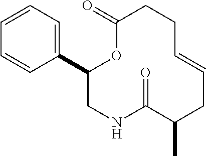 [SMILES: C[C@@H]1C/C=C/CCC(=O)O[C@H](c2ccccc2)CNC1=O] | — | | |
| BQ | 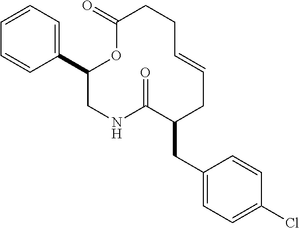 [SMILES: O=C1CC/C=C/C[C@@H](Cc2ccc(Cl)cc2)C(=O)NC[C@@H](c2ccccc2)O1] | *** | | |
| BR | 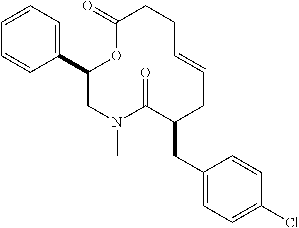 [SMILES: CN1C[C@@H](c2ccccc2)OC(=O)CC/C=C/C[C@@H](Cc2ccc(Cl)cc2)C1=O] | — | | |
| BS | 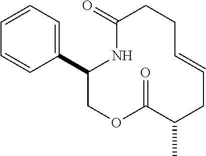 [SMILES: C[C@H]1C/C=C/CCC(=O)N[C@H](c2ccccc2)COC1=O] | — | | |
| BT | 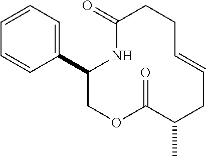 [SMILES: C[C@H]1C/C=C/CCC(=O)N[C@H](c2ccccc2)COC1=O] | | | |
| BU | 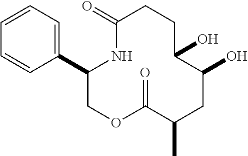 [SMILES: C[C@@H]1C[C@H](O)[C@@H](O)CCC(=O)N[C@H](c2ccccc2)COC1=O] | * | | |
| BV | 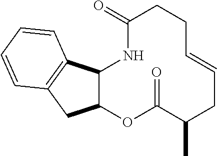 | ** | | |
| BW | 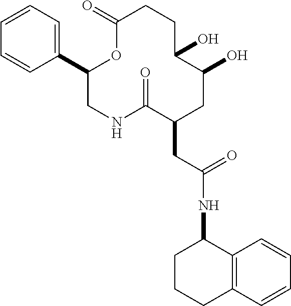 | | | |
| BX | 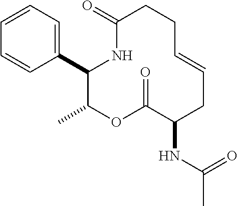 [SMILES: CC(=O)N[C@@H]1C/C=C/CCC(=O)N[C@H](c2ccccc2)[C@@H](C)OC1=O] | | | |
| BY | 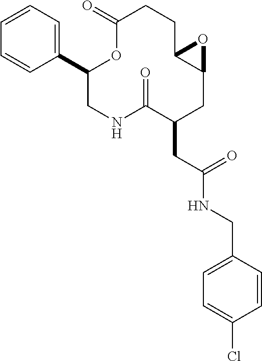 | ** | | *** |
| BZ | 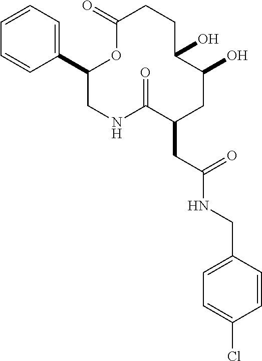 [SMILES: O=C(C[C@@H]1C[C@H](O)[C@@H](O)CCC(=O)O[C@H](c2ccccc2)CNC1=O)NCc1ccc(Cl)cc1] | * | | ** |
| CA | 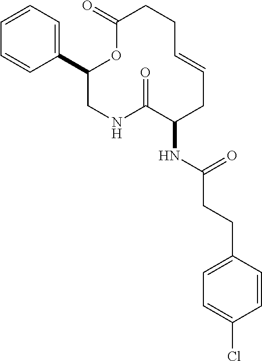 [SMILES: O=C(CCc1ccc(Cl)cc1)N[C@@H]1C/C=C/CCC(=O)O[C@H](c2ccccc2)CNC1=O] | | | — |
| CB | 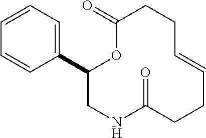 [SMILES: O=C1CC/C=C/CCC(=O)O[C@H](c2ccccc2)CN1] | — | | ** |
| CC | 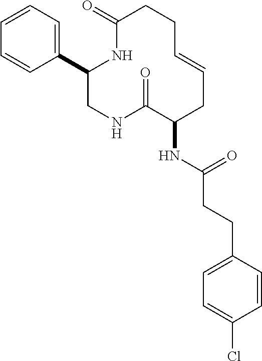 [SMILES: O=C1CC/C=C/C[C@@H](NC(=O)CCc2ccc(Cl)cc2)C(=O)NC[C@@H](c2ccccc2)N1] | — | | |
| CD | 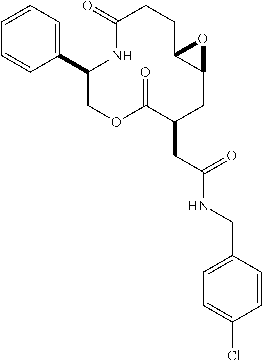 | * | | * |
| CE | 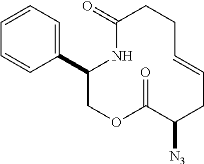 [SMILES: [N-]=[N+]=N[C@@H]1C/C=C/CCC(=O)N[C@H](c2ccccc2)COC1=O] | | | |
| CF | 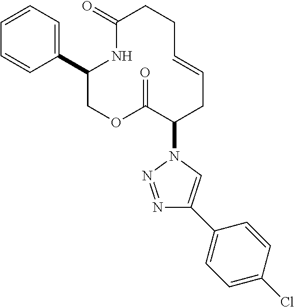 [SMILES: O=C1CC/C=C/C[C@@H](n2cc(-c3ccc(Cl)cc3)nn2)C(=O)OC[C@@H](c2ccccc2)N1] | | | |
| CG | 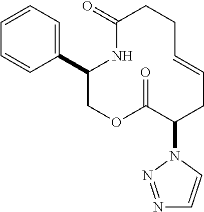 [SMILES: O=C1CC/C=C/C[C@@H](n2ccnn2)C(=O)OC[C@@H](c2ccccc2)N1] | | | |
| CH | 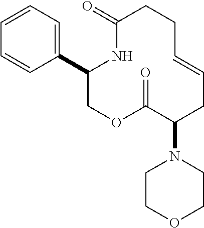 [SMILES: O=C1CC/C=C/C[C@@H](N2CCOCC2)C(=O)OC[C@@H](c2ccccc2)N1] | | | |
| CI | 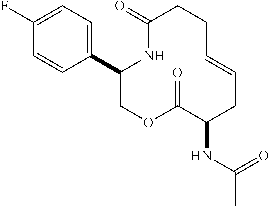 [SMILES: CC(=O)N[C@@H]1C/C=C/CCC(=O)N[C@H](c2ccc(F)cc2)COC1=O] | | | |
| CJ | 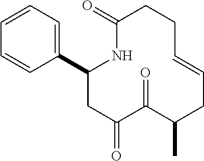 [SMILES: C[C@@H]1C/C=C/CCC(=O)N[C@H](c2ccccc2)CC(=O)C1=O] | | | |
| CK | 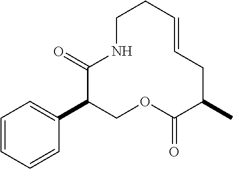 [SMILES: C[C@@H]1C/C=C/CCNC(=O)[C@H](c2ccccc2)COC1=O] | | | |
| CL | 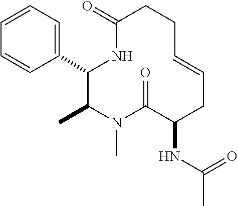 [SMILES: CC(=O)N[C@@H]1C/C=C/CCC(=O)N[C@@H](c2ccccc2)[C@H](C)N(C)C1=O] | | | |
| CM | 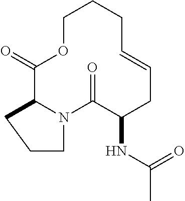 [SMILES: CC(=O)N[C@@H]1C/C=C/CCCOC(=O)[C@@H]2CCCN2C1=O] | | | |
| CN | 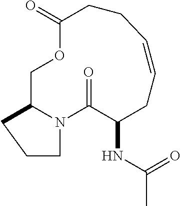 [SMILES: CC(=O)N[C@@H]1C/C=C\CCC(=O)OC[C@@H]2CCCN2C1=O] | | | |
| CO | 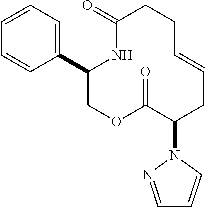 [SMILES: O=C1CC/C=C/C[C@@H](n2cccn2)C(=O)OC[C@@H](c2ccccc2)N1] | | | |
| CP | 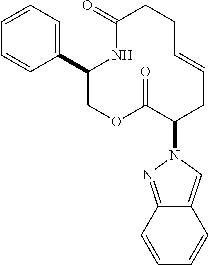 [SMILES: O=C1CC/C=C/C[C@@H](n2cc3ccccc3n2)C(=O)OC[C@@H](c2ccccc2)N1] | | | |
| CQ | 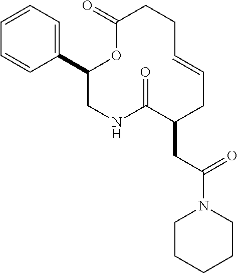 [SMILES: O=C1CC/C=C/C[C@@H](CC(=O)N2CCCCC2)C(=O)NC[C@@H](c2ccccc2)O1] | ** | *** | *** |
| CR | 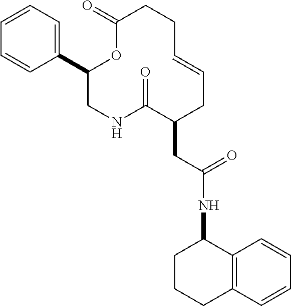 | *** | *** | *** |
| CS | 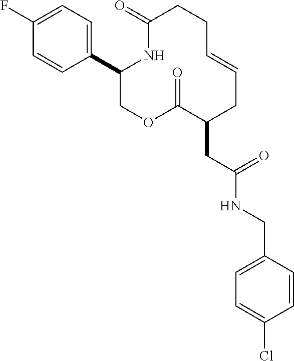 [SMILES: O=C(C[C@@H]1C/C=C/CCC(=O)N[C@H](c2ccc(F)cc2)COC1=O)NCc1ccc(Cl)cc1] | ** | *** | |
| CT | 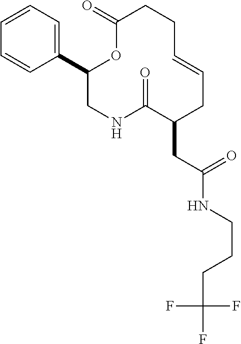 [SMILES: O=C(C[C@@H]1C/C=C/CCC(=O)O[C@H](c2ccccc2)CNC1=O)NCCCC(F)(F)F] | *** | *** | *** |
| CV | 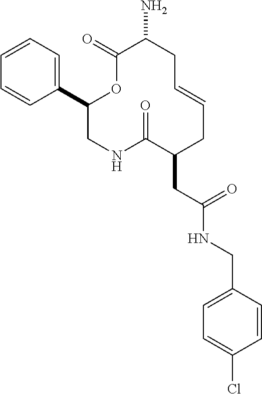 [SMILES: N[C@@H]1C/C=C/C[C@@H](CC(=O)NCc2ccc(Cl)cc2)C(=O)NC[C@@H](c2ccccc2)OC1=O] | *** | ** | *** |
| CX | 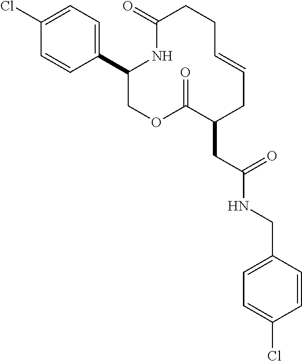 [SMILES: O=C(C[C@@H]1C/C=C/CCC(=O)N[C@H](c2ccc(Cl)cc2)COC1=O)NCc1ccc(Cl)cc1] | *** | ** | |
| CZ | 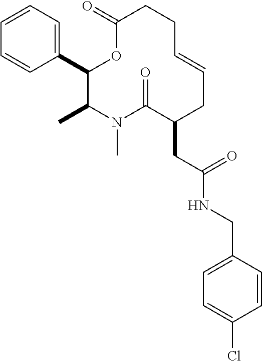 [SMILES: C[C@H]1[C@@H](c2ccccc2)OC(=O)CC/C=C/C[C@@H](CC(=O)NCc2ccc(Cl)cc2)C(=O)N1C] | *** | ** | ** |
| DA | 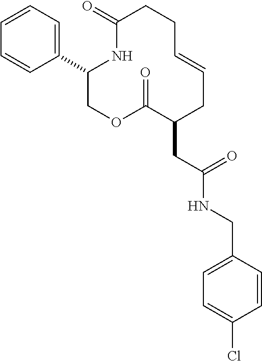 [SMILES: O=C(C[C@@H]1C/C=C/CCC(=O)N[C@@H](c2ccccc2)COC1=O)NCc1ccc(Cl)cc1] | *** | ** | *** |
| DB | 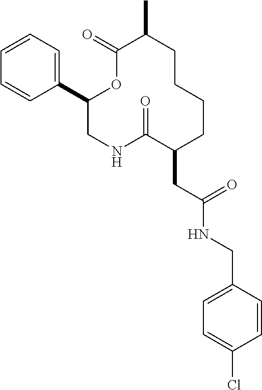 [SMILES: C[C@H]1CCCC[C@@H](CC(=O)NCc2ccc(Cl)cc2)C(=O)NC[C@@H](c2ccccc2)OC1=O] | *** | ** | |
| DC | 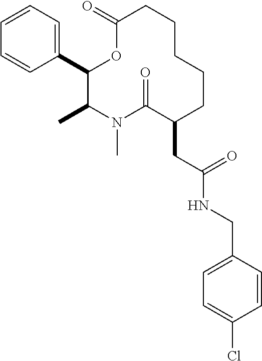 [SMILES: C[C@H]1[C@@H](c2ccccc2)OC(=O)CCCCC[C@@H](CC(=O)NCc2ccc(Cl)cc2)C(=O)N1C] | ** | ** | |
| DD | 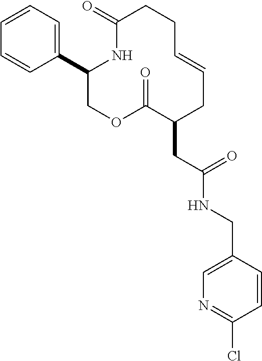 [SMILES: O=C(C[C@@H]1C/C=C/CCC(=O)N[C@H](c2ccccc2)COC1=O)NCc1ccc(Cl)nc1] | *** | ** | |
| DE | 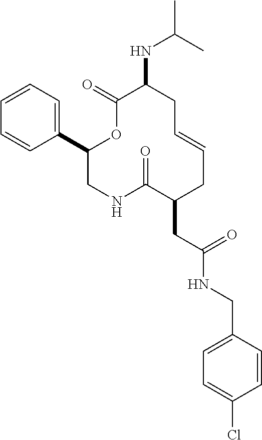 [SMILES: CC(C)N[C@H]1C/C=C/C[C@@H](CC(=O)NCc2ccc(Cl)cc2)C(=O)NC[C@@H](c2ccccc2)OC1=O] | ** | ** | |
| DF | 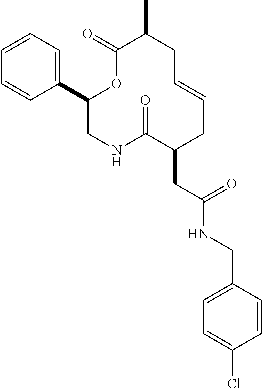 [SMILES: C[C@H]1C/C=C/C[C@@H](CC(=O)NCc2ccc(Cl)cc2)C(=O)NC[C@@H](c2ccccc2)OC1=O] | ** | ** | |
| DG | 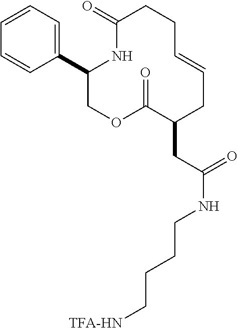 | | * | |
| DI | 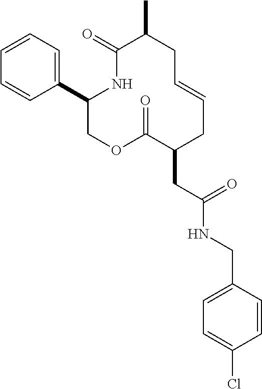 [SMILES: C[C@H]1C/C=C/C[C@@H](CC(=O)NCc2ccc(Cl)cc2)C(=O)OC[C@@H](c2ccccc2)NC1=O] | ** | * | |
| DJ | 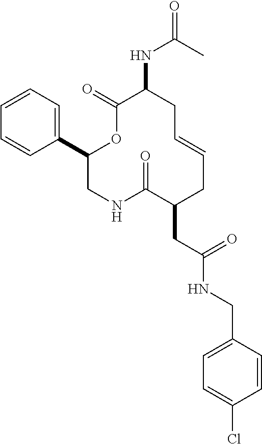 [SMILES: CC(=O)N[C@H]1C/C=C/C[C@@H](CC(=O)NCc2ccc(Cl)cc2)C(=O)NC[C@@H](c2ccccc2)OC1=O] | *** | ** | — |
| DK | 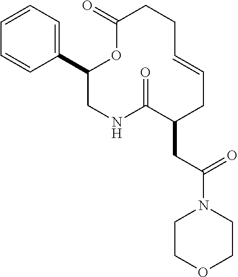 [SMILES: O=C1CC/C=C/C[C@@H](CC(=O)N2CCOCC2)C(=O)NC[C@@H](c2ccccc2)O1] | *** | * | ** |
| DM | 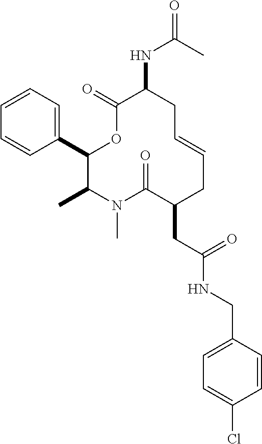 [SMILES: CC(=O)N[C@H]1C/C=C/C[C@@H](CC(=O)NCc2ccc(Cl)cc2)C(=O)N(C)[C@@H](C)[C@@H](c2ccccc2)OC1=O] | *** | * | ** |
| DN | 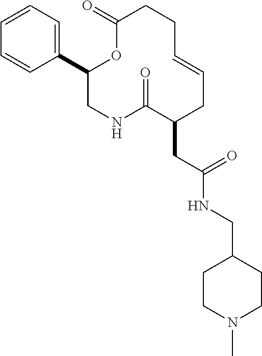 [SMILES: CN1CCC(CNC(=O)C[C@@H]2C/C=C/CCC(=O)O[C@H](c3ccccc3)CNC2=O)CC1] | *** | * | ** |
| DO | 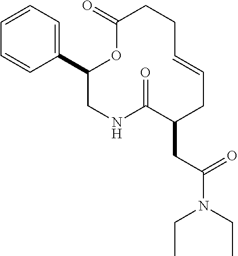 [SMILES: CCN(CC)C(=O)C[C@@H]1C/C=C/CCC(=O)O[C@H](c2ccccc2)CNC1=O] | *** | * | |
| DP | 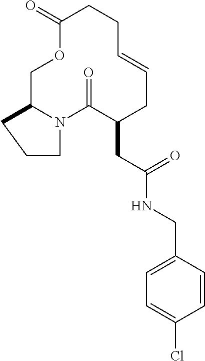 [SMILES: O=C(C[C@@H]1C/C=C/CCC(=O)OC[C@@H]2CCCN2C1=O)NCc1ccc(Cl)cc1] | * | * | |
| DQ | 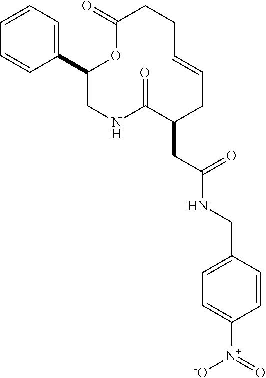 | * | — | |
| DR | 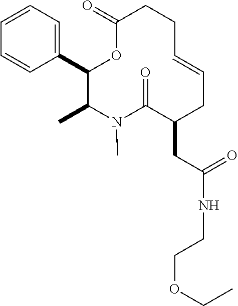 [SMILES: CCOCCNC(=O)C[C@@H]1C/C=C/CCC(=O)O[C@H](c2ccccc2)[C@H](C)N(C)C1=O] | | — | |
| DS | 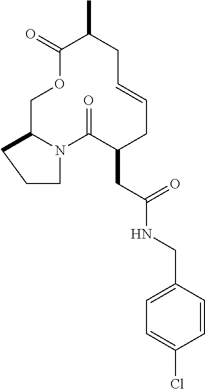 [SMILES: C[C@H]1C/C=C/C[C@@H](CC(=O)NCc2ccc(Cl)cc2)C(=O)N2CCC[C@H]2COC1=O] | | | |
| DT | 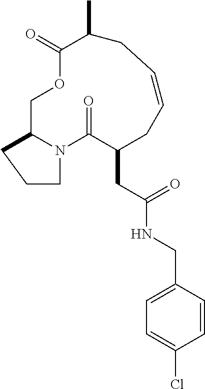 [SMILES: C[C@H]1C/C=C\C[C@@H](CC(=O)NCc2ccc(Cl)cc2)C(=O)N2CCC[C@H]2COC1=O] | | | |
| DU | 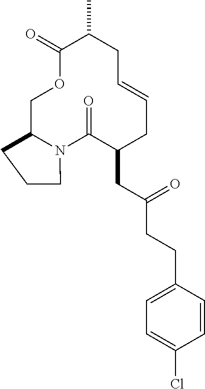 [SMILES: C[C@@H]1C/C=C/C[C@@H](CC(=O)CCc2ccc(Cl)cc2)C(=O)N2CCC[C@H]2COC1=O] | | | |
| DV | 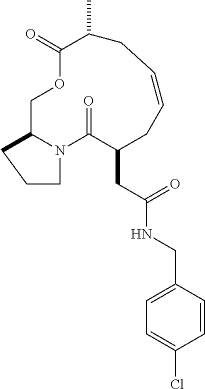 [SMILES: C[C@@H]1C/C=C\C[C@@H](CC(=O)NCc2ccc(Cl)cc2)C(=O)N2CCC[C@H]2COC1=O] | | | |
| DW | 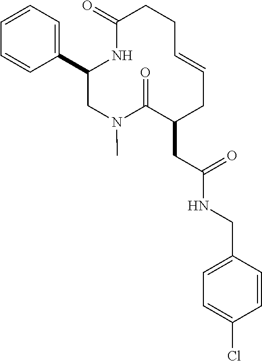 [SMILES: CN1C[C@@H](c2ccccc2)NC(=O)CC/C=C/C[C@@H](CC(=O)NCc2ccc(Cl)cc2)C1=O] | | | |
| DX | 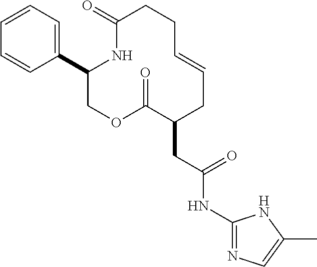 [SMILES: Cc1cnc(NC(=O)C[C@@H]2C/C=C/CCC(=O)N[C@H](c3ccccc3)COC2=O)[nH]1] | | | |
| DY | 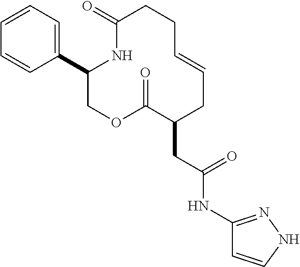 [SMILES: O=C(C[C@@H]1C/C=C/CCC(=O)N[C@H](c2ccccc2)COC1=O)Nc1cc[nH]n1] | | | |
| DZ | 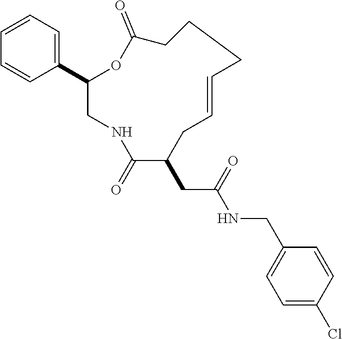 [SMILES: O=C(C[C@@H]1C/C=C/CCCC(=O)O[C@H](c2ccccc2)CNC1=O)NCc1ccc(Cl)cc1] | | | |
| EA | 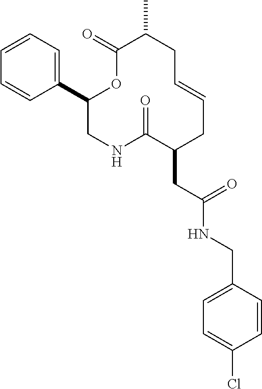 [SMILES: C[C@@H]1C/C=C/C[C@@H](CC(=O)NCc2ccc(Cl)cc2)C(=O)NC[C@@H](c2ccccc2)OC1=O] | ** | | |
| EB | 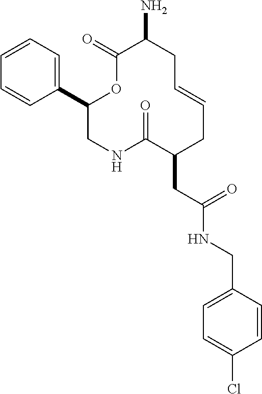 [SMILES: N[C@H]1C/C=C/C[C@@H](CC(=O)NCc2ccc(Cl)cc2)C(=O)NC[C@@H](c2ccccc2)OC1=O] | * | | |
| EC | 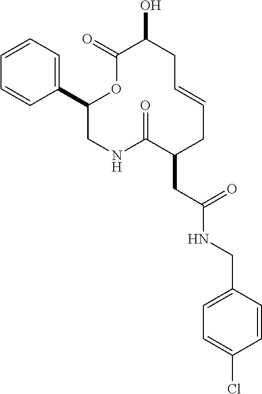 [SMILES: O=C(C[C@@H]1C/C=C/C[C@H](O)C(=O)O[C@H](c2ccccc2)CNC1=O)NCc1ccc(Cl)cc1] | *** | | |
| ED | 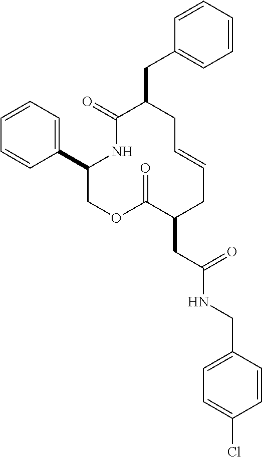 [SMILES: O=C(C[C@@H]1C/C=C/C[C@H](Cc2ccccc2)C(=O)N[C@H](c2ccccc2)COC1=O)NCc1ccc(Cl)cc1] | — | | |
| EE | 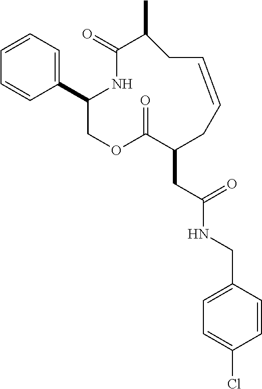 [SMILES: C[C@H]1C/C=C\C[C@@H](CC(=O)NCc2ccc(Cl)cc2)C(=O)OC[C@@H](c2ccccc2)NC1=O] | — | | |
| EF | 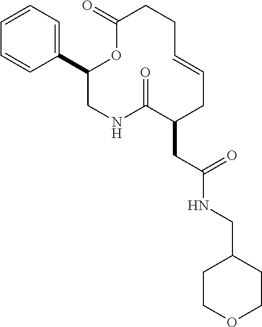 [SMILES: O=C(C[C@@H]1C/C=C/CCC(=O)O[C@H](c2ccccc2)CNC1=O)NCC1CCOCC1] | * | | * |
| EG | 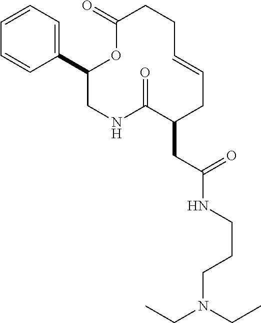 [SMILES: CCN(CC)CCCNC(=O)C[C@@H]1C/C=C/CCC(=O)O[C@H](c2ccccc2)CNC1=O] | — | | * |
| EH | 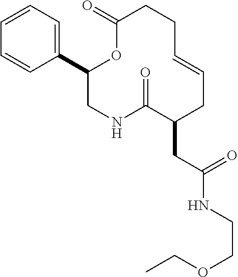 [SMILES: CCOCCNC(=O)C[C@@H]1C/C=C/CCC(=O)O[C@H](c2ccccc2)CNC1=O] | — | | * |
| EI | 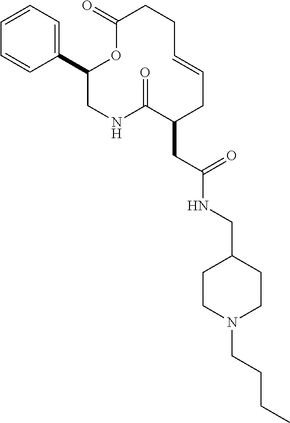 [SMILES: CCCCN1CCC(CNC(=O)C[C@@H]2C/C=C/CCC(=O)O[C@H](c3ccccc3)CNC2=O)CC1] | — | | * |
| EJ | 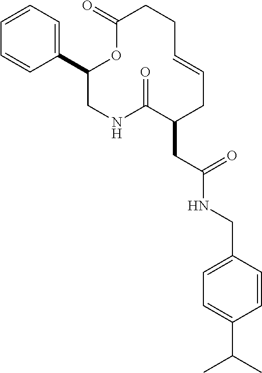 [SMILES: CC(C)c1ccc(CNC(=O)C[C@@H]2C/C=C/CCC(=O)O[C@H](c3ccccc3)CNC2=O)cc1] | * | | |
| EK | 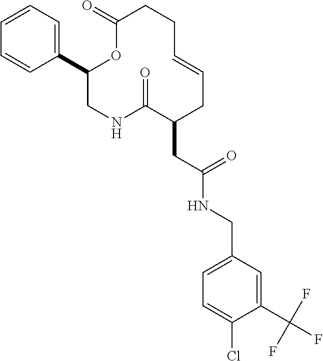 [SMILES: O=C(C[C@@H]1C/C=C/CCC(=O)O[C@H](c2ccccc2)CNC1=O)NCc1ccc(Cl)c(C(F)(F)F)c1] | — | | |
| EL | 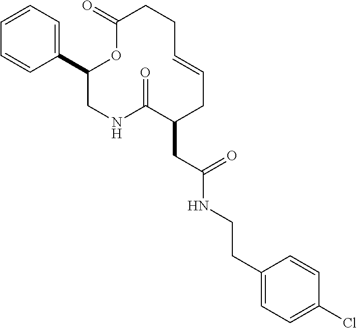 [SMILES: O=C(C[C@@H]1C/C=C/CCC(=O)O[C@H](c2ccccc2)CNC1=O)NCCc1ccc(Cl)cc1] | — | | |
| EM | 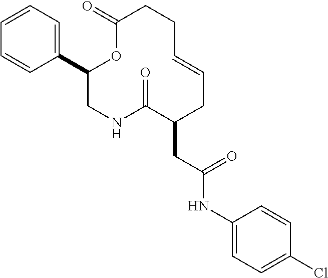 [SMILES: O=C(C[C@@H]1C/C=C/CCC(=O)O[C@H](c2ccccc2)CNC1=O)Nc1ccc(Cl)cc1] | * | | |
| EN | 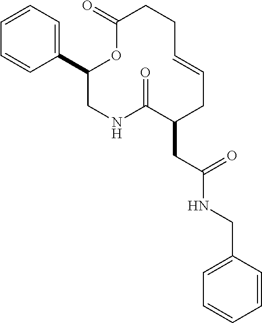 [SMILES: O=C(C[C@@H]1C/C=C/CCC(=O)O[C@H](c2ccccc2)CNC1=O)NCc1ccccc1] | * | | |
| EO | 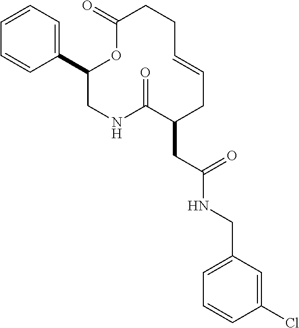 [SMILES: O=C(C[C@@H]1C/C=C/CCC(=O)O[C@H](c2ccccc2)CNC1=O)NCc1cccc(Cl)c1] | — | | |
| EP | 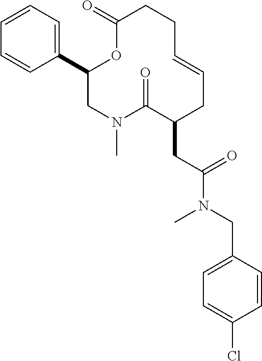 [SMILES: CN(Cc1ccc(Cl)cc1)C(=O)C[C@@H]1C/C=C/CCC(=O)O[C@H](c2ccccc2)CN(C)C1=O] | — | | |
| EQ | 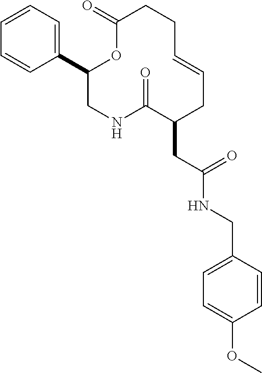 [SMILES: COc1ccc(CNC(=O)C[C@@H]2C/C=C/CCC(=O)O[C@H](c3ccccc3)CNC2=O)cc1] | * | | |
| ER | 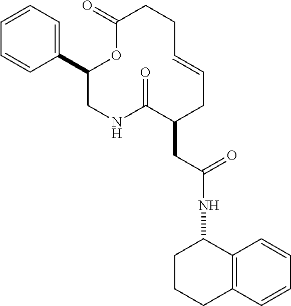 | * | | ** |
| ES | 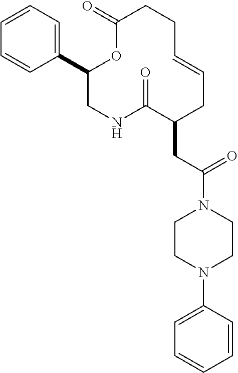 [SMILES: O=C1CC/C=C/C[C@@H](CC(=O)N2CCN(c3ccccc3)CC2)C(=O)NC[C@@H](c2ccccc2)O1] | ** | | ** |
| ET | 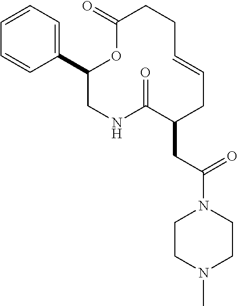 [SMILES: CN1CCN(C(=O)C[C@@H]2C/C=C/CCC(=O)O[C@H](c3ccccc3)CNC2=O)CC1] | * | | |
| EU | 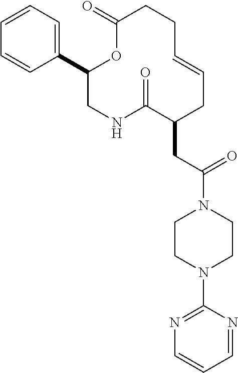 [SMILES: O=C1CC/C=C/C[C@@H](CC(=O)N2CCN(c3ncccn3)CC2)C(=O)NC[C@@H](c2ccccc2)O1] | * | | |
| EV | 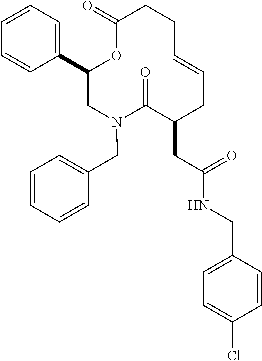 [SMILES: O=C(C[C@@H]1C/C=C/CCC(=O)O[C@H](c2ccccc2)CN(Cc2ccccc2)C1=O)NCc1ccc(Cl)cc1] | — | | |
| EW | 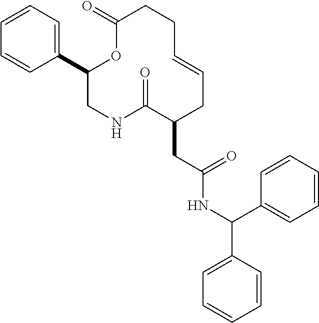 [SMILES: O=C(C[C@@H]1C/C=C/CCC(=O)O[C@H](c2ccccc2)CNC1=O)NC(c1ccccc1)c1ccccc1] | — | | |
| EX | 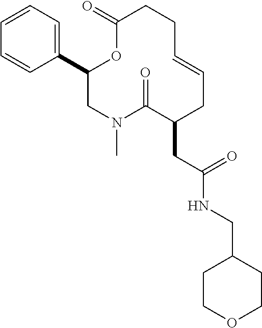 [SMILES: CN1C[C@@H](c2ccccc2)OC(=O)CC/C=C/C[C@@H](CC(=O)NCC2CCOCC2)C1=O] | * | | |
| EY | 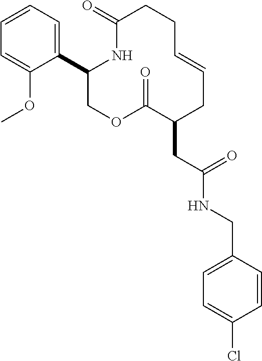 [SMILES: COc1ccccc1[C@@H]1COC(=O)[C@H](CC(=O)NCc2ccc(Cl)cc2)C/C=C/CCC(=O)N1] | — | | |
| EZ | 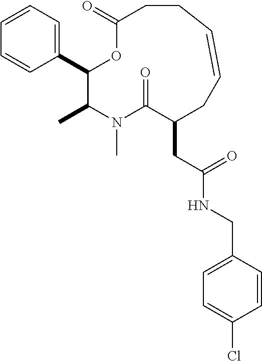 [SMILES: C[C@H]1[C@@H](c2ccccc2)OC(=O)CC/C=C\C[C@@H](CC(=O)NCc2ccc(Cl)cc2)C(=O)N1C] | — | | |
| FA | 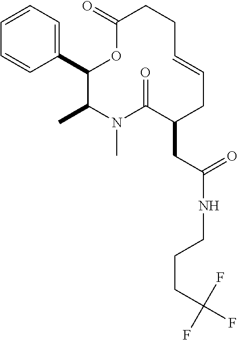 [SMILES: C[C@H]1[C@@H](c2ccccc2)OC(=O)CC/C=C/C[C@@H](CC(=O)NCCCC(F)(F)F)C(=O)N1C] | — | | |
| FB | 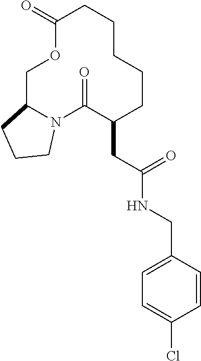 [SMILES: O=C(C[C@@H]1CCCCCC(=O)OC[C@@H]2CCCN2C1=O)NCc1ccc(Cl)cc1] | * | | |
| FC | 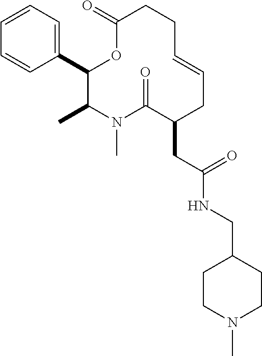 [SMILES: C[C@H]1[C@@H](c2ccccc2)OC(=O)CC/C=C/C[C@@H](CC(=O)NCC2CCN(C)CC2)C(=O)N1C] | | | |
| FF | 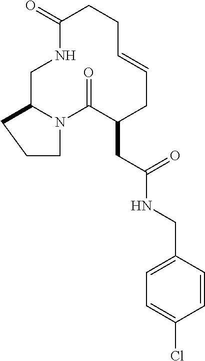 [SMILES: O=C1CC/C=C/C[C@@H](CC(=O)NCc2ccc(Cl)cc2)C(=O)N2CCC[C@H]2CN1] | | | |
| FG | 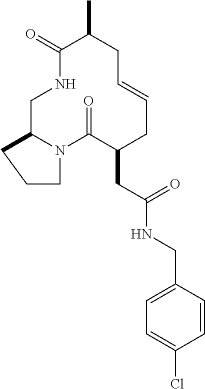 [SMILES: C[C@H]1C/C=C/C[C@@H](CC(=O)NCc2ccc(Cl)cc2)C(=O)N2CCC[C@H]2CNC1=O] | | | |
| FH | 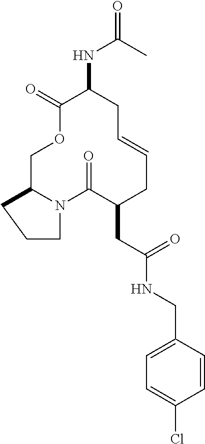 [SMILES: CC(=O)N[C@H]1C/C=C/C[C@@H](CC(=O)NCc2ccc(Cl)cc2)C(=O)N2CCC[C@H]2COC1=O] | | | |
| FI | 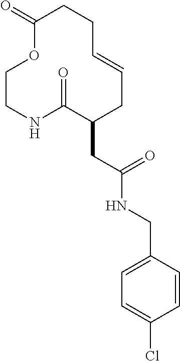 [SMILES: O=C(C[C@@H]1C/C=C/CCC(=O)OCCNC1=O)NCc1ccc(Cl)cc1] | | | |
| FK | 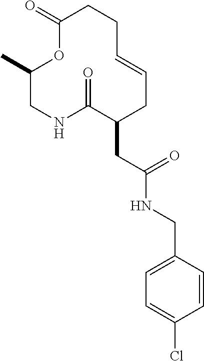 [SMILES: C[C@@H]1CNC(=O)[C@H](CC(=O)NCc2ccc(Cl)cc2)C/C=C/CCC(=O)O1] | | | |
| FM | 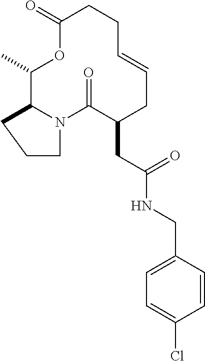 [SMILES: C[C@@H]1OC(=O)CC/C=C/C[C@@H](CC(=O)NCc2ccc(Cl)cc2)C(=O)N2CCC[C@@H]12] | | | |
| FN | 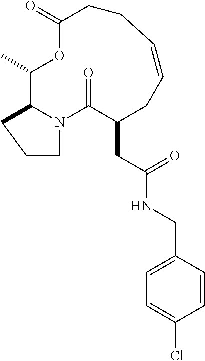 [SMILES: C[C@@H]1OC(=O)CC/C=C\C[C@@H](CC(=O)NCc2ccc(Cl)cc2)C(=O)N2CCC[C@@H]12] | | | |
| FO | 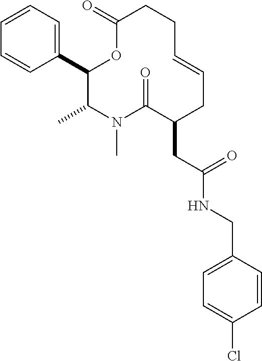 [SMILES: C[C@@H]1[C@@H](c2ccccc2)OC(=O)CC/C=C/C[C@@H](CC(=O)NCc2ccc(Cl)cc2)C(=O)N1C] | | | |
| FP | 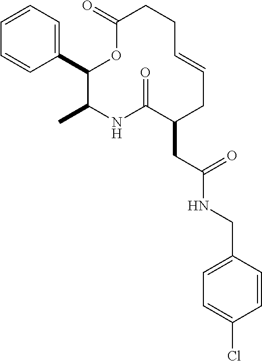 [SMILES: C[C@@H]1NC(=O)[C@H](CC(=O)NCc2ccc(Cl)cc2)C/C=C/CCC(=O)O[C@@H]1c1ccccc1] | — | | |
| FQ | 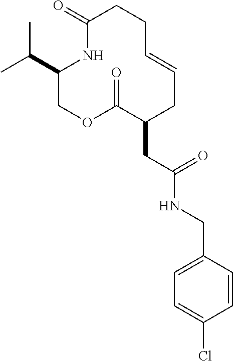 [SMILES: CC(C)[C@@H]1COC(=O)[C@H](CC(=O)NCc2ccc(Cl)cc2)C/C=C/CCC(=O)N1] | ** | | |
| FR | 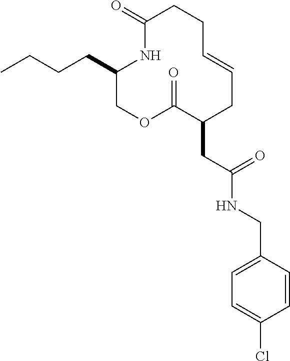 [SMILES: CCCC[C@@H]1COC(=O)[C@H](CC(=O)NCc2ccc(Cl)cc2)C/C=C/CCC(=O)N1] | * | | |
| FS | 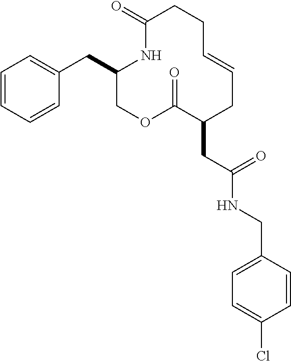 [SMILES: O=C(C[C@@H]1C/C=C/CCC(=O)N[C@H](Cc2ccccc2)COC1=O)NCc1ccc(Cl)cc1] | * | | |
| FT | 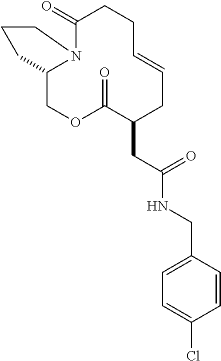 [SMILES: O=C(C[C@@H]1C/C=C/CCC(=O)N2CCC[C@H]2COC1=O)NCc1ccc(Cl)cc1] | * | | |
| FU | 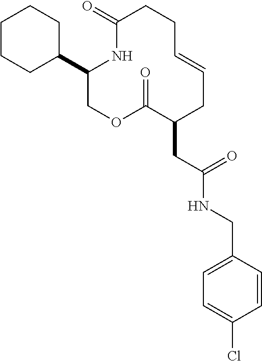 [SMILES: O=C(C[C@@H]1C/C=C/CCC(=O)N[C@H](C2CCCCC2)COC1=O)NCc1ccc(Cl)cc1] | — | | |
| FV | 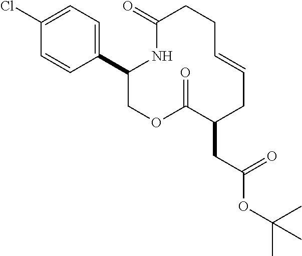 [SMILES: CC(C)(C)OC(=O)C[C@@H]1C/C=C/CCC(=O)N[C@H](c2ccc(Cl)cc2)COC1=O] | *** | | |
| FW | 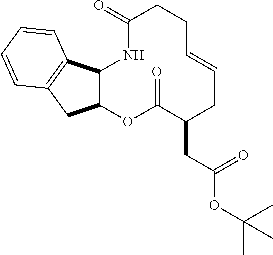 | *** | | |
| FX | 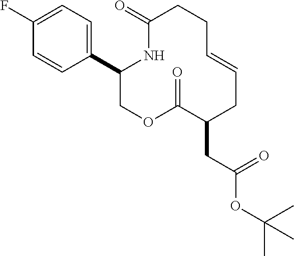 [SMILES: CC(C)(C)OC(=O)C[C@@H]1C/C=C/CCC(=O)N[C@H](c2ccc(F)cc2)COC1=O] | *** | | |
| FY | 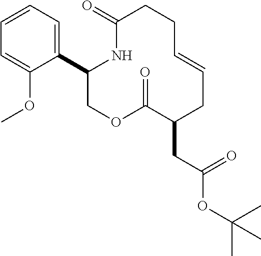 [SMILES: COc1ccccc1[C@@H]1COC(=O)[C@H](CC(=O)OC(C)(C)C)C/C=C/CCC(=O)N1] | *** | | |
| FZ | 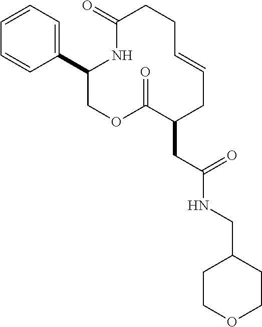 [SMILES: O=C(C[C@@H]1C/C=C/CCC(=O)N[C@H](c2ccccc2)COC1=O)NCC1CCOCC1] | * | | |
| GC | 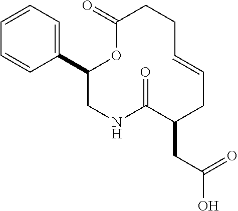 [SMILES: O=C(O)C[C@@H]1C/C=C/CCC(=O)O[C@H](c2ccccc2)CNC1=O] | — | | — |
| GE | 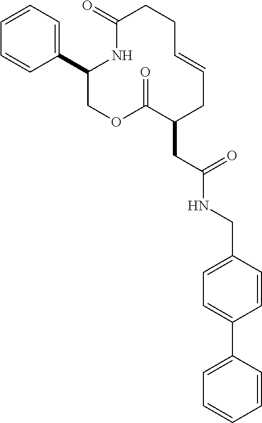 [SMILES: O=C(C[C@@H]1C/C=C/CCC(=O)N[C@H](c2ccccc2)COC1=O)NCc1ccc(-c2ccccc2)cc1] | — | | — |
| GG | 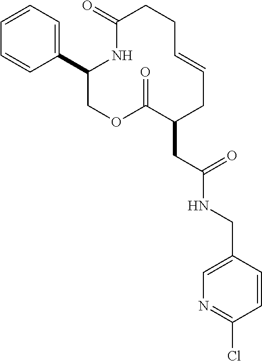 [SMILES: O=C(C[C@@H]1C/C=C/CCC(=O)N[C@H](c2ccccc2)COC1=O)NCc1ccc(Cl)nc1] | — | | |
| GH | 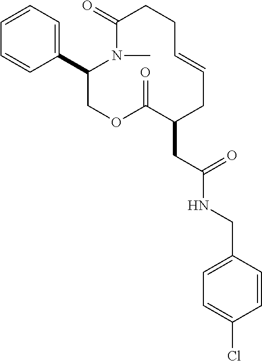 [SMILES: CN1C(=O)CC/C=C/C[C@@H](CC(=O)NCc2ccc(Cl)cc2)C(=O)OC[C@H]1c1ccccc1] | * | | * |
| GI | 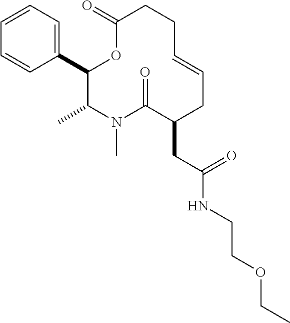 [SMILES: CCOCCNC(=O)C[C@@H]1C/C=C/CCC(=O)O[C@H](c2ccccc2)[C@@H](C)N(C)C1=O] | | | |
| GJ | 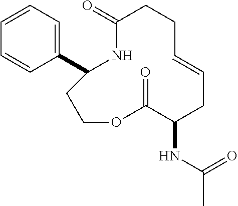 [SMILES: CC(=O)N[C@@H]1C/C=C/CCC(=O)N[C@H](c2ccccc2)CCOC1=O] | | | |
| GK | 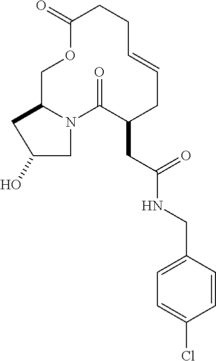 [SMILES: O=C(C[C@@H]1C/C=C/CCC(=O)OC[C@@H]2C[C@@H](O)CN2C1=O)NCc1ccc(Cl)cc1] | | | |
| GM | 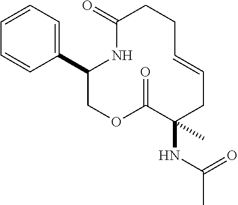 [SMILES: CC(=O)N[C@]1(C)C/C=C/CCC(=O)N[C@H](c2ccccc2)COC1=O] | | | |
| GN | 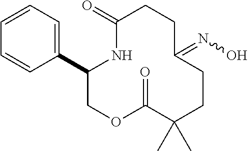 [SMILES: CC1(C)CCC(=NO)CCC(=O)N[C@H](c2ccccc2)COC1=O] | | | |
| GO | 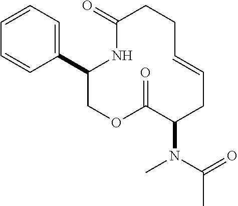 [SMILES: CC(=O)N(C)[C@@H]1C/C=C/CCC(=O)N[C@H](c2ccccc2)COC1=O] | | | |
| GP | 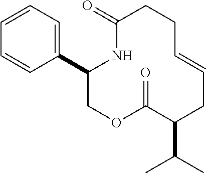 [SMILES: CC(C)[C@@H]1C/C=C/CCC(=O)N[C@H](c2ccccc2)COC1=O] | | | |
| GQ | 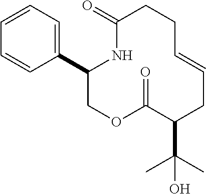 [SMILES: CC(C)(O)[C@@H]1C/C=C/CCC(=O)N[C@H](c2ccccc2)COC1=O] | | | |
| GR | 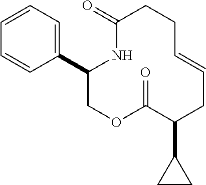 [SMILES: O=C1CC/C=C/C[C@@H](C2CC2)C(=O)OC[C@@H](c2ccccc2)N1] | | | |
| GS | 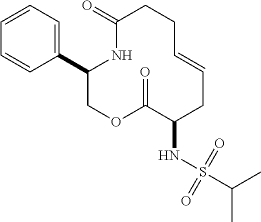 [SMILES: CC(C)S(=O)(=O)N[C@@H]1C/C=C/CCC(=O)N[C@H](c2ccccc2)COC1=O] | | | |
| GT | 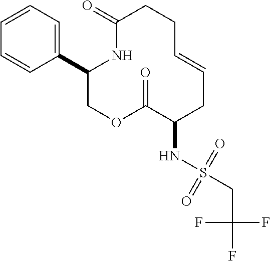 [SMILES: O=C1CC/C=C/C[C@@H](NS(=O)(=O)CC(F)(F)F)C(=O)OC[C@@H](c2ccccc2)N1] | | | |
| GS | 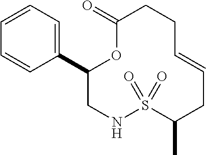 [SMILES: C[C@@H]1C/C=C/CCC(=O)O[C@H](c2ccccc2)CNS1(=O)=O] | | | |
| GT | 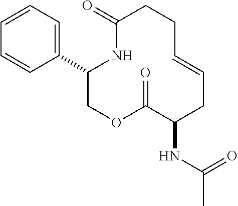 [SMILES: CC(=O)N[C@@H]1C/C=C/CCC(=O)N[C@@H](c2ccccc2)COC1=O] | | | |
| GU | 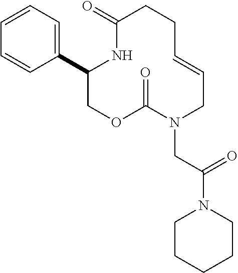 [SMILES: O=C1CC/C=C/CN(CC(=O)N2CCCCC2)C(=O)OC[C@@H](c2ccccc2)N1] | | | |

### EQUIVALENTS

Those skilled in the art will recognize, or be able to ascertain using no more than routine experimentation, numerous equivalents to the specific polypeptides, nucleic acids, methods, assays and reagents described herein. Such equivalents are considered to be within the scope of this invention and are covered by the following claims.

1. A compound of formula X: 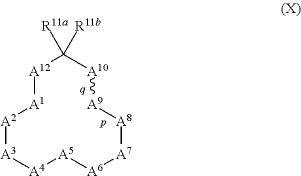
wherein
A¹ is CR^{1a}R^{1b}; O, C═O or NR^{1c};
A² is CR^{2a}R^{2b}, O, NR^{2c} or C═O;
A³ is (CR^{3a}R^{3b})ₐ—(CR^{3c}R^{3d})_{b};
A⁴ is CR^{4a}R^{4b}, O, C═O or NR^{4c};
A⁵ is CR^{5a}R^{5b}, O, C═O or NR^{5c};
A⁶ is CR^{6a}R^{6b}, O, C═O or NR^{6c};
A⁷ is (CR^{7a}R^{7b})ₑ—(CR^{7c}R^{7d})_{f};
A⁸ is CR^{8a}R^{8b}, C═O or C═NOR^{8c};
A⁹ is CR^{9a}R^{9b}, C═O or C═NOR^{9c};
A¹⁰ is (CR^{10a}R^{10b})_{c}—(CR^{10c}R^{10d})_{d} or NR^{10e};
A¹² is CR^{12a}R^{12b}, O, C═O or NR^{12c};
a, b, c, d, e and f are each independently 0 or 1;
p is a single bond when R^{8a} and R^{9a} are present or a double bond when R^{8a} and R^{9a} are absent;
q is a single bond when R^{9a} and R^{10a} are present or a double bond when R^{9a} and R^{10a} are absent; provided that both p and q are not both double bonds;
q is cis or trans to bond p when q is a single bond and p is a double bond;
p is cis or trans to bond q when p is a single bond and q is a double bond;
R^{1a}, R^{1b}, R^{2a}, R^{2b}, R^{4a}, R^{4b}, R^{5a}, R^{5b}, R^{6a}, R^{7a}; R^{7b}, R^{7c}, R^{7d}, R^{8b}, R^{9b}, R^{11a}, R^{11b}, R^{12a} and R^{12b} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R^{6b} is hydrogen, hydroxyl, alkyl alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R^{3a}, R^{3b}, R^{3c}, R^{3d}, R^{8a}, R^{9a}, R^{10a}, R^{10b}, R^{10c} and R^{10d} are each independently absent or hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R^{1c}, R^{4c}, R^{5c}, R^{6c}, R^{8c}, R^{9c}, R^{10e} and R^{12c} are each independently hydrogen, hydroxy, alkoxy, alkyl, alkenyl, alkynyl, aryl, carbonyl, carboxy, acyl or amino; or
R^{1c} and R^{2a}, or R^{2a} and R^{3a}, or R^{3a} and R^{4c}, or R^{8b} and R^{9b}, or R^{8b} and R^{10e}, together with the atoms to which they are attached, are linked to form a 3 to 10-membered carbocyclic, heterocyclic or aryl ring and pharmaceutically acceptable salt thereof;
provided that said alkyl is not substituted with C═XR^{6d}, wherein X is O, NR^{6e} or S; and R^{6d} is NR^{6′}R^{6″}, OR^{6′} or SR^{6′}; wherein R^{6e}, R^{6′} and R^{6″} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether.

2-169. (canceled)

170. The compound of claim 1, wherein the compound of formula X is a compound of formula Xa: 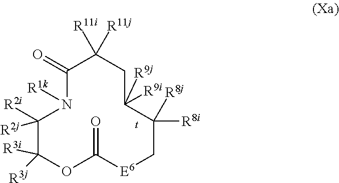
wherein
E⁶ is CR⁶ⁱR^{6j};
R^{1k} is hydrogen or alkyl;
R²ⁱ, R^{2j}, R³ⁱ, R^{3j}, R⁶ⁱ, R¹¹ⁱ and R^{11k} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;
R^{6j} is hydrogen, hydroxyl, C₁-C₁₂ unsubstituted alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;
R^{2j} and R^{3j}, together with the atoms to which they are attached, are linked to form a 3 to 10-membered carbocyclic, heterocyclic or aryl ring;
t is a single or double bond;
R⁸ⁱ and R⁹ⁱ are absent when t is a double bond or hydrogen when t is a single bond;
R^{8j} is hydrogen, hydroxyl, alkyl or amino;
R^{9j} is hydrogen, hydroxyl, or alkyl; or
R^{8j} and R^{9j}, together with the atoms to which they are attached, are linked to form a 3 to 10-membered carbocyclic, heterocyclic or aryl ring; or a pharmaceutically acceptable salt thereof.

171-177. (canceled)

178. The compound of claim 1, wherein the compound of formula X is a compound of formula Xb: 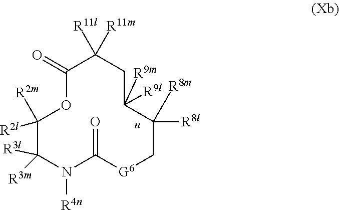
wherein
G⁶ is CR^{6l}R^{6m};
u is a single or double bond;
R^{2l}, R^{2m}, R^{3l}, R^{3m} R^{6l}, R^{8m}, R^{9m}, R^{11l} and R^{11m} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;
R^{6m} is hydrogen, hydroxyl, C₁-C₁₂ unsubstituted alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;
R^{8l} and R^{9l} are absent when u is a double bond or hydrogen when u is a single bond;
R⁴ⁿ is hydrogen or alkyl; or R^{3m} and R⁴ⁿ, together with the atoms to which they are attached are linked to form a 3 to 10-membered heterocyclic or aryl ring, or a pharmaceutically acceptable salt thereof.

179-181. (canceled)

182. The compound of claim 1, wherein the compound of formula X is a compound of Xc: 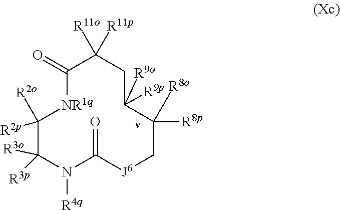
wherein
J⁶ is CR^{6o}R^{6p};
v is a single or double bond;
R^{2o}, R^{2p}, R^{3o}, R^{3p}, R^{6o}, R^{8p}, R^{9p}, R^{11o} and R^{11p} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;
R^{6p} is hydrogen, hydroxyl, C₁-C₁₂ unsubstituted alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether;
R^{8o} and R^{9o} are absent when u is a double bond or hydrogen when u is a single bond;
R^{1q} and R^{4q} are each independently hydrogen or alkyl; or R^{3p} and R^{4q}, together with the atoms to which they are attached are linked to form a 3 to 10-membered heterocyclic or aryl ring, or a pharmaceutically acceptable salt thereof.

183-185. (canceled)

186. The compound of claim 1, wherein the compound of formula X is a compound of formula Xd: 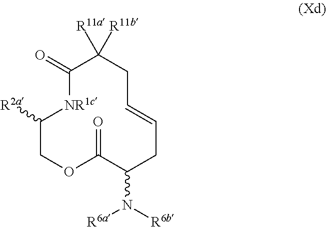
wherein
R^{1c′} is hydrogen or alkyl;
R^{2a′}, R^{11a′} and R^{11b′} are each independently hydrogen, hydroxyl, alkyl, alkenyl, alkynyl, aryl, amino, sulfonyl, carbonyl, carboxy, alkoxy, aryloxy, halogen, acyl, oximyl, azinyl, hydrazinyl, —NO₂, —CN, a heterocyclic moiety or thioether; and
R^{6a′} and R^{6b′} are each independently hydrogen, hydroxy, alkoxy, alkyl, alkenyl, alkynyl, aryl, carbonyl, carboxy, acyl or amino, or R^{6a′} and R^{6b′} together with the nitrogen to which they are attached are linked to form a 3 to 10-membered heterocyclic or aryl ring; and pharmaceutically acceptable salts thereof.

187-192. (canceled)

193. A compound of formula XI: 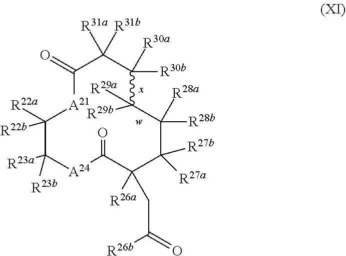
wherein
A²¹ is O or NR^{21c};
A²⁴ is O or NR^{24c};
w is a single bond when R^{28a} and R^{29a} are present or a double bond when R^{28a} and R^{29a} are absent;
x is cis or trans to w when w is a double bond;
R^{22a}, R^{22b}, R^{23a}, R^{23b}, R^{26a}, R^{27a}, R^{27b}, R^{28b}, R^{29b}, R^{30a}; R^{30b}, R^{31b} and R^{31b} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
R^{26b} is NR^{26c}R^{26d} or OR^{26e};
R^{28a} and R^{29a} are each independently absent when w is a double bond or hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether when w is a single bond;
R^{21c}, R^{24c}, R^{26c} and R^{26d} are each independently hydrogen, hydroxy, alkoxy, alkyl, alkenyl, alkynyl, aryl, carbonyl, carboxy, acyl, amino or a carbocyclic or heterocyclic moiety, or R^{26c} and R^{26d} are linked to form a 3 to 10-membered heterocyclic or aryl ring;
R^{26e} is hydrogen, alkyl, alkenyl, alkynyl, aryl, amino, carbonyl or a heterocyclic moiety; or
R^{21c} and R^{22a}, or R^{22a} and R^{23a}, or R^{23a} and R^{24c}, or R^{28b} and R^{29b}, together with the atoms to which they are attached, are linked to form a 3 to 10-membered carbocyclic, heterocyclic or aryl ring; and pharmaceutically acceptable salts thereof;
provided that when w is a double bond, then R^{21c} and R^{22a} or R^{22a} and R^{23a}, together with the atoms to which they are attached, are linked to form a 3 to 10-membered carbocyclic, heterocyclic or aryl ring; or
provided that when w is a double bond, then x is cis to w; or
provided that when w is a single bond, one of R^{28a} or R^{29a} is not hydrogen.

194-223. (canceled)

224. The compound of claim 193, wherein the compound of formula XI is a compound of formula XIa: 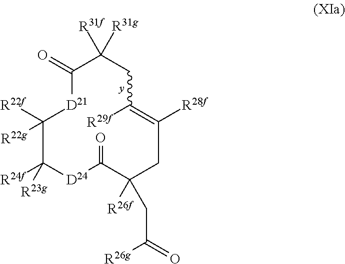
wherein
D²¹ is O or NR^{21h};
D²⁴ is O or NR^{24h};
R^{22f}, R^{23f}, R^{26f}, R^{28f}, R^{29f}, R^{31f} and R^{31g} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether;
y is cis or trans;
R^{22g} and R^{23g} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether when y is cis; or
R^{22g} and R^{23g} together with the atoms to which they are attached are linked to form a 3 to 10-membered carbocyclic, heterocyclic or aryl ring when y is cis or trans;
R^{21h} and R^{24h} are each independently hydrogen or alkyl;
R^{26g} is NR²⁶ⁱR^{26j} or OR^{26k};
R²⁶ⁱ and R^{26j} are each independently hydrogen, hydroxy, alkoxy, alkyl, alkenyl, alkynyl, aryl, carbonyl, carboxy, acyl, amino or a carbocyclic or heterocyclic moiety, or R²⁶ⁱ and R^{26j} are linked to form a 3-10 membered heterocyclic ring; and
R^{26k} is hydrogen, alkyl, alkenyl, alkynyl, aryl, amino, carbonyl or a heterocyclic moiety; or a pharmaceutically acceptable salt thereof.

225-226. (canceled)

227. The compound of claim 193, wherein the compound of formula XI is a compound of formula XIb: 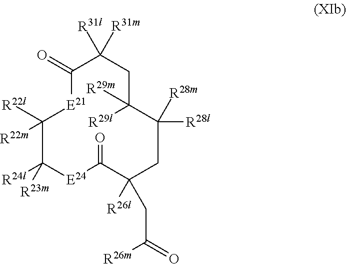
wherein
E²¹ is O or NR²¹ⁿ;
E²⁴ is O or NR²⁴ⁿ;
R^{22l}, R^{22m}, R^{23l}, R^{23m}, R^{26l}, R^{28l}, R^{28m}, R^{29l}, R^{29m}, R^{31l} and R^{31m} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether, provided that one of R^{28l} or R^{29l} are not hydrogen; or R^{28l} and R^{29l}, together with the atoms to which they are attached, are linked to form a 3 to 10-membered carbocyclic, heterocyclic or aromatic ring;
R²¹ⁿ and R²⁴ⁿ are each independently hydrogen or alkyl;
R^{26m} is NR^{26o}R^{26p} or OR^{26q};
R^{26o} and R^{26p} are each independently hydrogen, hydroxy, alkoxy, alkyl, alkenyl, alkynyl, aryl, carbonyl, carboxy, acyl, amino or a carbocyclic or heterocyclic moiety, or R^{26o} and R^{26p} together with the atoms to which they are attached are linked to form a 3 to 10 membered heterocyclic or aryl ring; and
R^{26q} is hydrogen, alkyl, alkenyl, alkynyl, aryl, amino, carbonyl or a heterocyclic moiety; or a pharmaceutically acceptable salt thereof.

228-233. (canceled)

234. A compound of formula XII: 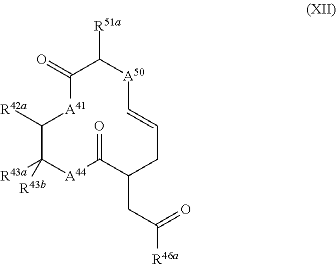
wherein
A⁴¹ is O or NR^{41c};
A⁴⁴ is O or NR^{44c};
R^{42a}, R^{43a}, R^{43b}, R^{46e} and R^{51a} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether; or
R^{43b} and R^{44c}, together with the atoms to which they are attached, are linked to form a 3-10-membered heterocyclic or aryl ring;
R^{46a} is NR^{46b}R^{46c} or OR^{46d};
R^{41c}, R^{44c}, R^{46b}, R^{46c} are each independently hydrogen, hydroxy, alkoxy, alkyl, alkenyl, alkynyl, aryl, carbonyl, carboxy, acyl, amino or CH₂R^{46e}; or R^{46b} and R^{46c} are linked to form a 3 to 10 membered heterocyclic or aryl ring;
R^{46d} is hydrogen, alkyl, alkenyl, alkynyl, aryl, amino, carbonyl or a heterocyclic moiety;
A⁵⁰ is (CH₂)ₑ—(CH₂)_{f}; and
e and f are each independently 0 or 1; and pharmaceutically acceptable salts thereof,
provided that when A⁴¹ is O; R^{42a} is unsubstituted phenyl; A⁴⁴ is NR^{44c}; e is 1; f is 0; R^{46a} is NR^{46b}R^{46c}; R^{43a}, R^{43b}, R^{44c}, R^{46b} and R^{51a} are each hydrogen and R^{46c} is CH₂R^{46e}, then R^{46e} is not 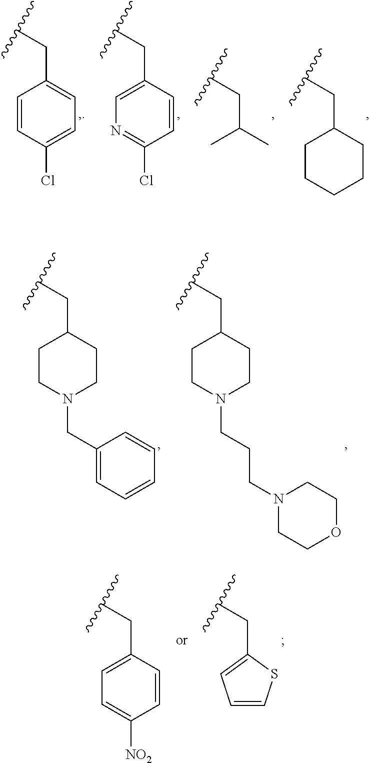
provided that when A⁴¹ is O; R^{42a} is unsubstituted phenyl; A⁴⁴ is NR^{44c}; R^{44c} is hydrogen or methyl; e is 1; f is 0; R^{46a} is OR^{46d}; R^{43a}, R^{43b} and R^{51a} are each hydrogen, then R^{46d} is not 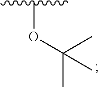 [SMILES: *OC(C)(C)C]
provided that when A⁴¹ is O; R^{42a} is unsubstituted phenyl; A⁴⁴ is NR⁴⁴; R^{44c} is methyl; e is 1; f is 0; R^{46a} is NR^{46b}R^{46c}; R^{43a}, R^{43b}, R^{46b} and R^{51a} are each hydrogen and R^{46c} is CH₂R^{46e}, then R^{46e} is not 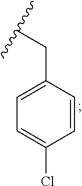 [SMILES: *Cc1ccc(Cl)cc1]
provided that when A⁴¹ is NR^{41c}; R^{41c} is hydrogen; R^{42a} is unsubstituted phenyl; A⁴⁴ is O; e is 1; f is 0; R^{46a} is OR^{46d}; R^{43a}, R^{43b} and R^{5a} are each hydrogen, then R^{46d} is not 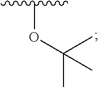 [SMILES: *OC(C)(C)C]
and
provided that when A⁴¹ is NR^{41c}; R^{41c} is hydrogen; R^{42a} is unsubstituted phenyl; A⁴⁴ is O; e is 1; f is 0; R^{46a} is NR^{46b}R^{46c}; R^{43a}, R^{43b}, R^{46b} and R^{51a} are each hydrogen and R^{46c} is CH₂R^{46e}, then R^{46e} is not

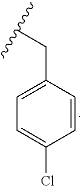 [SMILES: *Cc1ccc(Cl)cc1]

235-249. (canceled)

250. The compound of claim 234, wherein the compound is formula XII is a compound of formula XIIa: 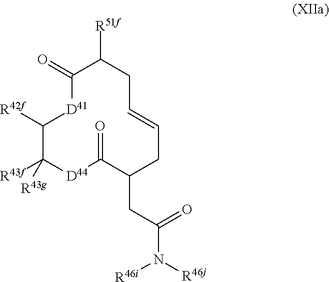
wherein
D⁴¹ is O or NR^{41h};
D⁴⁴ is O or NR^{44h};
R^{42f}, R^{43f}, R^{43g} and R^{51f} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether; or
R^{43h} and R^{44h} are linked to form a 3 to 10-membered heterocyclic or aryl ring;
R^{41h} and R^{44h} are each independently hydrogen or alkyl; and
R⁴⁶ⁱ and R^{46j} are each independently hydrogen, hydroxy, alkoxy, alkyl, alkenyl, alkynyl, aryl, carbonyl, carboxy, acyl, amino or a carbocyclic or heterocyclic moiety, or R⁴⁶ⁱ and R^{46j} are linked to form a 3 to 10-membered heterocyclic or aryl ring; and pharmaceutically acceptable salts thereof,
provided that when D⁴¹ is O; R^{42f} is unsubstituted phenyl; D⁴⁴ is NR^{44h}; R^{43f}, R^{43g}, R^{44h}, R⁴⁶ⁱ and R^{51f} are each hydrogen, then R^{46j} is not 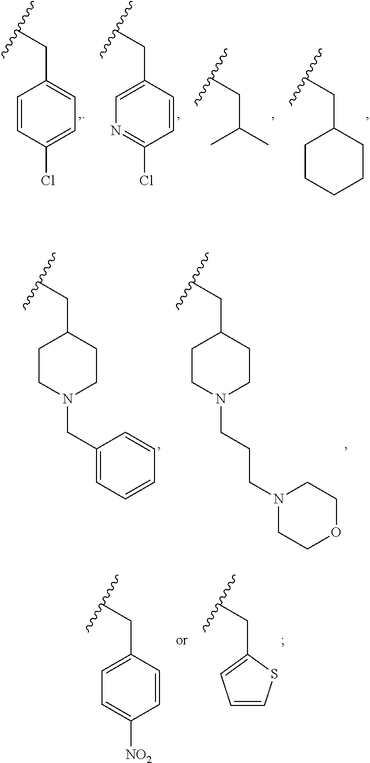
provided that when D⁴¹ is O; R^{42f} is unsubstituted phenyl; D⁴⁴ is NR^{44h}; R^{44h} is methyl; R^{43f}, R^{43g}, R⁴⁶ⁱ and R^{51f} are each hydrogen, then R^{46j} is not 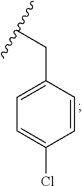 [SMILES: *Cc1ccc(Cl)cc1]
and
provided that when D⁴¹ is NR^{41h}; R^{41h} is hydrogen; R^{42f} is unsubstituted phenyl; D⁴⁴ is O; R^{43f}, R^{43g}, R⁴⁶ⁱ and R^{51f} are each hydrogen, then R^{46j} is not

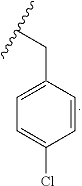 [SMILES: *Cc1ccc(Cl)cc1]

251-258. (canceled)

259. The compound of claim 234, wherein the compound of formula XII is a compound of formula XIIb: 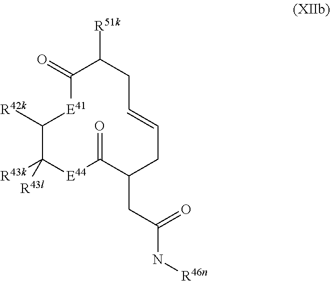
wherein
E⁴¹ is O or NR^{41m};
E⁴⁴ is O or NR^{44m};
R^{42k}, R^{43k}, R^{43l} and R^{51k} are each independently hydrogen, hydroxyl, alkyl, alkenyl, alkynyl, aryl, amino, sulfonyl, carbonyl, carboxy, alkoxy, aryloxy, halogen, acyl, oximyl, hydrazinyl, —NO₂, —CN, a heterocyclic moiety or thioether; or
R^{42k} and R^{43k} are linked to form a 3- to 10-membered carbocyclic, heterocyclic or aryl ring;
R⁴⁶ⁿ is hydrogen, alkyl, alkenyl, alkynyl, aryl, amino, carbonyl or a heterocyclic moiety; or a pharmaceutically acceptable salt thereof;
provided that when E⁴¹ is O; R^{42k} is unsubstituted phenyl; E⁴⁴ is NR^{44m}; R^{44m} is hydrogen or methyl and R^{51k} is hydrogen, then R⁴⁶ⁿ is not 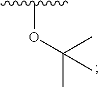 [SMILES: *OC(C)(C)C]
and
provided that when E⁴¹ is NR^{41m}; R^{41m} is hydrogen; R^{42k} is unsubstituted phenyl; E⁴⁴ is O and R^{51k} is hydrogen, then R⁴⁶ⁿ is not

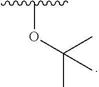 [SMILES: *OC(C)(C)C]

260-269. (canceled)

270. A pharmaceutical composition comprising a compound of claim 1, and a pharmaceutically acceptable carrier.

271. A method of treating a Sonic Hedgehog associated disorder in a subject comprising administering to said subject an effective amount of a compound of claim 1, or a pharmaceutically acceptable salt thereof, such that said subject is treated for said disorder.

272-277. (canceled)

278. A method of inhibiting Sonic Hedgehog protein induced transcription in cells by contacting the cells with an effective amount of a compound of claim 1, such that said transcription is inhibited.

279. (canceled)

280. A pharmaceutical composition comprising a compound of claim 193, and a pharmaceutically acceptable carrier.

281. A method of treating a Sonic Hedgehog associated disorder in a subject comprising administering to said subject an effective amount of a compound of claim 193, or a pharmaceutically acceptable salt thereof, such that said subject is treated for said disorder.

282. A method of inhibiting Sonic Hedgehog protein induced transcription in cells by contacting the cells with an effective amount of a compound of claim 193, such that said transcription is inhibited.

283. A pharmaceutical composition comprising a compound of claim 234, and a pharmaceutically acceptable carrier.

284. A method of treating a Sonic Hedgehog associated disorder in a subject comprising administering to said subject an effective amount of a compound of claim 234, or a pharmaceutically acceptable salt thereof, such that said subject is treated for said disorder.

285. A method of inhibiting Sonic Hedgehog protein induced transcription in cells by contacting the cells with an effective amount of a compound of claim 234, such that said transcription is inhibited.
